SEQUENCE LISTING (4 sequences)

SEQ ID NO: 1 - DNA, 2256 bp, Homo sapiens
  source 1..2256: mol_type = other DNA
atggccaaggagaggggcctaataagccccagtgattttgcccagctgcaaaaatacatggaatactccaccaaaaaggtcagtgatgtcctaaagctcttcgaggatggcgagatggctaaatatgtccaaggagatgccattgggtacgagggattccagcaattcctgaaaatctatctcgaagtggataatgttcccagacacctaagcctggcactgtttcaatcctttgagactggtcactgcttaaatgagacaaatgtgacaaaagatgtggtgtgtctcaatgatgtttcctgctacttttcccttctggagggtggtcggccagaagacaagttagaattcaccttcaagctgtacgacacggacagaaatgggatcctggacagctcagaagtggacaaaattatcctacagatgatgcgagtggctgaatacctggattgggatgtgtctgagctgaggccgattcttcaggagatgatgaaagagattgactatgatggcagtggctctgtctctcaagctgagtgggtccgggctggggccaccaccgtgccactgctagtgctgctgggtctggagatgactctgaaggacgacggacagcacatgtggaggcccaagaggttccccagaccagtctactgcaatctgtgcgagtcaagcattggtcttggcaaacagggactgagctgtaacctctgtaagtacactgttcacgaccagtgtgccatgaaagccctgccttgtgaagtcagcacctatgccaagtctcggaaggacattggtgtccaatcacatgtgtgggtgcgaggaggctgtgagtccgggcgctgcgaccgctgtcagaaaaagatccggatctaccacagtctgaccgggctgcattgtgtatggtgccacctagagatccacgatgactgcctgcaagcggtgggccatgagtgtgactgtgggctgctccgggatcacatcctgcctccatcttccatctatcccagtgtcctggcctctggaccggatcgtaaaaatagcaaaacaagccagaagaccatggatgatttaaatttgagcacctctgaggctctgcggattgaccctgttcctaacacccacccacttctcgtctttgtcaatcctaagagtggcgggaagcaggggcagagggtgctctggaagttccagtatatattaaaccctcgacaggtgttcaacctcctaaaggatggtcctgagatagggctccgattattcaaggatgttcctgatagccggattttggtgtgtggtggagacggcacagtaggctggattctagagaccattgacaaagctaacttgccagttttgcctcctgttgctgtgttgcccctgggtactggaaatgatctggctcgatgcctaagatggggaggaggttatgaaggacagaatctggcaaagatcctcaaggatttagagatgagtaaagtggtacatatggatcgatggtctgtggaggtgatacctcaacaaactgaagaaaaaagtgacccagtcccctttcaaatcatcaataactacttctctattggcgtggatgcctctattgctcatcgattccacatcatgcgagagaaatatccggagaagttcaacagcagaatgaagaacaagctatggtacttcgaatttgccacatctgaatccatcttctcaacatgcaaaaagctggaggagtctttgacagttgagatctgtgggaaaccgctggatctgagcaacctgtccctagaaggcatcgcagtgctaaacatccctagcatgcatggtggctccaacctctggggtgataccaggagaccccatggggatatctatgggatcaaccaggccttaggtgctacagctaaagtcatcaccgaccctgatatcctgaaaacctgtgtaccagacctaagtgacaagagactggaagtggttgggctggagggtgcaattgagatgggccaaatctataccaagctcaagaatgctggacgtcggctggccaagtgctctgagatcaccttccacaccacaaaaacccttcccatgcaaattgacggagaaccctggatgcagacgccctgtacaatcaagatcacccacaagaaccagatgcccatgctcatgggcccacccccccgctccaccaatttctttggcttcttgagcggatcctcggagacagtgcggtttcagggacaccaccaccatcaccactga

SEQ ID NO: 2 - protein, 737 aa, Homo sapiens
MASPSDFAQLQKYMEYSTKKVSDVLKLFEDGEMAKYVQGDAIGYEGFQQFLKIYLEVDNVPRHLSLALFQSFETGHCLNETNVTKDVVCLNDVSCYFSLLEGGRPEDKLEFTFKLYDTDRNGILDSSEVDKIILQMMRVAEYLDWDVSELRPILQEMMKEIDYDGSGSVSQAEWVRAGATTVPLLVLLGLEMTLKDDGQHMWRPKRFPRPVYCNLCESSIGLGKQGLSCNLCKYTVHDQCAMKALPCEVSTYAKSRKDIGVQSHVWVRGGCESGRCDRCQKKIRIYHSLTGLHCVWCHLEIHDDCLQAVGHECDCGLLRDHILPPSSIYPSVLASGPDRKNSKTSQKTMDDLNLSTSEALRIDPVPNTHPLLVFVNPKSGGKQGQRVLWKFQYILNPRQVFNLLKDGPEIGLRLFKDVPDSRILVCGGDGTVGWILETIDKANLPVLPPVAVLPLGTGNDLARCLRWGGGYEGQNLAKILKDLEMSKVVHMDRWSVEVIPQQTEEKSDPVPFQIINNYFSIGVDASIAHRFHIMREKYPEKFNSRMKNKLWYFEFATSESIFSTCKKLEESLTVEICGKPLDLSNLSLEGIAVLNIPSMHGGSNLWGDTRRPHGDIYGINQALGATAKVITDPDILKTCVPDLSDKRLEVVGLEGAIEMGQIYTKLKNAGRRLAKCSEITFHTTKTLPMQIDGEPWMQTPCTIKITHKNQMPMLMGPPPRSGSSETVRFQGHHHHHH

SEQ ID NO: 3 - protein, 2838 aa, Homo sapiens
ATGGAGCCGCGGGACGGTAGCCCCGAGGCCCGGAGCAGCGACTCCGAGTCGGCTTCCGCCTCGTCCAGCGGCTCCGAGCGCGACGCCGGTCCCGAGCCGGACAAGGCGCCGCGGCGACTCAACAAGCGGCGCTTCCCGGGGCTGCGGCTCTTCGGGCACAGGAAAGCCATCACGAAGTCGGGCCTCCAGCACCTGGCCCCCCCTCCGCCCACCCCTGGGGCCCCGTGCAGCGAGTCAGAGCGGCAGATCCGGAGTACAGTGGACTGGAGCGAGTCAGCGACATATGGGGAGCACATCTGGTTCGAGACCAACGTGTCCGGGGACTTCTGCTACGTTGGGGAGCAGTACTGTGTAGCCAGGATGCTGCAGAAGTCAGTGTCTCGAAGAAAGTGCGCAGCCTGCAAGATTGTGGTGCACACGCCCTGCATCGAGCAGCTGGAGAAGATAAATTTCCGCTGTAAGCCGTCCTTCCGTGAATCAGGCTCCAGGAATGTCCGCGAGCCAACCTTTGTACGGCACCACTGGGTACACAGACGACGCCAGGACGGCAAGTGTCGGCACTGTGGGAAGGGATTCCAGCAGAAGTTCACCTTCCACAGCAAGGAGATTGTGGCCATCAGCTGCTCGTGGTGCAAGCAGGCATACCACAGCAAGGTGTCCTGCTTCATGCTGCAGCAGATCGAGGAGCCGTGCTCGCTGGGGGTCCACGCAGCCGTGGTCATCCCGCCCACCTGGATCCTCCGCGCCCGGAGGCCCCAGAATACTCTGAAAGCAAGCAAGAAGAAGAAGAGGGCATCCTTCAAGAGGAAGTCCAGCAAGAAAGGGCCTGAGGAGGGCCGCTGGAGACCCTTCATCATCAGGCCCACCCCCTCCCCGCTCATGAAGCCCCTGCTGGTGTTTGTGAACCCCAAGAGTGGGGGCAACCAGGGTGCAAAGATCATCCAGTCTTTCCTCTGGTATCTCAATCCCCGACAAGTCTTCGACCTGAGCCAGGGAGGGCCCAAGGAGGCGCTGGAGATGTACCGCAAAGTGCACAACCTGCGGATCCTGGCGTGCGGGGGCGACGGCACGGTGGGCTGGATCCTCTCCACCCTGGACCAGCTACGCCTGAAGCCGCCACCCCCTGTTGCCATCCTGCCCCTGGGTACTGGCAACGACTTGGCCCGAACCCTCAACTGGGGTGGGGGCTACACAGATGAGCCTGTGTCCAAGATCCTCTCCCACGTGGAGGAGGGGAACGTGGTACAGCTGGACCGCTGGGACCTCCACGCTGAGCCCAACCCCGAGGCAGGGCCTGAGGACCGAGATGAAGGCGCCACCGACCGGTTGCCCCTGGATGTCTTCAACAACTACTTCAGCCTGGGCTTTGACGCCCACGTCACCCTGGAGTTCCACGAGTCTCGAGAGGCCAACCCAGAGAAATTCAACAGCCGCTTTCGGAATAAGATGTTCTACGCCGGGACAGCTTTCTCTGACTTCCTGATGGGCAGCTCCAAGGACCTGGCCAAGCACATCCGAGTGGTGTGTGATGGAATGGACTTGACTCCCAAGATCCAGGACCTGAAACCCCAGTGTGTTGTTTTCCTGAACATCCCCAGGTACTGTGCGGGCACCATGCCCTGGGGCCACCCTGGGGAGCACCACGACTTTGAGCCCCAGCGGCATGACGACGGCTACCTCGAGGTCATTGGCTTCACCATGACGTCGTTGGCCGCGCTGCAGGTGGGCGGACACGGCGAGCGGCTGACGCAGTGTCGCGAGGTGGTGCTCACCACATCCAAGGCCATCCCGGTGCAGGTGGATGGCGAGCCCTGCAAGCTTGCAGCCTCACGCATCCGCATCGCCCTGCGCAACCAGGCCACCATGGTGCAGAAGGCCAAGCGGCGGAGCGCCGCCCCCCTGCACAGCGACCAGCAGCCGGTGCCAGAGCAGTTGCGCATCCAGGTGAGTCGCGTCAGCATGCACGACTATGAGGCCCTGCACTACGACAAGGAGCAGCTCAAGGAGGCCTCTGTGCCGCTGGGCACTGTGGTGGTCCCAGGAGACAGTGACCTAGAGCTCTGCCGTGCCCACATTGAGAGACTCCAGCAGGAGCCCGATGGTGCTGGAGCCAAGTCCCCGACATGCCAGAAACTGTCCCCCAAGTGGTGCTTCCTGGACGCCACCACTGCCAGCCGCTTCTACAGGATCGACCGAGCCCAGGAGCACCTCAACTATGTGACTGAGATCGCACAGGATGAGATTTATATCCTGGACCCTGAGCTGCTGGGGGCATCGGCCCGGCCTGACCTCCCAACCCCCACTTCCCCTCTCCCCACCTCACCCTGCTCACCCACGCCCCGGTCACTGCAAGGGGATGCTGCACCCCCTCAAGGTGAAGAGCTGATTGAGGCTGCCAAGAGGAACGACTTCTGTAAGCTCCAGGAGCTGCACCGAGCTGGGGGCGACCTCATGCACCGAGACGAGCAGAGTCGCACGCTCCTGCACCACGCAGTCAGCACTGGCAGCAAGGATGTGGTCCGCTACCTGCTGGACCACGCCCCCCCAGAGATCCTTGATGCGGTGGAGGAAAACGGGGAGACCTGTTTGCACCAAGCAGCGGCCCTGGGCCAGCGCACCATCTGCCACTACATCGTGGAGGCCGGGGCCTCGCTCATGAAGACAGACCAGCAGGGCGACACTCCCCGGCAGCGGGCTGAGAAGGCTCAGGACACCGAGCTGGCCGCCTACCTGGAGAACCGGCAGCACTACCAGATGATCCAGCGGGAGGACCAGGAGACGGCTGTGGGATCCTCGGAGACAGTGCGGTTTCAGGGACACCACCACCATCACCACTGA

SEQ ID NO: 4 - protein, 945 aa, Homo sapiens
MEPRDGSPEARSSDSESASASSSGSERDAGPEPDKAPRRLNKRRFPGLRLFGHRKAITKSGLQHLAPPPPTPGAPCSESERQIRSTVDWSESATYGEHIWFETNVSGDFCYVGEQYCVARMLQKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAVGSSETVRFQGHHHHHH

(19) United States
(12) Patent Application Publication
(10) Pub. No.: US 2024/0400563 A1
(43) Pub. Date: Dec. 5, 2024

(54) SUBSTITUTED PYRIDOPYRIMIDINONYL COMPOUNDS USEFUL AS T CELL ACTIVATORS

(71) Applicant: BRISTOL-MYERS SQUIBB COMPANY, Princeton, NJ (US)

(72) Inventors: Upender Velaparthi, Princeton Junction, NJ (US); Richard E. Olson, Cambridge, MA (US); Chetan Padmakar Darne, Ewing, NJ (US); Bireshwar Dasgupta, Doylestown, PA (US); Jayakumar Sankara Warrier, Bangalore (IN); Hasibur Rahaman, Bangalore (IN); Prasada Rao Jalagam, Bangalore (IN); Saumya Roy, Bangalore (IN); Denise Christine Grunenfelder, Boston, MA (US)

(21) Appl. No.: 18/804,699

(22) Filed: Aug. 14, 2024

Related U.S. Application Data
(63) Continuation of application No. 17/983,097, filed on Nov. 8, 2022.
Application No. 17/983,097 is a continuation of application No. 17/004,058, filed on Aug. 27, 2020, now Pat. No. 11,584,747.
(60) Provisional application No. 62/892,799, filed on Aug. 28, 2019.

(51) Int. Cl. C07D 471/04

(52) U.S. Cl. CPC C07D 471/04

(57) ABSTRACT

Disclosed are compounds of Formula (I):

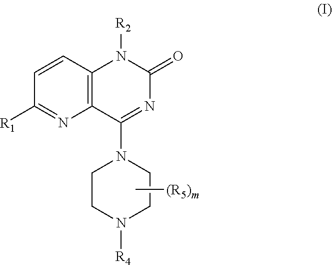

or a salt thereof, wherein: R₁, R₂, R₄, R₅, and m are defined herein. Also disclosed are methods of using such compounds to inhibit the activity of one or both of diacylglycerol kinase alpha (DGKα) and diacylglycerol kinase zeta (DGKζ), and pharmaceutical compositions comprising such compounds. These compounds are useful in the treatment of viral infections and proliferative disorders, such as cancer.

### CROSS REFERENCE

This application claims the benefit of U.S. Provisional Application Ser. No. 62/892,799 filed Aug. 28, 2019 which is incorporated herein in its entirety.

### DESCRIPTION

The present invention generally relates to substituted pyridopyrimidinonyl compounds that activate T cells, promote T cell proliferation, and/or exhibit antitumor activity. Provided herein are substituted pyridopyrimidinonyl compounds, compositions comprising such compounds, and methods of their use. The invention further pertains to pharmaceutical compositions comprising at least one compound according to the invention that are useful for the treatment of proliferative disorders, such as cancer, and viral infections.

### BACKGROUND OF THE INVENTION

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities. However, although an endogenous immune response to cancer is observed in preclinical models and patients, this response is ineffective, and established cancers are viewed as “self” and tolerated by the immune system. Contributing to this state of tolerance, tumors may exploit several distinct mechanisms to actively subvert anti-tumor immunity. These mechanisms include dysfunctional T-cell signaling (Mizoguchi et al., (1992) Science 258:1795-98), suppressive regulatory cells (Facciabene et al., (2012) Cancer Res. 72:2162-71), and the co-opting of endogenous “immune checkpoints”, which serve to down-modulate the intensity of adaptive immune responses and protect normal tissues from collateral damage, by tumors to evade immune destruction (Topalian et al., (2012) Curr. Opin. Immunol. 24:1-6; Mellman et al. (2011) Nature 480:480-489).

Diacylglycerol kinases (DGKs) are lipid kinases that mediate the conversion of diacylglycerol to phosphatidic acid thereby terminating T cell functions propagated through the TCR signaling pathway. Thus, DGKs serve as intracellular checkpoints and inhibition of DGKs are expected to enhance T cell signaling pathways and T cell activation. Supporting evidence include knock-out mouse models of either DGKα or DGKζ which show a hyper-responsive T cell phenotype and improved anti-tumor immune activity (Riese M. J. et al., Journal of Biological Chemistry, (2011) 7: 5254-5265; Zha Y et al., Nature Immunology, (2006) 12:1343; Olenchock B. A. et al., (2006) 11: 1174-81). Furthermore tumor infiltrating lymphocytes isolated from human renal cell carcinoma patients were observed to overexpress DGKα which resulted in inhibited T cell function (Prinz, P. U. et al., J Immunology (2012) 12:5990-6000). Thus, DGKα and DGKζ are viewed as targets for cancer immunotherapy (Riese M. J. et al., Front Cell Dev Biol. (2016) 4: 108; Chen, S. S. et al., Front Cell Dev Biol. (2016) 4: 130; Avila-Flores, A. et al., Immunology and Cell Biology (2017) 95: 549-563; Noessner, E., Front Cell Dev Biol. (2017) 5: 16; Krishna, S., et al., Front Immunology (2013) 4:178; Jing, W. et al., Cancer Research (2017) 77: 5676-5686.

There remains a need for compounds useful as inhibitors of one or both of DGKα and DGKζ. Additionally, there remains a need for compounds useful as inhibitors of one of both of DGKα and DGKζ that have selectivity over other diacylglycerol kinases, protein kinases, and/or other lipid kinases.

Accordingly, an agent that is safe and effective in restoring T cell activation, lowering antigen threshold, enhancing antitumor functionality, and/or overcoming the suppressive effects of one or more endogenous immune checkpoints, such as PD-1, LAG-3 and TGFβ, would be an important addition for the treatment of patients with proliferative disorders, such as cancer, as well as viral infections.

### SUMMARY OF THE INVENTION

Applicants have found compounds that have activity as inhibitors of one or both of DGKα and DGKζ. Further, applicants have found compounds that have activity as inhibitors of one or both of DGKα and DGKζ and have selectivity over other diacylglycerol kinases, protein kinases, and/or other lipid kinases. These compounds are provided to be useful as pharmaceuticals with desirable stability, bioavailability, therapeutic index, and toxicity values that are important to their druggability.

The present invention provides substituted pyridopyrimidinonyl compounds of Formula (I), which are useful as inhibitors of DGKα, DGKζ, or both DGKα and DGKζ, including salts and prodrugs thereof.

The present invention also provides pharmaceutical compositions comprising a compound of Formula (I) and/or a pharmaceutically acceptable salt thereof; and a pharmaceutically acceptable carrier.

The present invention also provides a method of treating a disease or disorder associated with the activity of DGKα, DGKζ, or both DGKα and DGKζ, the method comprising administering to a mammalian patient a compound of Formula (I) and/or a pharmaceutically acceptable salt thereof.

The present invention also provides processes and intermediates for making the compounds of Formula (I) and/or salts thereof.

The present invention also provides a compound of Formula (I) and/or a pharmaceutically acceptable salt thereof, for use in therapy.

The present invention also provides the use of the compounds of Formula (I) and/or pharmaceutically acceptable salts thereof, for the manufacture of a medicament for the treatment of proliferative disorders, such as cancer and viral infections.

The compounds of Formula (I) and compositions comprising the compounds of Formula (I) may be used in treating, preventing, or curing viral infections and various proliferative disorders, such as cancer. Pharmaceutical compositions comprising these compounds are useful in treating, preventing, or slowing the progression of diseases or disorders in a variety of therapeutic areas, such as viral infections and cancer.

These and other features of the invention will be set forth in expanded form as the disclosure continues.

### DETAILED DESCRIPTION

The first aspect of the present invention provides at least one compound of Formula (I):

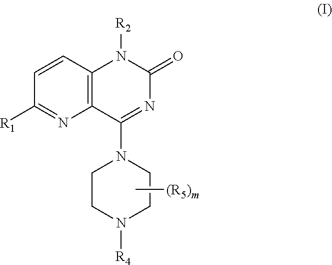

or a salt thereof, wherein:
R₁ is H, F, Cl, Br, —CN, —OH, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
   (i) —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
   (ii) C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or
   (iii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

The second aspect of the present invention provides at least one compound of Formula (I):

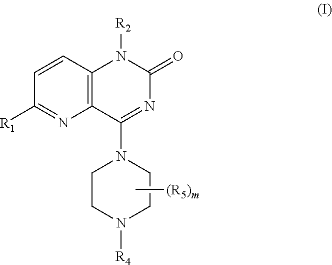

or a salt thereof, wherein:
R₁ is H, F, Cl, Br, —CN, C₁₋₃ alkyl substituted with zero to 4 R₁ₐ, C₃₋₄ cycloalkyl substituted with zero to 4 R₁ₐ, C₁₋₃ alkoxy substituted with zero to 4 R₁ₐ, —NRₐRₐ, —S(O)ₙRₑ, or —P(O)RₑRₑ;
each R₁ₐ is independently F, Cl, —CN, —OH, —OCH₃, or —NRₐRₐ;
each Rₐ is independently H or C₁₋₃ alkyl;
each Rₑ is independently C₃₋₄ cycloalkyl or C₁₋₃ alkyl substituted with zero to 4 R₁ₐ;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CH₂R₄ₐ, —CH₂CH₂R₄ₐ, —CH₂CHR₄ₐR_{4d}, —CHR₄ₐR_{4b}, or —CR₄ₐR_{4b}R_{4c};
R₄ₐ and R_{4b} are independently:
   (i) C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ;
   (ii) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or
   (iii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl;
or R₄ₐ and R_{4b} together with the carbon atom to which they are attached form a C₃₋₆ cycloalkyl or a 3- to 6-membered heterocyclyl, each substituted with zero to 3 R_{f};
each R_{f} is independently F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, and —NR_{c}R_{c};
R_{4c} is C₁₋₆ alkyl or C₃₋₆ cycloalkyl, each substituted with zero to 4 substituents independently selected from F, Cl, —OH, C₁₋₂ alkoxy, C₁₋₂ fluoroalkoxy, and —CN;
R_{4d} is —OCH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
m is zero, 1, 2, or 3; and
n is zero, 1, or 2.

In one embodiment, a compound of Formula (I) or a salt thereof is provided wherein: R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂; R₂ is H or C₁₋₂ alkyl substituted with zero to 2 R₂ₐ; each R₂ₐ is independently F, Cl, —CN, —OH, —O(C₁₋₂ alkyl), cyclopropyl, C₃₋₄ alkenyl, or C₃₋₄ alkynyl; R₄ₐ and R_{4b} are independently: (i) —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ; (ii) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or (iii) C₁₋₃ alkyl substituted with one cyclic group selected from C₃₋₆ cycloalkyl, 4- to 10-membered heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; or R₄ₐ and R_{4b} together with the carbon atom to which they are attached, form a C₃₋₆ cycloalkyl or a 3- to 6-membered heterocyclyl, each substituted with zero to 3 R_{f}; each R_{f} is independently F, Cl, Br, —OH, —CN, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c}; R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN; each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each Rₓ is independently H or —CH₃; and m is 1, 2, or 3.

In one embodiment, a compound of Formula (I) or a salt thereof is provided wherein: R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂; each R₁ₐ is independently F, Cl, or —CN; each Rₐ is independently H or C₁₋₃ alkyl; R₂ is H or C₁₋₂ alkyl substituted with zero to 2 R₂ₐ; each R₂ₐ is independently F, Cl, —CN, —OH, —O(C₁₋₂ alkyl), cyclopropyl, C₃₋₄ alkenyl, or C₃₋₄ alkynyl; R₄ₐ and R_{4b} are independently: (i) C₁₋₄ alkyl substituted with zero to 4 substituents independently selected from F, Cl, —CN, —OH, —OCH₃, —SCH₃, C₁₋₃ fluoroalkoxy, and —NRₐRₐ; (ii) C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (iii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; or R₄ₐ and R_{4b} together with the carbon atom to which they are attached, form a C₃₋₆ cycloalkyl or a 3- to 6-membered heterocyclyl, each substituted with zero to 3 R_{f}; each R_{f} is independently F, Cl, Br, —OH, —CN, ═O, C₁₋₄ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, or a cyclic group selected from C₃₋₆ cycloalkyl, 3- to 6-membered heterocyclyl, phenyl, monocyclic heteroaryl, and bicyclic heteroaryl, each cyclic group substituted with zero to 3 substituents independently selected from F, Cl, Br, —OH, —CN, C₁₋₄ alkyl, C₁₋₂ fluoroalkyl, C₁₋₃ alkoxy, C₁₋₂ fluoroalkoxy, and —NR_{c}R_{c}; R_{4c} is C₁₋₄ alkyl or C₃₋₆ cycloalkyl, each substituted with zero to 4 substituents independently selected from F, Cl, —OH, C₁₋₂ alkoxy, C₁₋₂ fluoroalkoxy, and —CN; each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂(heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —C(O)(C₁₋₄ alkyl), —(CH₂)₁₋₂NR_{c}S(O)₂(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl); and m is 1, 2, or 3.

In one embodiment, a compound of Formula (I) or a salt thereof is provided wherein: R₁ is Cl or —CN; R₂ is —CH₃; R₄ is —CH₂R₄ₐ or —CHR₄ₐR_{4b}; R₄ₐ is cyclopropyl, cyclobutyl, cyclohexyl, bicyclo[1.1.1]pentanyl, phenyl, pyridinyl, pyrimidinyl, oxadiazolyl, benzo[d][1,3]dioxolyl, or oxodihydrobenzo[d]oxazolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; R_{4b} is: (i) —CH₃ and —CH₂CH₃; or (ii) phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl; each R₅ is independently —CH₃, —CH₂CH₃, —CH₂OH, or —CH₂OCH₃; and m is 2.

In one embodiment, a compound of Formula (I) or a salt thereof is provided wherein: R₁ is Cl, —CN, —OH, —CHF₂, —CH₂OH, —CH₂OCH₃, —OCH₃, —OCH₂CH₃, —OCHF₂, —OCH₂CH₂OCH₃, or —OCH₂CH₂N(CH₃)₂; R₂ is H, —CH₃, or —CD₃; R₄ is —CH₂R₄ₐ or —CHR₄ₐR_{4b}; R₄ₐ is cyclohexyl, phenyl, pyridinyl, pyrimidinyl, oxadiazolyl, benzo[d][1,3]dioxolyl, or oxodihydrobenzo[d]oxazolyl, each substituted with zero to 3 substituents independently selected from F, Cl, Br, —CN, —CH₃, —CH(CH₃)₂, —C(CH₃)₃, —CH₂OH, —CHF₂, —CF₃, —CH₂Br, —CH₂NH₂, —CH₂NHC(O)OCH₃, —C(CH₃)₂CN, —OCH₃, —OCD₃, —OCH₂CH₃, —OCH(CH₃)₂, —OCHF₂, —OCF₃, —OCH₂CH₂CF₃, —OC(CH₃)₂CN, —OC(CH₃)₂CH₂OH, —OC(CH₃)₂CH₂OCH₃, —N(CH₃)₂, —C(O)OCH₃, cyclopropyl, cyanocyclopropyl, methylcyclopropyl, —O(cyclopropyl), —O((ethoxycarbonyl)cyclopropyl), morpholinyl, pyrrolidinonyl, tetrahydropyranyl, dioxolanyl, —CH₂(morpholinyl), —CH₂(difluoromorpholinyl), —CH₂(dimethylmorpholinyl), —CH₂(oxaazabicyclo[2.2.1]heptanyl), —CH₂(oxaazaspiro[3.3]heptanyl), —CH₂(methylpiperazinonyl), —CH₂(acetylpiperazinyl), —CH₂(piperidinyl), —CH₂(difluoropiperidinyl), —CH₂(methoxypiperidinyl), —CH₂(hydroxypiperidinyl), —C(CH₃)₂(morpholinyl), —OCH₂(cyclopropyl), —OCH₂(methylcyclopropyl), —OCH₂(methylazetidinyl), —OCH₂(oxetanyl), —OCH₂(tetrahydropyranyl), —OCH₂(thiazolyl), or —OCH₂CH₂(cyclopropyl); R_{4b} is: (i) —CN, —CH₃, —CH₂CH₃, —CH₂CH₂CH₃, or —CH(CH₃)₂; or (ii) phenyl, isoxazolyl, oxadiazolyl, thiazolyl, or triazolyl, each substituted with zero to 3 substituents independently selected from F, Cl, Br, —CH₃, —C(CH₃)₃, —CF₃, —OCF₃, and cyclopropyl; each R₅ is independently —CH₃, —CH₂CH₃, —CH₂CH₂CH₃, —CH₂OH, —CH₂OCH₃, —CH₂OCH₂CH₃, —CH₂NH₂, —CH₂N₃, or —CH₂NHC(O)OCH₃; and m is 2.

In one embodiment, a compound of Formula (I) or a salt thereof is provided wherein R₁ is H, F, Cl, Br, —CN, —OH, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂. Included in this embodiment are compounds in which R₁ is Cl, —CN, —OH, —CHF₂, —CH₂OH, —CH₂OCH₃, —OCH₃, —OCH₂CH₃, —OCHF₂, —OCH₂CH₂OCH₃, or —OCH₂CH₂N(CH₃)₂.

In one embodiment, a compound of Formula (I) or a salt thereof is provided wherein R₁ is H, F, Cl, Br, —CN, C₁₋₃ alkyl substituted with zero to 4 R₁ₐ, cyclopropyl substituted with zero to 3 R₁ₐ, C₁₋₃ alkoxy substituted with zero to 3 R₁ₐ, —NRₐRₐ, —S(O)ₙCH₃, or —P(O)(CH₃)₂. Included in this embodiment are compounds in which R₁ is H, F, Cl, Br, —CN, —CH₃, cyclopropyl, —OCH₃, or —NH₂. Also included in this embodiment are compounds in which R₁ is Cl or —CN. Further, included in this embodiment are compounds in which R₁ is —CN.

In one embodiment, a compound of Formula (I) or a salt thereof is provided wherein R₂ is H, C₁₋₂ alkyl substituted with zero to 4 R₂ₐ, or C₃₋₄ cycloalkyl substituted with zero to 2 R₂ₐ. Included in this embodiment are compounds in which R₂ is H or C₁₋₂ alkyl substituted with zero to 2 R₂ₐ. Also included in this embodiment are compounds in which R₂ is H or —CH₃. Further, included in this embodiment are compounds in which R₁ is —CH₃. Additionally, included in this embodiment are compounds in which R₂ is H, —CH₃, or —CD₃.

In one embodiment, a compound of Formula (I) or a salt thereof is provided wherein R₂ is H.

In one embodiment, a compound of Formula (I) or a salt thereof is provided wherein R₂ is —CH₃.

In one embodiment, a compound of Formula (I) or a salt thereof is provided wherein R₂ is —CD₃.

In one embodiment, a compound of Formula (I) or a salt thereof is provided wherein R₄ is —CH₂R₄ₐ or —CH₂CH₂R₄ₐ. Included in this embodiment are compounds in which R₄ is —CH₂R₄ₐ or —CD₂R₄ₐ. Also included in this embodiment are compounds in which R₄ₐ is phenyl, pyridinyl, tetrahydropyranyl, benzoxazinyl, benzo[d][1,3]dioxolyl, benzoxazinonyl, indazolyl, indolyl, or quinolinyl, each substituted with zero to 3 substituents independently selected from F, Cl, Br, —CN, —OH, —CH₃, —CH₂CH₃, —CH(CH₃)₂, —C(CH₃)₃, —CHF₂, —CF₃, —OCH₃, —OCH₂CH₃, —OCH(CH₃)₂, —OCHF₂, —OCF₃, —C(O)CH₃, —C(O)OC(CH₃)₃, —N(CH₃)₂, cyanocyclopropyl, and phenyl. Further, included in this embodiment are compounds in which R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl.

In one embodiment, a compound of Formula (I) or a salt thereof is provided wherein R₄ is —CH₂R₄ₐ. Included in this embodiment are compounds in which R₄ₐ is phenyl, pyridinyl, tetrahydropyranyl, benzoxazinyl, benzo[d][1,3]dioxolyl, benzoxazinonyl, indazolyl, indolyl, or quinolinyl, each substituted with zero to 3 substituents independently selected from F, Cl, Br, —CN, —OH, —CH₃, —CH₂CH₃, —CH(CH₃)₂, —C(CH₃)₃, —CHF₂, —CF₃, —OCH₃, —OCH₂CH₃, —OCH(CH₃)₂, —OCHF₂, —OCF₃, —C(O)CH₃, —C(O)OC(CH₃)₃, —N(CH₃)₂, cyanocyclopropyl, and phenyl. Also included in this embodiment are compounds in which R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl.

In one embodiment, a compound of Formula (I) or a salt thereof is provided wherein R₄ is —CH₂R₄ₐ; and R₄ₐ is C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}.

In one embodiment, a compound of Formula (I) or a salt thereof is provided wherein R₄ is —CH₂R₄ₐ; and R₄ₐ is C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}. Included in this embodiment are compounds in which R₄ₐ is cyclohexyl, phenyl, or benzo[d][1,3]dioxolyl, each substituted with 1 to 3 substituents independently selected from F, Cl, —CH(CH₃)₂, —CF₃, —OCH₂CH₃, —OCF₃, cyclopropyl, and —OCH₂(cyclopropyl).

In one embodiment, a compound of Formula (I) or a salt thereof is provided wherein R₄ is —CHR₄ₐR_{4b}; R₄ₐ is: (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₄ alkyl substituted with one cyclic group selected from C₃₋₆ cycloalkyl, heterocyclyl, aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is phenyl or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, and methylpiperidinyl. Included this embodiment are compounds in which R₄ₐ is (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl. Also included in this embodiment are compounds in which R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; and R_{4b} is phenyl, isoxazolyl, oxadiazolyl, or thiazolyl, each substituted with zero to 3 substituents independently selected from F, Cl, —CH₃, —C(CH₃)₃, —CF₃, —OCF₃, and cyclopropyl.

In one embodiment, a compound of Formula (I) or a salt thereof is provided wherein R₄ is —CHR₄ₐR_{4b}; R₄ₐ is: (i) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or (ii) C₁₋₄ alkyl substituted with one cyclic group selected from C₃₋₆ carbocyclyl, 4- to 10-membered heterocyclyl, 6- to 10-membered aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is phenyl, isoxazolyl, oxadiazolyl, thiazolyl, or triazolyl, each substituted with zero to 3 substituents independently selected from F, Cl, Br, —CH₃, —C(CH₃)₃, —CF₃, —OCF₃, and cyclopropyl.

In one embodiment, a compound of Formula (I) or a salt thereof is provided wherein R₄ is —CHR₄ₐR_{4b}; R₄ₐ is (i) C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, C₁₋₄ hydroxyalkyl, —(CH₂)₁₋₂O(C₁₋₃ alkyl), C₁₋₄ alkoxy, —O(C₁₋₄ hydroxyalkyl), —O(CH₂)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₃ alkyl)₂, —S(O)₂(C₁₋₃ alkyl), —O(CH₂)₁₋₂(C₃₋₆ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₄ alkyl substituted with one cyclic group selected from C₃₋₆ cycloalkyl, heterocyclyl, mono- or bicyclic aryl, and heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ. Included in this embodiment are compounds in which R₄ₐ is C₃₋₆ cycloalkyl, heterocyclyl, phenyl, or heteroaryl, each substituted with zero to 4 substituents independently selected from F, Cl, Br, —CN, —OH, C₁₋₆ alkyl, C₁₋₃ fluoroalkyl, —CH₂OH, —(CH₂)₁₋₂O(C₁₋₂ alkyl), C₁₋₄ alkoxy, —O(C₁₋₄ hydroxyalkyl), —O(CH₂)₁₋₂O(C₁₋₂ alkyl), C₁₋₃ fluoroalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —O(CH₂)₁₋₂(C₃₋₄ cycloalkyl), —O(CH₂)₁₋₂(morpholinyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, heterocyclyl, phenyl, and heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is C₁₋₄ alkyl substituted with zero to 4 substituents independently selected from F, Cl, —CN, —OH, —OCH₃, —SCH₃, C₁₋₃ fluoroalkoxy, and —NRₐRₐ. Also included in this embodiment are compounds in which R₄ₐ is phenyl, pyridinyl, or benzo[d][1,3]dioxolyl, each substituted with zero to 3 substituents independently selected from F, Cl, —CN, —CH₃, —CH(CH₃)₂, —CF₃, —OCH₃, —OCH(CH₃)₂, —OCHF₂, —OCF₃, —OCH₂(cyclopropyl), and cyclopropyl; and R_{4b} is —CH₃ and —CH₂CH₃.

In one embodiment, a compound of Formula (I) or a salt thereof is provided wherein R₄ is —CHR₄ₐR_{4b}; R₄ₐ is (i) C₃₋₈ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d}; or (ii) C₁₋₄ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₆ alkyl, C₁₋₃ fluoroalkyl, C₁₋₃ alkoxy, C₁₋₃ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₆ cycloalkyl; and R_{4b} is —CN or C₁₋₆ alkyl substituted with zero to 4 substituents independently selected from F, Cl, —CN, —OH, —OCH₃, —SCH₃, C₁₋₃ fluoroalkoxy, —NRₐRₐ, —S(O)₂Rₑ, or —NRₐS(O)₂Rₑ. Included in this embodiment are compounds in which R₄ₐ is (i) C₃₋₆ carbocyclyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or (ii) C₁₋₃ alkyl substituted with one cyclic group selected from C₃₋₆ cycloalkyl, 4- to 10-membered heterocyclyl, mono- or bicyclic aryl, or 5- to 10-membered heteroaryl, said cyclic group substituted with zero to 3 substituents independently selected from F, Cl, Br, —OH, —CN, C₁₋₃ alkyl, C₁₋₂ fluoroalkyl, C₁₋₃ alkoxy, C₁₋₂ fluoroalkoxy, —OCH₂CH═CH₂, —OCH₂C≡CH, —NR_{c}R_{c}, —NRₐS(O)₂(C₁₋₃ alkyl), —NRₐC(O)(C₁₋₃ alkyl), —NRₐC(O)O(C₁₋₄ alkyl), and C₃₋₄ cycloalkyl; and R_{4b} is —CN or C₁₋₄ alkyl substituted with zero to 4 substituents independently selected from F, Cl, —CN, —OH, —OCH₃, —SCH₃, C₁₋₃ fluoroalkoxy, and —NRₐRₐ.

In one embodiment, a compound of Formula (I) or a salt thereof is provided wherein R₄ is —CHR₄ₐR_{4b}; and R_{4b} is —CN, —CH₃, —CH₂CH₃, —CH₂CH₂CH₃, or —CH(CH₃)₂.

In one embodiment, a compound of Formula (I) or a salt thereof is provided wherein R₄ is —CHR₄ₐR_{4b}; and R_{4b} is —CN, —CH₃, or —CH₂CH₃.

In one embodiment, a compound of Formula (I) or a salt thereof is provided wherein R₄ is —CHR₄ₐR_{4b}; and R_{4b} is —CN.

In one embodiment, a compound of Formula (I) or a salt thereof is provided wherein R₄ is —CHR₄ₐR_{4b}; and R_{4b} is —CH₃ or —CH₂CH₃.

In one embodiment, a compound of Formula (I) or a salt thereof is provided wherein R₄ is —CHR₄ₐR_{4b}; and R_{4b} is —CH₃.

In one embodiment, a compound of Formula (I) or a salt thereof is provided wherein R₄ is —CHR₄ₐR_{4b}; and R_{4b} is —CH₂CH₃.

In one embodiment, a compound of Formula (I) or a salt thereof is provided wherein m is 1, 2, or 3; and each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl). Included in this embodiment are compounds in which each R₅ is independently —CH₃, —CH₂CH₃, —CH₂OH, or —CH₂OCH₃.

In one embodiment, a compound of Formula (I) or a salt thereof is provided wherein m is zero.

In one embodiment, a compound of Formula (I) or a salt thereof is provided wherein m is 1, 2, or 3. Included in this embodiment are compounds in which m is 1 or 2. Also included in this embodiment are compounds in which m is 1.

In one embodiment, a compound of Formula (I) or a salt thereof is provided wherein m is 2 or 3. Included in this embodiment are compounds in which m is 2.

In one embodiment, a compound of Formula (I) or a salt thereof is provided wherein m is 3.

In one embodiment, a compound of Formula (I) or a salt thereof is provided having the structure of Formula (II):

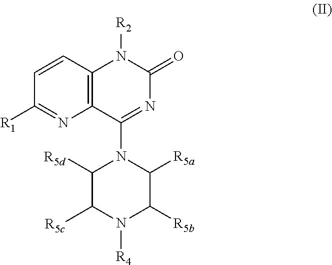

wherein one, two, or three of R₅ₐ, R_{5b}, R_{5c}, and R_{5d} are each R₅ and the remainder of R₅ₐ, R_{5b}, R_{5c}, and R_{5d} are each hydrogen. Included in this embodiment are compounds in which each R₅ is independently —CN, —CH₃, —CH₂CH₃, —CH(CH₃)₂, —CHC(CH₃)₂, —CH₂F, —C(CH₃)₂F, —CF(CH₃)CH(CH₃)₂, —CH₂OH, —C(CH₃)₂OH, —C(CH₃)(OH)CH(CH₃)₂, —CH₂OCH₃, —C(O)C(CH₃)₂, —C(O)OH, —C(O)OCH₃, —C(O)OC(CH₃)₂, —C(O)NH₂, —C(O)NH(cyclopropyl), —C(O)O(cyclopropyl), cyclopropyl, phenyl, methyloxadiazolyl, or methylpyridinyl. Also included in this embodiment are compounds in which each R₅ is independently —CH₃, —CH₂CH₃, —CH₂CH₂CH₃, —CH₂OH, —CH₂OCH₃, —CH₂OCH₂CH₃, —CH₂NH₂, —CH₂N₃, or —CH₂NHC(O)OCH₃.

In one embodiment, a compound of Formula (I) or a salt thereof is provided having the structure of Formula (III):

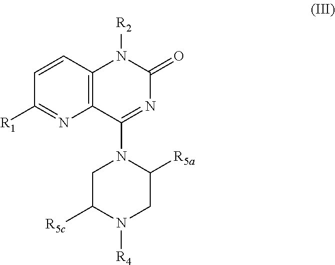

wherein R₅ₐ and R_{5c} are each R₅ and R_{5b} and R_{5d} are each hydrogen. Included in this embodiment are compounds in which (i) R₅ₐ is —CH₃ or —CH₂CH₃ and R_{5c} is —CH₃ or —CH₂CH₃; or (ii) R₅ₐ is —CH₃ and R_{5c} is —CH₂OH or —CH₂OCH₃.

In one embodiment, a compound of Formula (III) or a salt thereof is provided wherein R₅ₐ is —CH₃ and R_{5c} is —CH₃.

In one embodiment, a compound of Formula (III) or a salt thereof is provided wherein R₅ₐ is —CH₃ and R_{5c} is —CH₂CH₃.

In one embodiment, a compound of Formula (III) or a salt thereof is provided wherein R₅ₐ is —CH₂CH₃ and R_{5c} is —CH₃.

In one embodiment, a compound of Formula (III) or a salt thereof is provided wherein R₅ₐ is —CH₂CH₃ and R_{5c} is —CH₂CH₃.

In one embodiment, a compound of Formula (III) or a salt thereof is provided wherein R₅ₐ is —CH₃ and R_{5c} is —CH₂OH.

In one embodiment, a compound of Formula (III) or a salt thereof is provided wherein R₅ₐ is —CH₃ and R_{5c} is —CH₂OCH₃.

In one embodiment, a compound of Formula (III) or a salt thereof is provided wherein R₅ₐ is —CH₃ and R_{5c} is —CH₂OCH₂CH₃.

In one embodiment, a compound of Formula (III) or a salt thereof is provided wherein R₅ₐ is —CH₃ and R_{5c} is —CH₂CH₂CH₃.

In one embodiment, a compound of Formula (III) or a salt thereof is provided wherein R₅ₐ is —CH₃ and R_{5c} is —CH₂N₃.

In one embodiment, a compound of Formula (III) or a salt thereof is provided wherein R₅ₐ is —CH₃ and R_{5c} is —CH₂NH₂.

In one embodiment, a compound of Formula (III) or a salt thereof is provided wherein R₅ₐ is —CH₃ and R_{5c} is —CH₂NHC(O)OCH₃.

In one embodiment, a compound of Formula (III) or a salt thereof is provided wherein R₅ₐ is —CH₂OH and R_{5c} is —CH₃.

In one embodiment, a compound of Formula (III) or a salt thereof is provided wherein R₅ₐ is —CH₂OCH₃ and R_{5c} is —CH₃.

In one embodiment, a compound of Formula (I) or a salt thereof is provided having the structure:

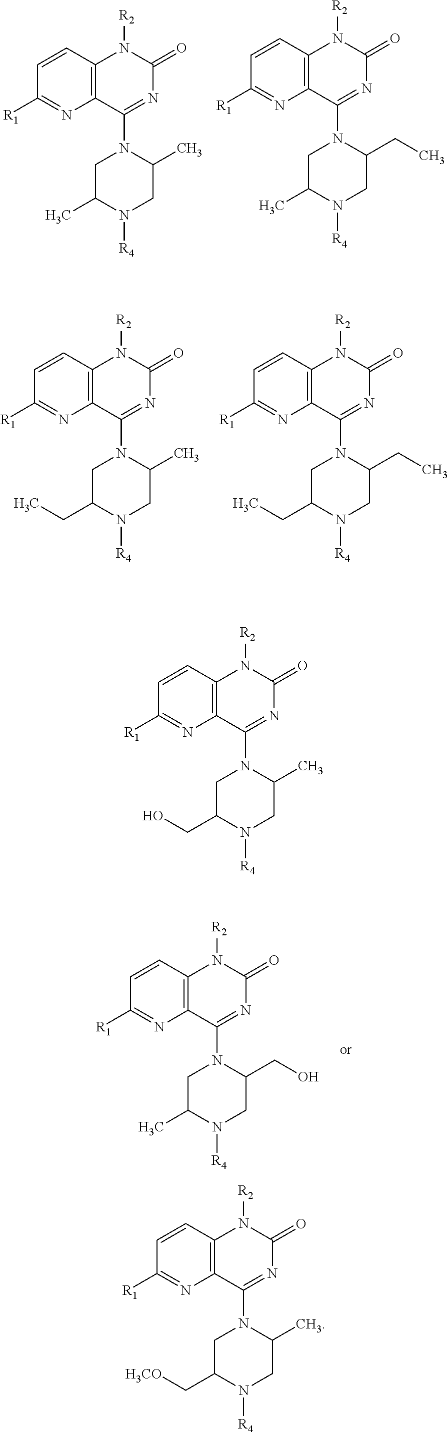

In one embodiment, a compound of Formula (I) or a salt thereof is provided having the structure:

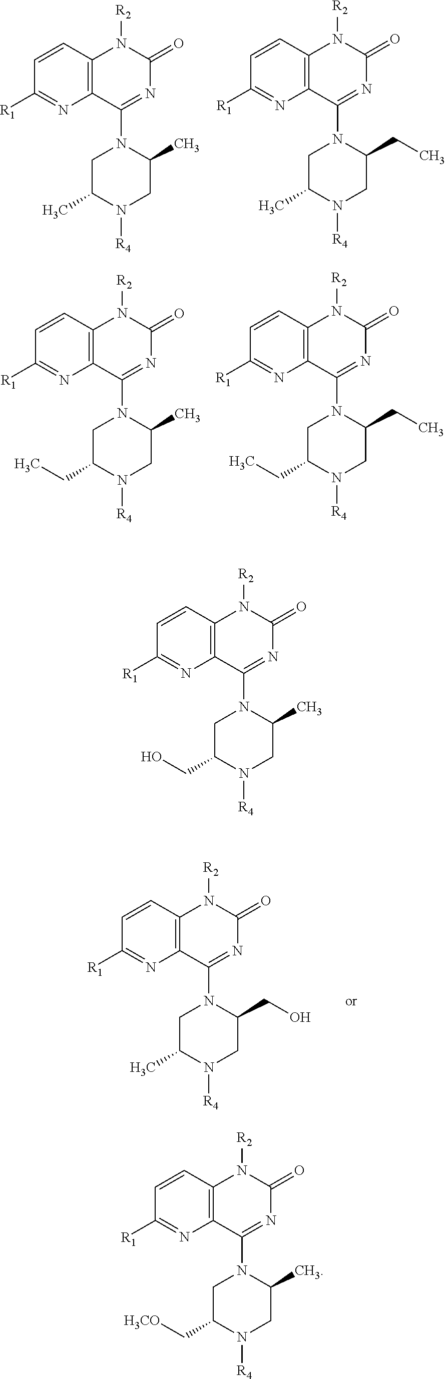

In one embodiment, a compound of Formula (I) or a salt thereof is provided having the structure:

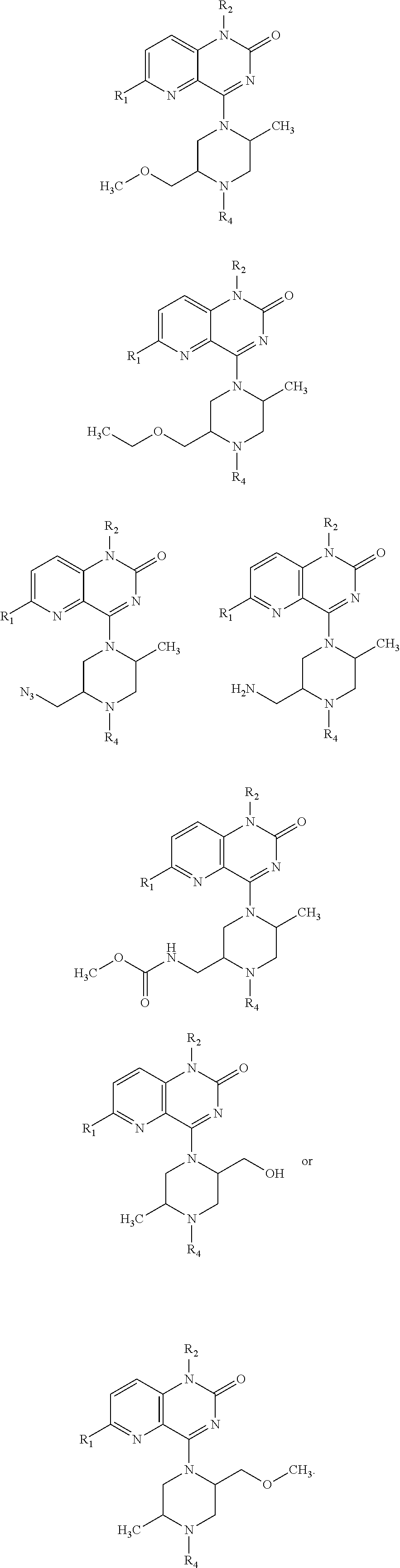

In one embodiment, a compound of Formula (I) or a salt thereof is provided having the structure:

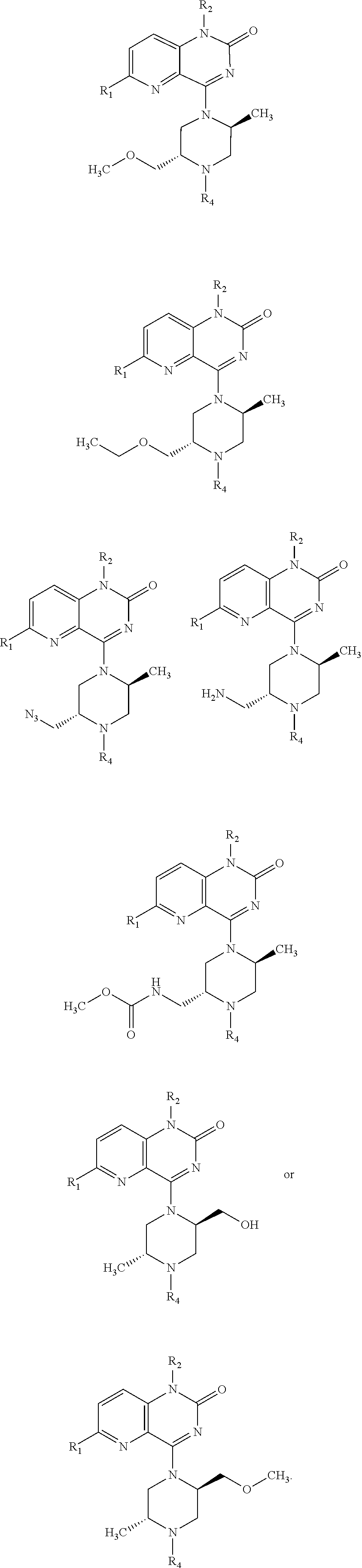

In one embodiment, a compound of Formula (I) or a salt thereof is provided wherein R₄ is:
(i)

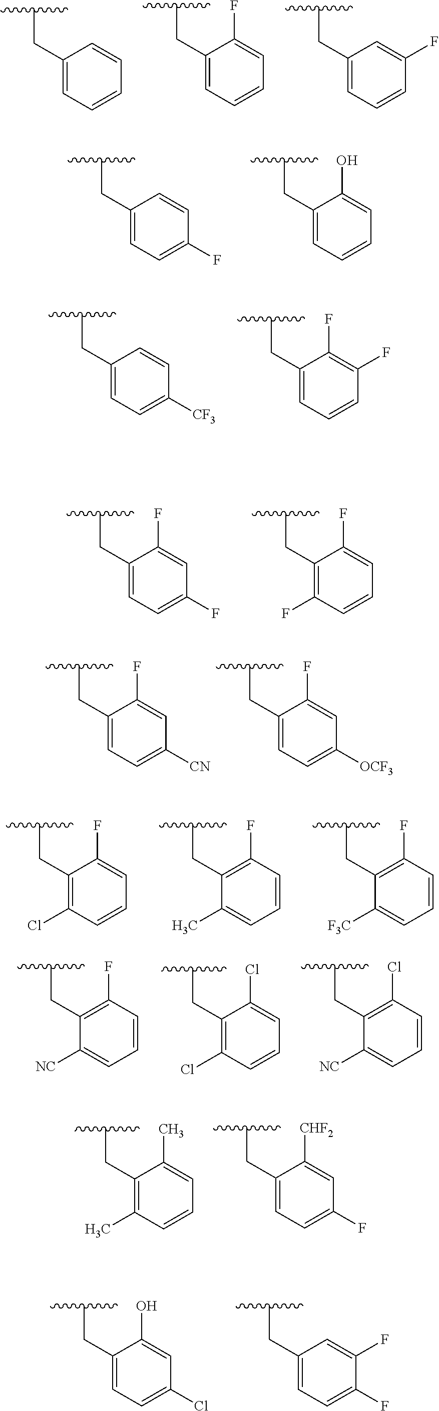

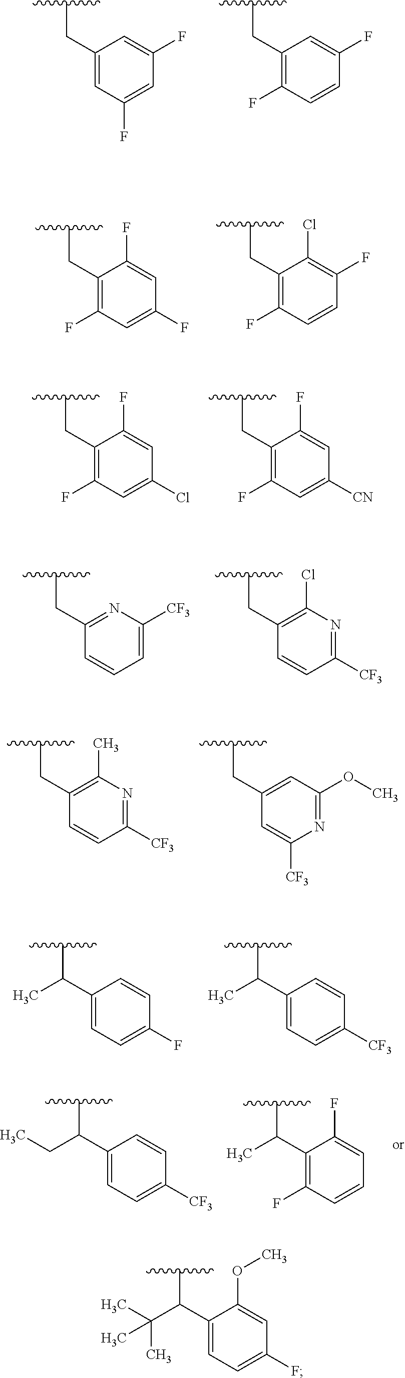

(ii)

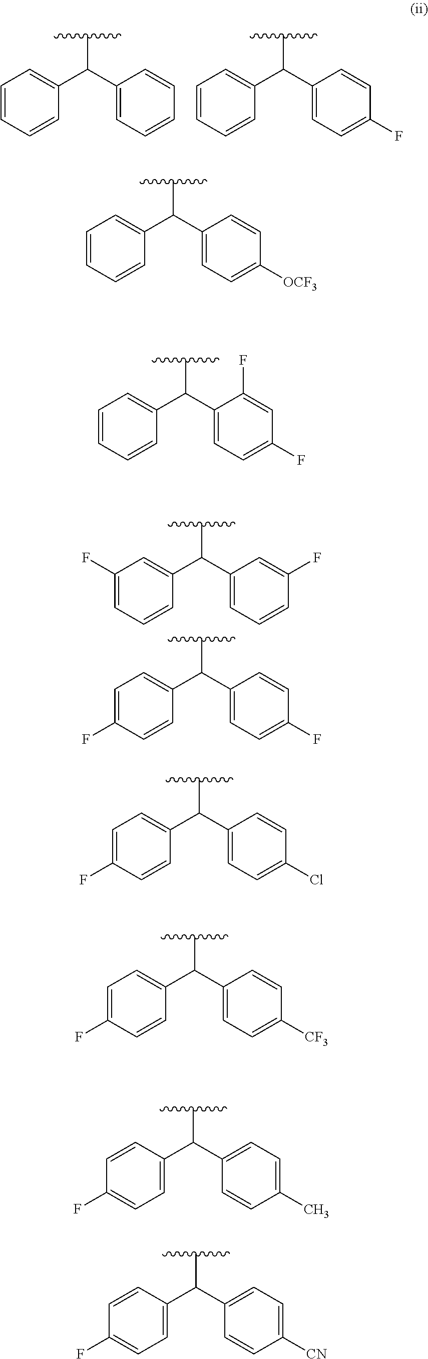

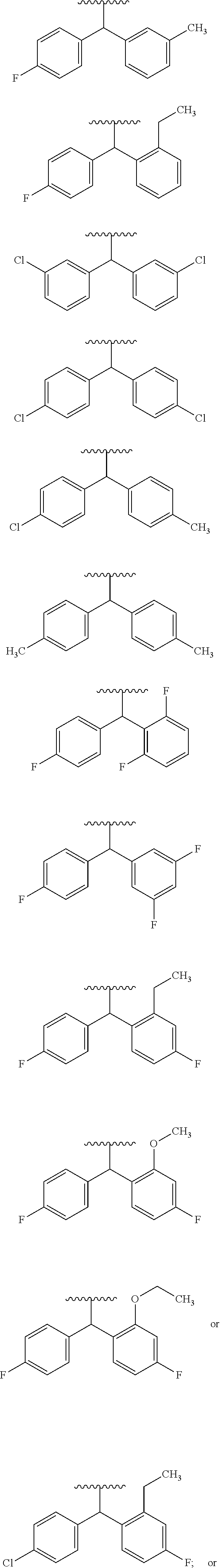

or
(iii)

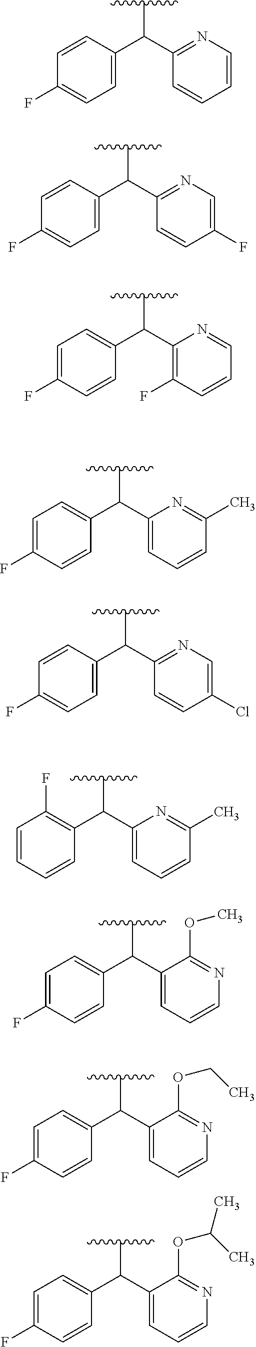

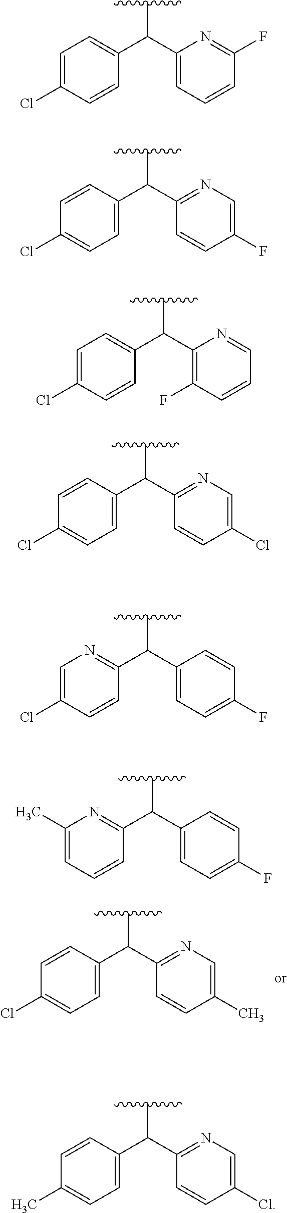

Included in this embodiment are compounds in which R₁ is H, Br, —CN, or —OCH₃; and R₂ is —CH₃.

In one embodiment, a compound of Formula (I) or a salt thereof is provided wherein R₄ is:

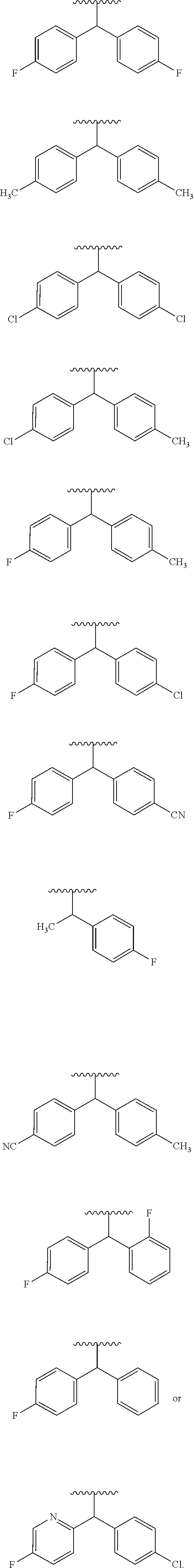

Included in this embodiment are compounds in which R₁ is H, Br, —CN, or —OCH₃; and R₂ is —CH₃. Also included in this embodiment are compounds in which R₁ is —CN; and R₂ is —CH₃.

In one embodiment, a compound of Formula (I) or a salt thereof is provided wherein R₁ is —CN; and R₂ is —CH₃.

In one embodiment, a compound of Formula (III) or a salt thereof is provided wherein R₁ is —CN; and R₂ is —CH₃.

In one embodiment, a compound of Formula (III) or a salt thereof is provided wherein R₁ is —CN; R₂ is —CH₃; R₅ₐ is —CH₃; and R_{5c} is —CH₃.

In one embodiment, a compound of Formula (III) or a salt thereof is provided wherein R₁ is —CN; R₂ is —CH₃; R₅ₐ is —CH₃; and R_{5c} is —CH₂CH₃.

In one embodiment, a compound of Formula (III) or a salt thereof is provided wherein R₁ is —CN; R₂ is —CH₃; R₅ₐ is —CH₃; and R_{5c} is —CH₂CH₂CH₃.

In one embodiment, a compound of Formula (III) or a salt thereof is provided wherein R₁ is —CN; R₂ is —CH₃; R₅ₐ is —CH₂CH₃; and R_{5c} is —CH₂CH₃.

In one embodiment, a compound of Formula (III) or a salt thereof is provided wherein R₁ is —CN; R₂ is —CH₃; R₅ₐ is —CH₃; and R_{5c} is —CH₃, —CH₂CH₃, or —CH₂CH₂CH₃.

In one embodiment, a compound of Formula (I) or a salt thereof is provided wherein R₁ is —CN; R₂ is —CH₃; R₄ is —CHR₄ₐR_{4b}; and R_{4b} is —CH₃.

In one embodiment, a compound of Formula (I) or a salt thereof is provided wherein R₁ is —CN; R₂ is —CH₃; R₄ is —CHR₄ₐR_{4b}; and R_{4b} is —CH₂CH₃.

In one embodiment, a compound of Formula (I) or a salt thereof is provided wherein R₁ is —CN; R₂ is —CH₃; R₄ is —CHR₄ₐR_{4b}; and R_{4b} is —CH₂CH₂CH₃.

In one embodiment, a compound of Formula (I) or a salt thereof is provided wherein R₁ is —CN; R₂ is —CH₃; R₄ is —CHR₄ₐR_{4b}; and R_{4b} is —CH₃, —CH₂CH₃, or —CH₂CH₂CH₃.

In one embodiment, a compound of Formula (I) or a salt thereof is provided wherein R₄ is —CHR₄ₐR_{4b}; and R₄ₐ is phenyl substituted with 1 to 2 substituents independently selected from F, Cl, —CF₃ —OCF₃, or —OCH₂(cyclopropyl). Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is phenyl substituted —CF₃ or —OCF₃; and R_{4b} is —CH₃, —CH₂CH₃, or —CH₂CH₂CH₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is phenyl substituted —CF₃ or —OCF₃; and R_{4b} is —CH₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is phenyl substituted —CF₃ or —OCF₃; and R_{4b} is —CH₂CH₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is phenyl substituted —CF₃ or —OCF₃; and R_{4b} is —CH₂CH₂CH₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is phenyl substituted with 1 to 2 substituents independently selected from F, Cl, —CF₃ —OCF₃, or —OCH₂(cyclopropyl); and R_{4b} is —CH₃, —CH₂CH₃, or —CH₂CH₂CH₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; and R₄ₐ is pyridinyl. Included in this embodiment are compounds in which R₁ is —CN; and R₂ is —CH₃.

In one embodiment, a compound of Formula (I) or a salt thereof is provided wherein R₄ is —CHR₄ₐR_{4b}; and R₄ₐ is pyridinyl substituted with —CF₃. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is pyridinyl; and R₄ₐ is phenyl substituted with Cl. Included in this embodiment are compounds in which R₁ is —CN; and R₂ is —CH₃.

In one embodiment, a compound of Formula (I) or a salt thereof is provided wherein R₄ is —CHR₄ₐR_{4b}; R₄ₐ is pyridinyl substituted with —CF₃; and R_{4b} is phenyl substituted with F. Included in this embodiment are compounds in which R₁ is —CN; and R₂ is —CH₃.

In one embodiment, a compound of Formula (I) or a salt thereof is provided wherein R₄ is —CHR₄ₐR_{4b}; one of R₄ₐ and R_{4b} is phenyl substituted with F; and the other of R₄ₐ and R_{4b} is oxadiazolyl substituted with cyclopropyl. Included in this embodiment are compounds in which R₁ is —CN; and R₂ is —CH₃.

In one embodiment, a compound of Formula (I) or a salt thereof is provided wherein said compound is: 4-((2S,5R)-4-((2,2-difluorobenzo[d][1,3]dioxol-5-yl)methyl)-2,5-diethylpiperazin-1-yl)-1-methyl-2-oxo-1,2-dihydropyrido[3,2-d]pyrimidine-6-carbonitrile (13); 4-((2S,5R)-2,5-diethyl-4-(2-fluoro-4-(trifluoromethoxy)benzyl) piperazin-1-yl)-1-methyl-2-oxo-1,2-dihydropyrido[3,2-d]pyrimidine-6-carbonitrile (27); 4-((2S,5R)-5-ethyl-2-methyl-4-(4-(trifluoromethoxy)benzyl)piperazin-1-yl)-1-methyl-2-oxo-1,2-dihydropyrido[3,2-d]pyrimidine-6-carbonitrile (54); 4-((2S,5R)-4-((2,2-difluorobenzo[d][1,3]dioxol-5-yl)methyl)-5-ethyl-2-methylpiperazin-1-yl)-1-methyl-2-oxo-1,2-dihydropyrido[3,2-d]pyrimidine-6-carbonitrile (55); 4-((2S,5R)-5-ethyl-4-(2-fluoro-4-(trifluoromethoxy)benzyl)-2-methylpiperazin-1-yl)-1-methyl-2-oxo-1,2-dihydropyrido[3,2-d]pyrimidine-6-carbonitrile (62); 4-((2S,5R)-2,5-dimethyl-4-(3,4,5-trifluorobenzyl)piperazin-1-yl)-1-methyl-2-oxo-1,2-dihydropyrido[3,2-d]pyrimidine-6-carbonitrile (130); 4-((2S,5R)-4-(3,4-difluorobenzyl)-2,5-dimethylpiperazin-1-yl)-1-methyl-2-oxo-1,2-dihydropyrido[3,2-d]pyrimidine-6-carbonitrile (133); 4-((2S,5R)-4-(2-chloro-4,5-difluorobenzyl)-2,5-dimethylpiperazin-1-yl)-1-methyl-2-oxo-1,2-dihydropyrido[3,2-d]pyrimidine-6-carbonitrile (134); 4-((2S,5R)-4-((2,2-difluorobenzo[d][1,3]dioxol-5-yl)methyl)-2,5-dimethylpiperazin-1-yl)-1-methyl-2-oxo-1,2-dihydropyrido[3,2-d]pyrimidine-6-carbonitrile (135); 4-((2S,5R)-4-(2-chloro-4-fluorobenzyl)-2,5-dimethylpiperazin-1-yl)-1-methyl-2-oxo-1,2-dihydropyrido[3,2-d] pyrimidine-6-carbonitrile (136); 4-((2S,5R)-4-(4-isopropylbenzyl)-2,5-dimethylpiperazin-1-yl)-1-methyl-2-oxo-1,2-dihydropyrido[3,2-d]pyrimidine-6-carbonitrile (137); 4-((2S,5R)-4-(4-(cyclopropylmethoxy)benzyl)-2,5-diethylpiperazin-1-yl)-1-methyl-2-oxo-1,2-dihydropyrido[3,2-d]pyrimidine-6-carbonitrile (185); 4-((2S,5R)-2,5-diethyl-4-(2-fluoro-4-(trifluoromethyl)benzyl)piperazin-1-yl)-1-methyl-2-oxo-1,2-dihydropyrido[3,2-d]pyrimidine-6-carbonitrile (204); 4-((2S,5R)-2,5-diethyl-4-(4-(trifluoromethyl)benzyl) piperazin-1-yl)-1-methyl-2-oxo-1,2-dihydropyrido[3,2-d]pyrimidine-6-carbonitrile (205); 4-((2S,5R)-4-(4-cyclopropyl-2-fluorobenzyl)-2,5-diethylpiperazin-1-yl)-1-methyl-2-oxo-1,2-dihydropyrido[3,2-d]pyrimidine-6-carbonitrile (242); 4-((2S,5R)-4-((4,4-difluorocyclohexyl)methyl)-2,5-diethylpiperazin-1-yl)-1-methyl-2-oxo-1,2-dihydropyrido[3,2-d]pyrimidine-6-carbonitrile (269); or 4-((2S,5R)-4-(4-ethoxybenzyl)-5-ethyl-2-methylpiperazin-1-yl)-1-methyl-2-oxo-1,2-dihydropyrido[3,2-d]pyrimidine-6-carbonitrile (310).

In one embodiment, a compound of Formula (I) or a salt thereof is provided wherein said compound is: 4-((2S,5R)-2,5-diethyl-4-((4-fluorophenyl)(5-(trifluoromethyl) pyridin-2-yl)methyl) piperazin-1-yl)-1-methyl-2-oxo-1,2-dihydropyrido[3,2-d] pyrimidine-6-carbonitrile (1-2); 4-((2S,5R)-2,5-diethyl-4-((4-fluorophenyl)(isoxazol-3-yl) methyl)piperazin-1-yl)-1-methyl-2-oxo-1,2-dihydropyrido[3,2-d]pyrimidine-6-carbonitrile (34-35); 4-((2S,5R)-4-((5-cyclopropylisoxazol-3-yl)(4-(trifluoromethoxy) phenyl)methyl)-2,5-diethylpiperazin-1-yl)-1-methyl-2-oxo-1,2-dihydropyrido[3,2-d] pyrimidine-6-carbonitrile (42-43); 4-((2S,5R)-5-ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile (46-47); 4-((2S,5R)-4-((4-cyclopropylthiazol-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile (50-51); 4-((2S,5R)-5-ethyl-4-((4-fluorophenyl)(6-(trifluoromethyl)pyridin-2-yl)methyl)-2-methylpiperazin-1-yl)-1-methyl-2-oxo-1,2-dihydropyrido[3,2-d]pyrimidine-6-carbonitrile (65-66); 4-((2S,5R)-5-ethyl-4-((4-fluorophenyl)(isoxazol-3-yl)methyl)-2-methylpiperazin-1-yl)-1-methyl-2-oxo-1,2-dihydropyrido[3,2-d]pyrimidine-6-carbonitrile (73-74); 4-((2S,5R)-4-((5-cyclopropylpyridin-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile (75-76); 4-((2S,5R)-5-ethyl-4-((4-fluorophenyl)(pyridin-2-yl)methyl)-2-methylpiperazin-1-yl)-1-methyl-2-oxo-1,2-dihydropyrido[3,2-d]pyrimidine-6-carbonitrile (87-88); 4-((2S,5R)-5-ethyl-2-methyl-4-(pyridin-2-yl(4-(trifluoromethoxy)phenyl)methyl)piperazin-1-yl)-1-methyl-2-oxo-1,2-dihydropyrido[3,2-d]pyrimidine-6-carbonitrile (93-94); 4-((2S,5R)-4-((4-chlorophenyl) (pyridin-2-yl)methyl)-5-ethyl-2-methylpiperazin-1-yl)-1-methyl-2-oxo-1,2-dihydropyrido[3,2-d]pyrimidine-6-carbonitrile (95-96); 4-((2S,5R)-5-ethyl-4-((4-fluorophenyl)(6-(trifluoromethyl)pyridin-3-yl)methyl)-2-methylpiperazin-1-yl)-1-methyl-2-oxo-1,2-dihydropyrido[3,2-d]pyrimidine-6-carbonitrile (97-98); 4-((2S,5R)-4-((5-cyclopropylisoxazol-3-yl)(4-(trifluoromethoxy)phenyl)methyl)-5-ethyl-2-methylpiperazin-1-yl)-1-methyl-2-oxo-1,2-dihydropyrido[3,2-d]pyrimidine-6-carbonitrile (99-100); 4-((2S,5R)-4-((5-cyclopropylisoxazol-3-yl)(4-fluorophenyl)methyl)-5-ethyl-2-methylpiperazin-1-yl)-1-methyl-2-oxo-1,2-dihydropyrido[3,2-d]pyrimidine-6-carbonitrile (101-102); 4-((2S,5R)-4-((2-cyclopropylthiazol-5-yl)(4-fluorophenyl)methyl)-5-ethyl-2-methylpiperazin-1-yl)-1-methyl-2-oxo-1,2-dihydropyrido[3,2-d]pyrimidine-6-carbonitrile (103-104); 6-chloro-4-((2S,5R)-4-((3-cyclopropyl-1,2,4-oxadiazol-5-yl)(4-fluorophenyl) methyl)-2,5-dimethylpiperazin-1-yl)-1-methylpyrido[3,2-d]pyrimidin-2(1H)-one (105); 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (106-107); 4-((2S,5R)-4-((3-(tert-butyl)-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (108-109); 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (110-111); 4-((2S,5R)-4-((3-cyclopropyl-1,2,4-oxadiazol-5-yl)(4-fluorophenyl)methyl)-5-ethyl-2-methylpiperazin-1-yl)-1-methyl-2-oxo-1,2-dihydropyrido[3,2-d]pyrimidine-6-carbonitrile (112-113); 4-((2S,5R)-2-ethyl-4-((4-fluorophenyl)(5-(trifluoromethyl)pyridin-2-yl)methyl)-5-methylpiperazin-1-yl)-1-methyl-2-oxo-1,2-dihydropyrido[3,2-d]pyrimidine-6-carbonitrile (114-115); 4-((2S,5R)-2-ethyl-4-((4-fluorophenyl)(6-(trifluoromethyl)pyridin-2-yl) methyl)-5-methylpiperazin-1-yl)-1-methyl-2-oxo-1,2-dihydropyrido[3,2-d]pyrimidine-6-carbonitrile (120-121); 4-((2S,5R)-4-(bis(4-fluorophenyl)methyl)-2,5-dimethylpiperazin-1-yl)-1-methyl-2-oxo-1,2-dihydropyrido[3,2-d]pyrimidine-6-carbonitrile (124-125); 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile (126-127); 4-((2S,5R)-4-((4-cyclopropylthiazol-2-yl)(4-fluorophenyl) methyl)-2,5-dimethylpiperazin-1-yl)-1-methyl-2-oxo-1,2-dihydropyrido[3,2-d] pyrimidine-6-carbonitrile (158-159); 4-((2S,5R)-4-((4-fluorophenyl)(isoxazol-3-yl) methyl)-2,5-dimethylpiperazin-1-yl)-1-methyl-2-oxo-1,2-dihydropyrido[3,2-d] pyrimidine-6-carbonitrile (162-163); 4-((2S,5R)-4-((5-cyclopropylisoxazol-3-yl)(4-(trifluoromethoxy)phenyl)methyl)-2,5-dimethylpiperazin-1-yl)-1-methyl-2-oxo-1,2-dihydropyrido[3,2-d]pyrimidine-6-carbonitrile (164-165); 4-((2S,5R)-4-((4-fluorophenyl) (2-(trifluoromethyl)thiazol-4-yl)methyl)-2,5-dimethylpiperazin-1-yl)-1-methyl-2-oxo-1,2-dihydropyrido[3,2-d]pyrimidine-6-carbonitrile (170-171); 4-((2S,5S)-4-((4-fluorophenyl) (5-(trifluoromethyl)pyridin-2-yl)methyl)-5-(methoxymethyl)-2-methylpiperazin-1-yl)-1-methyl-2-oxo-1,2-dihydropyrido[3,2-d]pyrimidine-6-carbonitrile (178-179); 6-chloro-4-((2S,5S)-4-((4-fluorophenyl)(5-(trifluoromethyl)pyridin-2-yl)methyl)-5-(hydroxymethyl)-2-methylpiperazin-1-yl)-1-methylpyrido[3,2-d]pyrimidin-2(1H)-one (180); 4-((2S,5S)-4-((4-fluorophenyl)(5-(trifluoromethyl)pyridin-2-yl)methyl)-5-(hydroxymethyl)-2-methylpiperazin-1-yl)-1-methyl-2-oxo-1,2-dihydropyrido[3,2-d]pyrimidine-6-carbonitrile (181-182); 4-((2S,5R)-2,5-diethyl-4-((4-fluorophenyl)(2-(trifluoromethyl) thiazol-4-yl)methyl)piperazin-1-yl)-1-methyl-2-oxo-1,2-dihydropyrido[3,2-d]pyrimidine-6-carbonitrile (210-211); 4-((2S,5R)-4-((6-(difluoromethyl)pyridin-2-yl)(4-fluorophenyl) methyl)-2,5-diethylpiperazin-1-yl)-1-methyl-2-oxo-1,2-dihydropyrido[3,2-d]pyrimidine-6-carbonitrile (257-258); 4-((2S,5R)-4-((3-bromo-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile (293); 4-((2S,5R)-4-((3-cyclopropyl-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile (294-295); 4-((2S,5R)-5-ethyl-4-((4-fluorophenyl)(2-(trifluoromethyl)thiazol-4-yl)methyl)-2-methylpiperazin-1-yl)-1-methyl-2-oxo-1,2-dihydropyrido[3,2-d]pyrimidine-6-carbonitrile (296-297); 4-((2S,5R)-4-((6-(difluoromethyl)pyridin-2-yl)(4-fluorophenyl)methyl)-5-ethyl-2-methylpiperazin-1-yl)-1-methyl-2-oxo-1,2-dihydropyrido[3,2-d]pyrimidine-6-carbonitrile (315-316); 6-chloro-4-((2S,5R)-4-((5-cyclopropyl-1,2,4-oxadiazol-3-yl)(4-fluorophenyl)methyl)-2,5-diethylpiperazin-1-yl)-1-methylpyrido[3,2-d]pyrimidin-2(1H)-one (370); 4-((2S,5R)-4-((5-cyclopropyl-1,2,4-oxadiazol-3-yl)(4-fluorophenyl)methyl)-2,5-diethylpiperazin-1-yl)-1-methyl-2-oxo-1,2-dihydropyrido[3,2-d]pyrimidine-6-carbonitrile (371-372); 4-((2S,5R)-4-(bis(4-chlorophenyl)methyl)-2,5-dimethylpiperazin-1-yl)-1-methyl-2-oxo-1,2-dihydropyrido[3,2-d]pyrimidine-6-carbonitrile (373-374); 4-((2S,5R)-4-((4-cyanophenyl)(4-fluorophenyl)methyl)-2,5-dimethylpiperazin-1-yl)-1-methyl-2-oxo-1,2-dihydropyrido[3,2-d]pyrimidine-6-carbonitrile (375); or 4-((2S,5R)-4-((4-fluorophenyl)(3-(trifluoromethyl)bicyclo[1.1.1]pentan-1-yl)methyl)-2,5-dimethylpiperazin-1-yl)-1-methyl-2-oxo-1,2-dihydropyrido[3,2-d]pyrimidine-6-carbonitrile (450-452).

In one embodiment, a compound of Formula (I) or a salt thereof is provided wherein said compound is: 4-((2S,5R)-4-(1-(4-cyclopropylphenyl)ethyl)-2, 5-dimethylpiperazin-1-yl)-1-methyl-2-oxo-1, 2-dihydropyrido[3, 2-d]pyrimidine-6-carbonitrile (3-4); 4-((2S,5R)-2,5-diethyl-4-(1-(4-(trifluoromethyl)phenyl)propyl) piperazin-1-yl)-1-methyl-2-oxo-1,2-dihydropyrido[3,2-d]pyrimidine-6-carbonitrile (5-6); 4-((2S,5R)-5-ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile (7-8); 4-((2S,5R)-2,5-diethyl-4-(1-(4-(trifluoromethoxy)phenyl)ethyl)piperazin-1-yl)-1-methyl-2-oxo-1,2-dihydropyrido[3,2-d]pyrimidine-6-carbonitrile (9-10); 4-((2S,5R)-2,5-diethyl-4-(1-(2-fluoro-4-methoxyphenyl)propyl)piperazin-1-yl)-1-methyl-2-oxo-1,2-dihydropyrido[3,2-d]pyrimidine-6-carbonitrile (11-12); 4-((2S,5R)-4-(1-(2,2-difluorobenzo[d][1,3]dioxol-5-yl)ethyl)-2,5-diethylpiperazin-1-yl)-1-methyl-2-oxo-1,2-dihydropyrido[3,2-d]pyrimidine-6-carbonitrile (14-15); 4-((2S,5R)-2,5-diethyl-4-(1-(4-methoxyphenyl)ethyl)piperazin-1-yl)-1-methyl-2-oxo-1,2-dihydropyrido[3,2-d]pyrimidine-6-carbonitrile (16-17); 4-((2S,5R)-2,5-diethyl-4-(1-(4-isopropoxyphenyl)ethyl)piperazin-1-yl)-1-methyl-2-oxo-1,2-dihydropyrido[3,2-d]pyrimidine-6-carbonitrile (18-19); 4-((2S,5R)-2,5-diethyl-4-(1-(4-(trifluoromethyl)phenyl)ethyl)piperazin-1-yl)-1-methyl-2-oxo-1,2-dihydropyrido[3,2-d] pyrimidine-6-carbonitrile (20-21); 4-((2S,5R)-2,5-diethyl-4-(4-(trifluoromethoxy)benzyl) piperazin-1-yl)-1-methyl-2-oxo-1,2-dihydropyrido[3,2-d]pyrimidine-6-carbonitrile (22); 4-((2S,5R)-4-(1-(4-(cyclopropylmethoxy)phenyl)ethyl)-2,5-diethylpiperazin-1-yl)-1-methyl-2-oxo-1,2-dihydropyrido[3,2-d]pyrimidine-6-carbonitrile (23-24); 4-((2S,5R)-2,5-diethyl-4-(1-(2-fluoro-4-(trifluoromethyl)phenyl)ethyl)piperazin-1-yl)-1-methyl-2-oxo-1,2-dihydropyrido[3,2-d]pyrimidine-6-carbonitrile (25-26); 4-((2S,5R)-4-(1-(4-cyclopropylphenyl)ethyl)-2,5-diethylpiperazin-1-yl)-1-methyl-2-oxo-1,2-dihydropyrido[3,2-d]pyrimidine-6-carbonitrile (28-29); 4-((2S,5R)-2,5-diethyl-4-(1-(3-fluoro-4-(trifluoromethoxy)phenyl)ethyl)piperazin-1-yl)-1-methyl-2-oxo-1,2-dihydropyrido[3,2-d]pyrimidine-6-carbonitrile (30-31); 4-((2S,5R)-4-(1-(4-(cyclopropylmethoxy)-2-fluorophenyl)ethyl)-2,5-diethylpiperazin-1-yl)-1-methyl-2-oxo-1,2-dihydropyrido[3,2-d]pyrimidine-6-carbonitrile (32-33); 4-((2S,5R)-2,5-diethyl-4-(1-(4-(trifluoromethoxy)phenyl)propyl)piperazin-1-yl)-1-methyl-2-oxo-1,2-dihydropyrido[3,2-d]pyrimidine-6-carbonitrile (36-37); 4-((2S,5R)-2,5-diethyl-4-(1-(2-fluoro-4-(trifluoromethoxy)phenyl)ethyl)piperazin-1-yl)-1-methyl-2-oxo-1,2-dihydropyrido[3,2-d]pyrimidine-6-carbonitrile (38-39); 4-((2S,5R)-4-(1-(4-cyclopropyl-2-fluorophenyl)ethyl)-2,5-diethylpiperazin-1-yl)-1-methyl-2-oxo-1,2-dihydropyrido[3,2-d]pyrimidine-6-carbonitrile (40-41); 4-((2S,5R)-2,5-diethyl-4-(1-(6-(trifluoromethyl) pyridin-3-yl)ethyl)piperazin-1-yl)-1-methyl-2-oxo-1,2-dihydropyrido[3,2-d]pyrimidine-6-carbonitrile (44-45); 4-((2S,5R)-5-ethyl-2-methyl-4-(1-(4-(trifluoromethoxy)phenyl) ethyl)piperazin-1-yl)-1-methyl-2-oxo-1,2-dihydropyrido[3,2-d]pyrimidine-6-carbonitrile (48-49); 4-((2S,5R)-5-ethyl-4-(1-(4-isopropoxyphenyl)ethyl)-2-methylpiperazin-1-yl)-1-methyl-2-oxo-1,2-dihydropyrido[3,2-d]pyrimidine-6-carbonitrile (52-53); 4-((2S,5R)-5-ethyl-4-(1-(4-methoxyphenyl)ethyl)-2-methylpiperazin-1-yl)-1-methyl-2-oxo-1,2-dihydropyrido[3,2-d]pyrimidine-6-carbonitrile (56-57); 4-((2S,5R)-5-ethyl-4-(1-(2-fluoro-4-(trifluoromethyl)phenyl)ethyl)-2-methylpiperazin-1-yl)-1-methyl-2-oxo-1,2-dihydropyrido[3,2-d]pyrimidine-6-carbonitrile (58-59); 4-((2S,5R)-4-(1-(4-cyanophenyl) ethyl)-5-ethyl-2-methylpiperazin-1-yl)-1-methyl-2-oxo-1,2-dihydropyrido[3,2-d] pyrimidine-6-carbonitrile (60-61); 4-((2S,5R)-4-(1-(4-cyclopropylphenyl)ethyl)-5-ethyl-2-methylpiperazin-1-yl)-1-methyl-2-oxo-1,2-dihydropyrido[3,2-d]pyrimidine-6-carbonitrile (63-64); 4-((2S,5R)-4-(1-(4-(cyclopropylmethoxy)phenyl)ethyl)-5-ethyl-2-methylpiperazin-1-yl)-1-methyl-2-oxo-1,2-dihydropyrido[3,2-d]pyrimidine-6-carbonitrile (67-68); 4-((2S,5R)-4-(1-(4-(cyclopropylmethoxy)-2-fluorophenyl)ethyl)-5-ethyl-2-methylpiperazin-1-yl)-1-methyl-2-oxo-1,2-dihydropyrido[3,2-d]pyrimidine-6-carbonitrile (69-70); 4-((2S,5R)-4-(1-(2,2-difluorobenzo[d][1,3]dioxol-5-yl)ethyl)-5-ethyl-2-methylpiperazin-1-yl)-1-methyl-2-oxo-1,2-dihydropyrido[3,2-d]pyrimidine-6-carbonitrile (71-72); 4-((2S,5R)-4-(1-(4-(difluoromethoxy)phenyl)propyl)-5-ethyl-2-methylpiperazin-1-yl)-1-methyl-2-oxo-1,2-dihydropyrido[3,2-d]pyrimidine-6-carbonitrile (77-78); 4-((2S,5R)-4-(1-(2,2-difluorobenzo[d][1,3]dioxol-5-yl)propyl)-5-ethyl-2-methylpiperazin-1-yl)-1-methyl-2-oxo-1,2-dihydropyrido[3,2-d]pyrimidine-6-carbonitrile (79-80); 4-((2S,5R)-5-ethyl-4-(1-(2-fluoro-4-(trifluoromethoxy)phenyl)ethyl)-2-methylpiperazin-1-yl)-1-methyl-2-oxo-1,2-dihydropyrido[3,2-d]pyrimidine-6-carbonitrile (81-82); 4-((2S,5R)-5-ethyl-2-methyl-4-(1-(4-(trifluoromethoxy)phenyl)propyl)piperazin-1-yl)-1-methyl-2-oxo-1,2-dihydropyrido[3,2-d]pyrimidine-6-carbonitrile (83-84); 4-((2S,5R)-5-ethyl-4-(1-(2-fluoro-4-(trifluoromethoxy)phenyl)propyl)-2-methylpiperazin-1-yl)-1-methyl-2-oxo-1,2-dihydropyrido[3,2-d]pyrimidine-6-carbonitrile (85-86); 4-((2S,5R)-5-ethyl-2-methyl-4-(1-(4-(trifluoromethyl)phenyl)propyl)piperazin-1-yl)-1-methyl-2-oxo-1,2-dihydropyrido[3,2-d]pyrimidine-6-carbonitrile (89-90); 4-((2S,5R)-4-(1-(4-cyclopropyl-2-fluorophenyl)ethyl)-5-ethyl-2-methylpiperazin-1-yl)-1-methyl-2-oxo-1,2-dihydropyrido[3,2-d]pyrimidine-6-carbonitrile (91-92); 4-((2S,5R)-2-ethyl-5-methyl-4-(1-(4-(trifluoromethyl)phenyl)ethyl)piperazin-1-yl)-1-methyl-2-oxo-1,2-dihydropyrido[3,2-d]pyrimidine-6-carbonitrile (116-117); 4-((2S,5R)-2-ethyl-4-(1-(2-fluoro-4-(trifluoromethyl)phenyl)ethyl)-5-methylpiperazin-1-yl)-1-methyl-2-oxo-1,2-dihydropyrido[3,2-d]pyrimidine-6-carbonitrile (118-119); 4-((2S,5R)-2-ethyl-5-methyl-4-(1-(4-(trifluoromethoxy)phenyl)ethyl)piperazin-1-yl)-1-methyl-2-oxo-1,2-dihydropyrido[3,2-d]pyrimidine-6-carbonitrile (122-123); 4-((2S,5R)-4-(1-(4-fluorophenyl)ethyl)-2,5-dimethylpiperazin-1-yl)-1-methyl-2-oxo-1,2-dihydropyrido[3,2-d]pyrimidine-6-carbonitrile (128-129); 4-((2S,5R)-2,5-dimethyl-4-(1-(4-(trifluoromethoxy)phenyl)ethyl)piperazin-1-yl)-1-methyl-2-oxo-1,2-dihydropyrido[3,2-d]pyrimidine-6-carbonitrile (131-132); 4-((2S,5R)-2,5-dimethyl-4-(1-(3,4,5-trifluorophenyl)ethyl)piperazin-1-yl)-1-methyl-2-oxo-1,2-dihydropyrido[3,2-d] pyrimidine-6-carbonitrile (138-139); 4-((2S,5R)-2,5-dimethyl-4-(1-(4-(trifluoromethyl) phenyl)ethyl)piperazin-1-yl)-1-methyl-2-oxo-1,2-dihydropyrido[3,2-d]pyrimidine-6-carbonitrile (140-141); 4-((2S,5R)-4-(1-(2-fluoro-4-(trifluoromethoxy)phenyl)ethyl)-2,5-dimethylpiperazin-1-yl)-1-methyl-2-oxo-1,2-dihydropyrido[3,2-d]pyrimidine-6-carbonitrile (142-143); 4-((2S,5R)-4-(1-(2,4-difluorophenyl)ethyl)-2,5-dimethylpiperazin-1-yl)-1-methyl-2-oxo-1,2-dihydropyrido[3,2-d]pyrimidine-6-carbonitrile (144-145); 4-((2S,5R)-4-(1-(4-chloro-2-fluorophenyl)ethyl)-2,5-dimethylpiperazin-1-yl)-1-methyl-2-oxo-1,2-dihydropyrido[3,2-d]pyrimidine-6-carbonitrile (146-147); 4-((2S,5R)-4-(1-(3,4-difluorophenyl)ethyl)-2,5-dimethylpiperazin-1-yl)-1-methyl-2-oxo-1,2-dihydropyrido[3,2-d]pyrimidine-6-carbonitrile (148-149); 4-((2S,5R)-4-(1-(2-fluoro-4-(trifluoromethyl)phenyl)ethyl)-2,5-dimethylpiperazin-1-yl)-1-methyl-2-oxo-1,2-dihydropyrido[3,2-d]pyrimidine-6-carbonitrile (150-151); 4-((2S,5R)-4-(1-(2-fluoro-4-(trifluoromethoxy)phenyl)propyl)-2,5-dimethylpiperazin-1-yl)-1-methyl-2-oxo-1,2-dihydropyrido[3,2-d]pyrimidine-6-carbonitrile (152-153); 4-((2S,5R)-2,5-dimethyl-4-(1-(4-(trifluoromethoxy)phenyl)propyl)piperazin-1-yl)-1-methyl-2-oxo-1,2-dihydropyrido[3,2-d]pyrimidine-6-carbonitrile (154-155); 4-((2S,5R)-4-(1-(4-(difluoromethoxy)phenyl)ethyl)-2,5-dimethylpiperazin-1-yl)-1-methyl-2-oxo-1,2-dihydropyrido[3,2-d]pyrimidine-6-carbonitrile (156-157); 4-((2S,5R)-4-(1-(3-fluoro-4-(trifluoromethoxy)phenyl)ethyl)-2,5-dimethylpiperazin-1-yl)-1-methyl-2-oxo-1,2-dihydropyrido[3,2-d]pyrimidine-6-carbonitrile (160-161); 4-((2S,5R)-4-(1-(2,2-difluorobenzo[d][1,3]dioxol-5-yl)ethyl)-2,5-dimethylpiperazin-1-yl)-1-methyl-2-oxo-1,2-dihydropyrido[3,2-d]pyrimidine-6-carbonitrile (166-167); 4-((2S,5R)-4-(1-(4-cyclopropyl-2-fluorophenyl)ethyl)-2,5-dimethylpiperazin-1-yl)-1-methyl-2-oxo-1,2-dihydropyrido[3,2-d]pyrimidine-6-carbonitrile (168-169); 6-chloro-4-((2S,5S)-5-(hydroxymethyl)-2-methyl-4-(1-(4-(trifluoromethyl)phenyl)ethyl) piperazin-1-yl)-1-methylpyrido[3,2-d]pyrimidin-2(1H)-one (172); 6-chloro-4-((2S,5S)-5-(methoxymethyl)-2-methyl-4-(1-(4-(trifluoromethyl)phenyl)ethyl) piperazin-1-yl)-1-methylpyrido[3,2-d] pyrimidin-2(1H)-one (173); 4-((2S,5S)-5-(methoxymethyl)-2-methyl-4-(1-(4-(trifluoromethyl)phenyl)ethyl)piperazin-1-yl)-1-methyl-2-oxo-1,2-dihydropyrido[3,2-d] pyrimidine-6-carbonitrile (174-175); 4-((2S,5S)-5-(methoxymethyl)-2-methyl-4-(1-(4-(trifluoromethoxy)phenyl)ethyl)piperazin-1-yl)-1-methyl-2-oxo-1,2-dihydropyrido[3,2-d] pyrimidine-6-carbonitrile (176-177); 4-((2S,5S)-5-(hydroxymethyl)-2-methyl-4-(1-(4-(trifluoromethoxy)phenyl)ethyl)piperazin-1-yl)-1-methyl-2-oxo-1,2-dihydropyrido[3,2-d] pyrimidine-6-carbonitrile (183-184); 6-chloro-4-((2S,5R)-2,5-diethyl-4-(1-(4-((1-hydroxy-2-methylpropan-2-yl)oxy)phenyl)ethyl)piperazin-1-yl)-1-methylpyrido[3,2-d] pyrimidin-2(1H)-one (186); 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one (187); 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one (188-189); 4-((2S,5R)-2,5-diethyl-4-(1-(4-(methoxy-d₃)phenyl)ethyl)piperazin-1-yl)-1-methyl-2-oxo-1,2-dihydropyrido[3,2-d]pyrimidine-6-carbonitrile (190-191); 4-((2S,5R)-4-(1-(6-cyclopropylpyridin-3-yl)ethyl)-2,5-diethylpiperazin-1-yl)-1-methyl-2-oxo-1,2-dihydropyrido[3,2-d]pyrimidine-6-carbonitrile (192-193); 4-((2S,5R)-2,5-diethyl-4-(1-(2-morpholino-4-(trifluoromethyl)phenyl)ethyl)piperazin-1-yl)-1-methyl-2-oxo-1,2-dihydropyrido[3,2-d]pyrimidine-6-carbonitrile (194-195); 4-((2S,5R)-4-(1-(4-(2-cyanopropan-2-yl)phenyl)ethyl)-2,5-diethylpiperazin-1-yl)-1-methyl-2-oxo-1,2-dihydropyrido[3,2-d]pyrimidine-6-carbonitrile (196-197); 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile (198-199); 4-((2S,5R)-4-(1-(4-cyclopropylphenyl)propyl)-2,5-diethylpiperazin-1-yl)-1-methyl-2-oxo-1,2-dihydropyrido[3,2-d]pyrimidine-6-carbonitrile (200-201); 4-((2S,5R)-2,5-diethyl-4-(1-(3-methyl-2-oxo-2,3-dihydrobenzo[d]oxazol-5-yl)ethyl)piperazin-1-yl)-1-methyl-2-oxo-1,2-dihydropyrido[3,2-d]pyrimidine-6-carbonitrile (202-203); 4-((2S,5R)-2,5-diethyl-4-(1-(2-morpholinopyrimidin-5-yl)ethyl)piperazin-1-yl)-1-methyl-2-oxo-1,2-dihydropyrido[3,2-d] pyrimidine-6-carbonitrile (206-207); 4-((2S,5R)-2,5-diethyl-4-(1-(4-(methoxy-d₃) phenyl)propyl)piperazin-1-yl)-1-methyl-2-oxo-1,2-dihydropyrido[3,2-d]pyrimidine-6-carbonitrile (208-209); 4-((2S,5R)-2,5-diethyl-4-(1-(4-methoxyphenyl)propyl)piperazin-1-yl)-1-methyl-2-oxo-1,2-dihydropyrido[3,2-d]pyrimidine-6-carbonitrile (212-213); 4-((2S,5R)-2,5-diethyl-4-(1-(6-methoxypyridin-2-yl)ethyl)piperazin-1-yl)-1-methyl-2-oxo-1,2-dihydropyrido[3,2-d]pyrimidine-6-carbonitrile (214-215); 4-((2S,5R)-4-(1-(4-(cyclopropylmethoxy)phenyl)propyl)-2,5-diethylpiperazin-1-yl)-1-methyl-2-oxo-1,2-dihydropyrido[3,2-d]pyrimidine-6-carbonitrile (216-217); 4-((2S,5R)-2,5-diethyl-4-(1-(4-(1-methylcyclopropyl)phenyl)ethyl)piperazin-1-yl)-1-methyl-2-oxo-1,2-dihydropyrido[3,2-d]pyrimidine-6-carbonitrile (218-219); 4-((2S,5R)-4-(1-(4-cyanophenyl)propyl)-2,5-diethylpiperazin-1-yl)-1-methyl-2-oxo-1,2-dihydropyrido[3,2-d] pyrimidine-6-carbonitrile (220-221); 4-((2S,5R)-4-(1-(4-(difluoromethoxy)phenyl) propyl)-2,5-diethylpiperazin-1-yl)-1-methyl-2-oxo-1,2-dihydropyrido[3,2-d]pyrimidine-6-carbonitrile (222-223); 4-((2S,5R)-2,5-diethyl-4-(1-(6-(trifluoromethyl)pyridin-3-yl) propyl)piperazin-1-yl)-1-methyl-2-oxo-1,2-dihydropyrido[3,2-d]pyrimidine-6-carbonitrile (224-225); 4-((2S,5R)-2,5-diethyl-4-(1-(2-(trifluoromethyl)pyrimidin-5-yl)ethyl) piperazin-1-yl)-1-methyl-2-oxo-1,2-dihydropyrido[3,2-d]pyrimidine-6-carbonitrile (226-227); 4-((2S,5R)-2,5-diethyl-4-(1-(6-methylpyridin-3-yl)ethyl)piperazin-1-yl)-1-methyl-2-oxo-1,2-dihydropyrido[3,2-d]pyrimidine-6-carbonitrile (228-229); 4-((2S,5R)-2,5-diethyl-4-(1-(4-(2-oxopyrrolidin-1-yl)phenyl)ethyl)piperazin-1-yl)-1-methyl-2-oxo-1,2-dihydropyrido[3,2-d]pyrimidine-6-carbonitrile (230-231); 4-((2S,5R)-4-(1-(4-(difluoromethoxy)-2-fluorophenyl)propyl)-2,5-diethylpiperazin-1-yl)-1-methyl-2-oxo-1,2-dihydropyrido[3,2-d]pyrimidine-6-carbonitrile (232-233); 4-((2S,5R)-2,5-diethyl-4-(1-(4-isopropoxyphenyl)propyl)piperazin-1-yl)-1-methyl-2-oxo-1,2-dihydropyrido[3,2-d] pyrimidine-6-carbonitrile (234-235); 4-((2S,5R)-2,5-diethyl-4-(1-(p-tolyl)propyl) piperazin-1-yl)-1-methyl-2-oxo-1,2-dihydropyrido[3,2-d]pyrimidine-6-carbonitrile (236-237); 4-((2S,5R)-4-(1-(4-chloro-2-fluorophenyl)propyl)-2,5-diethylpiperazin-1-yl)-1-methyl-2-oxo-1,2-dihydropyrido[3,2-d]pyrimidine-6-carbonitrile (238-239); 4-((2S,5R)-4-(1-(6-(difluoromethoxy)pyridin-2-yl)ethyl)-2,5-diethylpiperazin-1-yl)-1-methyl-2-oxo-1,2-dihydropyrido[3,2-d]pyrimidine-6-carbonitrile (240-241); 4-((2S,5R)-2,5-diethyl-4-(1-(4-(trifluoromethyl)phenyl)butyl)piperazin-1-yl)-1-methyl-2-oxo-1,2-dihydropyrido[3,2-d]pyrimidine-6-carbonitrile (243-244); 4-((2S,5R)-2,5-diethyl-4-(1-(6-(trifluoromethoxy)pyridin-2-yl)ethyl)piperazin-1-yl)-1-methyl-2-oxo-1,2-dihydropyrido[3,2-d]pyrimidine-6-carbonitrile (245-246); 4-((2S,5R)-2,5-diethyl-4-(1-(4-(2-morpholinopropan-2-yl)phenyl)ethyl)piperazin-1-yl)-1-methyl-2-oxo-1,2-dihydropyrido[3,2-d]pyrimidine-6-carbonitrile (247-248); 4-((2S,5R)-2,5-diethyl-4-(1-(4-methoxyphenyl)-2-methylpropyl)piperazin-1-yl)-1-methyl-2-oxo-1,2-dihydropyrido[3,2-d]pyrimidine-6-carbonitrile (249-250); 4-((2S,5R)-4-(1-(4-(2-cyanopropan-2-yl)phenyl) propyl)-2,5-diethylpiperazin-1-yl)-1-methyl-2-oxo-1,2-dihydropyrido[3,2-d]pyrimidine-6-carbonitrile (251-252); 4-((2S,5R)-4-(1-(2-cyclopropylbenzo[d]oxazol-5-yl)ethyl)-2,5-diethylpiperazin-1-yl)-1-methyl-2-oxo-1,2-dihydropyrido[3,2-d]pyrimidine-6-carbonitrile (253-254); 4-((2S,5R)-4-(1-(4-cyclopropoxyphenyl)propyl)-2,5-diethylpiperazin-1-yl)-1-methyl-2-oxo-1,2-dihydropyrido[3,2-d]pyrimidine-6-carbonitrile (255-256); ethyl (1S,2S)-2-(4-(1-((2R,5S)-4-(6-cyano-1-methyl-2-oxo-1,2-dihydropyrido[3,2-d]pyrimidin-4-yl)-2,5-diethylpiperazin-1-yl)ethyl)phenoxy)cyclopropane-1-carboxylate (259-260); 4-((2S,5R)-2,5-diethyl-4-(1-(4-isopropoxyphenyl)-2-methylpropyl)piperazin-1-yl)-1-methyl-2-oxo-1,2-dihydropyrido[3,2-d]pyrimidine-6-carbonitrile (261-262); 4-((2S,5R)-4-(1-(4-(1-cyanocyclopropyl)phenyl)ethyl)-2,5-diethylpiperazin-1-yl)-1-methyl-2-oxo-1,2-dihydropyrido[3,2-d]pyrimidine-6-carbonitrile (263-264); methyl 4-(1-((2R,5S)-4-(6-cyano-1-methyl-2-oxo-1,2-dihydropyrido[3,2-d]pyrimidin-4-yl)-2,5-diethylpiperazin-1-yl)ethyl)benzoate (265-266); 4-((2S,5R)-2,5-diethyl-4-(1-(4-(morpholinomethyl)phenyl) propyl)piperazin-1-yl)-1-methyl-2-oxo-1,2-dihydropyrido[3,2-d]pyrimidine-6-carbonitrile (267-268); 4-((2S,5R)-2,5-diethyl-4-(1-(4-(hydroxymethyl)phenyl)ethyl)piperazin-1-yl)-1-methyl-2-oxo-1,2-dihydropyrido[3,2-d]pyrimidine-6-carbonitrile (270-271); 4-((2S,5R)-4-(1-(4-(bromomethyl)phenyl)ethyl)-2,5-diethylpiperazin-1-yl)-1-methyl-2-oxo-1,2-dihydropyrido[3,2-d]pyrimidine-6-carbonitrile (272); 4-((2S,5R)-2,5-diethyl-4-(1-(4-((4-methoxypiperidin-1-yl)methyl)phenyl)ethyl)piperazin-1-yl)-1-methyl-2-oxo-1,2-dihydropyrido[3,2-d]pyrimidine-6-carbonitrile (273-274); 4-((2S,5R)-4-(1-(4-((2,2-dimethylmorpholino)methyl)phenyl)ethyl)-2,5-diethylpiperazin-1-yl)-1-methyl-2-oxo-1,2-dihydropyrido[3,2-d]pyrimidine-6-carbonitrile (275-276); 4-((2S,5R)-4-(1-(4-((4,4-difluoropiperidin-1-yl)methyl)phenyl)ethyl)-2,5-diethylpiperazin-1-yl)-1-methyl-2-oxo-1,2-dihydropyrido[3,2-d]pyrimidine-6-carbonitrile (277-278); 4-((2S,5R)-4-(1-(4-((2-oxa-6-azaspiro[3.3]heptan-6-yl)methyl)phenyl)ethyl)-2,5-diethylpiperazin-1-yl)-1-methyl-2-oxo-1,2-dihydropyrido[3,2-d]pyrimidine-6-carbonitrile (279-280); 4-((2S,5R)-2,5-diethyl-4-(1-(4-(piperidin-1-ylmethyl)phenyl)ethyl)piperazin-1-yl)-1-methyl-2-oxo-1,2-dihydropyrido[3,2-d]pyrimidine-6-carbonitrile (281-282); 4-((2S,5R)-4-(1-(4-((4-acetylpiperazin-1-yl)methyl)phenyl)ethyl)-2,5-diethylpiperazin-1-yl)-1-methyl-2-oxo-1,2-dihydropyrido[3,2-d]pyrimidine-6-carbonitrile (283-284); 4-((2S,5R)-2,5-diethyl-4-(1-(4-((4-hydroxypiperidin-1-yl)methyl)phenyl)ethyl)piperazin-1-yl)-1-methyl-2-oxo-1,2-dihydropyrido[3,2-d]pyrimidine-6-carbonitrile (285-286); 4-((2S,5R)-2,5-diethyl-4-(1-(4-((4-methyl-3-oxopiperazin-1-yl)methyl)phenyl)ethyl)piperazin-1-yl)-1-methyl-2-oxo-1,2-dihydropyrido[3,2-d]pyrimidine-6-carbonitrile (287-288); 4-((2S,5R)-2,5-diethyl-4-(1-(4-(((R)-3-hydroxypiperidin-1-yl)methyl)phenyl)ethyl)piperazin-1-yl)-1-methyl-2-oxo-1,2-dihydropyrido[3,2-d]pyrimidine-6-carbonitrile (289-290); 4-((2S,5R)-4-(1-(4-(((2S,6R)-2,6-dimethylmorpholino)methyl)phenyl)ethyl)-2,5-diethylpiperazin-1-yl)-1-methyl-2-oxo-1,2-dihydropyrido[3,2-d]pyrimidine-6-carbonitrile (291-292); 4-((2S,5R)-5-ethyl-2-methyl-4-(1-(3-methyl-2-oxo-2,3-dihydrobenzo[d]oxazol-5-yl)ethyl)piperazin-1-yl)-1-methyl-2-oxo-1,2-dihydropyrido[3,2-d]pyrimidine-6-carbonitrile (298-299); 4-((2S,5R)-4-(1-(4-cyclopropylphenyl)propyl)-5-ethyl-2-methylpiperazin-1-yl)-1-methyl-2-oxo-1,2-dihydropyrido[3,2-d]pyrimidine-6-carbonitrile (300-301); 4-((2S,5R)-4-(1-(4-cyclopropyl-2-fluorophenyl)propyl)-5-ethyl-2-methylpiperazin-1-yl)-1-methyl-2-oxo-1,2-dihydropyrido[3,2-d]pyrimidine-6-carbonitrile (302-303); 4-((2S,5R)-5-ethyl-4-(1-(4-methoxyphenyl)propyl)-2-methylpiperazin-1-yl)-1-methyl-2-oxo-1,2-dihydropyrido[3,2-d]pyrimidine-6-carbonitrile (304-305); 4-((2S,5R)-4-(1-(4-ethoxyphenyl)propyl)-5-ethyl-2-methylpiperazin-1-yl)-1-methyl-2-oxo-1,2-dihydropyrido[3,2-d]pyrimidine-6-carbonitrile (306-307); 4-((2S,5R)-4-(1-(4-(cyclopropylmethoxy)phenyl)propyl)-5-ethyl-2-methylpiperazin-1-yl)-1-methyl-2-oxo-1,2-dihydropyrido[3,2-d]pyrimidine-6-carbonitrile (308-309); 4-((2S,5R)-4-(1-(4-cyclopropoxyphenyl)propyl)-5-ethyl-2-methylpiperazin-1-yl)-1-methyl-2-oxo-1,2-dihydropyrido[3,2-d]pyrimidine-6-carbonitrile (311-312); 4-((2S,5R)-5-ethyl-4-(1-(4-methoxyphenyl)-2-methylpropyl)-2-methylpiperazin-1-yl)-1-methyl-2-oxo-1,2-dihydropyrido[3,2-d]pyrimidine-6-carbonitrile (313-314); 4-((2S,5R)-4-(1-(4-(2-cyanopropan-2-yl)phenyl)propyl)-5-ethyl-2-methylpiperazin-1-yl)-1-methyl-2-oxo-1,2-dihydropyrido[3,2-d]pyrimidine-6-carbonitrile (317-318); 4-((2S,5R)-4-(1-(3,4-difluorophenyl)propyl)-5-ethyl-2-methylpiperazin-1-yl)-1-methyl-2-oxo-1,2-dihydropyrido[3,2-d]pyrimidine-6-carbonitrile (319-320); 4-((2S,5R)-4-(1-(4-bromophenyl)propyl)-5-ethyl-2-methylpiperazin-1-yl)-1-methyl-2-oxo-1,2-dihydropyrido[3,2-d]pyrimidine-6-carbonitrile (321-322); 4-((2S,5R)-5-ethyl-4-(1-(4-isopropoxyphenyl)-2-methylpropyl)-2-methylpiperazin-1-yl)-1-methyl-2-oxo-1,2-dihydropyrido[3,2-d]pyrimidine-6-carbonitrile (323-324); 4-((2S,5R)-4-(1-(4-(1-cyanocyclopropyl)phenyl)ethyl)-5-ethyl-2-methylpiperazin-1-yl)-1-methyl-2-oxo-1,2-dihydropyrido[3,2-d]pyrimidine-6-carbonitrile (325-326); 4-((2S,5R)-4-(1-(4-(cyclopropylmethoxy)-2,6-difluorophenyl)propyl)-5-ethyl-2-methylpiperazin-1-yl)-1-methyl-2-oxo-1,2-dihydropyrido[3,2-d]pyrimidine-6-carbonitrile (327-328); 4-((2S,5R)-5-ethyl-2-methyl-4-(1-(4-(tetrahydro-2H-pyran-4-yl)phenyl)ethyl)piperazin-1-yl)-1-methyl-2-oxo-1,2-dihydropyrido[3,2-d]pyrimidine-6-carbonitrile (329-330); 4-((2S,5R)-4-(1-(4-(1,3-dioxolan-2-yl)phenyl)propyl)-5-ethyl-2-methylpiperazin-1-yl)-1-methyl-2-oxo-1,2-dihydropyrido[3,2-d]pyrimidine-6-carbonitrile (331-332); 4-((2S,5R)-5-ethyl-4-(1-(4-isopropoxyphenyl)propyl)-2-methylpiperazin-1-yl)-1-methyl-2-oxo-1,2-dihydropyrido[3,2-d]pyrimidine-6-carbonitrile (330-334); 4-((2S,5R)-5-ethyl-2-methyl-4-(1-(4-(3,3,3-trifluoropropoxy)phenyl)propyl)piperazin-1-yl)-1-methyl-2-oxo-1,2-dihydropyrido[3,2-d]pyrimidine-6-carbonitrile (335-336); 4-((2S,5R)-5-ethyl-2-methyl-4-(1-(4-((tetrahydro-2H-pyran-4-yl)methoxy)phenyl)propyl)piperazin-1-yl)-1-methyl-2-oxo-1,2-dihydropyrido[3,2-d]pyrimidine-6-carbonitrile (337-338); 4-((2S,5R)-4-(1-(4-(2-cyclopropylethoxy)phenyl)propyl)-5-ethyl-2-methylpiperazin-1-yl)-1-methyl-2-oxo-1,2-dihydropyrido[3,2-d]pyrimidine-6-carbonitrile (339-340); 4-((2S,5R)-5-ethyl-2-methyl-4-(1-(4-(oxetan-3-ylmethoxy)phenyl)propyl)piperazin-1-yl)-1-methyl-2-oxo-1,2-dihydropyrido[3,2-d]pyrimidine-6-carbonitrile (341-342); 4-((2S,5R)-5-ethyl-2-methyl-4-(1-(4-((1-methylazetidin-3-yl)methoxy)phenyl)propyl)piperazin-1-yl)-1-methyl-2-oxo-1,2-dihydropyrido[3,2-d]pyrimidine-6-carbonitrile (343-344); 4-((2S,5R)-5-ethyl-2-methyl-4-(1-(4-((1-methylcyclopropyl)methoxy)phenyl)propyl)piperazin-1-yl)-1-methyl-2-oxo-1,2-dihydropyrido[3,2-d]pyrimidine-6-carbonitrile (345-346); 4-((2S,5R)-5-ethyl-2-methyl-4-(1-(4-(thiazol-2-ylmethoxy)phenyl)propyl)piperazin-1-yl)-1-methyl-2-oxo-1,2-dihydropyrido[3,2-d]pyrimidine-6-carbonitrile (347-348); 4-((2S,5R)-4-(1-(3-bromo-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile (349); 4-((2S,5R)-5-ethyl-2-methyl-4-(1-(3-(morpholinomethyl)-4-(trifluoromethyl)phenyl) ethyl)piperazin-1-yl)-1-methyl-2-oxo-1,2-dihydropyrido[3,2-d]pyrimidine-6-carbonitrile (350-351); 4-((2S,5R)-4-(1-(3-((dimethylamino)methyl)-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile (352-353); 4-((2S,5R)-5-ethyl-2-methyl-4-(1-(3-(piperidin-1-ylmethyl)-4-(trifluoromethyl)phenyl) ethyl)piperazin-1-yl)-1-methyl-2-oxo-1,2-dihydropyrido[3,2-d]pyrimidine-6-carbonitrile (354-355); 4-((2S,5R)-4-(1-(3-cyano-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile (356-357); 4-((2S,5R)-4-(1-(4-(aminomethyl)phenyl)ethyl)-5-ethyl-2-methylpiperazin-1-yl)-1-methyl-2-oxo-1,2-dihydropyrido[3,2-d]pyrimidine-6-carbonitrile (358); methyl (4-(1-((2R,5S)-4-(6-cyano-1-methyl-2-oxo-1,2-dihydropyrido[3,2-d]pyrimidin-4-yl)-2-ethyl-5-methylpiperazin-1-yl)ethyl)benzyl)carbamate (359-360); 4-((2S,5R)-4-(1-(3-cyclopropyl-1,2,4-oxadiazol-5-yl)ethyl)-2,5-diethylpiperazin-1-yl)-1-methyl-2-oxo-1,2-dihydropyrido[3,2-d]pyrimidine-6-carbonitrile (361-362); 4-((2S,5R)-4-(1-(3-cyclopropyl-1,2,4-oxadiazol-5-yl)propyl)-2,5-diethylpiperazin-1-yl)-1-methyl-2-oxo-1,2-dihydropyrido[3,2-d]pyrimidine-6-carbonitrile (363-364); 4-((2S,5R)-4-(1-(3-cyclopropyl-1,2,4-oxadiazol-5-yl)-2-methylpropyl)-5-ethyl-2-methylpiperazin-1-yl)-1-methyl-2-oxo-1,2-dihydropyrido[3,2-d]pyrimidine-6-carbonitrile (365-366); 4-((2S,5R)-4-(1-(5-cyclopropyl-1,3,4-oxadiazol-2-yl)-2-methylpropyl)-5-ethyl-2-methylpiperazin-1-yl)-1-methyl-2-oxo-1,2-dihydropyrido[3,2-d]pyrimidine-6-carbonitrile (367-368); 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)acetonitrile (369); 4-((2S,5R)-4-(1-(4-((2-cyanopropan-2-yl)oxy)phenyl)ethyl)-2,5-dimethylpiperazin-1-yl)-1-methyl-2-oxo-1,2-dihydropyrido[3,2-d]pyrimidine-6-carbonitrile (376-377); 4-((2S,5R)-4-(1-(4-cyclopropylphenyl)propyl)-2,5-dimethylpiperazin-1-yl)-1-methyl-2-oxo-1,2-dihydropyrido[3,2-d]pyrimidine-6-carbonitrile (378-379); 4-((2S,5R)-4-(1-(4-cyclopropyl-2-fluorophenyl)propyl)-2,5-dimethylpiperazin-1-yl)-1-methyl-2-oxo-1,2-dihydropyrido[3,2-d]pyrimidine-6-carbonitrile (380-381); 6-chloro-4-((2S,5R)-4-(1-(4-(hydroxymethyl)phenyl)ethyl)-2,5-dimethylpiperazin-1-yl)-1-methylpyrido[3,2-d] pyrimidin-2(1H)-one (382); 4-((2S,5R)-4-(1-(4-(hydroxymethyl)phenyl)ethyl)-2,5-dimethylpiperazin-1-yl)-1-methyl-2-oxo-1,2-dihydropyrido[3,2-d]pyrimidine-6-carbonitrile (383); 4-((2S,5R)-4-(1-(4-(bromomethyl)phenyl)ethyl)-2,5-dimethylpiperazin-1-yl)-1-methyl-2-oxo-1,2-dihydropyrido[3,2-d]pyrimidine-6-carbonitrile (384); 4-((2S,5R)-4-(1-(4-((2,2-dimethylmorpholino)methyl)phenyl)ethyl)-2,5-dimethylpiperazin-1-yl)-1-methyl-2-oxo-1,2-dihydropyrido[3,2-d]pyrimidine-6-carbonitrile (385-386); 4-((2S,5R)-4-(1-(4-(((1S,4S)-2-oxa-5-azabicyclo[2.2.1]heptan-5-yl)methyl)phenyl)ethyl)-2,5-dimethylpiperazin-1-yl)-1-methyl-2-oxo-1,2-dihydropyrido[3,2-d]pyrimidine-6-carbonitrile (387-388); 4-((2S,5R)-4-(1-(4-(((2S,6R)-2,6-dimethylmorpholino)methyl)phenyl)ethyl)-2,5-dimethylpiperazin-1-yl)-1-methyl-2-oxo-1,2-dihydropyrido[3,2-d]pyrimidine-6-carbonitrile (389); 4-((2S,5R)-4-(1-(4-((4,4-difluoropiperidin-1-yl)methyl)phenyl)ethyl)-2,5-dimethylpiperazin-1-yl)-1-methyl-2-oxo-1,2-dihydropyrido[3,2-d]pyrimidine-6-carbonitrile (390-391); 4-((2S,5R)-4-(1-(4-((2-oxa-6-azaspiro[3.3]heptan-6-yl)methyl)phenyl)ethyl)-2,5-dimethylpiperazin-1-yl)-1-methyl-2-oxo-1,2-dihydropyrido[3,2-d]pyrimidine-6-carbonitrile (392-393); 6-chloro-1-methyl-4-((2S,5R)-2-methyl-5-propyl-4-(1-(4-(trifluoromethyl)phenyl)ethyl)piperazin-1-yl) pyrido[3,2-d]pyrimidin-2(1H)-one (394); 1-methyl-4-((2S,5R)-2-methyl-5-propyl-4-(1-(4-(trifluoromethyl)phenyl)ethyl)piperazin-1-yl)-2-oxo-1,2-dihydropyrido[3,2-d] pyrimidine-6-carbonitrile (395-396); 6-chloro-4-((2S,5S)-5-(methoxymethyl)-2-methyl-4-(1-(4-(trifluoromethyl)phenyl) propyl)piperazin-1-yl)-1-methylpyrido[3,2-d]pyrimidin-2(1H)-one (397); 4-((2S,5S)-5-(methoxymethyl)-2-methyl-4-(1-(4-(trifluoromethyl) phenyl)propyl) piperazin-1-yl)-1-methyl-2-oxo-1,2-dihydropyrido[3,2-d]pyrimidine-6-carbonitrile (398-399); 4-((2S,5S)-5-(ethoxymethyl)-2-methyl-4-(1-(4-(trifluoromethyl) phenyl)propyl)piperazin-1-yl)-1-methyl-2-oxo-1,2-dihydropyrido[3,2-d]pyrimidine-6-carbonitrile (400-401); 4-((2S,5S)-5-(azidomethyl)-2-methyl-4-(1-(4-(trifluoromethyl) phenyl)ethyl)piperazin-1-yl)-6-chloro-1-methylpyrido[3,2-d]pyrimidin-2(1H)-one (402); 4-((2S,5R)-5-(aminomethyl)-2-methyl-4-(1-(4-(trifluoromethyl)phenyl)ethyl)piperazin-1-yl)-6-chloro-1-methylpyrido[3,2-d]pyrimidin-2(1H)-one (403); 4-((2S,5S)-5-(methoxymethyl)-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile (404-405); 4-((2R,5R)-2-(hydroxymethyl)-5-methyl-4-(1-(4-(trifluoromethyl)phenyl)ethyl) piperazin-1-yl)-1-methyl-2-oxo-1,2-dihydropyrido[3,2-d]pyrimidine-6-carbonitrile (406-407); 6-chloro-4-((2R,5R)-2-(methoxymethyl)-5-methyl-4-(1-(4-(trifluoromethyl)phenyl)ethyl) piperazin-1-yl)-1-methylpyrido[3,2-d]pyrimidin-2(1H)-one (408); 4-((2R,5R)-2-(methoxymethyl)-5-methyl-4-(1-(4-(trifluoromethyl)phenyl)ethyl) piperazin-1-yl)-1-methyl-2-oxo-1,2-dihydropyrido[3,2-d]pyrimidine-6-carbonitrile (409-410); 4-((2R,5R)-5-ethyl-2-(hydroxymethyl)-4-(1-(4-(trifluoromethyl)phenyl)ethyl)piperazin-1-yl)-1-methyl-2-oxo-1,2-dihydropyrido[3,2-d]pyrimidine-6-carbonitrile (411-412); 4-((2S,5R)-5-ethyl-2-methyl-4-(1-(4-(trifluoromethyl)phenyl)ethyl)piperazin-1-yl)-2-oxo-1,2-dihydropyrido[3,2-d]pyrimidine-6-carbonitrile (413-414); 4-((2S,5R)-2,5-diethyl-4-(1-(4-(trifluoromethyl)phenyl)propyl)piperazin-1-yl)-2-oxo-1,2-dihydropyrido[3,2-d] pyrimidine-6-carbonitrile (415-416); 6-chloro-4-((2S,5R)-5-ethyl-2-methyl-4-(1-(4-(trifluoromethyl)phenyl)ethyl)piperazin-1-yl)-1-(methyl-d₃)pyrido[3,2-d]pyrimidin-2(1H)-one (417-418); 4-((2S,5R)-5-ethyl-2-methyl-4-(1-(4-(trifluoromethyl)phenyl) ethyl)piperazin-1-yl)-1-(methyl-d₃)-2-oxo-1,2-dihydropyrido[3,2-d]pyrimidine-6-carbonitrile (419-420); 6-chloro-4-((2S,5R)-2,5-diethyl-4-(1-(4-(trifluoromethyl)phenyl) propyl)piperazin-1-yl)-1-methylpyrido[3,2-d]pyrimidin-2(1H)-one (421-422); 4-((2S,5R)-2,5-diethyl-4-(1-(4-(trifluoromethyl)phenyl)ethyl)piperazin-1-yl)-6-(hydroxymethyl)-1-methylpyrido[3,2-d]pyrimidin-2(1H)-one (423); 4-((2S,5R)-2,5-diethyl-4-(1-(4-(trifluoromethyl)phenyl)ethyl)piperazin-1-yl)-6-(methoxymethyl)-1-methylpyrido[3,2-d] pyrimidin-2(1H)-one (424-425); 6-chloro-4-((2S,5R)-2,5-diethyl-4-(1-(4-(trifluoromethyl) phenyl)ethyl)piperazin-1-yl)-1-methylpyrido[3,2-d]pyrimidin-2(1H)-one (426); 4-((2S,5R)-2,5-diethyl-4-(1-(4-(trifluoromethyl)phenyl)ethyl)piperazin-1-yl)-6-methoxy-1-methylpyrido[3,2-d]pyrimidin-2(1H)-one (427-428); 4-((2S,5R)-2,5-diethyl-4-(1-(4-(trifluoromethyl)phenyl)ethyl)piperazin-1-yl)-6-ethoxy-1-methylpyrido[3,2-d]pyrimidin-2(1H)-one (429-430); 4-((2S,5R)-2,5-diethyl-4-(1-(4-(trifluoromethyl)phenyl)ethyl) piperazin-1-yl)-6-(2-(dimethylamino)ethoxy)-1-methylpyrido[3,2-d]pyrimidin-2(1H)-one (431-432); 4-((2S,5R)-2,5-diethyl-4-(1-(4-(trifluoromethyl)phenyl)ethyl)piperazin-1-yl)-6-(2-methoxyethoxy)-1-methylpyrido[3,2-d]pyrimidin-2(1H)-one (433-434); 4-((2S,5R)-2,5-diethyl-4-(1-(4-(trifluoromethyl)phenyl)propyl)piperazin-1-yl)-6-methoxy-1-methylpyrido[3,2-d]pyrimidin-2(1H)-one (435-436); 4-((2S,5R)-2,5-diethyl-4-(1-(4-(trifluoromethyl)phenyl)propyl)piperazin-1-yl)-6-ethoxy-1-methylpyrido[3,2-d] pyrimidin-2(1H)-one (437-438); 4-((2S,5R)-2,5-diethyl-4-(1-(4-(trifluoromethyl)phenyl) ethyl)piperazin-1-yl)-6-(difluoromethyl)-1-methylpyrido[3,2-d]pyrimidin-2(1H)-one (439-440); 6-(difluoromethyl)-4-((2S,5R)-5-ethyl-2-methyl-4-(1-(4-(trifluoromethyl) phenyl)ethyl)piperazin-1-yl)-1-methylpyrido[3,2-d]pyrimidin-2(1H)-one (441-442); 4-((2S,5R)-2,5-diethyl-4-(1-(4-(trifluoromethyl)phenyl)ethyl)piperazin-1-yl)-6-hydroxy-1-methylpyrido[3,2-d]pyrimidin-2(1H)-one (443); 4-((2S,5R)-2,5-diethyl-4-(1-(4-(trifluoromethyl)phenyl)ethyl)piperazin-1-yl)-6-(difluoromethoxy)-1-methylpyrido[3,2-d] pyrimidin-2(1H)-one (444-445); 6-chloro-4-((2S,5R)-2,5-dimethyl-4-(1-(3-(trifluoromethyl)bicyclo[1.1.1]pentan-1-yl)propyl)piperazin-1-yl)-1-methylpyrido[3,2-d] pyrimidin-2(1H)-one (diastereomeric mixture) (446); 4-((2S,5R)-2,5-dimethyl-4-(1-(3-(trifluoromethyl)bicyclo[1.1.1]pentan-1-yl)propyl) piperazin-1-yl)-1-methyl-2-oxo-1,2-dihydropyrido[3,2-d]pyrimidine-6-carbonitrile (447-449); 4-((2S,5R)-4-(1-cyclopropylpropyl)-2,5-dimethylpiperazin-1-yl)-1-methyl-2-oxo-1,2-dihydropyrido[3,2-d]pyrimidine-6-carbonitrile (453-455); 4-((2S,5R)-4-(1-(3,3-difluorocyclobutyl)propyl)-2,5-dimethylpiperazin-1-yl)-1-methyl-2-oxo-1,2-dihydropyrido[3,2-d]pyrimidine-6-carbonitrile (456-458); or 4-((2S,5R)-4-(1-(4,4-difluorocyclohexyl)propyl)-2,5-dimethylpiperazin-1-yl)-1-methyl-2-oxo-1,2-dihydropyrido[3,2-d]pyrimidine-6-carbonitrile (459-460).

In one embodiment, a compound of Formula (I) or a salt thereof is provided wherein said compound is:

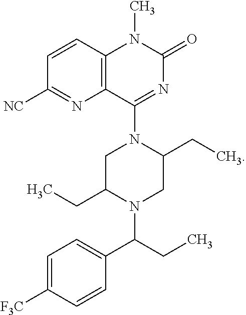 [SMILES: CCC1CN(C(CC)c2ccc(C(F)(F)F)cc2)C(CC)CN1c1nc(=O)n(C)c2ccc(C#N)nc12]

In one embodiment, a compound of Formula (I) or a salt thereof is provided wherein said compound is 4-((2S,5R)-2,5-diethyl-4-(1-(4-(trifluoromethyl)phenyl)propyl) piperazin-1-yl)-1-methyl-2-oxo-1,2-dihydropyrido[3,2-d]pyrimidine-6-carbonitrile (5-6).

In one embodiment, a compound of Formula (I) or a salt thereof is provided wherein said compound is 4-((2S,5R)-2,5-diethyl-4-((S)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-2,5-diethyl-4-((R)-1-(4-(trifluoromethyl)phenyl) propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

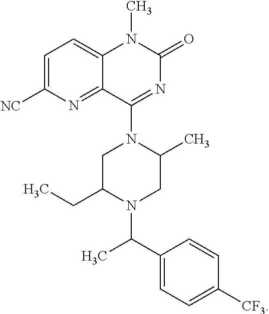 [SMILES: CCC1CN(c2nc(=O)n(C)c3ccc(C#N)nc23)C(C)CN1C(C)c1ccc(C(F)(F)F)cc1]

In one embodiment, a compound of Formula (I) or a salt thereof is provided wherein said compound is 4-((2S,5R)-5-ethyl-2-methyl-4-(1-(4-(trifluoromethyl)phenyl) ethyl)piperazin-1-yl)-1-methyl-2-oxo-1,2-dihydropyrido[3,2-d]pyrimidine-6-carbonitrile (7-8).

In one embodiment, a compound of Formula (I) or a salt thereof is provided wherein said compound is 4-((2S,5R)-5-ethyl-2-methyl-4-((S)-1-(4-(trifluoromethyl) phenyl)ethyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-5-ethyl-2-methyl-4-((R)-1-(4-(trifluoromethyl) phenyl)ethyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

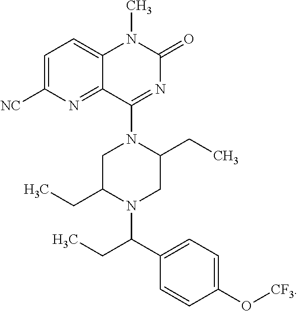 [SMILES: CCC1CN(C(CC)c2ccc(OC(F)(F)F)cc2)C(CC)CN1c1nc(=O)n(C)c2ccc(C#N)nc12]

In one embodiment, a compound of Formula (I) or a salt thereof is provided wherein said compound is 4-((2S,5R)-2,5-diethyl-4-(1-(4-(trifluoromethoxy)phenyl) propyl)piperazin-1-yl)-1-methyl-2-oxo-1,2-dihydropyrido[3,2-d]pyrimidine-6-carbonitrile (36-37).

In one embodiment, a compound of Formula (I) or a salt thereof is provided wherein said compound is 4-((2S,5R)-2,5-diethyl-4-((S)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-2,5-diethyl-4-((R)-1-(4-(trifluoromethoxy)phenyl) propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

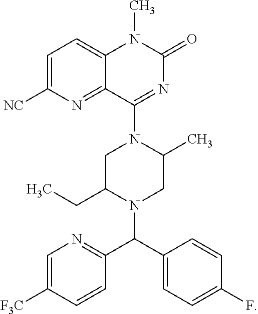 [SMILES: CCC1CN(c2nc(=O)n(C)c3ccc(C#N)nc23)C(C)CN1C(c1ccc(F)cc1)c1ccc(C(F)(F)F)cn1]

In one embodiment, a compound of Formula (I) or a salt thereof is provided wherein said compound is 4-((2S,5R)-5-ethyl-4-((4-fluorophenyl)(5-(trifluoromethyl) pyridin-2-yl)methyl)-2-methylpiperazin-1-yl)-1-methyl-2-oxo-1,2-dihydropyrido[3,2-d]pyrimidine-6-carbonitrile (46-47).

In one embodiment, a compound of Formula (I) or a salt thereof is provided wherein said compound is 4-((2S,5R)-5-ethyl-4-((S)-(4-fluorophenyl)(5-(trifluoromethyl) pyridin-2-yl)methyl)-2-methylpiperazin-1-yl)-1-methyl-2-oxo-1,2-dihydropyrido[3,2-d] pyrimidine-6-carbonitrile.

In one embodiment, a compound of Formula (I) or a salt thereof is provided wherein said compound is 4-((2S,5R)-5-ethyl-4-((R)-(4-fluorophenyl)(5-(trifluoromethyl) pyridin-2-yl)methyl)-2-methylpiperazin-1-yl)-1-methyl-2-oxo-1,2-dihydropyrido[3,2-d] pyrimidine-6-carbonitrile.

In one embodiment, a compound of Formula (I) or a salt thereof is provided wherein said compound is:

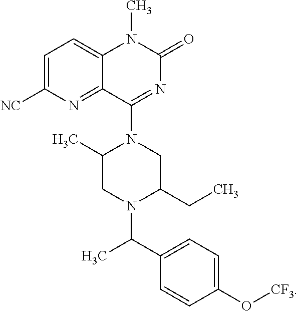 [SMILES: CCC1CN(c2nc(=O)n(C)c3ccc(C#N)nc23)C(C)CN1C(C)c1ccc(OC(F)(F)F)cc1]

In one embodiment, a compound of Formula (I) or a salt thereof is provided wherein said compound is 4-((2S,5R)-5-ethyl-2-methyl-4-(1-(4-(trifluoromethoxy) phenyl)ethyl)piperazin-1-yl)-1-methyl-2-oxo-1,2-dihydropyrido[3,2-d]pyrimidine-6-carbonitrile (48-49).

In one embodiment, a compound of Formula (I) or a salt thereof is provided wherein said compound is 4-((2S,5R)-5-ethyl-2-methyl-4-((S)-1-(4-(trifluoromethoxy) phenyl)ethyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-5-ethyl-2-methyl-4-((R)-1-(4-(trifluoromethoxy) phenyl)ethyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

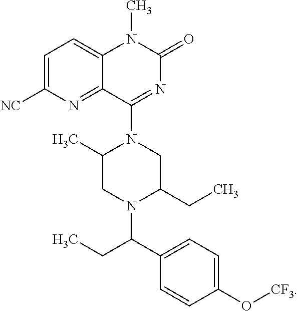 [SMILES: CCC1CN(c2nc(=O)n(C)c3ccc(C#N)nc23)C(C)CN1C(CC)c1ccc(OC(F)(F)F)cc1]

In one embodiment, a compound of Formula (I) or a salt thereof is provided wherein said compound is 4-((2S,5R)-5-ethyl-2-methyl-4-(1-(4-(trifluoromethoxy) phenyl)propyl)piperazin-1-yl)-1-methyl-2-oxo-1,2-dihydropyrido[3,2-d]pyrimidine-6-carbonitrile (83-84).

In one embodiment, a compound of Formula (I) or a salt thereof is provided wherein said compound is 4-((2S,5R)-5-ethyl-2-methyl-4-((S)-1-(4-(trifluoromethoxy) phenyl)propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-5-ethyl-2-methyl-4-((R)-1-(4-(trifluoromethoxy) phenyl)propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

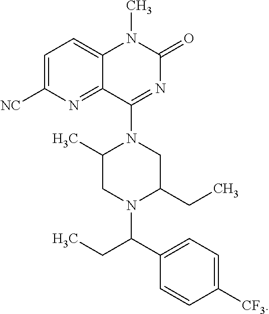 [SMILES: CCC1CN(c2nc(=O)n(C)c3ccc(C#N)nc23)C(C)CN1C(CC)c1ccc(C(F)(F)F)cc1]

In one embodiment, a compound of Formula (I) or a salt thereof is provided wherein said compound is 4-((2S,5R)-5-ethyl-2-methyl-4-(1-(4-(trifluoromethyl)phenyl) propyl)piperazin-1-yl)-1-methyl-2-oxo-1,2-dihydropyrido[3,2-d]pyrimidine-6-carbonitrile (89-90).

In one embodiment, a compound of Formula (I) or a salt thereof is provided wherein said compound is 4-((2S,5R)-5-ethyl-2-methyl-4-((S)-1-(4-(trifluoromethyl) phenyl)propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-5-ethyl-2-methyl-4-((R)-1-(4-(trifluoromethyl) phenyl)propyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

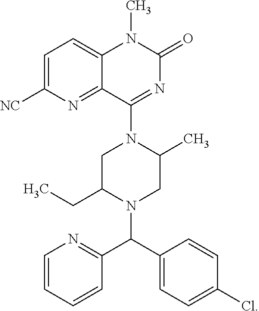 [SMILES: CCC1CN(c2nc(=O)n(C)c3ccc(C#N)nc23)C(C)CN1C(c1ccc(Cl)cc1)c1ccccn1]

In one embodiment, a compound of Formula (I) or a salt thereof is provided wherein said compound is 4-((2S,5R)-4-((4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile (95-96).

In one embodiment, a compound of Formula (I) or a salt thereof is provided wherein said compound is 4-((2S,5R)-4-((R)-(4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-4-((S)-(4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

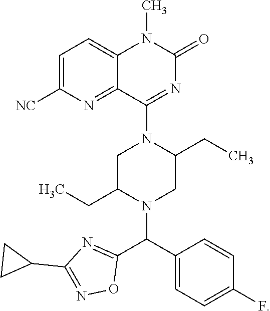 [SMILES: CCC1CN(C(c2ccc(F)cc2)c2nc(C3CC3)no2)C(CC)CN1c1nc(=O)n(C)c2ccc(C#N)nc12]

In one embodiment, a compound of Formula (I) or a salt thereof is provided wherein said compound is 4-((2S,5R)-4-((3-cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile (110-111).

In one embodiment, a compound of Formula (I) or a salt thereof is provided wherein said compound is 4-((2S,5R)-4-((R)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-4-((S)-(3-cyclopropyl-1,2,4-oxadiazol-5-yl)(4-fluorophenyl)methyl)-2,5-diethylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

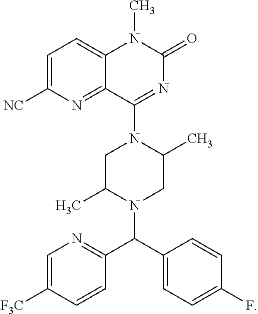 [SMILES: CC1CN(C(c2ccc(F)cc2)c2ccc(C(F)(F)F)cn2)C(C)CN1c1nc(=O)n(C)c2ccc(C#N)nc12]

In one embodiment, a compound of Formula (I) or a salt thereof is provided wherein said compound is 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile (126-127).

In one embodiment, a compound of Formula (I) or a salt thereof is provided wherein said compound is 4-((2S,5R)-4-((S)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d] pyrimidine-6-carbonitrile.

In one embodiment, a compound of Formula (I) or a salt thereof is provided wherein said compound is 4-((2S,5R)-4-((R)-(4-fluorophenyl)(5-(trifluoromethyl)pyridin-2-yl)methyl)-2,5-dimethylpiperazin-1-yl)-1-methyl-2-oxo-1,2-dihydropyrido[3,2-d] pyrimidine-6-carbonitrile.

In one embodiment, a compound of Formula (I) or a salt thereof is provided wherein said compound is:

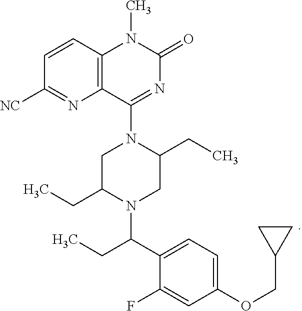 [SMILES: CCC1CN(C(CC)c2ccc(OCC3CC3)cc2F)C(CC)CN1c1nc(=O)n(C)c2ccc(C#N)nc12]

In one embodiment, a compound of Formula (I) or a salt thereof is provided wherein said compound is 4-((2S,5R)-4-(1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile (198-199).

In one embodiment, a compound of Formula (I) or a salt thereof is provided wherein said compound is 4-((2S,5R)-4-((S)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-4-((R)-1-(4-(cyclopropylmethoxy)-2-fluorophenyl) propyl)-2,5-diethylpiperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

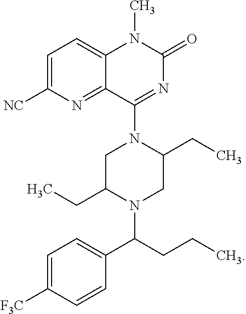 [SMILES: CCCC(c1ccc(C(F)(F)F)cc1)N1CC(CC)N(c2nc(=O)n(C)c3ccc(C#N)nc23)CC1CC]

In one embodiment, a compound of Formula (I) or a salt thereof is provided wherein said compound is 4-((2S,5R)-2,5-diethyl-4-(1-(4-(trifluoromethyl)phenyl) butyl)piperazin-1-yl)-1-methyl-2-oxo-1,2-dihydropyrido[3,2-d]pyrimidine-6-carbonitrile (243-244).

In one embodiment, a compound of Formula (I) or a salt thereof is provided wherein said compound is 4-((2S,5R)-2,5-diethyl-4-((S)-1-(4-(trifluoromethyl)phenyl) butyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is 4-((2S,5R)-2,5-diethyl-4-((R)-1-(4-(trifluoromethyl)phenyl) butyl)piperazin-1-yl)-1-methyl-2-oxo-1,2-dihydropyrido[3,2-d]pyrimidine-6-carbonitrile.

In one embodiment, a compound of Formula (I) or a salt thereof is provided wherein said compound is:

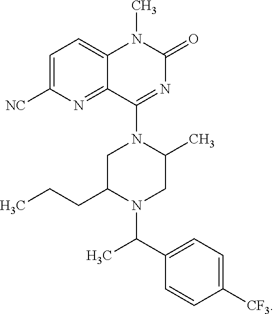 [SMILES: CCCC1CN(c2nc(=O)n(C)c3ccc(C#N)nc23)C(C)CN1C(C)c1ccc(C(F)(F)F)cc1]

In one embodiment, a compound of Formula (I) or a salt thereof is provided wherein said compound is 1-methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile (395-396).

In one embodiment, a compound of Formula (I) or a salt thereof is provided wherein said compound is 1-methyl-4-((2S,5R)-2-methyl-5-propyl-4-((S)-1-(4-(trifluoromethyl)phenyl)ethyl)piperazin-1-yl)-2-oxo-1,2-dihydropyrido[3,2-d]pyrimidine-6-carbonitrile.

In one embodiment, a compound of Formula (I) or a salt thereof is provided wherein said compound is 1-methyl-4-((2S,5R)-2-methyl-5-propyl-4-((R)-1-(4-(trifluoromethyl)phenyl)ethyl)piperazin-1-yl)-2-oxo-1,2-dihydropyrido[3,2-d]pyrimidine-6-carbonitrile.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

### Definitions

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof. Embodiments identified herein as exemplary or preferred are intended to be illustrative and not limiting.

Unless specifically stated otherwise herein, references made in the singular may also include the plural. For example, “a” and “an” may refer to either one, or one or more.

As used herein, the phrase “compounds and/or salts thereof” refers to at least one compound, at least one salt of the compounds, or a combination thereof. For example, compounds of Formula (I) and/or salts thereof includes a compound of Formula (I); two compounds of Formula (I); a salt of a compound of Formula (I); a compound of Formula (I) and one or more salts of the compound of Formula (I); and two or more salts of a compound of Formula (I).

Unless otherwise indicated, any atom with unsatisfied valences is assumed to have hydrogen atoms sufficient to satisfy the valences.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

Throughout the specification, groups and substituents thereof may be chosen by one skilled in the field to provide stable moieties and compounds.

In accordance with a convention used in the art,

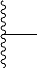

is used in structural formulas herein to depict the bond that is the point of attachment of the moiety or substituent to the core or backbone structure.

The terms “halo” and “halogen,” as used herein, refer to F, Cl, Br, and I.

The term “cyano” refers to the group —CN.

The term “amino” refers to the group —NH₂.

The term “azido” refers to the group —N₃.

The term “oxo” refers to the group ═O.

The term “alkyl” as used herein, refers to both branched and straight-chain saturated aliphatic hydrocarbon groups containing, for example, from 1 to 12 carbon atoms, from 1 to 6 carbon atoms, and from 1 to 4 carbon atoms. Examples of alkyl groups include, but are not limited to, methyl (Me), ethyl (Et), propyl (e.g., n-propyl and i-propyl), butyl (e.g., n-butyl, i-butyl, sec-butyl, and t-butyl), and pentyl (e.g., n-pentyl, isopentyl, neopentyl), n-hexyl, 2-methylpentyl, 2-ethylbutyl, 3-methylpentyl, and 4-methylpentyl. When numbers appear in a subscript after the symbol “C”, the subscript defines with more specificity the number of carbon atoms that a particular group may contain. For example, “C₁₋₄ alkyl” denotes straight and branched chain alkyl groups with one to four carbon atoms.

The term “fluoroalkyl” as used herein is intended to include both branched and straight-chain saturated aliphatic hydrocarbon groups substituted with one or more fluorine atoms. For example, “C₁₋₄ fluoroalkyl” is intended to include C₁, C₂, C₃, and C₄ alkyl groups substituted with one or more fluorine atoms. Representative examples of fluoroalkyl groups include, but are not limited to, —CF₃ and —CH₂CF₃.

The term “bromoalkyl” as used herein is intended to include both branched and straight-chain saturated aliphatic hydrocarbon groups substituted with one or more bromine atoms. For example, “C₁₋₄ bromoalkyl” is intended to include C₁, C₂, C₃, and C₄ alkyl groups substituted with one or more bromine atoms. Representative examples of fluoroalkyl groups include, but are not limited to, —CH₂Br and —CH₂CH₂Br.

The term “hydroxyalkyl” includes both branched and straight-chain saturated alkyl groups substituted with one or more hydroxyl groups. For example, “hydroxyalkyl” includes —CH₂OH, —CH₂CH₂OH, and C₁₋₄ hydroxyalkyl.

The term “cyanoalkyl” includes both branched and straight-chain saturated alkyl groups substituted with one or more cyano groups. For example, “cyanoalkyl” includes —CH₂CN, —CH₂CH₂CN, and C₁₋₃ cyanoalkyl.

The term “alkenyl” refers to a straight or branched chain hydrocarbon radical containing from 2 to 12 carbon atoms and at least one carbon-carbon double bond. Exemplary such groups include ethenyl or allyl. For example, “C₂₋₆ alkenyl” denotes straight and branched chain alkenyl groups with two to six carbon atoms.

The term “alkynyl” refers to a straight or branched chain hydrocarbon radical containing from 2 to 12 carbon atoms and at least one carbon to carbon triple bond. Exemplary such groups include ethynyl. For example, “C₂₋₆ alkynyl” denotes straight and branched chain alkynyl groups with two to six carbon atoms.

The term “cycloalkyl” as used herein, refers to a group derived from a non-aromatic monocyclic hydrocarbon molecule by removal of one hydrogen atom from a saturated ring carbon atom. Representative examples of cycloalkyl groups include, but are not limited to, cyclopropyl, cyclopentyl, and cyclohexyl. When numbers appear in a subscript after the symbol “C”, the subscript defines with more specificity the number of carbon atoms that a particular cycloalkyl group may contain. For example, “C₃₋₆ cycloalkyl” denotes cycloalkyl groups with three to six carbon atoms.

The term “fluorocycloalkyl” as used herein is intended to include a cycloalkyl group substituted with one or more fluorine atoms.

The term “carbocyclyl” as used herein, refers to a group derived from a non-aromatic monocyclic or polycyclic hydrocarbon molecule by removal of one hydrogen atom from a saturated ring carbon atom. Representative examples of carbocyclyl groups include, but are not limited to, cycloalkyls and bicycloalkyls such as bicyclo[1.1.1]pentanyl, bicyclohexanes, bicycloheptanes, and bicyclooctanes.

The term “alkoxy,” as used herein, refers to an alkyl group attached to the parent molecular moiety through an oxygen atom, for example, methoxy group (—OCH₃). For example, “C₁₋₃ alkoxy” denotes alkoxy groups with one to three carbon atoms.

The terms “fluoroalkoxy” and “—O(fluoroalkyl)” represent a fluoroalkyl group as defined above attached through an oxygen linkage (—O—). For example, “C₁₋₄ fluoroalkoxy” is intended to include C₁, C₂, C₃, and C₄ fluoroalkoxy groups.

The terms “cyanoalkoxy” and “—O(cyanoalkyl)” represent a cyanoalkyl group as defined above attached through an oxygen linkage (—O—). For example, “C₁₋₃ cyanoalkoxy” is intended to include C₁, C₂, and C₃ cyanoalkoxy groups.

The terms “carbocyclo”, “carbocyclic” or “carbocyclyl” may be used interchangeably and refer to cyclic groups having at least one saturated or partially saturated non-aromatic ring wherein all atoms of all rings are carbon. The carbocyclyl ring may be unsubstituted or may contain one or more substituents as valence allows. Thus, the term includes nonaromatic rings such as for example, cycloalkyl, cycloalkenyl, and cycloalkynyl rings. Exemplary bicyclic carbocyclyl groups include, indanyl, indenyl, dihydronaphthalenyl, tetrahydronaphthenyl, hexahydronaphthalenyl, octahydronaphthalenyl, decahydronaphthalenyl, bicycloheptanyl, bicyclooctanyl, and bicyclononanyl.

The term “aryl” as used herein, refers to a group of atoms derived from a molecule containing aromatic carbon ring(s) by removing one hydrogen that is bonded to the aromatic ring(s). Bicyclic aryl groups include aryl groups with two aromatic carbon rings and aryl groups with one aromatic carbon ring and one non-aromatic carbon ring. Representative examples of aryl groups include monocyclic aryl groups such as phenyl, and bicyclic aryl groups such as naphthalenyl, dihydronaphthalenyl, tetrahydronaphthalenyl, indenyl, and indanyl. The aryl ring may be unsubstituted or may contain one or more substituents as valence allows.

The term “benzyl,” as used herein, refers to a methyl group in which one of the hydrogen atoms is replaced by a phenyl group. The phenyl ring may be unsubstituted or may contain one or more substituents as valence allows.

The term “heteroatom” refers to oxygen (O), sulfur (S), and nitrogen (N).

The terms “heterocyclo”, “heterocyclic”, or “heterocyclyl” may be used interchangeably and refer to cyclic groups having saturated or partially saturated non-aromatic ring(s) and wherein one or more of the rings have at least one heteroatom (O, S or N), said heteroatom containing ring preferably having 1 to 4 heteroatoms independently selected from O, S, and/or N. The ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The heterocyclo group may be attached at any available nitrogen or carbon atom. The heterocyclo ring may be unsubstituted or may contain one or more substituents as valence allows.

Exemplary monocyclic heterocyclyl groups include azetidinyl, pyrrolidinyl, imidazolinyl, oxazolidinyl, isoxazolinyl, thiazolidinyl, isothiazolidinyl, tetrahydrofuranyl, piperidinyl, piperazinyl, piperazinonyl, piperidinonyl, pyrrolidinonyl, azepinyl, azepinonyl, tetrahydropyranyl, morpholinyl, thiamorpholinyl, thiamorpholinyl sulfoxide, thiamorpholinyl sulfone, dioxolanyl, and tetrahydro-1,1-dioxothienyl.

The term “heteroaryl” refers to substituted and unsubstituted aromatic 5- or 6-membered monocyclic groups and 9- or 10-membered bicyclic groups that have at least one heteroatom (O, S or N) in at least one of the rings, said heteroatom-containing ring preferably having 1, 2, 3, or 4 heteroatoms independently selected from O, S, and/or N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. Bicyclic heteroaryl groups include heteroaryl groups with two aromatic rings in which one or both of the rings include at least one heteroatom; and heteroaryl groups with one aromatic ring and one non-aromatic ring in which one or both of the rings include at least one heteroatom. The nitrogen and sulfur atoms may optionally be oxidized and the nitrogen atoms may optionally be quaternized. The heteroaryl group may be attached at any available nitrogen or carbon atom of any ring. The heteroaryl ring system may be unsubstituted or may contain one or more substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, and pyrrolopyridyl.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compounds of Formula (I) can form salts which are also within the scope of this invention. Unless otherwise indicated, reference to an inventive compound is understood to include reference to one or more salts thereof. The term “salt(s)” denotes acidic and/or basic salts formed with inorganic and/or organic acids and bases. In addition, the term “salt(s) may include zwitterions (inner salts), e.g., when a compound of Formula (I) contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of the formula (I) may be formed, for example, by reacting a compound of the Formula (I) with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, maleates (formed with maleic acid), 2-hydroxyethanesulfonates, lactates, methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

The compounds of Formula (I) can be provided as amorphous solids or crystalline solids. Lyophilization can be employed to provide the compounds of Formula (I) as a solid.

It should further be understood that solvates (e.g., hydrates) of the Compounds of Formula (I) are also within the scope of the present invention. The term “solvate” means a physical association of a compound of Formula (I) with one or more solvent molecules, whether organic or inorganic. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

Various forms of prodrugs are known in the art and are described in Rautio, J. et al., Nature Review Drug Discovery, 17, 559-587 (2018).

In addition, compounds of Formula (I), subsequent to their preparation, can be isolated and purified to obtain a composition containing an amount by weight equal to or greater than 99% of a compound of Formula (I) (“substantially pure”), which is then used or formulated as described herein. Such “substantially pure” compounds of Formula (I) are also contemplated herein as part of the present invention.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor of DGKα and/or DGKζ, or effective to treat or prevent viral infections and proliferative disorders, such as cancer.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

The compounds of the present invention are intended to include all isotopes of atoms occurring in the present compounds. Isotopes include those atoms having the same atomic number but different mass numbers. By way of general example and without limitation, isotopes of hydrogen include deuterium (D) and tritium (T). Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

Compounds in accordance with Formula (I) and/or pharmaceutically acceptable salts thereof can be administered by any means suitable for the condition to be treated, which can depend on the need for site-specific treatment or quantity of Formula (I) compound to be delivered.

Also embraced within this invention is a class of pharmaceutical compositions comprising a compound of Formula (I) and/or pharmaceutically acceptable salts thereof; and one or more non-toxic, pharmaceutically-acceptable carriers and/or diluents and/or adjuvants (collectively referred to herein as “carrier” materials) and, if desired, other active ingredients. The compounds of Formula (I) may be administered by any suitable route, preferably in the form of a pharmaceutical composition adapted to such a route, and in a dose effective for the treatment intended. The compounds and compositions of the present invention may, for example, be administered orally, mucosally, or parentally including intravascularly, intravenously, intraperitoneally, subcutaneously, intramuscularly, and intrasternally in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g. magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) and/or at least one pharmaceutically acceptable salt thereof can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compounds of Formula (I) and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and/or at least one pharmaceutically acceptable salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

### Utility

The compounds of Formula (I) are useful for the treatment of cancer.

In another embodiment, the present invention provides a combined preparation of a compound of Formula (I), and/or a pharmaceutically acceptable salt thereof, a stereoisomer thereof or a tautomer thereof, and additional therapeutic agent(s) for simultaneous, separate or sequential use in the treatment and/or prophylaxis of multiple diseases or disorders associated with DGK target inhibition in T cells.

In another aspect, the invention provides a method of treating a patient suffering from or susceptible to a medical condition that is associated with DGK target inhibition in T cells. A number of medical conditions can be treated. The method comprises administering to the patient a therapeutically effective amount of a composition comprising a compound of Formula (I) and/or a pharmaceutically acceptable salt thereof, a stereoisomer thereof or a tautomer thereof. For example, the compounds described herein may be used to treat or prevent viral infections and proliferative diseases such as cancer.

The compounds for Formula (I) and pharmaceutical compositions comprising at least one compound of Formula (I) are useful in treating or preventing any disease or conditions that are associated with DGK target inhibition in T cells. These include viral and other infections (e.g., skin infections, GI infection, urinary tract infections, genito-urinary infections, systemic infections), and proliferative diseases (e.g., cancer). The compounds of Formula (I) and pharmaceutical compositions comprising in at least one compound of Formula (I) may be administered to animals, preferably mammals (e.g. domesticated animals, cats, dogs, mice, rats), and more preferably humans. Any method of administration may be used to deliver the compound or pharmaceutical composition to the patient. In certain embodiments, the compound of Formula (I) or pharmaceutical composition comprising at least compound of Formula (I) is administered orally. In other embodiments, the Formula (I) or pharmaceutical composition comprising at least compound of Formula (I) is administered parenterally.

The compounds of Formula (I) can inhibit activity of the diacylglycerol kinase alpha and zeta (DGKα/ζ). For example, the compounds of Formula (I) can be used to inhibit activity of DGKα and DGKζ in a cell or in an individual in need of modulation of DGKα and DGKζ by administering an inhibiting amount of a compound of Formula (I) or a salt thereof.

The present invention further provides methods of treating diseases associated with activity or expression, including abnormal activity and/or overexpression, of DGKα and DGKζ in an individual (e.g., patient) by administering to the individual in need of such treatment a therapeutically effective amount or dose of a compound of Formula (I) or a pharmaceutical composition thereof. Example diseases can include any disease, disorder or condition that is directly or indirectly linked to expression or activity of DGKα and DGKζ enzyme, such as over expression or abnormal activity. A DGKα and DGKζ-associated disease can also include any disease, disorder or condition that can be prevented, ameliorated, or cured by modulating DGKα and DGKζ enzyme activity. Examples of DGKα and DGKζ associated diseases include cancer and viral infections such as HIV infection, hepatitis B, and hepatitis C.

In one aspect, the compound(s) of Formula (I) are sequentially administered prior to administration of the immuno-oncology agent. In another aspect, compound(s) of Formula (I) are administered concurrently with the immuno-oncology agent. In yet another aspect, compound(s) of Formula (I) are sequentially administered after administration of the immuno-oncology agent.

In another aspect, compounds of Formula (I) may be co-formulated with an immuno-oncology agent.

Immuno-oncology agents include, for example, a small molecule drug, antibody, or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In one aspect, T cell responses can be stimulated by a combination of a compound of Formula (I) and one or more of (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

Other agents that can be combined with compounds of Formula (I) for the treatment of cancer include antagonists of inhibitory receptors on NK cells or agonists of activating receptors on NK cells. For example, compounds of Formula (I) can be combined with antagonists of KIR, such as lirilumab.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In another aspect, compounds of Formula (I) can be used with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In one aspect, the immuno-oncology agent is a CTLA-4 antagonist, such as an antagonistic CTLA-4 antibody. Suitable CTLA-4 antibodies include, for example, YERVOY (ipilimumab) or tremelimumab.

In another aspect, the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224

In another aspect, the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO2007/005874), and MSB0010718C (WO2013/79174).

In another aspect, the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In another aspect, the immuno-oncology agent is a CD137 (4-1BB) agonist, such as an agonistic CD137 antibody. Suitable CD137 antibodies include, for example, urelumab and PF-05082566 (WO12/32433).

In another aspect, the immuno-oncology agent is a GITR agonist, such as an agonistic GITR antibody. Suitable GITR antibodies include, for example, BMS-986153, BMS-986156, TRX-518 (WO06/105021, WO09/009116) and MK-4166 (WO11/028683).

In another aspect, the immuno-oncology agent is an IDO antagonist. Suitable IDO antagonists include, for example, INCB-024360 (WO2006/122150, WO07/75598, WO08/36653, WO08/36642), indoximod, BMS-986205, or NLG-919 (WO09/73620, WO09/1156652, WO11/56652, WO12/142237).

In another aspect, the immuno-oncology agent is an OX40 agonist, such as an agonistic OX40 antibody. Suitable OX40 antibodies include, for example, MEDI-6383 or MEDI-6469.

In another aspect, the immuno-oncology agent is an OX40L antagonist, such as an antagonistic OX40 antibody. Suitable OX40L antagonists include, for example, RG-7888 (WO06/029879).

In another aspect, the immuno-oncology agent is a CD40 agonist, such as an agonistic CD40 antibody. In yet another embodiment, the immuno-oncology agent is a CD40 antagonist, such as an antagonistic CD40 antibody. Suitable CD40 antibodies include, for example, lucatumumab or dacetuzumab.

In another aspect, the immuno-oncology agent is a CD27 agonist, such as an agonistic CD27 antibody. Suitable CD27 antibodies include, for example, varlilumab.

In another aspect, the immuno-oncology agent is MGA271 (to B7H3) (WO11/109400).

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the DGKα and DGKζ enzyme with a compound of Formula (I) includes the administration of a compound of the present invention to an individual or patient, such as a human, having DGKα and DGKζ, as well as, for example, introducing a compound of Formula (I) into a sample containing a cellular or purified preparation containing DGKα and DGKζ enzyme.

The term “DGKα and DGKζ inhibitor” refers to an agent capable of inhibiting the activity of diacylglycerol kinase alpha and/or diacylglycerol kinase zeta (DGKα and DGKζ) in T cells resulting in T cell stimulation. The DGKα and DGKζ inhibitor may be a reversible or irreversible DGKα and DGKζ inhibitor. “A reversible DGKα and DGKζ inhibitor” is a compound that reversibly inhibits DGKα and DGKζ enzyme activity either at the catalytic site or at a non-catalytic site and “an irreversible DGKα and DGKζ inhibitor” is a compound that irreversibly destroys DGKα and DGKζ enzyme activity by forming a covalent bond with the enzyme.

Types of cancers that may be treated with the compound of Formula (I) include, but are not limited to, brain cancers, skin cancers, bladder cancers, ovarian cancers, breast cancers, gastric cancers, pancreatic cancers, prostate cancers, colon cancers, blood cancers, lung cancers and bone cancers. Examples of such cancer types include neuroblastoma, intestine carcinoma such as rectum carcinoma, colon carcinoma, familiar adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchymal carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, diffuse large B-cell lymphoma (DLBCL), hepatocellular carcinoma, gall bladder carcinoma, bronchial carcinoma, small cell lung carcinoma, non-small cell lung carcinoma, multiple myeloma, basalioma, teratoma, retinoblastoma, choroid melanoma, seminoma, rhabdomyosarcoma, craniopharyngioma, osteosarcoma, chondrosarcoma, myosarcoma, liposarcoma, fibrosarcoma, Ewing sarcoma and plasmocytoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compounds of Formula (I) for treatment of DGKα and DGKζ associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compounds of Formula (I) include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compounds of Formula (I) may also be combined with immunotherapy drugs, including cytokines such as interferon alpha, interleukin 2, and tumor necrosis factor (TNF) in the treatment of melanoma.

Compounds of Formula (I) may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compounds of Formula (I), using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-a), etoposide, and teniposide.

Other cytotoxic agents include navelbene, CPT-11, anastrazole, letrazole, capecitabine, reloxafine, and droloxafine.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-1O or TGF-β).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

The pharmaceutical composition of the invention may optionally include at least one signal transduction inhibitor (STI). A “signal transduction inhibitor” is an agent that selectively inhibits one or more vital steps in signaling pathways, in the normal function of cancer cells, thereby leading to apoptosis. Suitable STIs include, but are not limited to: (i) bcr/abl kinase inhibitors such as, for example, STI 571 (GLEEVEC®); (ii) epidermal growth factor (EGF) receptor inhibitors such as, for example, kinase inhibitors (IRESSA®, SSI-774) and antibodies (Imclone: C225 [Goldstein et al., Clin. Cancer Res., 1:1311-1318 (1995)], and Abgenix: ABX-EGF); (iii) her-2/neu receptor inhibitors such as farnesyl transferase inhibitors (FTI) such as, for example, L-744,832 (Kohl et al., Nat. Med., 1(8):792-797 (1995)); (iv) inhibitors of Akt family kinases or the Akt pathway, such as, for example, rapamycin (see, for example, Sekulic et al., Cancer Res., 60:3504-3513 (2000)); (v) cell cycle kinase inhibitors such as, for example, flavopiridol and UCN-O1 (see, for example, Sausville, Curr. Med. Chem. Anti-Canc. Agents, 3:47-56 (2003)); and (vi) phosphatidyl inositol kinase inhibitors such as, for example, LY294002 (see, for example, Vlahos et al., J. Biol. Chem., 269:5241-5248 (1994)). Alternatively, at least one STI and at least one compound of Formula (I) may be in separate pharmaceutical compositions. In a specific embodiment of the present invention, at least one compound of Formula (I) and at least one STI may be administered to the patient concurrently or sequentially. In other words, at least one compound of Formula (I) may be administered first, at least one STI may be administered first, or at least one compound of Formula (I) and at least one STI may be administered at the same time. Additionally, when more than one compound of Formula (I) and/or STI is used, the compounds may be administered in any order.

The present invention further provides a pharmaceutical composition for the treatment of a chronic viral infection in a patient comprising at least one compound of Formula (I), optionally, at least one chemotherapeutic drug, and, optionally, at least one antiviral agent, in a pharmaceutically acceptable carrier.

Also provided is a method for treating a chronic viral infection in a patient by administering an effective amount of the above pharmaceutical composition.

In a specific embodiment of the present invention, at least one compound of Formula (I) and at least one chemotherapeutic agent are administered to the patient concurrently or sequentially. In other words, at least one compound of Formula (I) may be administered first, at least one chemotherapeutic agent may be administered first, or at least one compound of Formula (I) and the at least one STI may be administered at the same time. Additionally, when more than one compound of Formula (I) and/or chemotherapeutic agent is used, the compounds may be administered in any order. Similarly, any antiviral agent or STI may also be administered at any point in comparison to the administration of the compound of Formula (I).

Chronic viral infections that may be treated using the present combinatorial treatment include, but are not limited to, diseases caused by: hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV). Notably, parasitic infections (e.g., malaria) may also be treated by the above methods wherein compounds known to treat the parasitic conditions are optionally added in place of the antiviral agents.

Suitable antiviral agents contemplated for use in combination with the compound of Formula (I) can comprise nucleoside and nucleotide reverse transcriptase inhibitors (NRTIs), non-nucleoside reverse transcriptase inhibitors (NNRTIs), protease inhibitors and other antiviral drugs.

Examples of suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir; BCH-I0652; emitricitabine [(−)-FTC]; beta-L-FD4 (also called beta-L-D4C and named beta-L-2′,3′-dicleoxy-5-fluoro-cytidene); DAPD, ((−)-beta-D-2,6-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir; DMP-450; BMS-2322623; ABT-378; and AG-1549. Other antiviral agents include hydroxyurea, ribavirin, IL-2, IL-12, pentafuside and Yissum Project No. 11607.

The present invention also includes pharmaceutical kits useful, for example, in the treatment or prevention of DGKα and DGKζ-associated diseases or disorders, and other diseases referred to herein which include one or more containers containing a pharmaceutical composition comprising a therapeutically effective amount of a compound of Formula (I). Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy also can embrace the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The invention also provides pharmaceutically acceptable compositions which comprise a therapeutically effective amount of one or more of the compounds of Formula (I), formulated together with one or more pharmaceutically acceptable carriers (additives) and/or diluents, and optionally, one or more additional therapeutic agents described above.

The compounds of this invention can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms; and not injurious to the patient.

The term “pharmaceutical composition” means a composition comprising a compound of the invention in combination with at least one additional pharmaceutically acceptable carrier.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compounds of the present invention (250 mg), lactose (75 mg), and magnesium stearate (15 mg). The mixture is passed through a 60 mesh sieve and packed into a No. L gelatin capsule.

A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compounds of the present invention, alone or in combination with a pharmaceutical carrier. Optionally, compounds of the present invention can be used alone, in combination with other compounds of the invention, or in combination with one or more other therapeutic agent(s), e.g., an anticancer agent or other pharmaceutically active material.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the therapeutic effect and gradually increase the dosage until the effect is achieved.

In general, a suitable daily dose of a compound of the invention will be that amount of the compound which is the lowest dose effective to produce a therapeutic effect. Such an effective dose will generally depend upon the factors described above. Generally, oral, intravenous, intracerebroventricular and subcutaneous doses of the compounds of this invention for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for a compound of the present invention to be administered alone, it is preferable to administer the compound as a pharmaceutical formulation (composition).

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Methods of Preparation

The compounds of the present invention may be synthesized by many methods available to those skilled in the art of organic chemistry. General synthetic schemes for preparing compounds of the present invention are described below. These schemes are illustrative and are not meant to limit the possible techniques one skilled in the art may use to prepare the compounds disclosed herein. Different methods to prepare the compounds of the present invention will be evident to those skilled in the art. Examples of compounds of the present invention prepared by methods described in the general schemes are given in the Examples section set out hereinafter. Preparation of homochiral examples may be carried out by techniques known to one skilled in the art. For example, homochiral compounds may be prepared by separation of racemic products or diastereomers by chiral phase preparative HPLC. Alternatively, the example compounds may be prepared by methods known to give enantiomerically or diastereomerically enriched products.

The reactions and techniques described in this section are performed in solvents appropriate to the reagents and materials employed and are suitable for the transformations being effected. Also, in the description of the synthetic methods given below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art, with alternatives required when incompatible substituents are present. This will sometimes require a judgment to modify the order of the synthetic steps or to select one particular process scheme over another in order to obtain a compound of the invention. It will also be recognized that another major consideration in the planning of any synthetic route in this field is the judicious choice of a protecting group used for protection of reactive functional groups present in the compounds described in this invention. An authoritative account describing the many alternatives to the trained practitioner is Wuts and Greene, Greene's Protective Groups in Organic Synthesis, Fourth Edition, Wiley and Sons (2007).

### EXAMPLES

The following examples illustrate the particular and preferred embodiments of the present invention and do not limit the scope of the present invention. Chemical abbreviations and symbols as well as scientific abbreviations and symbols have their usual and customary meanings unless otherwise specified. Additional abbreviations employed in the Examples and elsewhere in this application are defined below. Common intermediates are generally useful for the preparation of more than one Example and are identified sequentially (e.g., Intermediate 1, Intermediate 2, etc.) and are abbreviated as Int. 1 or I1, Int. 2 or I2, etc. Compounds of the Examples are identified by the example and step in which they were prepared (e.g., “1-A” denotes the Example 1, step A), or by the example only where the compound is the title compound of the example (for example, “1” denotes the title compound of Example 1). In some instances alternate preparations of intermediates or examples are described. Frequently chemists skilled in the art of synthesis may devise alternative preparations which may be desirable based on one or more considerations such as shorter reaction time, less expensive starting materials, ease of operation or isolation, improved yield, amenable to catalysis, avoidance of toxic reagents, accessibility of specialized instrumentation, and decreased number of linear steps, etc. The intent of describing alternative preparations is to further enable the preparation of the examples of this invention. In some instances some functional groups in the outlined examples and claims may be replaced by well-known bioisosteric replacements known in the art, for example, replacement of a carboxylic acid group with a tetrazole or a phosphate moiety. ¹H NMR data collected in deuterated dimethyl sulfoxide used water suppression in the data processing. The reported spectra are uncorrected for the effects of water suppression. Protons adjacent to the water suppression frequency of 3.35 ppm exhibit diminished signal intensity.

### Abbreviations

Ac acetyl
anhyd. anhydrous
aq. aqueous
Bn benzyl
Boc-anhydride di-tert-butyl dicarbonate
BOC-D-ABU-OH N-(tert-butoxycarbonyl)-D-alanine
BOP benzotriazol-1-yloxytris-(dimethylamino)-phosphonium hexafluorophosphate
Bu butyl
CDI carbonyldiimidazole
DCM dichloromethane
DEA diethylamine
DIEA or DIPEA diisopropylethylamine
DMAP dimethylamino pyridine
DMF dimethylformamide
DMSO dimethyl sulfoxide
dppf 1,1′-bis(diphenylphosphino)ferrocene
EDC 1-ethyl-3-(3-dimethylaminopropyl)carbodiimide
Et ethyl
EtOAc ethyl acetate
EtOH ethanol
h, hours or hrs hour(s)
H homochiral
H-ABU-OME HCl methyl (2S)-2-aminobutanoate, HCl
HATU (1-[bis(dimethylamino)methylene]-1H-1,2,3-triazolo[4,5-b] pyridinium 3-oxide hexafluorophosphate)
HCl hydrochloric acid
HPLC high pressure liquid chromatography
LC liquid chromatography
LCMS liquid chromatography-mass spectrometry
M molar
mM millimolar
Me methyl
MeOH methanol
Mesyl-Cl methanesulfonyl chloride
MHz megahertz
mins minute(s)
M⁺¹ (M+H)⁺
MS mass spectrometry
n or N normal
NH₄OAc ammonium acetate
nM nanomolar
NMP N-methylpyrrolidinone
Pd₂(dba)₃ tris-(dibenzylideneacetone)dipalladium
PdCl₂(dppf) [1,1′-Bis(diphenylphosphino)ferrocene]dichloropalladium(II)
pet ether petroleum ether
Ph phenyl
PMB para-methoxy benzyl
POCl₃ phosphorous oxychloride
rt or Ret time retention time
sat. saturated
TBAF tetrabutylammonium fluoride
t-BuXphos 2-Di-tert-butylphosphino-2′,4′,6′-triisopropylbiphenyl
t-BuXphos-Rd-G1 chloro[2-(di-tert-butylphosphino)-2′,4′,6′-triisopropyl-1,1′-biphenyl][2-(2-aminoethyl)phenyl)]palladium(II)
TEA triethylamine
TFA trifluoroacetic acid
THF tetrahydrofuran
Xphos 2-dicyclohexylphosphino-2′,4′,6′-triisopropylbiphenyl

### LCMS Conditions:

Method A: Column: XBridge BEH XP C18 (50×2.1 mm, 2.5 μm); mobile phase A: 10 mM NH₄OAc in water:acetonitrile (95:5); mobile phase B: 10 mM NH₄OAc:acetonitrile (5:95); Gradient=0-100% B over 3 minutes; Temperature: 50° C.; Flow rate: 1.1 mL/min; Detection: UV at 220 nm.

Method B: Column: XBridge BEH XP C18 (50×2 mm, 2.5 μm); mobile phase A: 0.1% TFA in water:acetonitrile (95:5); mobile phase B: 0.1% TFA in water:acetonitrile (5:95); Gradient=0-100% B over 3 minutes; Temperature: 50° C.; Flow rate: 1.1 mL/min; Detection: UV at 220 nm.

### Intermediate 1

### 6-Chloro-3-(methylamino)picolinamide

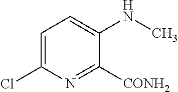

Intermediate 1 was prepared according to the method described in Adams et al., Bioorganic & Medicinal Chemistry Letters 26 (2016) 1086-1089.

### Intermediate 2

### 6-Chloro-4-hydroxy-1-methylpyrido[3,2-d]pyrimidin-2(1H)-one

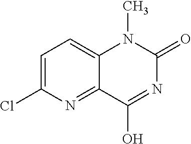

To a solution of 6-chloro-3-(methylamino)picolinamide (3 g, 16.16 mmol) in N,N-dimethylformamide (40 mL) was added NaH (1.29 g, 32.3 mmol, 60% w/w) at 0° C. The reaction mixture was stirred for 1 hour at room temperature. A solution of CDI (3.93 g, 24.24 mmol) in dimethylformamide (5 mL) was added and the reaction mixture was heated at 70° C. for 2 h. The reaction mixture was cooled to room temperature. The solid product separated was filtered, washed with water and dried under reduced pressure to afford 6-chloro-4-hydroxy-1-methylpyrido[3,2-d]pyrimidin-2(1H)-one (3.4 g, 85% yield). LCMS: m/z=212.1 (M+H); retention time 0.53 min. (Column: AQUITY UPLC BEH C18 (3.0×50 mm) 1.7 μm M. phase A: 10 mM NH₄OAc:ACN (95:5) M. phase B: 10 mM NH₄OAc:ACN (5:95) Description: Method: % B: 0 min-20:2 min-100:2.3 min-100, Flow: 0.7 mL/min). ¹H NMR (300 MHz, DMSO-d₆) δ 11.89 (br s, 1H), 8.00 (br d, J=8.4 Hz, 1H), 7.85 (br d, J=8.3 Hz, 1H), 3.42 (br s, 3H).

### Intermediate 3

### 4, 6-Dichloro-1-methylpyrido[3,2-d]pyrimidin-2(1H)-one

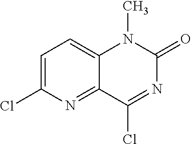

To a suspension of 6-chloro-4-hydroxy-1-methylpyrido[3,2-d]pyrimidin-2(1H)-one (2.0 g, 9.45 mmol) in dry toluene (20 mL) were added POCl₃ (4.40 mL, 47.3 mmol) and DIPEA (4.13 mL, 23.63 mmol) at room temperature. The reaction mixture was heated at 110° C. for 16 h. The reaction mixture was cooled to room temperature and the solvent was removed under reduced pressure. The crude product was dissolved in ethyl acetate and added K₂CO₃ (1.0 g), stirred for 5 min, diluted with water and extracted with ethyl acetate. The aqueous layer was basified and re-extracted with ethyl acetate (3×500 mL). The combined organic layer was dried over anhydrous Na₂SO₄, filtered and evaporated under reduced pressure to afford 4,6-dichloro-1-methylpyrido[3,2-d] pyrimidin-2(1H)-one (1.9 g, 32% yield) as a light brown semi-solid. LCMS: m/z=229.9 (M+H); rt 0.81 min. (LCMS Method: Column: Waters Acquity UPLC BEH C18 (2.1×50 mm) 1.7 μm, Mobile phase A: 10 mM ammonium acetate:acetonitrile (95:5); Mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

### Intermediate 4

### tert-Butyl (2R,5S)-4-(6-chloro-1-methyl-2-oxo-1,2-dihydropyrido[3,2-d]pyrimidin-4-yl)-2,5-diethylpiperazine-1-carboxylate

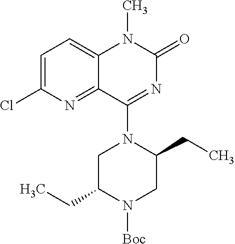 [SMILES: CC[C@@H]1CN(c2nc(=O)n(C)c3ccc(Cl)nc23)[C@@H](CC)CN1C(=O)OC(C)(C)C]

To a stirred solution of tert-butyl (2R,5S)-2,5-diethylpiperazine-1-carboxylate (1.37 g, 5.65 mmol) in acetonitrile (10 mL) were added DIPEA (2.3 mL, 13.04 mmol) and 4,6-dichloro-1-methylpyrido[3,2-d]pyrimidin-2(1H)-one (1.0 g, 4.35 mmol) at room temperature. The reaction mixture was heated at 85° C. for 6 h. The reaction mixture was cooled to room temperature and concentrated under reduced pressure to obtain the crude product, which was purified using silica gel column chromatography (60-70% EtOAc/petroleum ether; 40 g column) to afford tert-butyl (2R,5S)-4-(6-chloro-1-methyl-2-oxo-1,2-dihydropyrido[3,2-d]pyrimidin-4-yl)-2,5-diethylpiperazine-1-carboxylate (1.00 g, 47% yield). LCMS: m/z=436.2 (M+H); rt 1.82 min. LCMS Method: Column: Waters Acquity UPLC BEH C18 (2.1×50 mm) 1.7 μm, Mobile phase A: 10 mM ammonium acetate:acetonitrile (95:5); Mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

### Intermediate 5

### tert-Butyl (2R,5S)-4-(6-cyano-1-methyl-2-oxo-1,2-dihydropyrido[3,2-d]pyrimidin-4-yl)-2,5-diethylpiperazine-1-carboxylate

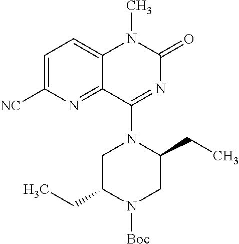 [SMILES: CC[C@@H]1CN(c2nc(=O)n(C)c3ccc(C#N)nc23)[C@@H](CC)CN1C(=O)OC(C)(C)C]

To a stirred solution of tert-butyl (2R,5S)-4-(6-chloro-1-methyl-2-oxo-1,2-dihydropyrido[3,2-d]pyrimidin-4-yl)-2,5-diethylpiperazine-1-carboxylate (1 g, 2.29 mmol) in NMP (5 mL) were added dppf (0.254 g, 0.459 mmol), zinc (0.15 g, 2.29 mmol) and zinc cyanide (0.54 g, 4.59 mmol). The reaction mixture was degassed for 5 min. and Pd₂(dba)₃ (0.21 g, 0.23 mmol) was added. The reaction mixture was heated at 90° C. overnight. The reaction mixture was cooled to room temperature, diluted with ethyl acetate and filtered through Celite pad. The filtrate was washed with water, brine and the organic layer was dried over anhydrous Na₂SO₄, filtered and evaporated under reduced pressure to obtain crude compound. The crude residue was purified using silica gel chromatography (70-80% EtOAc/petroleum ether; 40 g column) to afford the tert-butyl (2R,5S)-4-(6-chloro-1-methyl-2-oxo-1,2-dihydropyrido[3,2-d]pyrimidin-4-yl)-2,5-diethylpiperazine-1-carboxylate (0.8 g, 75% yield). LCMS: m/z=427.2 (M+H); rt 1.56 min. LCMS Method: Column: Waters Acquity UPLC BEH C18 (2.1×50 mm) 1.7 μm, Mobile phase A: 10 mM ammonium acetate:acetonitrile (95:5); Mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

### Intermediate 6

### 4-((2S,5R)-2,5-Diethylpiperazin-1-yl)-1-methyl-2-oxo-1,2-dihydropyrido[3,2-d] pyrimidine-6-carbonitrile, TFA

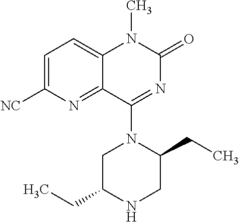 [SMILES: CC[C@@H]1CN(c2nc(=O)n(C)c3ccc(C#N)nc23)[C@@H](CC)CN1]

To a stirred solution of tert-butyl (2R,5S)-4-(6-cyano-1-methyl-2-oxo-1,2-dihydropyrido[3,2-d]pyrimidin-4-yl)-2,5-diethylpiperazine-1-carboxylate (0.46 g, 1.08 mmol) in dry DCM (10 mL) was added TFA (3.5 mL, 45.4 mmol) at room temperature. The reaction mixture was stirred for 2 h. The solvent was removed under reduced pressure to afford 4-((2S,5R)-2,5-diethylpiperazin-1-yl)-1-methyl-2-oxo-1,2-dihydropyrido[3,2-d]pyrimidine-6-carbonitrile, TFA (0.46 g, 55% yield). LCMS: m/z=327.2 (M+H); rt 0.61 min. LCMS Method: Column: Waters Acquity UPLC BEH C18 (2.1×50 mm) 1.7 μm, Mobile phase A: 10 mM ammonium acetate:acetonitrile (95:5); Mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

### Examples 1 and 2

### 4-((2S,5R)-2,5-Diethyl-4-((4-fluorophenyl)(5-(trifluoromethyl)pyridin-2-yl)methyl) piperazin-1-yl)-1-methyl-2-oxo-1,2-dihydropyrido[3,2-d]pyrimidine-6-carbonitrile

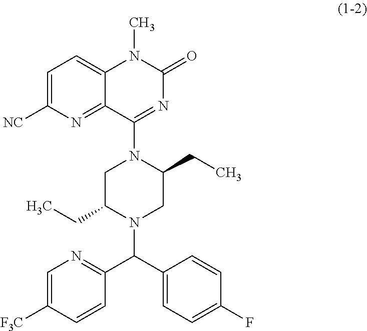 [SMILES: CC[C@H]1CN(C(c2ccc(F)cc2)c2ccc(C(F)(F)F)cn2)[C@H](CC)CN1c1nc(=O)n(C)c2ccc(C#N)nc12]

Coupling Method A: To a stirred solution of 4-((2S,5R)-2,5-diethylpiperazin-1-yl)-1-methyl-2-oxo-1,2-dihydropyrido[3,2-d]pyrimidine-6-carbonitrile, TFA (100 mg, 0.23 mmol) in acetonitrile (10 mL) was added DIPEA (0.12 mL, 0.68 mmol), followed by 2-(bromo(4-fluorophenyl)methyl)-5-(trifluoromethyl)pyridine (0.23 g, 0.68 mmol). The reaction mixture was heated at 85° C. overnight. The reaction mixture was cooled to room temperature and the solvent was removed under reduced pressure to yield the crude product, which was purified by preparative HPLC (HPLC Method: Column: Sunfire C18, 150×19 mm ID, 5 μm; Mobile Phase A: 10 mM ammonium acetate in water; Mobile Phase B: acetonitrile; Gradient: 0-100% B over 18 minutes, then a 5 minute hold at 100% B; Flow: 17 mL/min), fractions were concentrated under reduced pressure and lyophilized from (EtOH/H₂O, 1:5) to yield Example 1 and Example 2.

Example 1: 23 mg, 17% yield; LCMS: m/z=580.3 (M+H); rt 3.46 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.91 (s, 1H), 8.29-8.15 (m, 2H), 8.02-7.91 (m, 2H), 7.68-7.60 (m, 2H), 7.23-7.13 (m, 2H), 5.95-5.85 (m, 0.5H), 5.42-5.32 (m, 0.5H), 5.05-5.01 (m, 1H), 4.97-4.89 (m, 1H), 3.70-3.63 (m, 1H), 3.46-3.40 (m, 4H), 2.71-2.65 (m, 2H), 2.36-2.29 (m, 1H), 2.09-1.89 (m, 1H), 1.68-1.38 (m, 2H), 0.84-0.73 (m, 3H), 0.70-0.48 (m, 3H).

Example 2: 11 mg, 8% yield; LCMS: m/z=580.3 (M+H); rt 3.46 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.91 (s, 1H), 8.31-8.11 (m, 2H), 8.05-7.86 (m, 2H), 7.63-7.574 (m, 2H), 7.27-7.06 (m, 2H), 5.91-5.36 (m, 1H), 5.02 (s, 1H), 4.98-4.90 (m, 1H), 3.70-3.61 (m, 1H), 3.43-3.89 (m, 4H), 2.70-2.62 (m, 2H), 2.33-2.37 (m, 1H), 2.09-1.93 (m, 1H), 1.68-1.42 (m, 2H), 0.79 (q, J=7.2 Hz, 3H), 0.70-0.48 (m, 3H)

### Examples 3 and 4

### 4-((2S, 5R)-4-(1-(4-Cyclopropylphenyl)ethyl)-2, 5-dimethylpiperazin-1-yl)-1-methyl-2-oxo-1, 2-dihydropyrido[3, 2-d]pyrimidine-6-carbonitrile

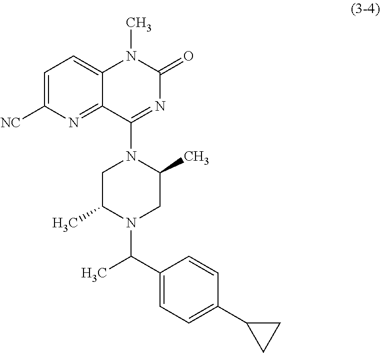 [SMILES: CC(c1ccc(C2CC2)cc1)N1C[C@H](C)N(c2nc(=O)n(C)c3ccc(C#N)nc23)C[C@H]1C]

Coupling Method B: To a stirred solution of 4-((2S, 5R)-2, 5-dimethylpiperazin-1-yl)-1-methyl-2-oxo-1, 2-dihydropyrido[3, 2-d]pyrimidine-6-carbonitrile (60 mg, 0.20 mmol) in acetonitrile (8 mL) were added DIPEA (0.11 mL, 0.60 mmol), 1-(1-chloroethyl)-4-cyclopropylbenzene (43.6 mg, 0.24 mmol) and sodium iodide (15.1 mg, 0.10 mmol). The reaction mixture was heated at 85° C. for 16 h. The reaction mixture was cooled to room temperature and the solvent was removed under reduced pressure and the residue was dissolved in ethyl acetate, washed with brine, dried over Na₂SO₄ and concentrated under reduced pressure to yield the crude product, which was purified by preparative HPLC (HPLC Method: Column: Gemini NX (250 mm×21.2 mm ID, 5 μm) Mobile phase A=10 mM ammonium acetate in water Mobile phase B=acetonitrile:MeOH (1:1) Gradient: 0-100% B over 16 minutes, then a 5 minute hold at 100% B; Flow: 19 mL/min) to yield a diastereomeric mixture of product, which was purified by preparative chiral HPLC (Chiral HPLC Method: Column: Cellulose-5 (250×19 ID) 5 micron Mobile Phase A: 10 mM NH₄OAc in MeOH Flow: 25 mL/min). The fractions were concentrated under reduced pressure and lyophilized from EtOH/H₂O (1:5) to yield Example 3 and Example 4.

Example 3 (1.5 mg, 2% yield). LCMS: m/z=443.3 (M+H); rt 2.25 min; (LCMS method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50° C.; Time (min): 0-3; % B: 0-100%). ¹H NMR; (400 MHz, DMSO-d₆) δ ppm 8.22 (br d, J=8.6 Hz, 1H), 8.00-7.94 (m, 1H), 7.28-7.22 (m, 2H), 7.07-7.02 (m, 2H), 5.79-4.12 (m, 2H), 3.57-3.52 (m, 2H), 3.44-3.41 (s, 3H), 2.89-2.76 (m, 3H), 1.94-1.84 (m, 1H), 1.52-1.37 (m, 3H), 1.25-1.19 (m, 3H), 0.95-0.84 (m, 5H), 0.70-0.62 (m, 2H).

Example 4 (2.0 mg, 2% yield). LCMS: m/z=443.3 (M+H); rt 2.26 min; (LCMS method: Column: XBridge BEH XP C18 (50×2.1 mm), 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50° C.; Time (min): 0-3; % B: 0-100%). ¹H NMR (400 MHz, DMSO-d₆) δ ppm 8.23 (d, J=8.6 Hz, 1H), 7.99 (d, J=8.8 Hz, 1H), 7.24 (d, J=8.1 Hz, 2H), 7.03 (d, J=8.3 Hz, 2H), 5.75-5.38 (m, 0.5H), 5.06-4.68 (m, 0.5H), 3.58-3.53 (m, 1H), 3.44 (s, 3H), 3.42-3.38 (m, 1H), 2.59-2.54 (m, 3H), 2.25-2.16 (m, 1H), 1.94-1.84 (m, 1H), 1.37-1.19 (m, 6H), 1.12-0.98 (m, 3H), 0.96-0.89 (m, 2H), 0.69-0.62 (m, 2H).

### Examples 5 and 6

### 4-((2S,5R)-2,5-Diethyl-4-(1-(4-(trifluoromethyl)phenyl)propyl)piperazin-1-yl)-1-methyl-2-oxo-1,2-dihydropyrido[3,2-d]pyrimidine-6-carbonitrile

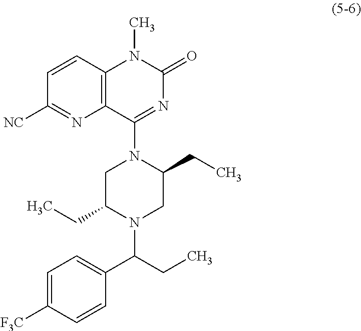 [SMILES: CCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, TFA (0.12 g, 0.27 mmol) in acetonitrile (10 mL) were added DIPEA (0.14 mL, 0.82 mmol), 1-(1-chloropropyl)-4-(trifluoromethyl)benzene (0.12 g, 0.55 mmol), and sodium iodide (0.04 g, 0.27 mmol). The reaction mixture was heated at 85° C. for 16 h. The reaction mixture was cooled to room temperature and the solvent was removed under reduced pressure to yield the crude product, which was purified by preparative HPLC [HPLC Method: Column: Sunfire C18, 150×19 mm ID, 5 μm; Mobile Phase A: 10 mM ammonium acetate in water; Mobile Phase B: acetonitrile; Gradient: 0-100% B over 18 minutes, then a 5 minute hold at 100% B; Flow: 17 mL/min]. The fractions were concentrated under reduced pressure and lyophilized from EtOH/H₂O (1:5) to yield Examples 5 and 6.

Example 5: (10 mg, 7% yield); LCMS: m/z=513.3 (M+H); rt 2.52 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50° C.; Time (min): 0-4; % B: 0-100; ¹H NMR (400 MHz, DMSO-d₆) δ 8.24 (d, J=6.6 Hz, 1H), 7.98 (d, J=9.0 Hz, 1H), 7.73 (d, J=8.1 Hz, 2H), 7.56 (d, J=7.1 Hz, 2H), 5.83-5.48 (m, 1H), 4.98-4.86 (m, 1H), 3.64 (br. s., 1H), 3.43 (s, 3H), 3.08 (d, J=9.8 Hz, 1H), 2.93-2.82 (m, 2H), 2.42-2.26 (m, 1H), 2.13-2.08 (m, 1H), 1.98-1.82 (m, 3H), 1.66-1.54 (m, 1H), 1.44-1.31 (m, 1H), 0.98-0.91 (br. s., 3H), 0.69-0.53 (m, 6H).

Example 6: (3 mg, 2% yield); LCMS: m/z=513.3 (M+H); rt 2.54 min; (LCMS method: Column: XBridge BEH XP C18 (50×2.1) mm, 2.5 μm Mobile phase A: 95% water: 5% acetonitrile; 10 mM ammonium formate; Mobile phase B: 5% Water: 95% acetonitrile; 10 mM ammonium formate; Flow: 1.1 mL/min; Temp: 50° C.; Time (min): 0-4; % B: 0-100; ¹H NMR (400 MHz, DMSO-d₆) δ 8.28-8.19 (m, 1H), 8.01-7.95 (m, 1H), 7.72 (d, J=7.8 Hz, 2H), 7.58 (d, J=8.6 Hz, 2H), 6.06-5.28 (m, 1H), 5.08-4.76 (m, 1H), 3.64-3.50 (m, 2H), 3.43 (s, 3H), 3.16-3.08 (m, 1H), 2.25-2.14 (m, 2H), 2.00-1.83 (m, 3H), 1.57-1.53 (m, 3H), 1.03-0.89 (m, 3H), 0.65-0.54 (m, 6H).

### Examples 7 and 8

### 4-((2S,5R)-5-Ethyl-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

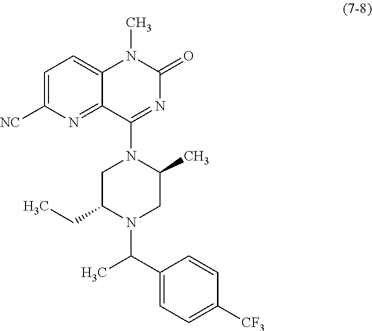 [SMILES: CC[C@@H]1CN(c2nc(=O)n(C)c3ccc(C#N)nc23)[C@@H](C)CN1C(C)c1ccc(C(F)(F)F)cc1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (70 mg, 0.22 mmol) in acetonitrile (2 mL) at room temperature were added DIPEA (0.12 mL, 0.67 mmol), 1-(1-chloroethyl)-4-(trifluoromethyl)benzene (93 mg, 0.45 mmol), sodium iodide (33.6 mg, 0.22 mmol) and heated at 85° C. for 16 h. The reaction mixture cooled to room temperature and the solvent was removed under reduced pressure, the residue was dissolved in ethyl acetate (100 mL). The organic layer was washed with brine, dried over Na₂SO₄ and concentrated under reduced pressure to yield the crude product, which was purified by preparative HPLC [HPLC Method: Column: Sunfire C18 (150 mm×19.2 mm ID, 5 μm), Mobile phase A=10 mM ammonium acetate in water, Mobile phase B=acetonitrile, Flow: 19 mL/min], fractions were concentrated under reduced pressure, diluted with EtOH/H₂O (1:5), and lyophilized to yield Examples 7 and 8.

Example 7: (9 mg, 8% yield); LCMS: m/z=485.1 (M+H); rt 2.34 min; (LCMS method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50° C.; Time (min): 0-3; % B: 0-100. ¹H NMR (400 MHz, DMSO-d₆) δ ppm 8.32-8.17 (m, 1H), 8.05-7.94 (m, 1H), 7.76-7.66 (m, 2H), 7.66-7.55 (m, 2H), 6.11-5.42 (m, 1H), 5.10-4.79 (m, 1H), 3.78-3.59 (m, 2H), 3.44 (s, 3H), 3.17-3.05 (m, 1H), 2.64-2.55 (m, 1H), 2.26-2.09 (m, 1H), 1.65-1.34 (m, 3H), 1.31-1.16 (m, 5H), 1.01 (br t, J=7.1 Hz, 3H)

Example 8: (9 mg, 8% yield); LCMS: m/z=485.1 (M+H); rt 2.29 min; (LCMS Method: Column: XBridge BEH XP C18 (50×2.1 mm), 2.5 μm; Mobile phase A: 95% water: 5% acetonitrile; 10 mM ammonium acetate; Mobile phase B: 5% Water: 95% acetonitrile; 10 mM ammonium acetate; Flow: 1.1 mL/min; Temp: 50° C.; Time (min): 0-3; % B: 0-100%). ¹H NMR (400 MHz, DMSO-d₆) δ ppm 8.24 (br d, J=8.6 Hz, 1H), 7.99 (d, J=9.0 Hz, 1H), 7.73 (d, J=8.3 Hz, 2H), 7.61 (br d, J=8.3 Hz, 2H), 5.87-5.63 (m, 1H), 5.10-4.79 (m, 1H), 3.90-3.80 (m, 1H), 3.44 (s, 3H), 3.46-3.15 (m, 1H), 2.89-2.73 (m, 2H), 2.41-2.34 (m, 1H), 1.63-1.34 (m, 5H), 1.29 (br d, J=6.1 Hz, 3H), 0.79-0.64 (m, 3H)

The examples in the Table 1 were prepared according to the general procedure described in Examples 1 to 4, using the appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond. Coupling Method A is described in the general procedure for the preparation of Examples 1 and 2. Coupling Method B is described in the general procedure for the preparation of Examples 3 and 4.

TABLE 1

| Ex. | | Stereo | LCMS | LCMS | | Coupling |
|---|---|---|---|---|---|---|
| No. | Structure | Chem. | Method | RT | M + H | Method |
| 9 | 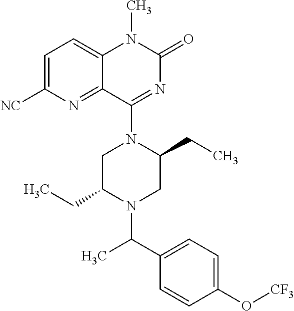 [SMILES: CC[C@H]1CN(C(C)c2ccc(OC(F)(F)F)cc2)[C@H](CC)CN1c1nc(=O)n(C)c2ccc(C#N)nc12] | H | A | 2.45 | 515.2 | B |
| 10 | 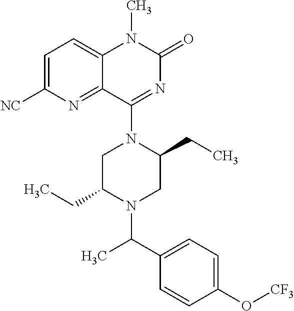 [SMILES: CC[C@H]1CN(C(C)c2ccc(OC(F)(F)F)cc2)[C@H](CC)CN1c1nc(=O)n(C)c2ccc(C#N)nc12] | H | A | 2.47 | 515.3 | B |
| 11 | 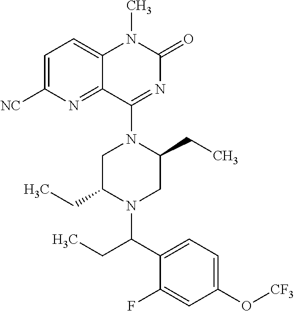 [SMILES: CCC(c1ccc(OC(F)(F)F)cc1F)N1C[C@H](CC)N(c2nc(=O)n(C)c3ccc(C#N)nc23)C[C@H]1CC] | H | A | 2.56 | 547.2 | B |
| 12 | 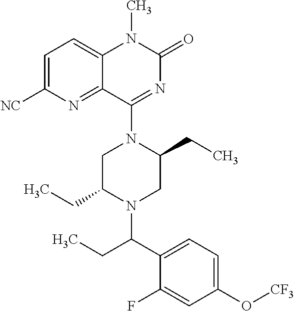 [SMILES: CCC(c1ccc(OC(F)(F)F)cc1F)N1C[C@H](CC)N(c2nc(=O)n(C)c3ccc(C#N)nc23)C[C@H]1CC] | H | A | 2.59 | 547.2 | B |
| 13 | 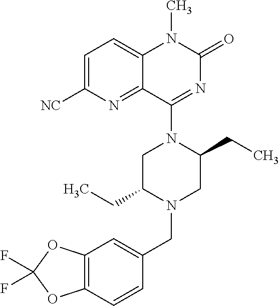 [SMILES: CC[C@@H]1CN(c2nc(=O)n(C)c3ccc(C#N)nc23)[C@@H](CC)CN1Cc1ccc2c(c1)OC(F)(F)O2] | H | A | 2.33 | 497.2 | A |
| 14 | 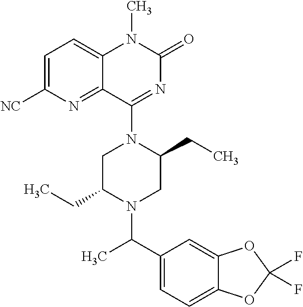 [SMILES: CC[C@H]1CN(C(C)c2ccc3c(c2)OC(F)(F)O3)[C@H](CC)CN1c1nc(=O)n(C)c2ccc(C#N)nc12] | H | A | 2.43 | 511.3 | B |
| 15 | 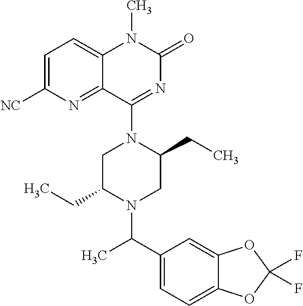 [SMILES: CC[C@H]1CN(C(C)c2ccc3c(c2)OC(F)(F)O3)[C@H](CC)CN1c1nc(=O)n(C)c2ccc(C#N)nc12] | H | A | 2.44 | 511.3 | B |
| 16 | 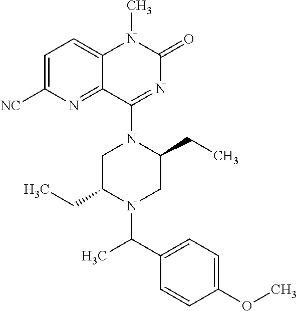 [SMILES: CC[C@H]1CN(C(C)c2ccc(OC)cc2)[C@H](CC)CN1c1nc(=O)n(C)c2ccc(C#N)nc12] | H | A | 2.16 | 461.3 | B |
| 17 | 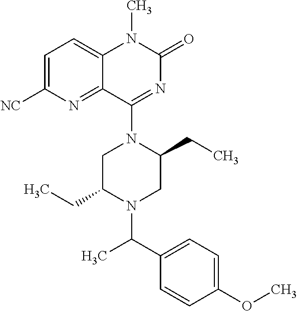 [SMILES: CC[C@H]1CN(C(C)c2ccc(OC)cc2)[C@H](CC)CN1c1nc(=O)n(C)c2ccc(C#N)nc12] | H | A | 2.19 | 461.3 | B |
| 18 | 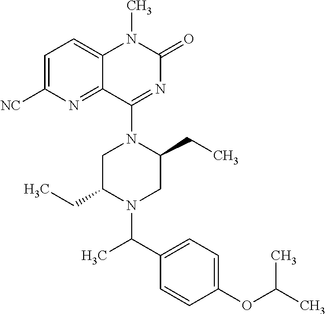 [SMILES: CC[C@H]1CN(C(C)c2ccc(OC(C)C)cc2)[C@H](CC)CN1c1nc(=O)n(C)c2ccc(C#N)nc12] | H | A | 2.43 | 489.3 | B |
| 19 | 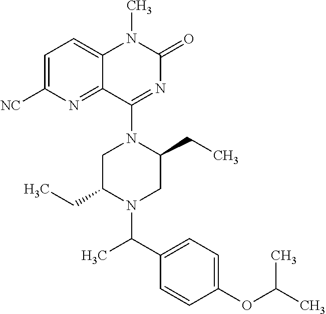 [SMILES: CC[C@H]1CN(C(C)c2ccc(OC(C)C)cc2)[C@H](CC)CN1c1nc(=O)n(C)c2ccc(C#N)nc12] | H | A | 2.41 | 489.3 | B |
| 20 | 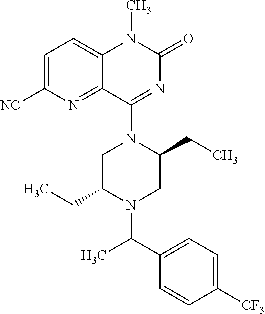 [SMILES: CC[C@H]1CN(C(C)c2ccc(C(F)(F)F)cc2)[C@H](CC)CN1c1nc(=O)n(C)c2ccc(C#N)nc12] | H | A | 2.42 | 499.1 | B |
| 21 | 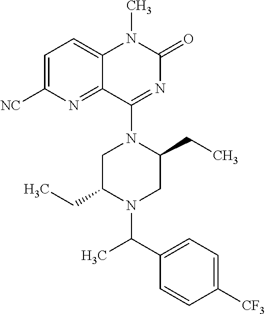 [SMILES: CC[C@H]1CN(C(C)c2ccc(C(F)(F)F)cc2)[C@H](CC)CN1c1nc(=O)n(C)c2ccc(C#N)nc12] | H | A | 1.45 | 499.3 | B |
| 22 | 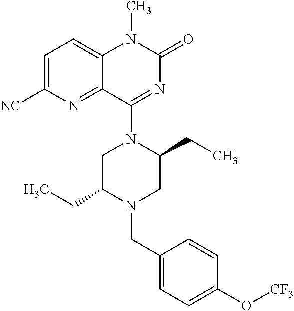 [SMILES: CC[C@@H]1CN(c2nc(=O)n(C)c3ccc(C#N)nc23)[C@@H](CC)CN1Cc1ccc(OC(F)(F)F)cc1] | H | A | 2.37 | 501.1 | A |
| 23 | 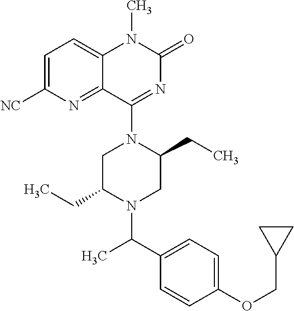 [SMILES: CC[C@H]1CN(C(C)c2ccc(OCC3CC3)cc2)[C@H](CC)CN1c1nc(=O)n(C)c2ccc(C#N)nc12] | H | A | 2.41 | 501.1 | B |
| 24 | 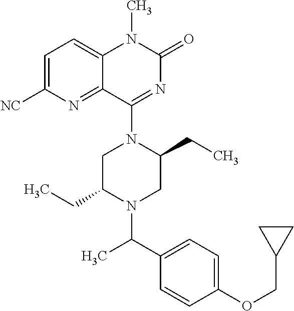 [SMILES: CC[C@H]1CN(C(C)c2ccc(OCC3CC3)cc2)[C@H](CC)CN1c1nc(=O)n(C)c2ccc(C#N)nc12] | H | A | 2.43 | 501.2 | B |
| 25 | 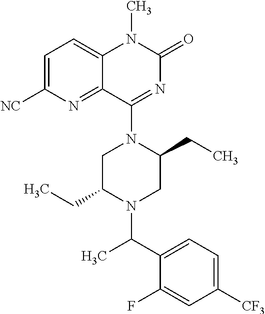 [SMILES: CC[C@H]1CN(C(C)c2ccc(C(F)(F)F)cc2F)[C@H](CC)CN1c1nc(=O)n(C)c2ccc(C#N)nc12] | H | A | 2.47 | 517.3 | B |
| 26 | 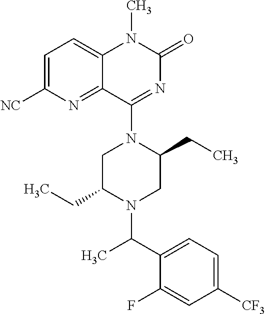 [SMILES: CC[C@H]1CN(C(C)c2ccc(C(F)(F)F)cc2F)[C@H](CC)CN1c1nc(=O)n(C)c2ccc(C#N)nc12] | H | A | 2.45 | 517.3 | B |
| 27 | 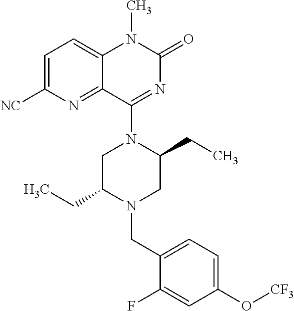 [SMILES: CC[C@@H]1CN(c2nc(=O)n(C)c3ccc(C#N)nc23)[C@@H](CC)CN1Cc1ccc(OC(F)(F)F)cc1F] | H | A | 2.41 | 519.3 | A |
| 28 | 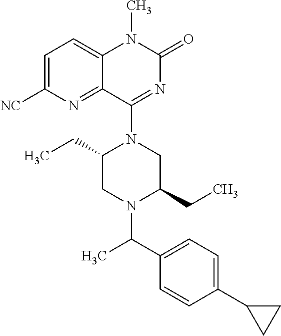 [SMILES: CC[C@H]1CN(C(C)c2ccc(C3CC3)cc2)[C@H](CC)CN1c1nc(=O)n(C)c2ccc(C#N)nc12] | H | A | 2.5 | 471.3 | B |
| 29 | 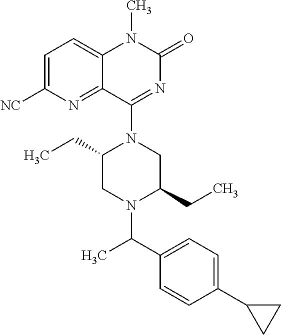 [SMILES: CC[C@H]1CN(C(C)c2ccc(C3CC3)cc2)[C@H](CC)CN1c1nc(=O)n(C)c2ccc(C#N)nc12] | H | A | 2.48 | 471.3 | B |
| 30 | 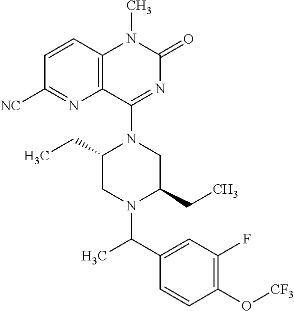 [SMILES: CC[C@H]1CN(C(C)c2ccc(OC(F)(F)F)c(F)c2)[C@H](CC)CN1c1nc(=O)n(C)c2ccc(C#N)nc12] | H | A | 2.47 | 533.3 | B |
| 31 | 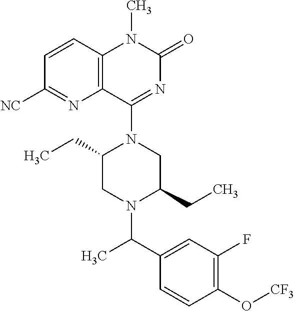 [SMILES: CC[C@H]1CN(C(C)c2ccc(OC(F)(F)F)c(F)c2)[C@H](CC)CN1c1nc(=O)n(C)c2ccc(C#N)nc12] | H | A | 2.49 | 533.3 | B |
| 32 | 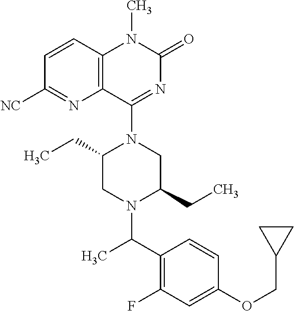 [SMILES: CC[C@H]1CN(C(C)c2ccc(OCC3CC3)cc2F)[C@H](CC)CN1c1nc(=O)n(C)c2ccc(C#N)nc12] | H | A | 2.46 | 519.3 | B |
| 33 | 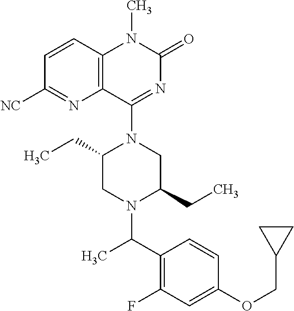 [SMILES: CC[C@H]1CN(C(C)c2ccc(OCC3CC3)cc2F)[C@H](CC)CN1c1nc(=O)n(C)c2ccc(C#N)nc12] | H | A | 2.48 | 519.4 | B |
| 34 | 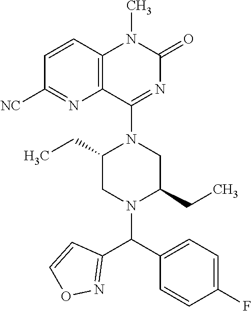 [SMILES: CC[C@H]1CN(C(c2ccc(F)cc2)c2ccon2)[C@H](CC)CN1c1nc(=O)n(C)c2ccc(C#N)nc12] | H | A | 1.98 | 502.3 | A |
| 35 | 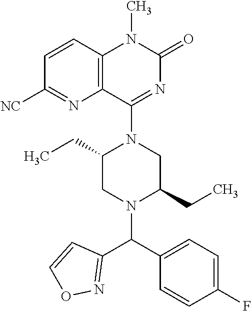 [SMILES: CC[C@H]1CN(C(c2ccc(F)cc2)c2ccon2)[C@H](CC)CN1c1nc(=O)n(C)c2ccc(C#N)nc12] | H | A | 1.96 | 502.3 | A |
| 36 | 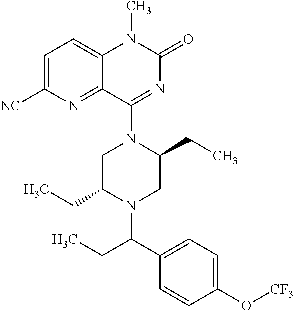 [SMILES: CCC(c1ccc(OC(F)(F)F)cc1)N1C[C@H](CC)N(c2nc(=O)n(C)c3ccc(C#N)nc23)C[C@H]1CC] | H | A | 2.57 | 529.3 | B |
| 37 | 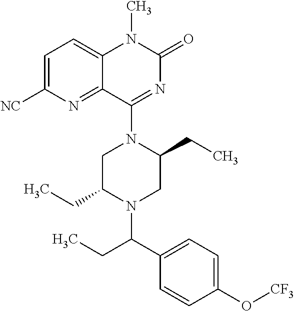 [SMILES: CCC(c1ccc(OC(F)(F)F)cc1)N1C[C@H](CC)N(c2nc(=O)n(C)c3ccc(C#N)nc23)C[C@H]1CC] | H | A | 2.59 | 529.3 | B |
| 38 | 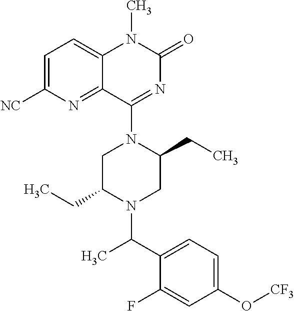 [SMILES: CC[C@H]1CN(C(C)c2ccc(OC(F)(F)F)cc2F)[C@H](CC)CN1c1nc(=O)n(C)c2ccc(C#N)nc12] | H | A | 2.49 | 533.3 | B |
| 39 | 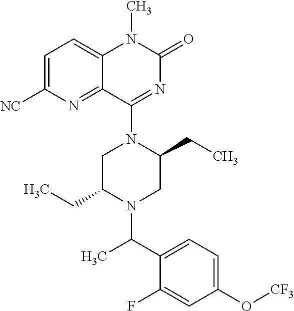 [SMILES: CC[C@H]1CN(C(C)c2ccc(OC(F)(F)F)cc2F)[C@H](CC)CN1c1nc(=O)n(C)c2ccc(C#N)nc12] | H | A | 2.52 | 533.3 | B |
| 40 | 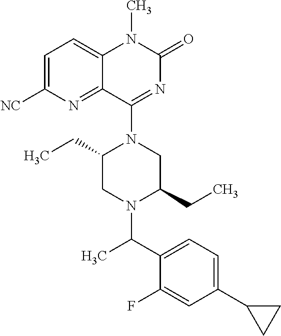 [SMILES: CC[C@H]1CN(C(C)c2ccc(C3CC3)cc2F)[C@H](CC)CN1c1nc(=O)n(C)c2ccc(C#N)nc12] | H | A | 2.47 | 489.4 | B |
| 41 | 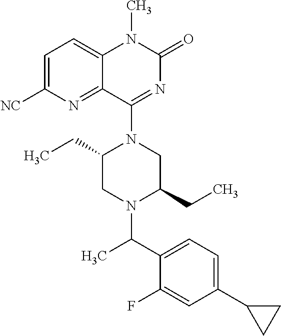 [SMILES: CC[C@H]1CN(C(C)c2ccc(C3CC3)cc2F)[C@H](CC)CN1c1nc(=O)n(C)c2ccc(C#N)nc12] | H | A | 2.49 | 489.3 | B |
| 42 | 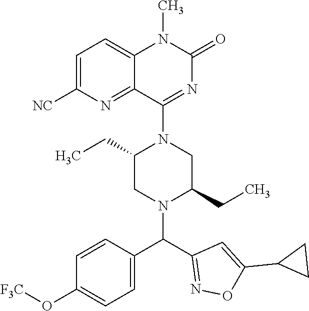 [SMILES: CC[C@H]1CN(C(c2ccc(OC(F)(F)F)cc2)c2cc(C3CC3)on2)[C@H](CC)CN1c1nc(=O)n(C)c2ccc(C#N)nc12] | H | A | 2.38 | 608.3 | A |
| 43 | 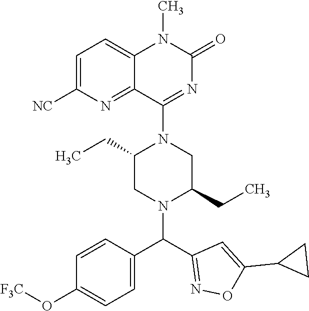 [SMILES: CC[C@H]1CN(C(c2ccc(OC(F)(F)F)cc2)c2cc(C3CC3)on2)[C@H](CC)CN1c1nc(=O)n(C)c2ccc(C#N)nc12] | H | A | 2.39 | 608.3 | A |
| 44 | 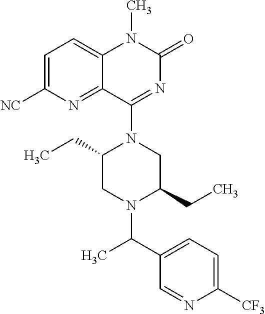 [SMILES: CC[C@H]1CN(C(C)c2ccc(C(F)(F)F)nc2)[C@H](CC)CN1c1nc(=O)n(C)c2ccc(C#N)nc12] | H | A | 3.07 | 500.2 | B |
| 45 | 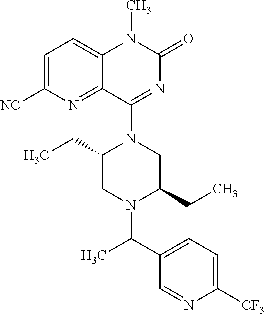 [SMILES: CC[C@H]1CN(C(C)c2ccc(C(F)(F)F)nc2)[C@H](CC)CN1c1nc(=O)n(C)c2ccc(C#N)nc12] | H | A | 3.05 | 500.2 | B |

### Examples 46 and 47

### 4-((2S,5R)-5-Ethyl-4-((4-fluorophenyl)(5-(trifluoromethyl)pyridin-2-yl)methyl)-2-methylpiperazin-1-yl)-1-methyl-2-oxo-1,2-dihydropyrido[3,2-d]pyrimidine-6-carbonitrile

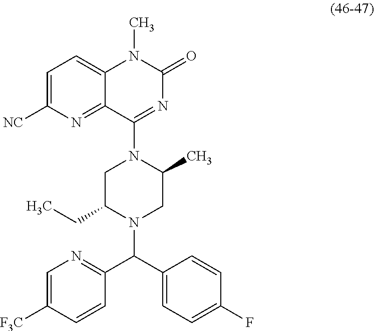 [SMILES: CC[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile, TFA (0.5 g, 1.17 mmol) in acetonitrile (10 mL) was added DIPEA (1.02 mL, 5.86 mmol), followed by 2-(bromo(4-fluorophenyl)methyl)-5-(trifluoromethyl)pyridine (0.78 mg, 2.35 mmol). The reaction mixture was heated at 80° C. for 3 h. The reaction mixture was cooled to room temperature and the solvent was removed under reduced pressure to yield the crude product, which was purified by preparative HPLC (HPLC Method: Column: INERTSIL ODS 21.2×250 mm, 5 μm; Mobile Phase A: 0.1% TFA in water; Mobile Phase B: acetonitrile; Gradient: 30-80% B over 14 minutes, then a 5 minute hold at 100% B; Flow: 17 mL/min), fractions were concentrated under reduced pressure and lyophilized from (EtOH/H₂O, 1:5) to yield Example 46 and Example 47.

Example 46: 140 mg, 21% yield; LCMS: m/z=566.2 (M+H); rt 3.26 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.83 (br s, 1H), 8.19-8.31 (m, 2H), 7.95-8.12 (m, 2H), 7.53-7.63 (m, 2H), 7.12-7.26 (m, 2H), 5.41-6.26 (m, 1H), 4.79-5.20 (m, 2H), 3.60-3.74 (m, 1H), 3.44 (s, 3H), 2.73-2.87 (m, 1H), 2.22-2.42 (m, 2H), 1.40-1.68 (m, 5H), 0.53-0.71 (m, 3H).

Example 47: 155 mg, 23% yield; LCMS: m/z=566.2 (M+H); rt 3.25 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.92 (s, 1H), 8.17-8.27 (m, 2H), 7.90-8.02 (m, 2H), 7.60-7.67 (m, 2H), 7.14-7.22 (m, 2H), 5.52-6.07 (m, 1H), 4.87-5.08 (m, 2H), 3.39-3.71 (m, 4H), 2.69-2.78 (m, 1H), 2.37-2.45 (m, 1H), 1.37-1.69 (m, 5H), 0.58-0.77 (m, 3H).

### Examples 96 and 97

### 4-((2S,5R)-4-((4-chlorophenyl)(pyridin-2-yl)methyl)-5-ethyl-2-methylpiperazin-1-yl)-1-methyl-2-oxo-1,2-dihydropyrido[3,2-d]pyrimidine-6-carbonitrile

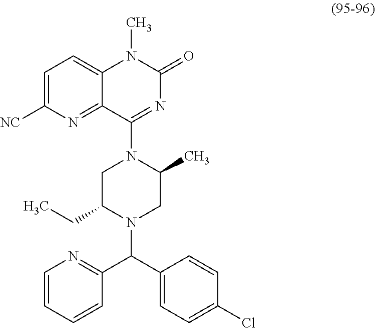 [SMILES: CC[C@@H]1CN(c2nc(=O)n(C)c3ccc(C#N)nc23)[C@@H](C)CN1C(c1ccc(Cl)cc1)c1ccccn1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile (100 mg, 0.32 mmol) in acetonitrile (5 mL) was added DIPEA (0.3 mL, 1.60 mmol), followed by 2-(bromo(4-chlorophenyl)methyl)pyridine (181 mg, 0.64 mmol). The reaction mixture was heated at 80° C. for 3 h. The reaction mixture was cooled to room temperature and the solvent was removed under reduced pressure to yield the crude product, which was purified by preparative HPLC (HPLC Method: Column: Cellulose-5 (250*20 ID) 5 micron; Mobile Phase A: 0.1% DEA in IPA; Mobile Phase B: 0.1% DEA in ACN; Gradient: 90% of B, then a 5 minute hold at 100% B; Flow: 18 mL/min), fractions were concentrated under reduced pressure and lyophilized from (EtOH/H₂O, 1:5) to yield Example 95 and Example 96.

Example 95: 24 mg, 14% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.52 (d, J=4.5 Hz, 1H), 8.23 (d, J=9.0 Hz, 1H), 7.96-8.02 (m, 1H), 7.75-7.81 (m, 1H), 7.59-7.68 (m, 3H), 7.39 (d, J=8.5 Hz, 2H), 7.22-7.29 (m, 1H), 5.54-5.95 (m, 1H), 4.81-5.07 (m, 2H), 3.39-3.68 (m, 5H), 2.69-2.76 (m, 1H), 2.35-2.44 (m, 1H), 1.37-1.67 (m, 5H), 0.58-0.67 (m, 3H).

Example 96: 22 mg, 13% yield; LCMS: m/z=514.2 (M+H); rt 2.94 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆): δ ppm 8.41-8.45 (m, 1H), 8.23 (d, J=9.0 Hz, 1H), 7.96-8.02 (m, 1H), 7.78-7.85 (m, 2H), 7.53-7.61 (m, 2H), 7.40 (d, J=8.5 Hz, 2H), 7.20-7.26 (m, 1H), 5.52-5.97 (m, 1H), 4.87-5.04 (m, 1H), 4.78-4.86 (m, 1H), 3.37-3.71 (m, 4H), 2.72-2.78 (m, 1H), 2.54-2.63 (m, 1H), 2.35-2.46 (m, 1H), 1.40-1.64 (m, 5H), 0.58-0.70 (m, 3H).

The examples in the Table 2 were prepared according to the general procedures described in Examples 1 to 4, using the appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 2

| Ex. | | Stereo | LCMS | LCMS | | Coupling |
|---|---|---|---|---|---|---|
| No. | Structure | chem. | Method | RT | M + H | Method |
| 48 | 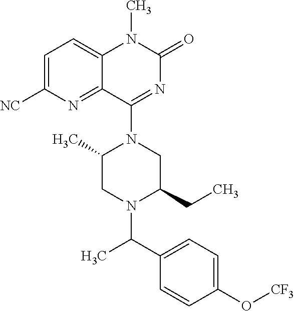 [SMILES: CC[C@@H]1CN(c2nc(=O)n(C)c3ccc(C#N)nc23)[C@@H](C)CN1C(C)c1ccc(OC(F)(F)F)cc1] | H | A | 2.34 | 501.2 | B |
| 49 | 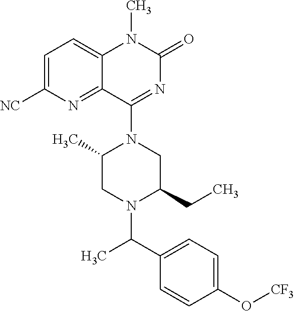 [SMILES: CC[C@@H]1CN(c2nc(=O)n(C)c3ccc(C#N)nc23)[C@@H](C)CN1C(C)c1ccc(OC(F)(F)F)cc1] | H | A | 2.39 | 501.2 | B |
| 50 | 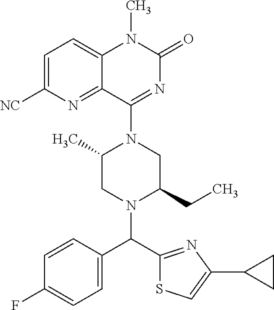 [SMILES: CC[C@@H]1CN(c2nc(=O)n(C)c3ccc(C#N)nc23)[C@@H](C)CN1C(c1ccc(F)cc1)c1nc(C2CC2)cs1] | H | A | 2.19 | 544.3 | A |
| 51 | 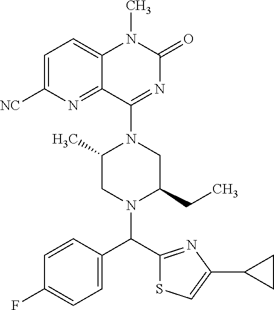 [SMILES: CC[C@@H]1CN(c2nc(=O)n(C)c3ccc(C#N)nc23)[C@@H](C)CN1C(c1ccc(F)cc1)c1nc(C2CC2)cs1] | H | A | 2.24 | 544.3 | A |
| 52 | 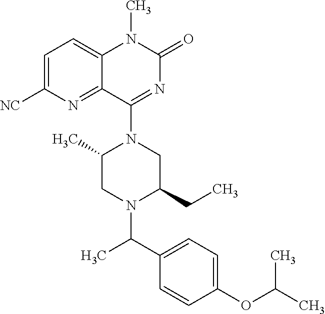 [SMILES: CC[C@@H]1CN(c2nc(=O)n(C)c3ccc(C#N)nc23)[C@@H](C)CN1C(C)c1ccc(OC(C)C)cc1] | H | A | 2.27 | 475.3 | B |
| 53 | 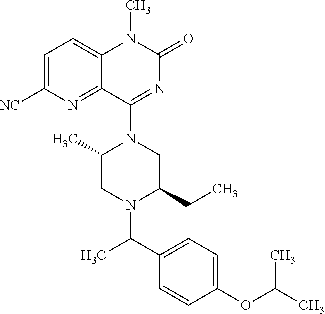 [SMILES: CC[C@@H]1CN(c2nc(=O)n(C)c3ccc(C#N)nc23)[C@@H](C)CN1C(C)c1ccc(OC(C)C)cc1] | H | A | 2.35 | 475.3 | B |
| 54 | 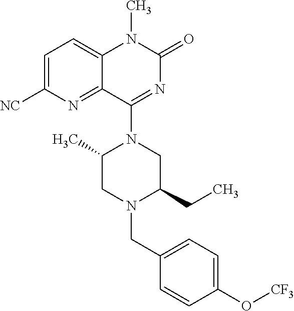 [SMILES: CC[C@@H]1CN(c2nc(=O)n(C)c3ccc(C#N)nc23)[C@@H](C)CN1Cc1ccc(OC(F)(F)F)cc1] | H | A | 2.29 | 487.2 | A |
| 55 | 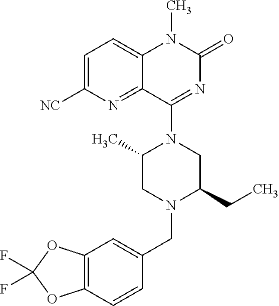 [SMILES: CC[C@@H]1CN(c2nc(=O)n(C)c3ccc(C#N)nc23)[C@@H](C)CN1Cc1ccc2c(c1)OC(F)(F)O2] | H | A | 2.24 | 483.2 | A |
| 56 | 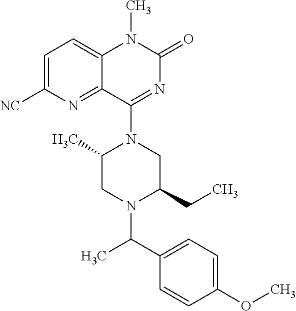 [SMILES: CC[C@@H]1CN(c2nc(=O)n(C)c3ccc(C#N)nc23)[C@@H](C)CN1C(C)c1ccc(OC)cc1] | H | A | 2.01 | 447.3 | B |
| 57 | 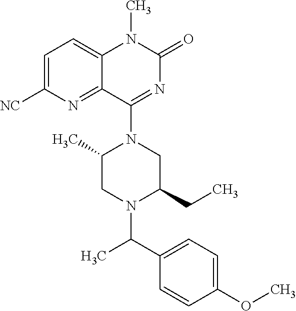 [SMILES: CC[C@@H]1CN(c2nc(=O)n(C)c3ccc(C#N)nc23)[C@@H](C)CN1C(C)c1ccc(OC)cc1] | H | A | 2.09 | 447.3 | B |
| 58 | 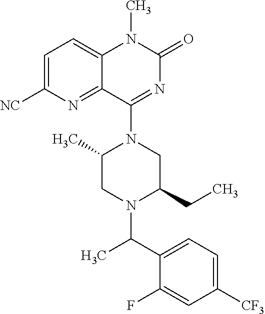 [SMILES: CC[C@@H]1CN(c2nc(=O)n(C)c3ccc(C#N)nc23)[C@@H](C)CN1C(C)c1ccc(C(F)(F)F)cc1F] | H | A | 2.34 | 503.3 | B |
| 59 | 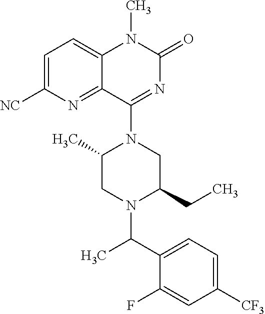 [SMILES: CC[C@@H]1CN(c2nc(=O)n(C)c3ccc(C#N)nc23)[C@@H](C)CN1C(C)c1ccc(C(F)(F)F)cc1F] | H | A | 2.39 | 503.2 | B |
| 60 | 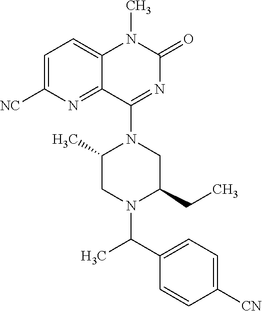 [SMILES: CC[C@@H]1CN(c2nc(=O)n(C)c3ccc(C#N)nc23)[C@@H](C)CN1C(C)c1ccc(C#N)cc1] | H | A | 1.89 | 442.3 | B |
| 61 | 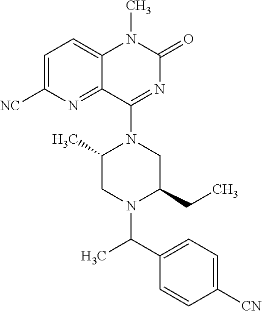 [SMILES: CC[C@@H]1CN(c2nc(=O)n(C)c3ccc(C#N)nc23)[C@@H](C)CN1C(C)c1ccc(C#N)cc1] | H | A | 1.96 | 442.3 | B |
| 62 | 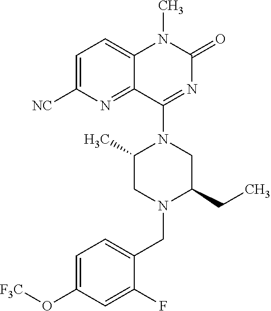 [SMILES: CC[C@@H]1CN(c2nc(=O)n(C)c3ccc(C#N)nc23)[C@@H](C)CN1Cc1ccc(OC(F)(F)F)cc1F] | H | A | 2.29 | 505.3 | A |
| 63 | 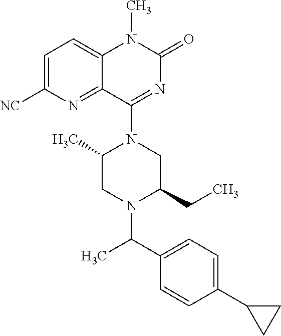 [SMILES: CC[C@@H]1CN(c2nc(=O)n(C)c3ccc(C#N)nc23)[C@@H](C)CN1C(C)c1ccc(C2CC2)cc1] | H | A | 2.34 | 457.3 | B |
| 64 | 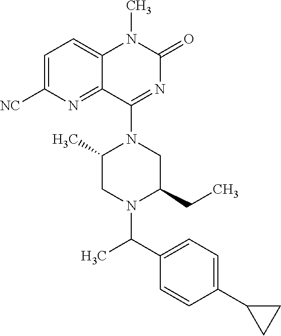 [SMILES: CC[C@@H]1CN(c2nc(=O)n(C)c3ccc(C#N)nc23)[C@@H](C)CN1C(C)c1ccc(C2CC2)cc1] | H | A | 2.41 | 457.3 | B |
| 65 | 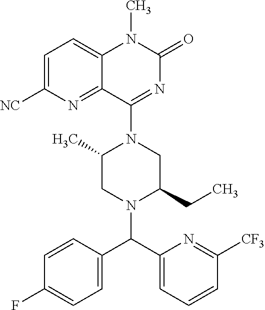 [SMILES: CC[C@@H]1CN(c2nc(=O)n(C)c3ccc(C#N)nc23)[C@@H](C)CN1C(c1ccc(F)cc1)c1cccc(C(F)(F)F)n1] | H | A | 2.24 | 566.2 | A |
| 66 | 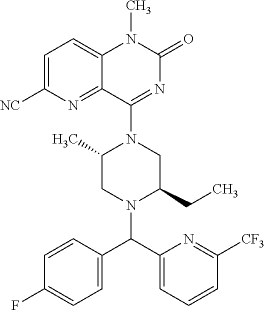 [SMILES: CC[C@@H]1CN(c2nc(=O)n(C)c3ccc(C#N)nc23)[C@@H](C)CN1C(c1ccc(F)cc1)c1cccc(C(F)(F)F)n1] | H | A | 2.25 | 566.2 | A |
| 67 | 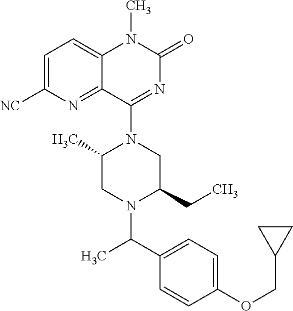 [SMILES: CC[C@@H]1CN(c2nc(=O)n(C)c3ccc(C#N)nc23)[C@@H](C)CN1C(C)c1ccc(OCC2CC2)cc1] | H | A | 1.42 | 487.3 | B |
| 68 | 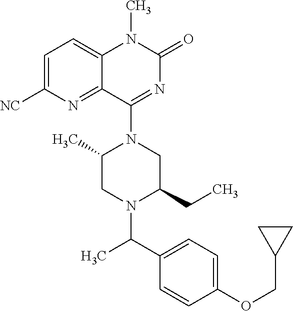 [SMILES: CC[C@@H]1CN(c2nc(=O)n(C)c3ccc(C#N)nc23)[C@@H](C)CN1C(C)c1ccc(OCC2CC2)cc1] | H | A | 2.36 | 487.3 | B |
| 69 | 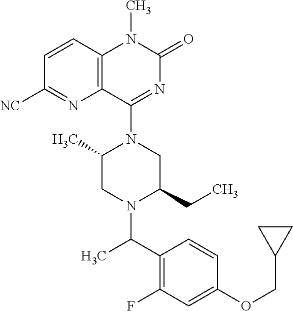 [SMILES: CC[C@@H]1CN(c2nc(=O)n(C)c3ccc(C#N)nc23)[C@@H](C)CN1C(C)c1ccc(OCC2CC2)cc1F] | H | A | 2.33 | 505.3 | B |
| 70 | 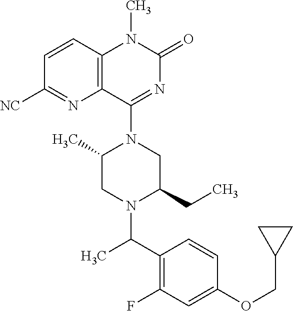 [SMILES: CC[C@@H]1CN(c2nc(=O)n(C)c3ccc(C#N)nc23)[C@@H](C)CN1C(C)c1ccc(OCC2CC2)cc1F] | H | A | 2.41 | 505.3 | B |
| 71 | 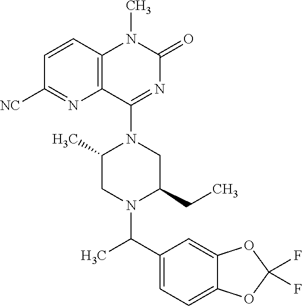 [SMILES: CC[C@@H]1CN(c2nc(=O)n(C)c3ccc(C#N)nc23)[C@@H](C)CN1C(C)c1ccc2c(c1)OC(F)(F)O2] | H | A | 2.32 | 497.3 | B |
| 72 | 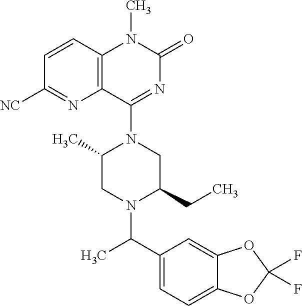 [SMILES: CC[C@@H]1CN(c2nc(=O)n(C)c3ccc(C#N)nc23)[C@@H](C)CN1C(C)c1ccc2c(c1)OC(F)(F)O2] | H | A | 2.37 | 497.3 | B |
| 73 | 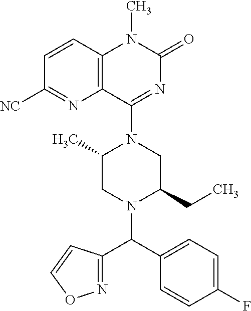 [SMILES: CC[C@@H]1CN(c2nc(=O)n(C)c3ccc(C#N)nc23)[C@@H](C)CN1C(c1ccc(F)cc1)c1ccon1] | H | A | 1.88 | 488.3 | A |
| 74 | 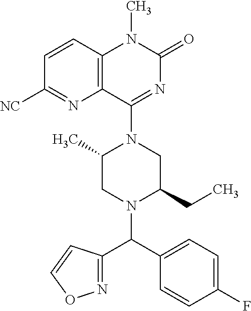 [SMILES: CC[C@@H]1CN(c2nc(=O)n(C)c3ccc(C#N)nc23)[C@@H](C)CN1C(c1ccc(F)cc1)c1ccon1] | H | A | 1.89 | 488.3 | A |
| 75 | 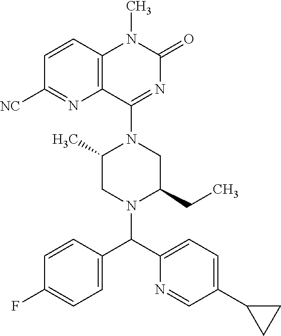 [SMILES: CC[C@@H]1CN(c2nc(=O)n(C)c3ccc(C#N)nc23)[C@@H](C)CN1C(c1ccc(F)cc1)c1ccc(C2CC2)cn1] | H | A | 2.12 | 538.3 | A |
| 76 | 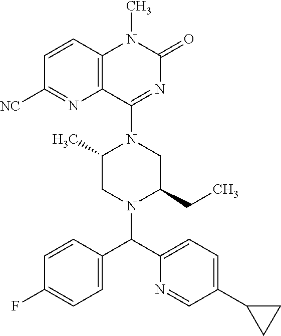 [SMILES: CC[C@@H]1CN(c2nc(=O)n(C)c3ccc(C#N)nc23)[C@@H](C)CN1C(c1ccc(F)cc1)c1ccc(C2CC2)cn1] | H | A | 2.12 | 538.3 | A |
| 77 | 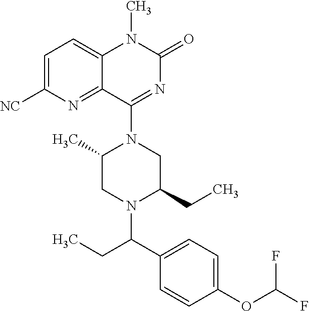 [SMILES: CCC(c1ccc(OC(F)F)cc1)N1C[C@H](C)N(c2nc(=O)n(C)c3ccc(C#N)nc23)C[C@H]1CC] | H | A | 2.28 | 497.3 | B |
| 78 | 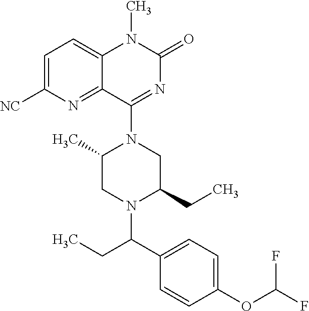 [SMILES: CCC(c1ccc(OC(F)F)cc1)N1C[C@H](C)N(c2nc(=O)n(C)c3ccc(C#N)nc23)C[C@H]1CC] | H | A | 2.27 | 497.3 | B |
| 79 | 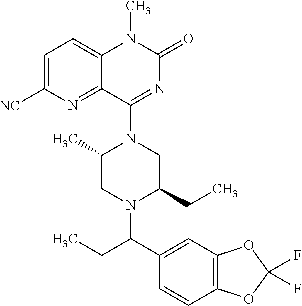 [SMILES: CCC(c1ccc2c(c1)OC(F)(F)O2)N1C[C@H](C)N(c2nc(=O)n(C)c3ccc(C#N)nc23)C[C@H]1CC] | H | A | 2.41 | 511.3 | B |
| 80 | 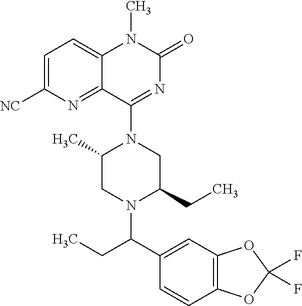 [SMILES: CCC(c1ccc2c(c1)OC(F)(F)O2)N1C[C@H](C)N(c2nc(=O)n(C)c3ccc(C#N)nc23)C[C@H]1CC] | H | A | 2.47 | 511.3 | B |
| 81 | 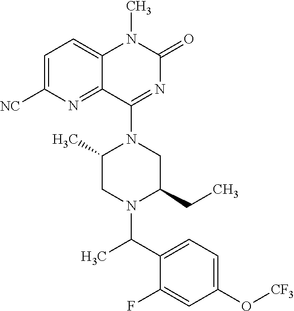 [SMILES: CC[C@@H]1CN(c2nc(=O)n(C)c3ccc(C#N)nc23)[C@@H](C)CN1C(C)c1ccc(OC(F)(F)F)cc1F] | H | A | 2.37 | 519.3 | B |
| 82 | 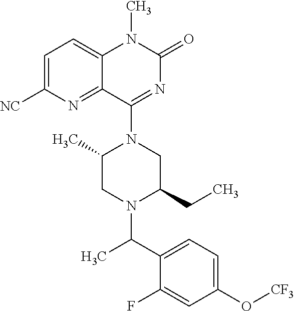 [SMILES: CC[C@@H]1CN(c2nc(=O)n(C)c3ccc(C#N)nc23)[C@@H](C)CN1C(C)c1ccc(OC(F)(F)F)cc1F] | H | A | 2.43 | 519.3 | B |
| 83 | 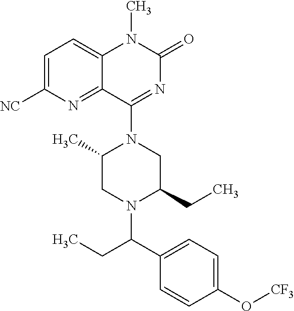 [SMILES: CCC(c1ccc(OC(F)(F)F)cc1)N1C[C@H](C)N(c2nc(=O)n(C)c3ccc(C#N)nc23)C[C@H]1CC] | H | A | 2.45 | 515.3 | B |
| 84 | 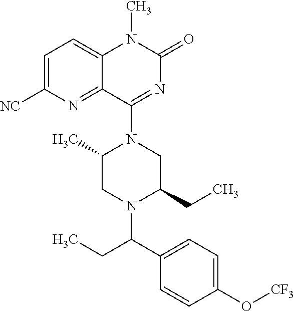 [SMILES: CCC(c1ccc(OC(F)(F)F)cc1)N1C[C@H](C)N(c2nc(=O)n(C)c3ccc(C#N)nc23)C[C@H]1CC] | H | A | 2.51 | 515.3 | B |
| 85 | 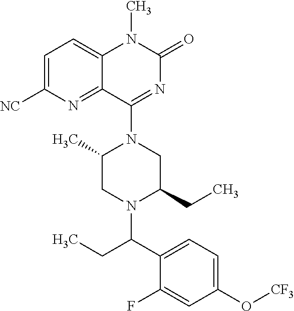 [SMILES: CCC(c1ccc(OC(F)(F)F)cc1F)N1C[C@H](C)N(c2nc(=O)n(C)c3ccc(C#N)nc23)C[C@H]1CC] | H | A | 2.47 | 533.3 | B |
| 86 | 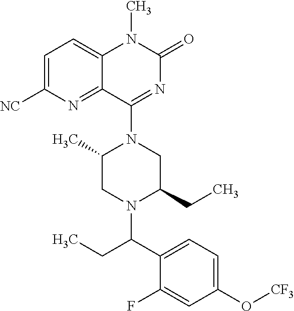 [SMILES: CCC(c1ccc(OC(F)(F)F)cc1F)N1C[C@H](C)N(c2nc(=O)n(C)c3ccc(C#N)nc23)C[C@H]1CC] | H | A | 2.53 | 533.3 | B |
| 87 | 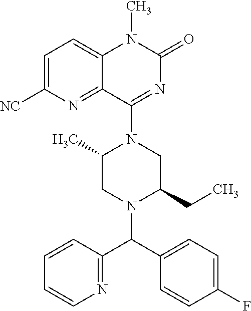 [SMILES: CC[C@@H]1CN(c2nc(=O)n(C)c3ccc(C#N)nc23)[C@@H](C)CN1C(c1ccc(F)cc1)c1ccccn1] | H | A | 1.88 | 498.3 | A |
| 88 | 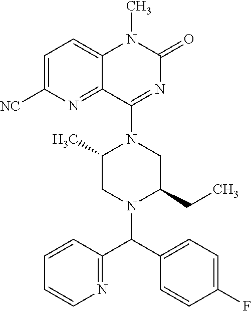 [SMILES: CC[C@@H]1CN(c2nc(=O)n(C)c3ccc(C#N)nc23)[C@@H](C)CN1C(c1ccc(F)cc1)c1ccccn1] | H | A | 1.87 | 498.3 | A |
| 89 | 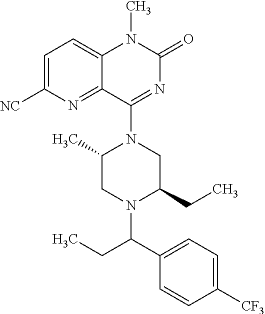 [SMILES: CCC(c1ccc(C(F)(F)F)cc1)N1C[C@H](C)N(c2nc(=O)n(C)c3ccc(C#N)nc23)C[C@H]1CC] | H | A | 2.41 | 499.3 | B |
| 90 | 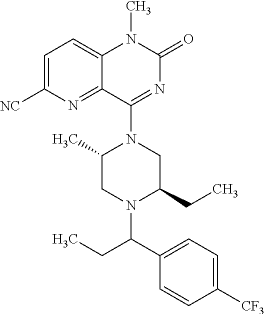 [SMILES: CCC(c1ccc(C(F)(F)F)cc1)N1C[C@H](C)N(c2nc(=O)n(C)c3ccc(C#N)nc23)C[C@H]1CC] | H | A | 2.47 | 499.3 | B |
| 91 | 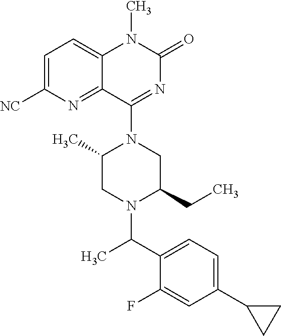 [SMILES: CC[C@@H]1CN(c2nc(=O)n(C)c3ccc(C#N)nc23)[C@@H](C)CN1C(C)c1ccc(C2CC2)cc1F] | H | A | 2.35 | 475.3 | B |
| 92 | 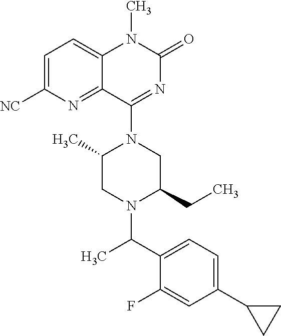 [SMILES: CC[C@@H]1CN(c2nc(=O)n(C)c3ccc(C#N)nc23)[C@@H](C)CN1C(C)c1ccc(C2CC2)cc1F] | H | A | 2.41 | 475.3 | B |
| 93 | 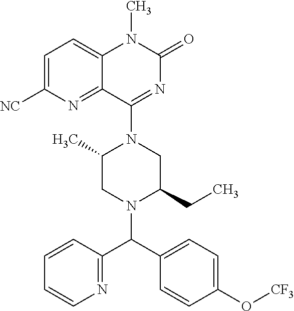 [SMILES: CC[C@@H]1CN(c2nc(=O)n(C)c3ccc(C#N)nc23)[C@@H](C)CN1C(c1ccc(OC(F)(F)F)cc1)c1ccccn1] | H | A | 2.1 | 564.3 | A |
| 94 | 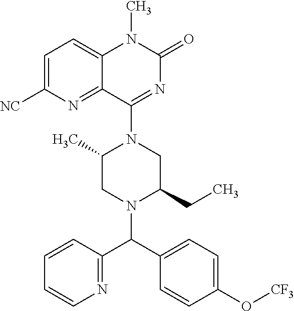 [SMILES: CC[C@@H]1CN(c2nc(=O)n(C)c3ccc(C#N)nc23)[C@@H](C)CN1C(c1ccc(OC(F)(F)F)cc1)c1ccccn1] | H | A | 2.1 | 564.3 | A |
| 97 | 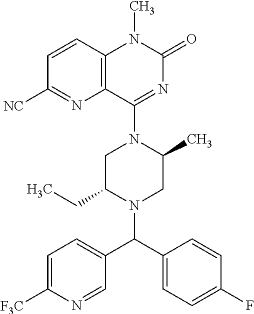 [SMILES: CC[C@@H]1CN(c2nc(=O)n(C)c3ccc(C#N)nc23)[C@@H](C)CN1C(c1ccc(F)cc1)c1ccc(C(F)(F)F)nc1] | H | A | 2.16 | 566.2 | A |
| 98 | 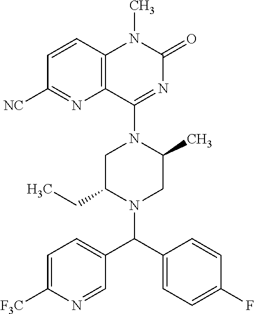 [SMILES: CC[C@@H]1CN(c2nc(=O)n(C)c3ccc(C#N)nc23)[C@@H](C)CN1C(c1ccc(F)cc1)c1ccc(C(F)(F)F)nc1] | H | A | 2.17 | 566.2 | A |
| 99 | 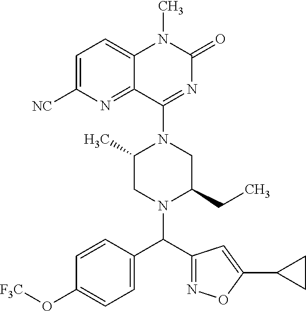 [SMILES: CC[C@@H]1CN(c2nc(=O)n(C)c3ccc(C#N)nc23)[C@@H](C)CN1C(c1ccc(OC(F)(F)F)cc1)c1cc(C2CC2)on1] | A | 2.32 | 594.3 | A | |
| 100 | 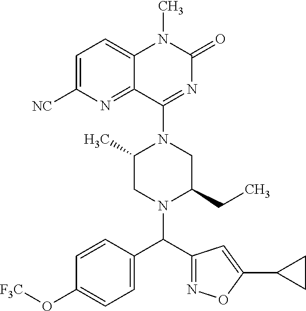 [SMILES: CC[C@@H]1CN(c2nc(=O)n(C)c3ccc(C#N)nc23)[C@@H](C)CN1C(c1ccc(OC(F)(F)F)cc1)c1cc(C2CC2)on1] | H | A | 2.31 | 594.3 | A |
| 101 | 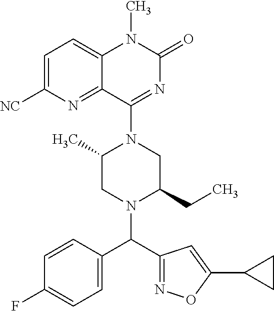 [SMILES: CC[C@@H]1CN(c2nc(=O)n(C)c3ccc(C#N)nc23)[C@@H](C)CN1C(c1ccc(F)cc1)c1cc(C2CC2)on1] | H | A | 2.10 | 528.3 | A |
| 102 | 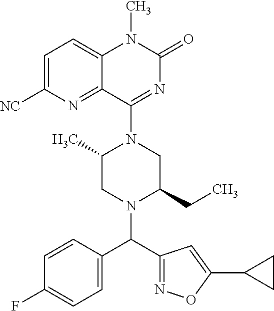 [SMILES: CC[C@@H]1CN(c2nc(=O)n(C)c3ccc(C#N)nc23)[C@@H](C)CN1C(c1ccc(F)cc1)c1cc(C2CC2)on1] | H | A | 2.09 | 528.3 | A |
| 103 | 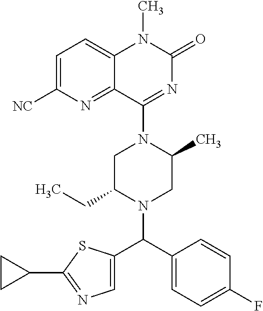 [SMILES: CC[C@@H]1CN(c2nc(=O)n(C)c3ccc(C#N)nc23)[C@@H](C)CN1C(c1ccc(F)cc1)c1cnc(C2CC2)s1] | H | A | 3.15 | 544.2 | A |
| 104 | 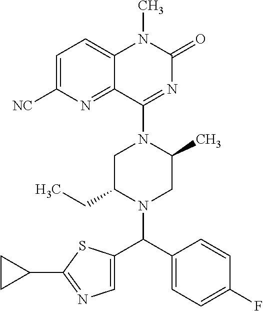 [SMILES: CC[C@@H]1CN(c2nc(=O)n(C)c3ccc(C#N)nc23)[C@@H](C)CN1C(c1ccc(F)cc1)c1cnc(C2CC2)s1] | H | A | 3.14 | 544.2 | A |

### Example 105

### 6-Chloro-4-((2S,5R)-4-((3-cyclopropyl-1,2,4-oxadiazol-5-yl)(4-fluorophenyl)methyl)-2,5-dimethylpiperazin-1-yl)-1-methylpyrido[3,2-d]pyrimidin-2(1H)-one

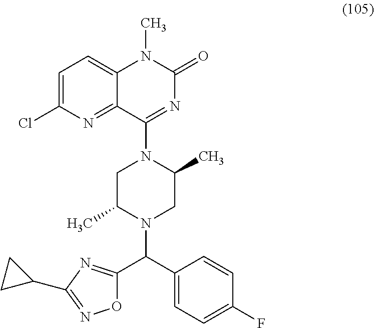 [SMILES: C[C@@H]1CN(c2nc(=O)n(C)c3ccc(Cl)nc23)[C@@H](C)CN1C(c1ccc(F)cc1)c1nc(C2CC2)no1]

To a stirred solution of 4,6-dichloro-1-methylpyrido[3,2-d]pyrimidin-2(1H)-one (100 mg, 0.44 mmol), 3-cyclopropyl-5-(((2R,5S)-2,5-dimethylpiperazin-1-yl)(4-fluorophenyl)methyl)-1,2,4-oxadiazole, TFA (213 mg, 0.48 mmol) in acetonitrile (2 mL) was added DIPEA (0.23 mL, 1.30 mmol). The reaction mixture was heated to 85° C. for 3 h. The reaction mixture was cooled to room temperature and concentrated under reduced pressure to give the crude product, which was purified by flash chromatography (24 g silica gel) by eluting with 10% MeOH in DCM to yield 6-chloro-4-((2S,5R)-4-((3-cyclopropyl-1,2,4-oxadiazol-5-yl)(4-fluorophenyl)methyl)-2,5-dimethylpiperazin-1-yl)-1-methylpyrido[3,2-d]pyrimidin-2(1H)-one (255 mg, 56% yield). LCMS: m/z, 524.3 (M+H); retention time 2.0 min; LCMS Method: Column: AQUITY UPLC BEH C18 (3.0×50 mm) 1.7 μm; Mobile phase A: 10 mM ammonium acetate:acetonitrile (98:2), Mobile phase B: 10 mM ammonium acetate:acetonitrile (2:98), Gradient=20-100% B over 4 minutes, then a 0.6 minute hold at 100% B; Temperature: 27° C.; Flow rate: 0.7 mL/min; Detection: UV at 220 nm).

### Examples 106 and 107

### 4-((2S,5R)-4-((3-Cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile

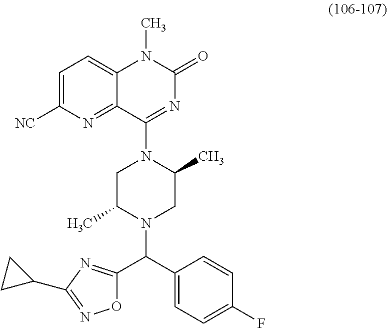 [SMILES: C[C@@H]1CN(c2nc(=O)n(C)c3ccc(C#N)nc23)[C@@H](C)CN1C(c1ccc(F)cc1)c1nc(C2CC2)no1]

To a stirred solution of 6-chloro-4-((2S,5R)-4-((3-cyclopropyl-1,2,4-oxadiazol-5-yl)(4-fluorophenyl)methyl)-2,5-dimethylpiperazin-1-yl)-1-methylpyrido[3,2-d]pyrimidin-2(1H)-one (150 mg, 0.29 mmol) in DMF (2.0 mL) were added zinc (28.1 mg, 0.43 mmol) and TEA (0.12 mL, 0.86 mmol). The reaction mixture was degassed with argon gas for 5 min followed by the addition of zinc cyanide (101 mg, 0.86 mmol) and dichloro[9,9-dimethyl-4,5-bis(diphenylphosphino)xanthene]palladium(II) (87 mg, 0.12 mmol). The reaction mixture heated at 85° C. for 16 h. The reaction mixture was cooled to room temperature and concentrated under reduced pressure to afford a residue which was purified via preparative HPLC (Chiral Separation Method: COLUMN: Chiralpak-ADH (250×4.6 mm), 5 μm; % CO₂=70%; Co solvent: 30% of acetonitrile:methanol (50:50), Total Flow: 80.0 g/min. Back pressure: 100 bar; temperature: 30° C.; UV detection: 215 nm).

Example 106: (4.5 mg, 3% yield): LCMS: m/z, 515.2 (M+H); rt 2.90 min; (LCMS method: Column: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Flow: 1.5 mL/min; Gradient: 100-20% B over 0.1 minutes then 0.3 min hold at 20% B, Flow: 1.5 mL/min. ¹H NMR (400 MHz, DMSO-d₆) δ ppm 8.23-8.21 (m, 1H), 7.99-7.97 (m, 1H), 7.65-7.62 (m, 2H), 7.28-7.24 (m, 2H), 5.21 (s, 1H), 3.43 (s, 3H), 3.03-2.94 (m, 2H), 2.23-2.08 (m, 2H), 1.14-1.05 (m, 3H), 1.05-1.03 (m, 2H), 0.90-0.88 (m, 5H), 3H obscured with moisture peak.

Example 107: (4.5 mg, 3% yield): LCMS: m/z, 515.2 (M+H); rt 2.90 min; (LCMS method: Column: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Flow: 1.5 mL/min; Gradient: 100-20% B over 0.1 minutes then 0.3 min hold at 20% B, Flow: 1.5 mL/min. ¹H NMR (400 MHz, DMSO-d₆) δ ppm 8.23-8.21 (m, 1H), 7.99-7.97 (m, 1H), 7.65-7.62 (m, 2H), 7.28-7.24 (m, 2H), 5.21 (s, 1H), 3.43 (s, 3H), 3.00-2.94 (m, 1H), 2.80-2.70 (m, 1H), 2.38-2.25 (m, 1H), 2.21-2.10 (m, 1H), 1.33-1.25 (m, 3H), 1.07-1.05 (m, 5H), 0.91-0.90 (m, 2H), 3H obscured with moisture peak.

The examples in the Table 3 were prepared according to the general procedure described in Examples 106 and 107, using the appropriate amidoxime. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 3

| Ex. | | Stereo | LCMS | LCMS | |
|---|---|---|---|---|---|
| No. | Structure | chem. | Method | RT | M + H |
| 108 | 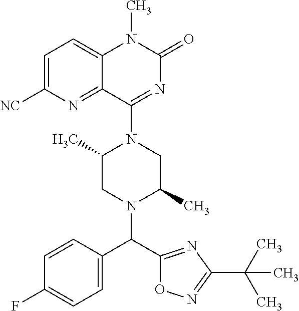 [SMILES: C[C@@H]1CN(c2nc(=O)n(C)c3ccc(C#N)nc23)[C@@H](C)CN1C(c1ccc(F)cc1)c1nc(C(C)(C)C)no1] | H | A | 2.25 | 531.2 |
| 109 | 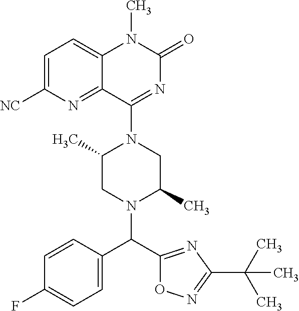 [SMILES: C[C@@H]1CN(c2nc(=O)n(C)c3ccc(C#N)nc23)[C@@H](C)CN1C(c1ccc(F)cc1)c1nc(C(C)(C)C)no1] | H | A | 2.24 | 531.2 |

### Intermediate 13

### 2-((2R,5S)-4-(6-Cyano-1-methyl-2-oxo-1,2-dihydropyrido[3,2-d]pyrimidin-4-yl)-2,5-diethylpiperazin-1-yl)-2-(4-fluorophenyl)acetic Acid

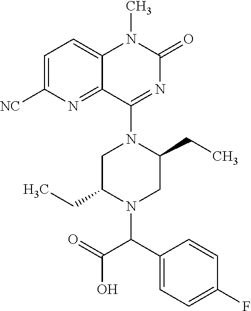 [SMILES: CC[C@H]1CN(C(C(=O)O)c2ccc(F)cc2)[C@H](CC)CN1c1nc(=O)n(C)c2ccc(C#N)nc12]

To a stirred solution of 4-((2S,5R)-2,5-diethylpiperazin-1-yl)-1-methyl-2-oxo-1,2-dihydropyrido[3,2-d]pyrimidine-6-carbonitrile (0.03 g, 0.09 mmol) in acetonitrile (2 mL) were added DIPEA (0.05 mL, 0.29 mmol) and 2-bromo-2-(4-fluorophenyl)acetic acid (0.03 g, 0.11 mmol). The reaction mixture was heated at 85° C. for 16 h. The reaction mixture cooled to room temperature and concentrated under reduced pressure to yield 2-((2R,5S)-4-(6-cyano-1-methyl-2-oxo-1,2-dihydropyrido[3,2-d]pyrimidin-4-yl)-2,5-diethylpiperazin-1-yl)-2-(4-fluorophenyl)acetic acid (44 mg, 17% yield) as a gummy liquid. LCMS: m/z 479.2 (M+H); retention time 0.80 min. LC-MS Method: Column-AQUITY UPLC BEH C18 (3.0×50 mm) 1.7 μm; Mobile phase A: Buffer:acetonitrile (95:5); Mobile phase B:Buffer: acetonitrile (5:95), Buffer: 10 mM ammonium acetate; Gradient: 20-100% B over 2.0 minutes, then a 0.2 minute hold at 100% B, flow rate 0.7 mL/min.

### Examples 110 and 111

### 4-((2S,5R)-4-((3-Cyclopropyl-1,2,4-oxadiazol-5-yl)(4-fluorophenyl)methyl)-2,5-dimethylpiperazin-1-yl)-1-methyl-2-oxo-1,2-dihydropyrido[3,2-d]pyrimidine-6-carbonitrile

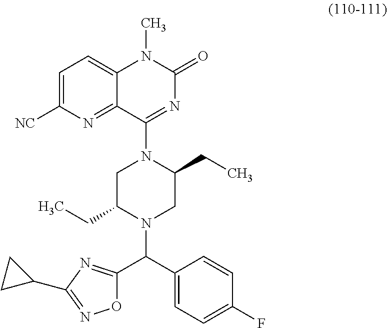 [SMILES: CC[C@H]1CN(C(c2ccc(F)cc2)c2nc(C3CC3)no2)[C@H](CC)CN1c1nc(=O)n(C)c2ccc(C#N)nc12]

To a stirred solution of 2-((2R,5S)-4-(6-cyano-1-methyl-2-oxo-1,2-dihydropyrido[3,2-d]pyrimidin-4-yl)-2,5-diethylpiperazin-1-yl)-2-(4-fluorophenyl)acetic acid, (0.045 g, 0.09 mmol), N-hydroxycyclopropanecarboximidamide (9.4 mg, 0.09 mmol) in DMF (2 mL), BOP (0.01 g, 0.23 mmol) and triethylamine (0.04 mL, 0.23 mmol) were added at room temperature. After 2 hours, the reaction mixture was heated at 110° C. for 3 h. The reaction mixture was cooled to room temperature and evaporated under reduced pressure to yield crude product, which was purified via preparative HPLC. Chiral Separation Method: Column: DAD-1-Cellulose-2 (250×4.6 mm), 5 micron. Mobile Phase: 0.10% DEA in acetonitrile, Flow: 2.0 mL\min.

Example 110: (1.9 mg, 6% yield): LCMS: m/z, 543.3 (M+H); rt 2.21 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50° C.; Time (min) Time (min): 0-3; % B: 0-100%). ¹H NMR (400 MHz, DMSO-d₆) δ ppm 8.29-8.16 (m, 1H), 8.06-7.92 (m, 1H), 7.75-7.58 (m, 2H), 7.26 (m, 2H), 6.01-5.32 (m, 1H), 5.28 (br s, 1H), 5.00-4.79 (m, 1H), 3.66-3.56 (m, 1H), 3.43 (s, 3H), 2.65-2.57 (m, 1H), 2.44-2.34 (m, 2H), 2.18-2.00 (m, 1H), 1.95-1.74 (m, 2H), 1.68-1.34 (m, 2H), 1.15-1.02 (m, 2H), 0.93-0.83 (m, 2H), 0.81-0.62 (m, 6H).

Example 111: (1.0 mg, 3% yield): LCMS: m/z, 543.3 (M+H); rt 2.20 min; LCMS method: Column: XBridge BEH XP C18 (50×2.1) mm, 2.5 μm; Mobile phase A: 95% water: 5% acetonitrile; 10 mM ammonium acetate; Mobile phase B: 5% water: 95% acetonitrile; 10 mM ammonium acetate; Flow: 1.1 mL/min; Temp: 50° C.; Time (min) Time (min): 0-3; % B: 0-100%). ¹H NMR (400 MHz, DMSO-d₆) δ ppm 8.23 (d, J=8.8 Hz, 1H), 8.06-7.91 (m, 1H), 7.62 (dd, J=6.2, 7.5 Hz, 2H), 7.26 (t, J=8.8 Hz, 2H), 5.92-5.31 (m, 1H), 5.29 (s, 1H), 4.96-4.78 (m, 1H), 3.60-3.50 (m, 1H), 3.43 (s, 3H), 3.25-3.10 (m, 1H), 2.97-2.75 (m, 2H), 2.27-1.65 (m, 3H), 1.49-1.24 (m, 2H), 1.11-0.97 (m, 2H), 0.94-0.75 (m, 5H), 0.74-0.50 (m, 3H).

The examples in the Table 4 were prepared according to the general procedure described in Examples 110 and 111, using the appropriate piperazine. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 4

| Ex. | | LCMS | LCMS | |
|---|---|---|---|---|
| No. | Structure | Method | RT | M + H |
| 112 | 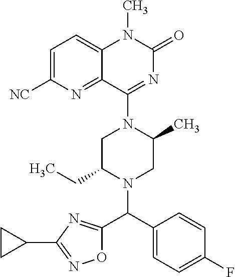 [SMILES: CC[C@@H]1CN(c2nc(=O)n(C)c3ccc(C#N)nc23)[C@@H](C)CN1C(c1ccc(F)cc1)c1nc(C2CC2)no1] | A | 2.75 | 529.2 |
| 113 | 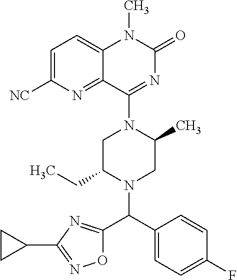 [SMILES: CC[C@@H]1CN(c2nc(=O)n(C)c3ccc(C#N)nc23)[C@@H](C)CN1C(c1ccc(F)cc1)c1nc(C2CC2)no1] | A | 2.75 | 529.2 |

The examples in the Table 5 were prepared according to the general procedure described in Examples 1 and 2, using appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 5

| Ex. | | LCMS | LCMS | | Coupling |
|---|---|---|---|---|---|
| No. | Structure | Method | RT | M + H | Method |
| 114 | 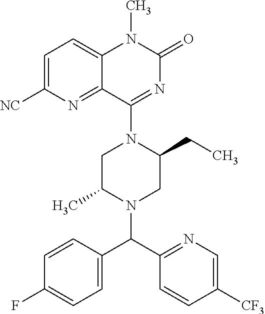 [SMILES: CC[C@H]1CN(C(c2ccc(F)cc2)c2ccc(C(F)(F)F)cn2)[C@H](C)CN1c1nc(=O)n(C)c2ccc(C#N)nc12] | A | 2.21 | 566.3 | A |
| 115 | 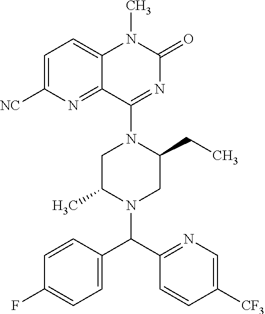 [SMILES: CC[C@H]1CN(C(c2ccc(F)cc2)c2ccc(C(F)(F)F)cn2)[C@H](C)CN1c1nc(=O)n(C)c2ccc(C#N)nc12] | A | 2.22 | 566.3 | A |
| 116 | 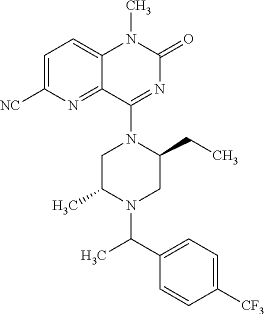 [SMILES: CC[C@H]1CN(C(C)c2ccc(C(F)(F)F)cc2)[C@H](C)CN1c1nc(=O)n(C)c2ccc(C#N)nc12] | A | 2.32 | 485.3 | B |
| 117 | 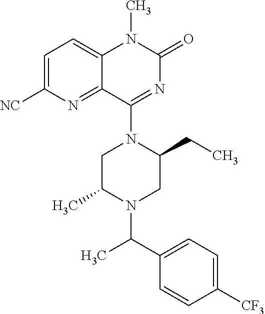 [SMILES: CC[C@H]1CN(C(C)c2ccc(C(F)(F)F)cc2)[C@H](C)CN1c1nc(=O)n(C)c2ccc(C#N)nc12] | A | 2.35 | 485.3 | B |
| 118 | 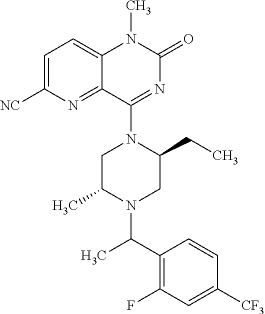 [SMILES: CC[C@H]1CN(C(C)c2ccc(C(F)(F)F)cc2F)[C@H](C)CN1c1nc(=O)n(C)c2ccc(C#N)nc12] | A | 2.37 | 503.3 | A |
| 119 | 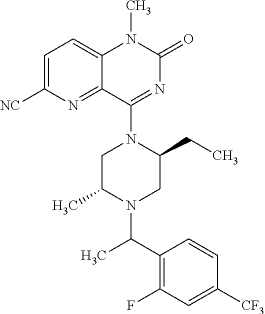 [SMILES: CC[C@H]1CN(C(C)c2ccc(C(F)(F)F)cc2F)[C@H](C)CN1c1nc(=O)n(C)c2ccc(C#N)nc12] | A | 2.34 | 503.2 | A |
| 120 | 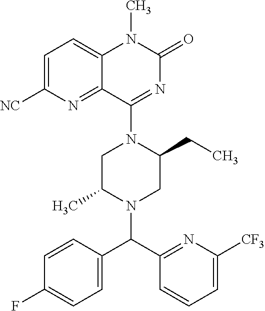 [SMILES: CC[C@H]1CN(C(c2ccc(F)cc2)c2cccc(C(F)(F)F)n2)[C@H](C)CN1c1nc(=O)n(C)c2ccc(C#N)nc12] | A | 2.24 | 566.3 | A |
| 121 | 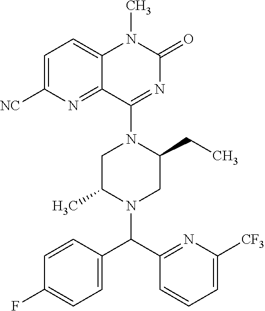 [SMILES: CC[C@H]1CN(C(c2ccc(F)cc2)c2cccc(C(F)(F)F)n2)[C@H](C)CN1c1nc(=O)n(C)c2ccc(C#N)nc12] | A | 2.23 | 566.2 | A |
| 122 | 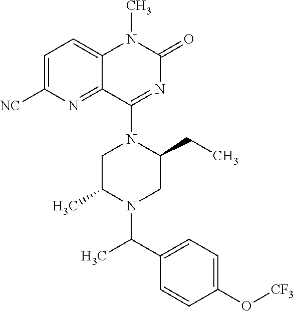 [SMILES: CC[C@H]1CN(C(C)c2ccc(OC(F)(F)F)cc2)[C@H](C)CN1c1nc(=O)n(C)c2ccc(C#N)nc12] | A | 2.39 | 501.3 | A |
| 123 | 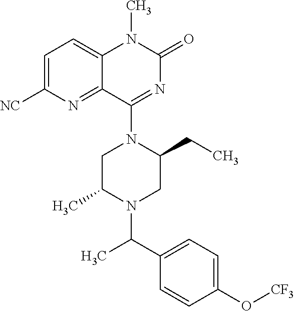 [SMILES: CC[C@H]1CN(C(C)c2ccc(OC(F)(F)F)cc2)[C@H](C)CN1c1nc(=O)n(C)c2ccc(C#N)nc12] | A | 2.36 | 501.3 | A |

### Examples 126 and 127

### 4-((2S,5R)-4-((4-fluorophenyl)(5-(trifluoromethyl)pyridin-2-yl)methyl)-2,5-dimethylpiperazin-1-yl)-1-methyl-2-oxo-1,2-dihydropyrido[3,2-d]pyrimidine-6-carbonitrile

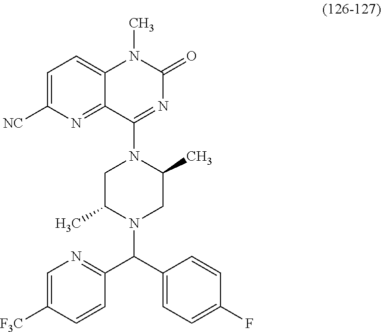 [SMILES: C[C@@H]1CN(c2nc(=O)n(C)c3ccc(C#N)nc23)[C@@H](C)CN1C(c1ccc(F)cc1)c1ccc(C(F)(F)F)cn1]

To a stirred solution of 4-((2S,5R)-2,5-dimethylpiperazin-1-yl)-1-methyl-2-oxo-1,2-dihydropyrido[3,2-d]pyrimidine-6-carbonitrile (1 g, 3.35 mmol) in acetonitrile (10 mL) was added DIPEA (5.9 mL, 33.5 mmol), followed by 2-(bromo(4-fluorophenyl) methyl)-5-(trifluoromethyl)pyridine (2.24 g, 6.70 mmol). The reaction mixture was heated at 80° C. for 4 h. The reaction mixture was cooled to room temperature and the solvent was removed under reduced pressure to yield the crude product, which was purified by preparative HPLC (HPLC Method: Column: Sunfire C18, 150×19 mm ID, 5 μm; Mobile Phase A: 0.1% TFA in water; Mobile Phase B: Acetonitrile:MeOH (1:1); Gradient: 50-100% B over 20 minutes, then a 5 minute hold at 100% B; Flow: 19 mL/min), fractions were concentrated under reduced pressure and lyophilized from (EtOH/H₂O, 1:5) to yield Example 126 and Example 127.

Example 126: 110 mg, 6% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 20-100%). ¹H NMR (400 MHz, DMSO-d₆) δ ppm 8.83 (s, 1H), 8.22 (d, J=9.0 Hz, 2H), 8.11-7.95 (m, 2H), 7.71-7.58 (m, 2H), 7.25-7.13 (m, 2H), 5.76-5.44 (m, 1H), 5.13-4.67 (m, 2H), 3.86-3.49 (m, 1H), 3.44 (s, 3H), 3.19-3.08 (m, 1H), 2.84 (dd, J=3.8, 12.3 Hz, 1H), 2.38-2.26 (m, 1H), 1.67-1.39 (m, 3H), 1.11-0.86 (m, 3H).

Example 127: 145 mg, 8% yield; LCMS: m/z=552.2 (M+H); rt 3.09 min; (LCMS method: Column: Column-Kinetex XB-C18 (75×3 mm-2.6 μm), Mobile phase A: 98% water: 2% acetonitrile; 10 mM ammonium formate; Mobile phase B: 2% Water: 98% acetonitrile; 10 mM ammonium formate; Flow: 1.0 mL/min; Temp: 50° C.; Time (min): 0-4; % B: 0-100%). ¹H NMR (400 MHz, DMSO-d₆) δ ppm 8.91 (s, 1H), 8.27-8.16 (m, 2H), 7.99 (d, J=9.0 Hz, 2H), 7.69-7.57 (m, 2H), 7.23-7.13 (m, 2H), 5.77-5.41 (m, 1H), 5.09-4.62 (m, 2H), 3.90-3.65 (m, 1H), 3.44 (s, 3H), 3.14-3.02 (m, 1H), 2.80-2.74 (m, 1H), 1.61-1.40 (m, 3H), 1.10-0.93 (m, 3H) [1H obscured with solvent peak].

The examples in the Table 6 were prepared from general procedure described in Examples 1 and 2, using appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 6

| Ex. | | Stereo | LCMS | LCMS | | Coupling |
|---|---|---|---|---|---|---|
| No. | Structure | chem. | Method | RT | M + H | Method |
| 124 | 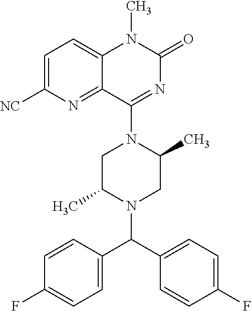 [SMILES: C[C@@H]1CN(c2nc(=O)n(C)c3ccc(C#N)nc23)[C@@H](C)CN1C(c1ccc(F)cc1)c1ccc(F)cc1] | H | B | 3.39 | 501.2 | A |
| 125 | 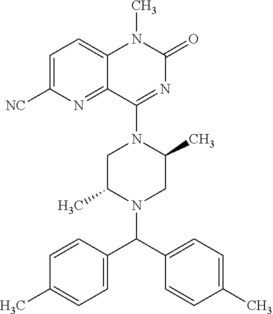 | H | B | 2.93 | 493.2 | A |
| 128 | 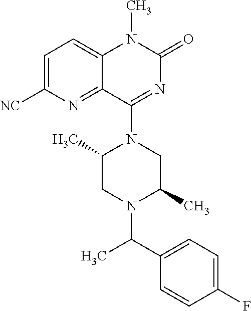 [SMILES: CC(c1ccc(F)cc1)N1C[C@H](C)N(c2nc(=O)n(C)c3ccc(C#N)nc23)C[C@H]1C] | H | A | 2.01 | 421.1 | A |
| 129 | 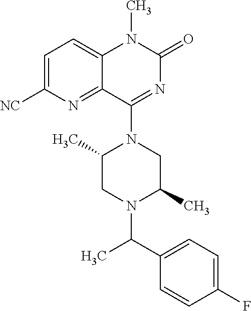 [SMILES: CC(c1ccc(F)cc1)N1C[C@H](C)N(c2nc(=O)n(C)c3ccc(C#N)nc23)C[C@H]1C] | H | A | 1.99 | 421.1 | A |
| 130 | 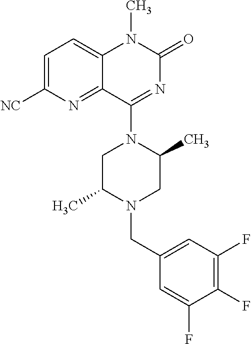 [SMILES: C[C@@H]1CN(c2nc(=O)n(C)c3ccc(C#N)nc23)[C@@H](C)CN1Cc1cc(F)c(F)c(F)c1] | H | A | 2.05 | 443.2 | A |
| 131 | 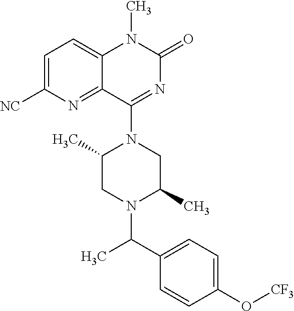 [SMILES: CC(c1ccc(OC(F)(F)F)cc1)N1C[C@H](C)N(c2nc(=O)n(C)c3ccc(C#N)nc23)C[C@H]1C] | H | A | 1.35 | 487.2 | B |
| 132 | 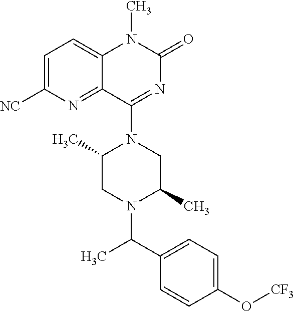 [SMILES: CC(c1ccc(OC(F)(F)F)cc1)N1C[C@H](C)N(c2nc(=O)n(C)c3ccc(C#N)nc23)C[C@H]1C] | H | A | 2.28 | 487.2 | B |
| 133 | 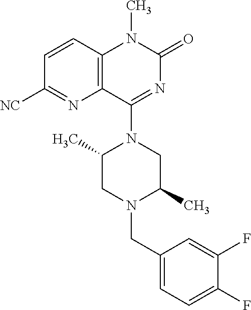 [SMILES: C[C@@H]1CN(c2nc(=O)n(C)c3ccc(C#N)nc23)[C@@H](C)CN1Cc1ccc(F)c(F)c1] | H | A | 1.97 | 425.2 | A |
| 134 | 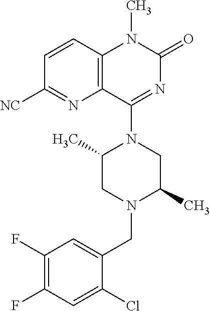 [SMILES: C[C@@H]1CN(c2nc(=O)n(C)c3ccc(C#N)nc23)[C@@H](C)CN1Cc1cc(F)c(F)cc1Cl] | H | A | 2.13 | 459.1 | A |
| 135 | 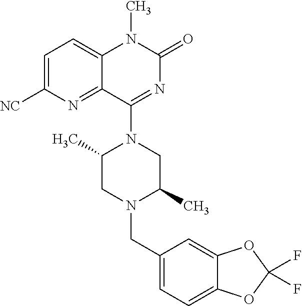 [SMILES: C[C@@H]1CN(c2nc(=O)n(C)c3ccc(C#N)nc23)[C@@H](C)CN1Cc1ccc2c(c1)OC(F)(F)O2] | H | A | 2.13 | 469.2 | A |
| 136 | 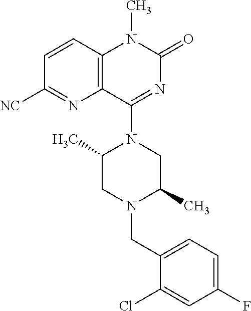 [SMILES: C[C@@H]1CN(c2nc(=O)n(C)c3ccc(C#N)nc23)[C@@H](C)CN1Cc1ccc(F)cc1Cl] | H | A | 1.07 | 441.2 | A |
| 137 | 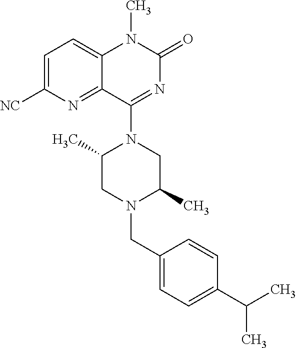 [SMILES: CC(C)c1ccc(CN2C[C@H](C)N(c3nc(=O)n(C)c4ccc(C#N)nc34)C[C@H]2C)cc1] | H | A | 2.29 | 431.2 | A |
| 138 | 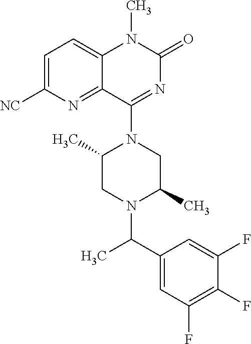 [SMILES: CC(c1cc(F)c(F)c(F)c1)N1C[C@H](C)N(c2nc(=O)n(C)c3ccc(C#N)nc23)C[C@H]1C] | H | A | 2.14 | 457.2 | A |
| 139 | 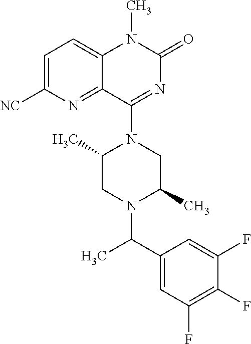 [SMILES: CC(c1cc(F)c(F)c(F)c1)N1C[C@H](C)N(c2nc(=O)n(C)c3ccc(C#N)nc23)C[C@H]1C] | H | A | 2.15 | 457.2 | A |
| 140 | 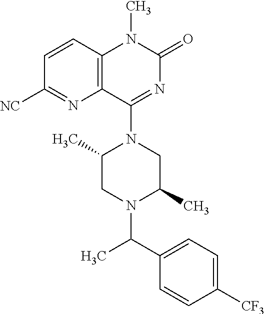 [SMILES: CC(c1ccc(C(F)(F)F)cc1)N1C[C@H](C)N(c2nc(=O)n(C)c3ccc(C#N)nc23)C[C@H]1C] | H | A | 2.23 | 471.2 | A |
| 141 | 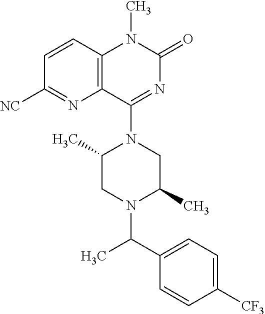 [SMILES: CC(c1ccc(C(F)(F)F)cc1)N1C[C@H](C)N(c2nc(=O)n(C)c3ccc(C#N)nc23)C[C@H]1C] | H | A | 2.23 | 471.2 | A |
| 142 | 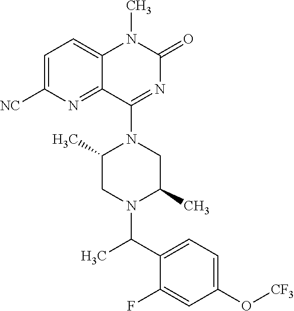 [SMILES: CC(c1ccc(OC(F)(F)F)cc1F)N1C[C@H](C)N(c2nc(=O)n(C)c3ccc(C#N)nc23)C[C@H]1C] | H | A | 2.3 | 505.2 | B |
| 143 | 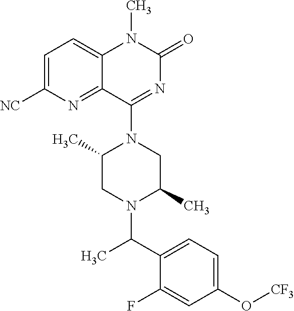 [SMILES: CC(c1ccc(OC(F)(F)F)cc1F)N1C[C@H](C)N(c2nc(=O)n(C)c3ccc(C#N)nc23)C[C@H]1C] | H | A | 2.3 | 505.2 | B |
| 144 | 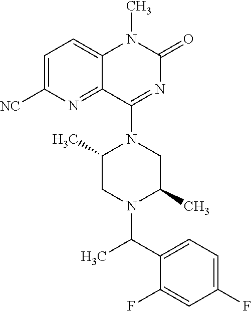 [SMILES: CC(c1ccc(F)cc1F)N1C[C@H](C)N(c2nc(=O)n(C)c3ccc(C#N)nc23)C[C@H]1C] | H | A | 2.06 | 439.2 | A |
| 145 | 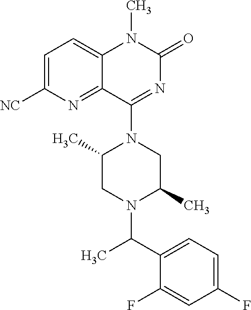 [SMILES: CC(c1ccc(F)cc1F)N1C[C@H](C)N(c2nc(=O)n(C)c3ccc(C#N)nc23)C[C@H]1C] | H | A | 2.06 | 439.1 | A |
| 146 | 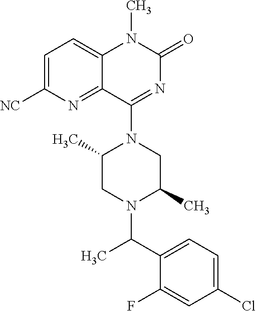 [SMILES: CC(c1ccc(Cl)cc1F)N1C[C@H](C)N(c2nc(=O)n(C)c3ccc(C#N)nc23)C[C@H]1C] | H | A | 2.23 | 455.1 | A |
| 147 | 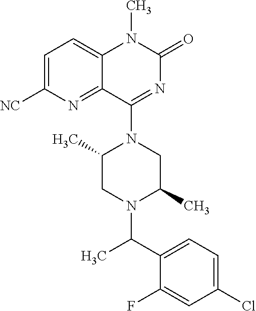 [SMILES: CC(c1ccc(Cl)cc1F)N1C[C@H](C)N(c2nc(=O)n(C)c3ccc(C#N)nc23)C[C@H]1C] | H | A | 2.23 | 455.2 | A |
| 148 | 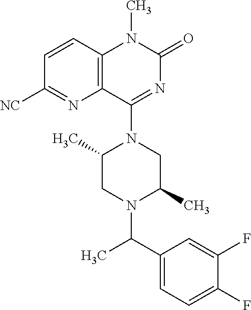 [SMILES: CC(c1ccc(F)c(F)c1)N1C[C@H](C)N(c2nc(=O)n(C)c3ccc(C#N)nc23)C[C@H]1C] | H | A | 2.07 | 439.2 | A |
| 149 | 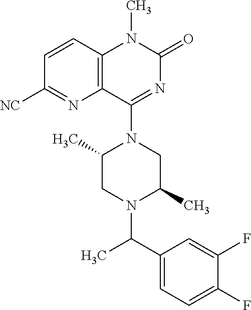 [SMILES: CC(c1ccc(F)c(F)c1)N1C[C@H](C)N(c2nc(=O)n(C)c3ccc(C#N)nc23)C[C@H]1C] | H | A | 2.08 | 439.2 | A |
| 150 | 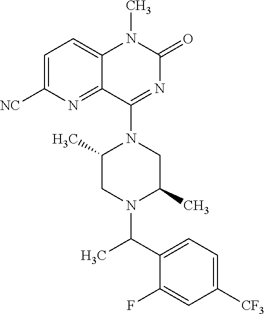 [SMILES: CC(c1ccc(C(F)(F)F)cc1F)N1C[C@H](C)N(c2nc(=O)n(C)c3ccc(C#N)nc23)C[C@H]1C] | H | A | 2.26 | 489.2 | A |
| 151 | 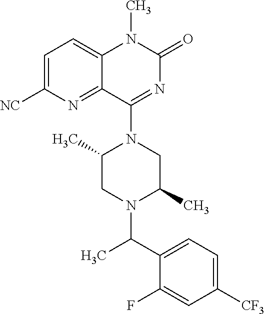 [SMILES: CC(c1ccc(C(F)(F)F)cc1F)N1C[C@H](C)N(c2nc(=O)n(C)c3ccc(C#N)nc23)C[C@H]1C] | H | A | 2.26 | 489.2 | A |
| 152 | 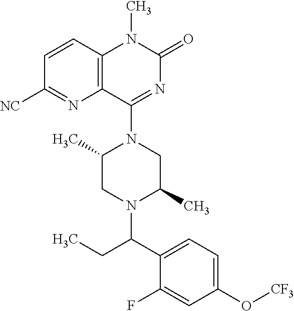 [SMILES: CCC(c1ccc(OC(F)(F)F)cc1F)N1C[C@H](C)N(c2nc(=O)n(C)c3ccc(C#N)nc23)C[C@H]1C] | H | A | 2.45 | 519.2 | A |
| 153 | 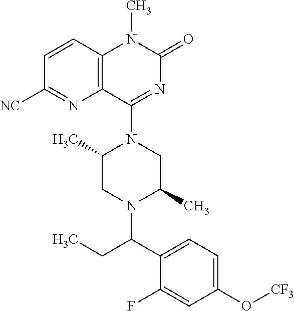 [SMILES: CCC(c1ccc(OC(F)(F)F)cc1F)N1C[C@H](C)N(c2nc(=O)n(C)c3ccc(C#N)nc23)C[C@H]1C] | H | A | 2.47 | 519.2 | A |
| 154 | 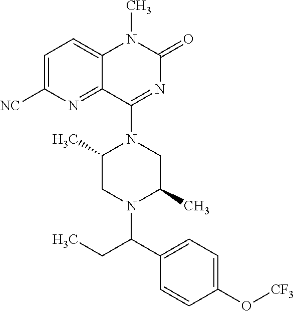 [SMILES: CCC(c1ccc(OC(F)(F)F)cc1)N1C[C@H](C)N(c2nc(=O)n(C)c3ccc(C#N)nc23)C[C@H]1C] | H | A | 2.36 | 501.2 | A |
| 155 | 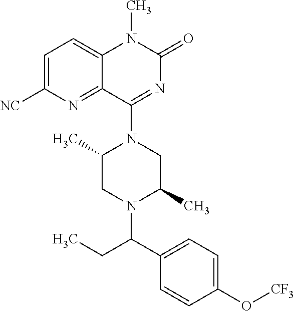 [SMILES: CCC(c1ccc(OC(F)(F)F)cc1)N1C[C@H](C)N(c2nc(=O)n(C)c3ccc(C#N)nc23)C[C@H]1C] | H | A | 2.38 | 501.2 | A |
| 156 | 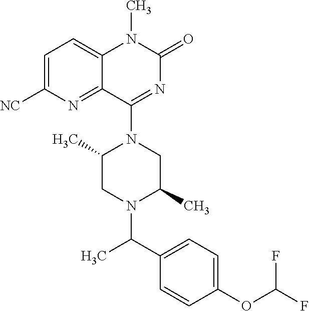 [SMILES: CC(c1ccc(OC(F)F)cc1)N1C[C@H](C)N(c2nc(=O)n(C)c3ccc(C#N)nc23)C[C@H]1C] | H | A | 2.02 | 469.2 | A |
| 157 | 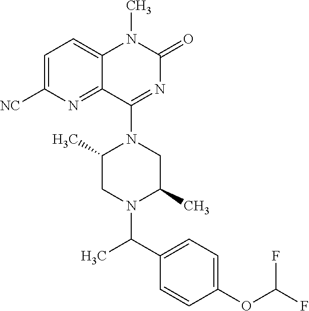 [SMILES: CC(c1ccc(OC(F)F)cc1)N1C[C@H](C)N(c2nc(=O)n(C)c3ccc(C#N)nc23)C[C@H]1C] | H | A | 2.05 | 469.2 | A |
| 158 | 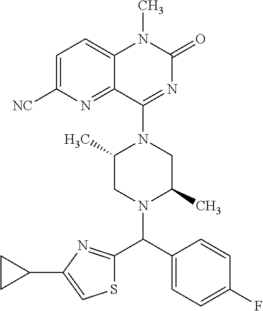 [SMILES: C[C@@H]1CN(c2nc(=O)n(C)c3ccc(C#N)nc23)[C@@H](C)CN1C(c1ccc(F)cc1)c1nc(C2CC2)cs1] | H | A | 2.11 | 530.3 | A |
| 159 | 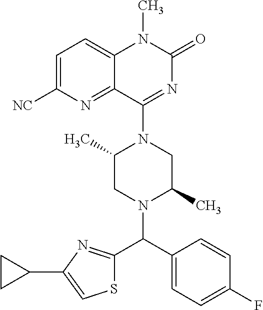 [SMILES: C[C@@H]1CN(c2nc(=O)n(C)c3ccc(C#N)nc23)[C@@H](C)CN1C(c1ccc(F)cc1)c1nc(C2CC2)cs1] | H | A | 2.14 | 530.3 | A |
| 160 | 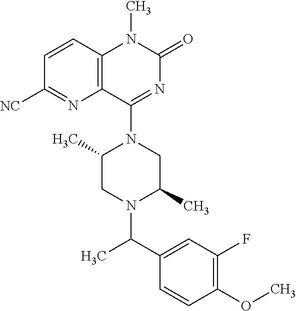 [SMILES: COc1ccc(C(C)N2C[C@H](C)N(c3nc(=O)n(C)c4ccc(C#N)nc34)C[C@H]2C)cc1F] | H | A | 2.28 | 505.2 | A |
| 161 | 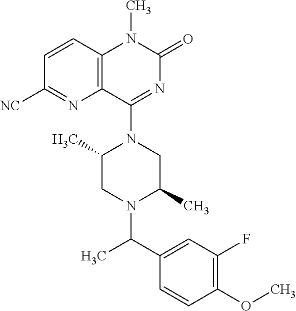 [SMILES: COc1ccc(C(C)N2C[C@H](C)N(c3nc(=O)n(C)c4ccc(C#N)nc34)C[C@H]2C)cc1F] | H | A | 2.29 | 505.2 | A |
| 162 | 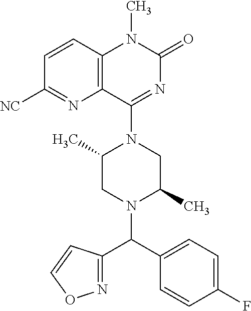 [SMILES: C[C@@H]1CN(c2nc(=O)n(C)c3ccc(C#N)nc23)[C@@H](C)CN1C(c1ccc(F)cc1)c1ccon1] | H | A | 1.80 | 474.2 | A |
| 163 | 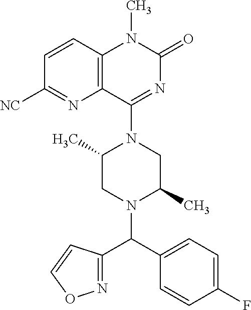 [SMILES: C[C@@H]1CN(c2nc(=O)n(C)c3ccc(C#N)nc23)[C@@H](C)CN1C(c1ccc(F)cc1)c1ccon1] | H | A | 1.79 | 474.2 | A |
| 164 | 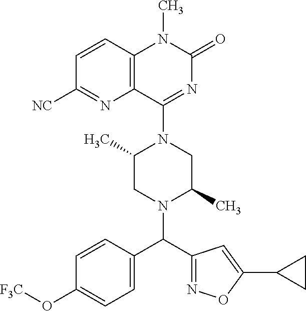 [SMILES: C[C@@H]1CN(c2nc(=O)n(C)c3ccc(C#N)nc23)[C@@H](C)CN1C(c1ccc(OC(F)(F)F)cc1)c1cc(C2CC2)on1] | H | A | 2.25 | 580.3 | A |
| 165 | 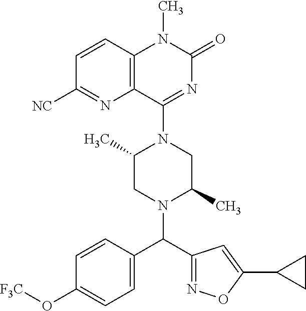 [SMILES: C[C@@H]1CN(c2nc(=O)n(C)c3ccc(C#N)nc23)[C@@H](C)CN1C(c1ccc(OC(F)(F)F)cc1)c1cc(C2CC2)on1] | H | A | 2.24 | 580.3 | A |
| 166 | 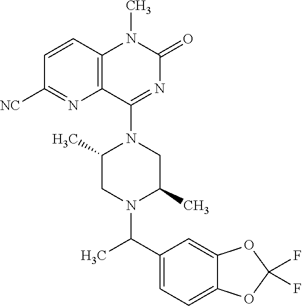 [SMILES: CC(c1ccc2c(c1)OC(F)(F)O2)N1C[C@H](C)N(c2nc(=O)n(C)c3ccc(C#N)nc23)C[C@H]1C] | H | A | 2.24 | 483.2 | A |
| 167 | 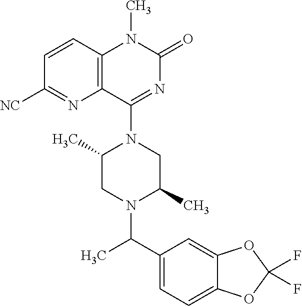 [SMILES: CC(c1ccc2c(c1)OC(F)(F)O2)N1C[C@H](C)N(c2nc(=O)n(C)c3ccc(C#N)nc23)C[C@H]1C] | H | A | 2.24 | 483.3 | A |
| 168 | 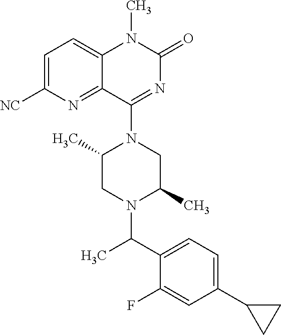 [SMILES: CC(c1ccc(C2CC2)cc1F)N1C[C@H](C)N(c2nc(=O)n(C)c3ccc(C#N)nc23)C[C@H]1C] | H | A | 2.27 | 461.3 | B |
| 169 | 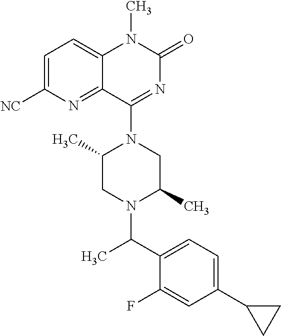 [SMILES: CC(c1ccc(C2CC2)cc1F)N1C[C@H](C)N(c2nc(=O)n(C)c3ccc(C#N)nc23)C[C@H]1C] | H | A | 2.27 | 461.3 | B |
| 170 | 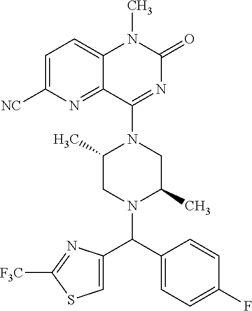 [SMILES: C[C@@H]1CN(c2nc(=O)n(C)c3ccc(C#N)nc23)[C@@H](C)CN1C(c1ccc(F)cc1)c1csc(C(F)(F)F)n1] | H | A | 2.19 | 558.2 | A |
| 171 | 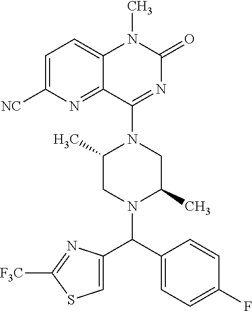 [SMILES: C[C@@H]1CN(c2nc(=O)n(C)c3ccc(C#N)nc23)[C@@H](C)CN1C(c1ccc(F)cc1)c1csc(C(F)(F)F)n1] | H | A | 2.2 | 558.2 | A |

### Intermediate 14

### tert-Butyl (2S,5S)-5-(hydroxymethyl)-2-methyl-4-(1-(4-(trifluoromethyl)phenyl)ethyl)piperazine-1-carboxylate (Diastereomeric Mixture)

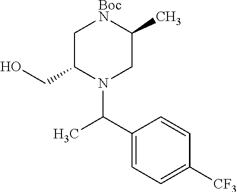 [SMILES: CC(c1ccc(C(F)(F)F)cc1)N1C[C@H](C)N(C(=O)OC(C)(C)C)C[C@H]1CO]

To a stirred solution of tert-butyl (2S,5S)-5-(hydroxymethyl)-2-methylpiperazine-1-carboxylate (2 g, 8.68 mmol) in acetonitrile (20 mL), DIPEA (7.6 mL, 43.4 mmol) and 1-(1-chloroethyl)-4-(trifluoromethyl)benzene (1.8 g, 8.68 mmol) were added sequentially at room temperature, followed by heating at 80° C. for 3 h. The reaction mixture was cooled to room temperature and solvent was removed under reduced pressure to give the crude product, which was purified by silica gel column chromatography using a 24 g silica gel flash column, eluting with 40-60% EtOAc in hexane to afford tert-butyl (2S,5S)-5-(hydroxymethyl)-2-methyl-4-(1-(4-(trifluoromethyl)phenyl)ethyl)piperazine-1-carboxylate (2.5 g, 61% yield) as an off-white solid. LCMS: m/z, 403.2 (M+H); rt 3.48 min. (LCMS Method: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Detection: UV at 220 nm).

### Intermediate 15

### ((2S,5S)-5-methyl-1-(1-(4-(trifluoromethyl)phenyl)ethyl)piperazin-2-yl)methanol·HCl Salt (Diastereomeric Mixture)

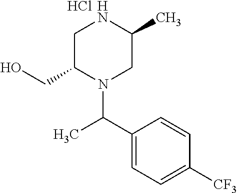 [SMILES: Cl ; CC(c1ccc(C(F)(F)F)cc1)N1C[C@H](C)NC[C@H]1CO]

To a stirred solution of tert-butyl (2S,5S)-5-(hydroxymethyl)-2-methyl-4-(1-(4-(trifluoromethyl)phenyl)ethyl)piperazine-1-carboxylate (0.7 g, 1.74 mmol) in DCM (10 mL), HCl (4 N in dioxane) (0.53 mL, 17.4 mmol) was added drop wise at room temperature. The reaction mixture was stirred for 3 h. Solvent was removed under reduced pressure, the solids were co-distilled with acetonitrile (3×10 mL), and dried to afford ((2S,5S)-5-methyl-1-(1-(4-(trifluoromethyl)phenyl)ethyl)piperazin-2-yl)methanol, HCl salt (0.5 g, 86% yield) as an off-white solid. LCMS: m/z, 303.2 (M+H); rt 1.77 & 2.07 min. (LCMS Method: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Detection: UV at 220 nm).

### Example 172

### 6-chloro-4-((2S,5S)-5-(hydroxymethyl)-2-methyl-4-(1-(4-(trifluoromethyl)phenyl)ethyl) piperazin-1-yl)-1-methylpyrido[3,2-d]pyrimidin-2(1H)-one (Diastereomeric Mixture)

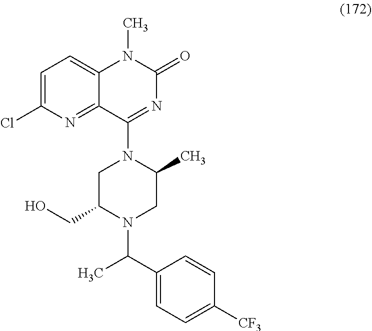 [SMILES: CC(c1ccc(C(F)(F)F)cc1)N1C[C@H](C)N(c2nc(=O)n(C)c3ccc(Cl)nc23)C[C@H]1CO]

To a stirred solution of ((2S,5S)-5-methyl-1-(1-(4-(trifluoromethyl)phenyl)ethyl) piperazin-2-yl)methanol, HCl salt (150 mg, 0.50 mmol) in acetonitrile (5 mL), DIPEA (0.44 mL, 2.48 mmol) and 4,6-dichloro-1-methylpyrido[3,2-d]pyrimidin-2(1H)-one (114 mg, 0.50 mmol) were added sequentially at room temperature followed by heating at 80° C. for 12 h. The reaction mixture was cooled to room temperature and solvent was removed under reduced pressure to give the crude product, which was purified by silica gel column chromatography using 12 g flash column, eluting with 0-10% MeOH in CHCl₃ to afford 6-chloro-4-((2S,5S)-5-(hydroxymethyl)-2-methyl-4-(1-(4-(trifluoromethyl)phenyl)ethyl)piperazin-1-yl)-1-methylpyrido[3,2-d]pyrimidin-2(1H)-one (150 mg, 57% yield). LCMS: m/z, 496.2 (M+H); rt 1.99 min. (LCMS Method: Column: Waters Acquity UPLC BEH C18 (3.0×50 mm) 1.7 μm, Mobile phase A: 10 mM ammonium acetate:acetonitrile (95:5); Mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Example 173

### 6-Chloro-4-((2S,5S)-5-(methoxymethyl)-2-methyl-4-(1-(4-(trifluoromethyl)phenyl)ethyl) piperazin-1-yl)-1-methylpyrido[3,2-d]pyrimidin-2(1H)-one (Diastereomeric Mixture)

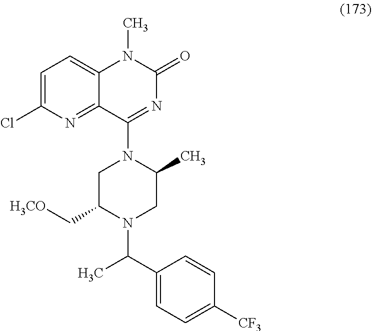 [SMILES: COC[C@@H]1CN(c2nc(=O)n(C)c3ccc(Cl)nc23)[C@@H](C)CN1C(C)c1ccc(C(F)(F)F)cc1]

To a stirred suspension of NaH (40.3 mg, 1.01 mmol, 60% w/w) in THF (5 mL) was added 6-chloro-4-((2S,5S)-5-(hydroxymethyl)-2-methyl-4-(1-(4-(trifluoromethyl) phenyl)ethyl)piperazin-1-yl)-1-methylpyrido[3,2-d]pyrimidin-2(1H)-one (250 mg, 0.50 mmol) at 0° C. After 5 minutes, a solution of methyl iodide (0.06 mL, 1.01 mmol) in THF (2 mL) was added and the reaction mixture was stirred for 2 h at room temperature. The reaction mixture was cooled to 0° C., quenched with ice cold water and extracted with EtOAc (2×50 mL). The combined organic extracts were washed with brine, dried over Na₂SO₄ and concentrated to give the crude product, which was purified by silica gel flash column chromatography (5-10% MeOH in DCM; 12 g column) to afford 6-chloro-4-((2S,5S)-5-(methoxymethyl)-2-methyl-4-(1-(4-(trifluoromethyl)phenyl)ethyl)piperazin-1-yl)-1-methylpyrido[3,2-d]pyrimidin-2(1H)-one (200 mg, 78% yield). LCMS: m/z, 510.1 (M+H); rt 1.08 and 1.11 min. (LCMS Method: Column: AQUITY UPLC BEH C18 (3.0×50 mm) 1.7 μm; Mobile phase A: 10 mM ammonium acetate:acetonitrile (95:5), Mobile phase B: 10 mM ammonium acetate:acetonitrile (5:95), Method: % B: 0 min-20: 2 min-100: 2.3 min-100, Temperature: 27° C.; Flow rate: 0.7 mL/min; Detection: UV at 220 nm).

### Examples 174 and 175

### 4-((2S,5S)-5-(Methoxymethyl)-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

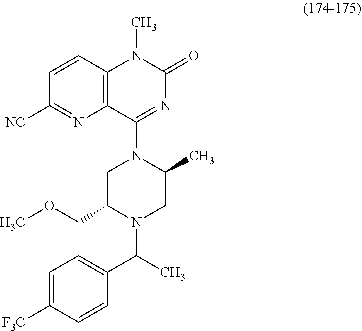

To a stirred solution of 6-chloro-4-((2S,5S)-5-(methoxymethyl)-2-methyl-4-(1-(4-(trifluoromethyl)phenyl)ethyl)piperazin-1-yl)-1-methylpyrido[3,2-d]pyrimidin-2(1H)-one (140 mg, 0.27 mmol) in DMF (5 mL) were added zinc (27 mg, 0.41 mmol) and TEA (0.15 mL, 1.08 mmol). The reaction mixture was degassed with argon gas for 5 min., followed by the addition of zinc cyanide (97 mg, 0.82 mmol) and dichloro[9,9-dimethyl-4,5-bis(diphenylphosphino)xanthene]palladium(II) (21 mg, 0.03 mmol). The reaction mixture was heated at 90° C. for 12 h. The reaction mixture was cooled to room temperature and concentrated under reduced pressure to afford a residue which was purified via preparative HPLC. (Chiral Separation Method: Column: Cellulose-5 (250*19 ID) 5 micron; Mobile Phase A: 10 mM NH₄OAc in MeOH; Flow: 20 mL/min; UV detection: 215 nm).

Example 174: (5 mg, 4% yield): LCMS: m/z, 501.3 (M+H); rt 2.11 min; (LCMS Method: Column: XBridge BEH XP C18 (2.1×50 mm), 2.5 μm; Mobile phase A: 10 mM ammonium acetate:acetonitrile (95:5), Mobile phase B: 10 mM ammonium acetate:acetonitrile (5:95), Gradient=0-100% B over 3 minutes; Temperature: 50° C.; Flow rate: 1.1 mL/min; Detection: UV at 220 nm). ¹H NMR (400 MHz, DMSO-d₆) δ ppm 8.15-8.28 (m, 1H), 7.97-8.02 (m, 1H), 7.73 (d, J=8.1 Hz, 2H), 7.59 (br d, J=8.1 Hz, 2H), 5.57-6.01 (m, 1H), 4.80-5.08 (m, 1H), 3.92-4.11 (m, 1H), 3.37-3.75 (m, 6H), 3.07-3.23 (m, 2H), 2.86-3.01 (m, 2H), 2.76-2.83 (m, 1H), 2.61-2.72 (m, 1H), 1.05-1.68 (m, 6H).

Example 175: (14 mg, 10% yield): LCMS: m/z, 501.3 (M+H); rt 2.14 min; (LCMS Method: Column: XBridge BEH XP C18 (2.1×50 mm), 2.5 μm; Mobile phase A: 10 mM ammonium acetate:acetonitrile (95:5), Mobile phase B: 10 mM ammonium acetate:acetonitrile (5:95), Gradient=0-100% B over 3 minutes; Temperature: 50° C.; Flow rate: 1.1 mL/min; Detection: UV at 220 nm). ¹H NMR (400 MHz, DMSO-d₆) δ ppm 8.25 (br d, J=9.0 Hz, 1H), 8.00 (d, J=9.0 Hz, 1H), 7.70 (in, J=8.1 Hz, 2H), 7.59-7.65 (m, 2H), 6.00-6.16 (m, 1H), 4.72-4.88 (m, 1H), 3.89-3.95 (m, 1H), 3.70-3.83 (m, 1H), 3.49-3.65 (m, 1H), 3.45 (s, 3H), 3.37-3.41 (m, 1H), 3.23-3.32 (m, 2H), 3.00-3.13 (m, 2H), 2.53-2.65 (m, 1H), 2.35-2.46 (m, 1H), 1.36 (d, J=6.4 Hz, 3H), 1.16-1.28 (m, 3H).

The examples in the Table 7 were prepared according to the general procedure described in Examples 174 and 175, using the appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 7

| Ex. | | LCMS | LCMS | | Stereo |
|---|---|---|---|---|---|
| No. | Structure | Method | RT | M + H | Chem. |
| 176 | 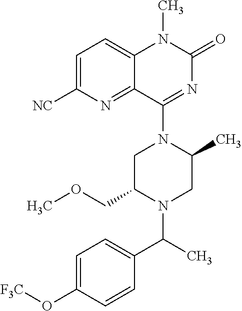 [SMILES: COC[C@@H]1CN(c2nc(=O)n(C)c3ccc(C#N)nc23)[C@@H](C)CN1C(C)c1ccc(OC(F)(F)F)cc1] | A | 2.15 | 517.3 | H |
| 177 | 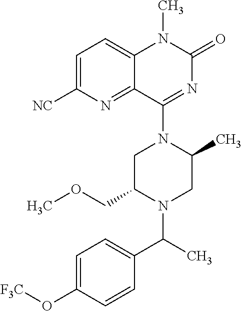 [SMILES: COC[C@@H]1CN(c2nc(=O)n(C)c3ccc(C#N)nc23)[C@@H](C)CN1C(C)c1ccc(OC(F)(F)F)cc1] | A | 2.19 | 517.3 | H |
| 178 | 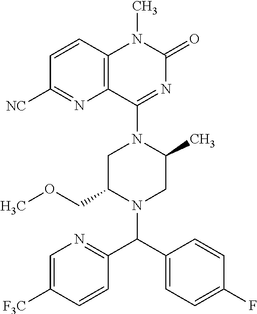 [SMILES: COC[C@@H]1CN(c2nc(=O)n(C)c3ccc(C#N)nc23)[C@@H](C)CN1C(c1ccc(F)cc1)c1ccc(C(F)(F)F)cn1] | A | 2.08 | 582.2 | H |
| 179 | 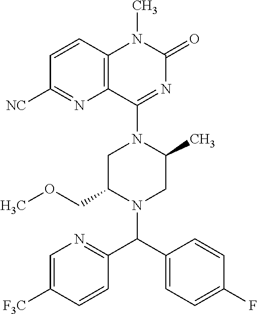 [SMILES: COC[C@@H]1CN(c2nc(=O)n(C)c3ccc(C#N)nc23)[C@@H](C)CN1C(c1ccc(F)cc1)c1ccc(C(F)(F)F)cn1] | A | 2.07 | 582.1 | H |

### Intermediate 18

### tert-Butyl(2S,5S)-4-((4-fluorophenyl)(5-(trifluoromethyl)pyridin-2-yl)methyl)-5-(hydroxymethyl)-2-methylpiperazine-1-carboxylate (Diastereomeric Mixture)

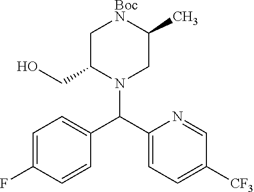 [SMILES: C[C@H]1CN(C(c2ccc(F)cc2)c2ccc(C(F)(F)F)cn2)[C@H](CO)CN1C(=O)OC(C)(C)C]

To a stirred solution of tert-butyl (2S,5S)-5-(hydroxymethyl)-2-methylpiperazine-1-carboxylate (1 g, 4.34 mmol) in acetonitrile (20 mL), DIPEA (3.0 mL, 17.4 mmol) and 2-(bromo(4-fluorophenyl)methyl)-5-(trifluoromethyl)pyridine (1.75 g, 5.21 mmol) were added sequentially at room temperature. The reaction mixture was heated at 80° C. for 3 h. The reaction mixture was cooled to room temperature and solvent was removed under reduced pressure to give the crude product, which was purified by silica gel column chromatography using a 24 g silica gel flash column, eluting with 3% MeOH in CHCl₃ to afford tert-butyl (2S,5S)-4-((4-fluorophenyl)(5-(trifluoromethyl)pyridin-2-yl)methyl)-5-(hydroxymethyl)-2-methylpiperazine-1-carboxylate (1.4 g, 67% yield). LCMS: m/z, 484.2 (M+H); rt 1.97 and 1.99 min. (LCMS Method: Column: Waters Acquity UPLC BEH C18 (3.0×50 mm) 1.7 μm, Mobile phase A: 10 mM ammonium acetate:acetonitrile (95:5); Mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Intermediate 19

### ((2S,5S)-1-((4-Fluorophenyl)(5-(trifluoromethyl)pyridin-2-yl)methyl)-5-methylpiperazin-2-yl)methanol, HCl (Diastereomeric Mixture)

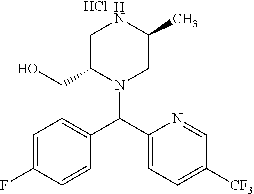 [SMILES: Cl ; C[C@H]1CN(C(c2ccc(F)cc2)c2ccc(C(F)(F)F)cn2)[C@H](CO)CN1]

To a stirred solution of tert-butyl (2S,5S)-4-((4-fluorophenyl)(5-(trifluoromethyl) pyridin-2-yl)methyl)-5-(hydroxymethyl)-2-methylpiperazine-1-carboxylate (1 g, 2.07 mmol) in DCM (20 mL), HCl (4 N in dioxane) (2.6 mL, 10.4 mmol) was added drop wise at room temperature. The reaction mixture was stirred for 3 h. Solvent was removed under reduced pressure, the solids were co-distilled with acetonitrile (3×10 mL), and dried to afford ((2S,5S)-1-((4-fluorophenyl)(5-(trifluoromethyl)pyridin-2-yl) methyl)-5-methylpiperazin-2-yl)methanol, HCl (800 mg, 92% yield) as an off-white solid. LCMS: m/z, 384.2 (M+H); rt 1.05 & 1.11 min. (LCMS Method: Column: Waters Acquity UPLC BEH C18 (3.0×50 mm) 1.7 μm, Mobile phase A: 10 mM ammonium acetate:acetonitrile (95:5); Mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Example 180

### 6-Chloro-4-((2S,5S)-4-((4-fluorophenyl)(5-(trifluoromethyl)pyridin-2-yl)methyl)-5-(hydroxymethyl)-2-methylpiperazin-1-yl)-1-methylpyrido[3,2-d]pyrimidin-2(1H)-one (Diastereomeric Mixture)

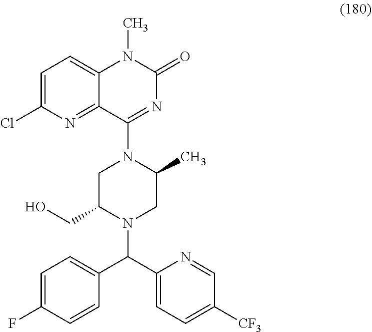 [SMILES: C[C@H]1CN(C(c2ccc(F)cc2)c2ccc(C(F)(F)F)cn2)[C@H](CO)CN1c1nc(=O)n(C)c2ccc(Cl)nc12]

To a stirred solution of ((2S,5S)-1-((4-fluorophenyl)(5-(trifluoromethyl)pyridin-2-yl)methyl)-5-methylpiperazin-2-yl)methanol, HCl (400 mg, 0.95 mmol) in acetonitrile (15 mL), DIPEA (0.8 mL, 4.8 mmol) and 4,6-dichloro-1-methylpyrido[3,2-d]pyrimidin-2(1H)-one (292 mg, 0.95 mmol) were added sequentially at room temperature followed by heating at 80° C. for 12 h. The reaction mixture was cooled to room temperature and solvent was removed under reduced pressure to give crude residue, which was purified by silica gel column chromatography using 12 g flash column, eluting with 3-7% MeOH in CHCl₃ to afford 6-chloro-4-((2S,5S)-4-((4-fluorophenyl)(5-(trifluoromethyl)pyridin-2-yl) methyl)-5-(hydroxymethyl)-2-methylpiperazin-1-yl)-1-methylpyrido[3,2-d]pyrimidin-2(1H)-one (450 mg, 82% yield). LCMS: m/z, 577.2 (M+H); rt 1.92 and 1.94 min. (LCMS Method: Column: Waters Acquity UPLC BEH C18 3.0×50 mm 1.7 μm, Mobile phase A: 10 mM ammonium acetate:acetonitrile (95:5); Mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Examples 181 and 182

### 4-((2S,5S)-4-((4-Fluorophenyl)(5-(trifluoromethyl)pyridin-2-yl)methyl)-5-(hydroxymethyl)-2-methylpiperazin-1-yl)-1-methyl-2-oxo-1,2-dihydropyrido[3,2-d] pyrimidine-6-carbonitrile

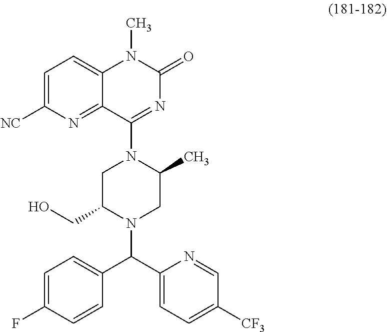 [SMILES: C[C@H]1CN(C(c2ccc(F)cc2)c2ccc(C(F)(F)F)cn2)[C@H](CO)CN1c1nc(=O)n(C)c2ccc(C#N)nc12]

To a stirred solution of 6-chloro-4-((2S,5S)-4-((4-fluorophenyl)(5-(trifluoromethyl)pyridin-2-yl)methyl)-5-(hydroxymethyl)-2-methylpiperazin-1-yl)-1-methylpyrido[3,2-d]pyrimidin-2(1H)-one (150 mg, 0.26 mmol) in DMF (5 mL) were added zinc (26 mg, 0.39 mmol) and TEA (0.15 mL, 1.08 mmol). The reaction mixture was degassed with argon gas for 5 min., followed by the addition of zinc cyanide (92 mg, 0.78 mmol) and dichloro[9,9-dimethyl-4,5-bis(diphenylphosphino)xanthene]palladium(II) (20 mg, 0.03 mmol). The reaction mixture heated at 90° C. for 12 h. The reaction mixture was cooled to room temperature and concentrated under reduced pressure to afford a residue which was purified via preparative HPLC (Chiral Separation Method: Column: Cellulose-5 (250*19 ID) 5 micron; Mobile Phase A: 10 mM NH₄OAc in MeOH; Flow: 20 mL/min; UV detection: 215 nm).

Example 181: (15 mg, 10% yield): LCMS: m/z, 568.2 (M+H); rt 2.74 min; (LCMS Method: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Detection: UV at 220 nm). ¹H NMR (400 MHz, DMSO-d₆) δ ppm 8.92 (s, 1H), 8.16-8.26 (m, 2H), 8.00 (br d, J=9.5 Hz, 1H), 7.86-7.96 (m, 1H), 7.53-7.60 (m, 2H), 7.10-7.22 (m, 2H), 5.50-5.86 (m, 1H), 5.24 (s, 1H), 5.02-5.15 (m, 1H), 4.65-4.89 (m, 1H), 4.17-4.54 (m, 1H), 3.56-3.92 (m, 2H), 3.45 (s, 3H), 2.77-2.92 (m, 2H), 2.52-2.61 (m, 1H), 1.31-1.59 (m, 3H).

Example 182: (17 mg, 11% yield): LCMS: m/z, 568.2 (M+H); rt 2.72 min; (LCMS Method: Column-Kinetex XB-C18 (75×3 mm-2.6 μm), Mphase A: 10 mM ammonium formate in water:acetonitrile (98:2); Mphase B: 10 mM ammonium formate in water:acetonitrile (2:98); Flow: 1 mL/min; Gradient: 20-100% B over 4 minutes then 0.6 min hold at 100% B, Detection: UV at 220 nm). ¹H NMR (400 MHz, DMSO-d₆) δ ppm 8.83 (br s, 1H), 8.23 (br d, J=5.5 Hz, 2H), 7.99 (br d, J=8.0 Hz, 2H), 7.50-7.68 (m, 2H), 7.18 (br t, J=8.8 Hz, 2H), 5.48-5.84 (m, 1H), 5.04-5.27 (m, 2H), 4.69-4.84 (m, 1H), 4.20-4.55 (m, 1H), 3.75-3.87 (m, 1H), 3.52-3.64 (m, 2H), 3.45 (s, 3H), 2.85-2.98 (m, 2H), 1.37-1.62 (m, 3H).

The examples in the Table 8 were prepared according to the general procedure described in Examples 181 and 182, using the appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 8

| Ex. | | LCMS | LCMS | | Stereo |
|---|---|---|---|---|---|
| No. | Structure | Method | RT | M + H | chem. |
| 183 | 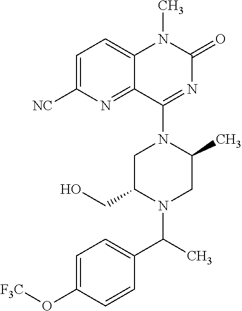 [SMILES: CC(c1ccc(OC(F)(F)F)cc1)N1C[C@H](C)N(c2nc(=O)n(C)c3ccc(C#N)nc23)C[C@H]1CO] | A | 1.98 | 503.3 | H |
| 184 | 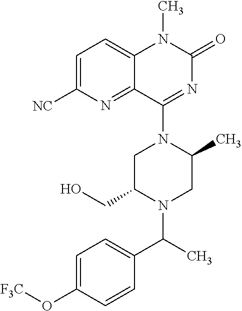 [SMILES: CC(c1ccc(OC(F)(F)F)cc1)N1C[C@H](C)N(c2nc(=O)n(C)c3ccc(C#N)nc23)C[C@H]1CO] | A | 1.99 | 503.3 | H |

### LCMS Conditions:

Method C: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm.

Method D: Column: XBridge BEH XP C18 (50×2 mm, 2.5 μm); mobile phase A: 0.1% TFA in water:acetonitrile (95:5); mobile phase B: 0.1% TFA in water:acetonitrile (5:95); Gradient=0-100% B over 3 minutes; Temperature: 50° C.; Flow rate: 1.1 mL/min; Detection: UV at 220 nm.

Method E: Column-KINETEX-XB-C18 (75×3 mm, 2.6 μm); mobile phase A: 10 mM NH₄OAc in water:acetonitrile (98:2); mobile phase B: 10 mM NH₄OAc in water:acetonitrile (02:98); Gradient=20-100% B over 4 minutes; Temperature: 50° C.; Flow rate: 1.1 mL/min; Detection: UV at 254 nm.

Method F: Column: XBridge C18 (50×2.1 mm, 1.7 μm); mobile phase A: 0.1% TFA in water:acetonitrile (95:5); mobile phase B: 0.1% TFA in water, acetonitrile (5:95); Gradient=0-100% B over 3 minutes; Temperature: 50° C.; Flow rate: 1.0 mL/min; Detection: UV at 220 nm.

### Intermediate 20

### Methyl (S)-2-(benzylamino)butanoate

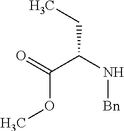

To a stirred solution of H-ABU-OME HCl (5 g, 32.6 mmol) in dry DCM (50 mL) was added benzaldehyde (4.3 mL, 42.3 mmol). The reaction mixture was stirred at room temperature for 16 h. The reaction mixture was cooled with an ice water bath and solid sodium triacetoxyborohydride (10.35 g, 48.8 mmol) was added in portion wise over ˜15 min. The cooling bath was removed and the milky white solution was stirred at room temperature for 16 h. The solvent was removed under reduced pressure and the resulting crude product was partitioned between EtOAc (˜100 mL) and 1 N HCl (˜200 mL). The layers were separated and the aqueous layer was extracted with EtOAc (2×500 mL). The aqueous layer was adjusted to pH ˜10 with 1 N NaOH (450 mL) and the milky aqueous layer was extracted immediately with EtOAc (3×150 mL). The combined organic layer was washed with brine (250 mL), dried over anhydrous MgSO₄, filtered and concentrated under reduced pressure to afford methyl (S)-2-(benzylamino)butanoate (4 g, 56% yield). LCMS: m/z=208.2 [M+H]⁺; retention time 2.28 min, LCMS Method: Kinetex XB-C18 (3×75 mm, 2.6 μm); mobile phase A: 10 mM ammonium formate in water:acetonitrile (98:2), mobile phase B: 10 mM ammonium formate in water:acetonitrile (2:98), Gradient=20-100% B over 4 minutes, then a 0.6 minute hold at 100% B; Temperature: 27° C.; Flow rate: 1.0 mL/min.

### Intermediate 21

### Methyl (S)-2-((R)—N-benzyl-2-((tert-butoxycarbonyl)amino)butanamido)butanoate

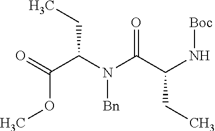 [SMILES: CC[C@@H](NC(=O)OC(C)(C)C)C(=O)N(Cc1ccccc1)[C@@H](CC)C(=O)OC]

To a stirred solution of BOC-D-ABU-OH (1 g, 4.92 mmol) in dry DMF (8 mL) was added HATU (3.74 g, 9.84 mmol) and DIPEA (2.1 mL, 12.3 mmol). The reaction mixture was stirred for 5 min. and methyl (S)-2-(benzylamino)butanoate (1.43 g, 6.89 mmol) was added. The reaction mixture was stirred at room temperature for 16 h. The reaction mixture was diluted with ethyl acetate and washed with water. The organic layer was dried over anhydrous Na₂SO₄, filtered and evaporated under reduced pressure to yield the crude compound, which was purified by silica gel chromatography (eluted with 20-30% ethyl acetate/pet ether) to yield methyl (S)-2-((R)—N-benzyl-2-((tert-butoxycarbonyl)amino)butanamido)butanoate (1 g, 52% yield). LCMS: m/z=393.2 [M+H]⁺; retention time 3.26 min, LCMS Method: Column: Kinetex XB-C18 (3×75 mm, 2.6 μm); mobile phase A: 10 mM ammonium formate in water:acetonitrile (98:2), mobile phase B: 10 mM ammonium formate in water:acetonitrile (2:98), Gradient=20-100% B over 4 minutes, then a 0.6 minute hold at 100% B; flow rate: 1.0 mL/min;

### Intermediate 22

### Methyl (S)-2-((R)-2-amino-N-benzylbutanamido)butanoate

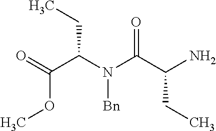 [SMILES: CC[C@@H](N)C(=O)N(Cc1ccccc1)[C@@H](CC)C(=O)OC]

To a stirred solution of methyl (S)-2-((R)—N-benzyl-2-((tert-butoxycarbonyl) amino)butanamido)butanoate (1 g, 2.55 mmol) in dry DCM (10 mL) was added TFA (5 mL, 64.9 mmol) at room temperature. The reaction mixture was stirred for 2 h. The solvent was removed under reduced pressure to yield methyl (S)-2-((R)-2-amino-N-benzylbutanamido)butanoate, TFA (1 g, 58% yield). LCMS: m/z=293.2 [M+H]⁺; retention time 1.08 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min, temperature: 27° C.; detection: UV at 220 nm.

### Intermediate 23

### (3R,6S)-1-Benzyl-3,6-diethylpiperazine-2,5-dione

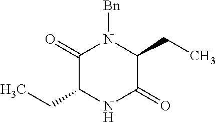 [SMILES: CC[C@H]1NC(=O)[C@H](CC)N(Cc2ccccc2)C1=O]

Methyl (S)-2-((R)-2-amino-N-benzylbutanamido)butanoate TFA (1 g, 3.42 mmol) was dissolved in MeOH (15 mL) and the reaction mixture was heated at 70° C. for 16 h. The reaction mixture was cooled to room temperature and the solvent was removed under reduced pressure to yield the crude compound, which was dissolved in DCM and washed with saturated aqueous NaHCO₃ solution. The combined organic layer was dried over anhydrous Na₂SO₄, filtered and evaporated under reduced pressure to yield (3R,6S)-1-benzyl-3,6-diethylpiperazine-2,5-dione (0.6 g, 46.0% yield). LCMS: m/z=261.2 [M+H]⁺; retention time 1.08 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min, temperature: 27° C.; detection: UV at 220 nm.

### Intermediate 24

### (2S,5R)-1-Benzyl-2,5-diethylpiperazine

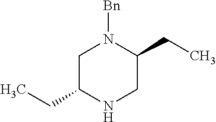 [SMILES: CC[C@@H]1CN(Cc2ccccc2)[C@@H](CC)CN1]

To a stirred solution of (3R,6S)-1-benzyl-3,6-diethylpiperazine-2,5-dione (0.63 g, 2.42 mmol) in dry tetrahydrofuran (15 mL) was slowly added BH₃·THF (1 M, 12.1 mL, 12.1 mmol) at 0° C. The reaction mixture heated at 70° C. for 16 h. The reaction mixture was cooled to room temperature. The reaction was quenched with the addition of methanol (20 mL) and aqueous 1.5 N HCl (1 mL, 32.9 mmol). The mixture was heated at 70° C. for 2 h, then cooled to room temperature and the solvent was removed under reduced pressure to yield the crude product, which was dissolved in DCM and washed with saturated aqueous NaHCO₃ solution. The organic layer was dried over anhydrous Na₂SO₄, filtered and evaporated under reduced pressure to yield (2S,5R)-1-benzyl-2,5-diethylpiperazine (0.51 g, 86% yield). LCMS: m/z=233.0 [M+H]⁺; retention time 0.454 min, LCMS Method: Kinetex XB-C18 (3×75 mm, 2.6 μm); mobile phase A: 0.1% TFA in water, mobile phase B: 0.1% TFA in acetonitrile, Gradient=20-100% B over 4 minutes, then a 0.6 minute hold at 100% B; Flow rate: 1.5 mL/min.

### Intermediate 25

### tert-Butyl (2R,5S)-4-benzyl-2,5-diethylpiperazine-1-carboxylate

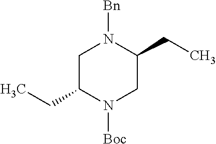 [SMILES: CC[C@H]1CN(C(=O)OC(C)(C)C)[C@H](CC)CN1Cc1ccccc1]

To a stirred solution of (2S,5R)-1-benzyl-2,5-diethylpiperazine (0.51 g, 2.19 mmol) in dry DCM (10 mL) was added TEA (0.8 mL, 5.49 mmol) and Boc-anhydride (0.8 mL, 3.29 mmol). The reaction mixture was stirred at room temperature for 2 h. The reaction mixture was diluted with DCM and washed with water, brine and dried over anhydrous Na₂SO₄. Evaporation of the solvent under reduced pressure provided the crude compound, which was purified by silica gel column chromatography (eluted with 5-10% ethyl acetate/pet ether) to yield tert-butyl (2R, 5S)-4-benzyl-2,5-diethylpiperazine-1-carboxylate (0.45 g, 60% yield). LCMS: m/z=333.2 [M+H]⁺; retention time 2.02 min, LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Flow rate: 1.0 mL/min.

### Intermediate 26

### tert-Butyl (2R,5S)-2,5-diethylpiperazine-1-carboxylate

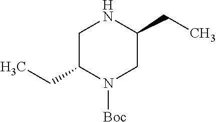 [SMILES: CC[C@H]1CN(C(=O)OC(C)(C)C)[C@H](CC)CN1]

tert-Butyl (2R,5S)-4-benzyl-2,5-diethylpiperazine-1-carboxylate (450 mg, 1.35 mmol) was dissolved in MeOH (10 mL) and to this solution was added acetic acid (0.1 mL, 1.35 mmol), followed by Pd/C (10% w/w) (100 mg, 0.026 mmol). The reaction mixture was stirred under H₂ at 70 psi for 16 hours. The reaction mixture was filtered through a Celite® pad, washed with methanol and evaporated under reduced pressure to yield tert-butyl (2R,5S)-2,5-diethylpiperazine-1-carboxylate, AcO— (0.32 g, 69% yield). LCMS: m/z=243.2 [M+H]⁺; retention time 0.78 min, LCMS Method: Column-Luna 3.0 C18 (2) 100 Å LC column (20×4.0 mm); mobile phase A: 0.1% TFA in water; mobile phase B: 0.1% TFA in acetonitrile, Gradient=20-100% B over 4 minutes, then a 0.6 minute hold at 100% B; Temperature: 27° C.; Flow rate: 1.0 mL/min; Detection: UV at 220 nm.

### Experimental Method C:

### Example 185

### 4-((2S,5R)-4-(4-(Cyclopropylmethoxy)benzyl)-2,5-diethylpiperazin-1-yl)-1-methyl-2-oxo-1,2-dihydropyrido[3,2-d]pyrimidine-6-carbonitrile

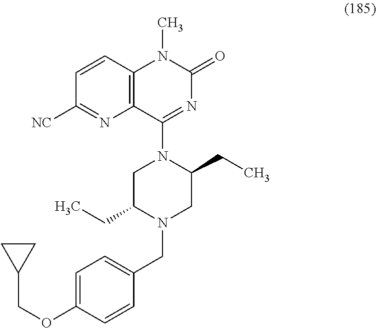 [SMILES: CC[C@@H]1CN(c2nc(=O)n(C)c3ccc(C#N)nc23)[C@@H](CC)CN1Cc1ccc(OCC2CC2)cc1]

To a solution of 4-((2S,5R)-2,5-diethylpiperazin-1-yl)-1-methyl-2-oxo-1,2-dihydropyrido[3,2-d]pyrimidine-6-carbonitrile (0.05 g, 0.15 mmol) in methanol (2 mL) were added 4-(cyclopropylmethoxy)benzaldehyde (0.040 g, 0.23 mmol), acetic acid (4.38 μL, 0.08 mmol) and magnesium sulfate (0.018 g, 0.15 mmol). The reaction mixture was stirred at room temperature for 16 h. Sodium cyanoborohydride (9.6 mg, 0.15 mmol) was added to the reaction mixture. The reaction mixture was stirred for 3 h. The reaction was quenched with the addition of water. The mixture was extracted with ethyl acetate (2×100 mL), the combined organic layer was washed with water, brine and dried over sodium sulfate. The solvent was evaporated under reduced pressure to yield the crude product, which was purified by preparative HPLC. HPLC Method: Column: XBridge C18 (19×50 mm, 5 μm); mobile phase A: 10 mM ammonium acetate in water; mobile phase B: acetonitrile; Gradient: 20-70% B over 20 minutes, then a 5 minute hold at 100% B; Flow: 15 mL/min), fractions were concentrated under reduced pressure and lyophilized from (EtOH/H₂O, 1:5) to yield Example 185 (6 mg, 8% yield); LCMS: m/z=487.3 [M+H]⁺; rt 2.310 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.23 (br d, J=8.6 Hz, 1H), 7.98 (br d, J=8.8 Hz, 1H), 7.23-7.25 (m, 2H), 6.99-6.74 (m, 2H), 6.01-5.29 (m, 1H), 4.92 (br s, 1H), 3.79 (d, J=7.1 Hz, 2H), 3.65-3.46 (m, 3H), 3.43 (s, 3H), 2.67-2.68 (m, 2H), 2.46-2.42 (m, 1H), 2.12-1.75 (m, 2H), 1.58-1.28 (m, 2H), 1.27-1.13 (m, 1H), 0.99-0.65 (m, 6H), 0.60-0.46 (m, 2H), 0.37-0.25 (m, 2H).

### Experimental Method D:

### Intermediate 27

### 6-Chloro-4-((2S,5R)-2,5-diethylpiperazin-1-yl)-1-methylpyrido[3,2-d]pyrimidin-2(1H)-one

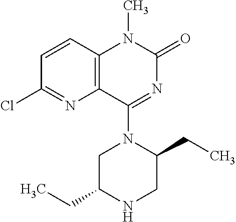 [SMILES: CC[C@@H]1CN(c2nc(=O)n(C)c3ccc(Cl)nc23)[C@@H](CC)CN1]

To a stirred solution tert-butyl (2R,5S)-4-(6-chloro-1-methyl-2-oxo-1,2-dihydropyrido[3,2-d]pyrimidin-4-yl)-2,5-diethylpiperazine-1-carboxylate (2.30 g, 5.28 mmol) in DCM (50 mL) was added TFA (8 mL, 104 mmol). The reaction mixture was stirred at room temperature for 4 hours, concentrated under reduced pressure to yield the TFA salt of 6-chloro-4-((2S,5R)-2,5-diethylpiperazin-1-yl)-1-methylpyrido[3,2-d] pyrimidin-2(1H)-one (1.55 g, 87% yield). LCMS: m/z=336.2 [M+H]⁺; rt=0.41 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Intermediate 28

### Ethyl 2-(4-(1-((2R,5S)-4-(6-chloro-1-methyl-2-oxo-1,2-dihydropyrido[3,2-d]pyrimidin-4-yl)-2,5-diethylpiperazin-1-yl)ethyl)phenoxy)-2-methylpropanoate

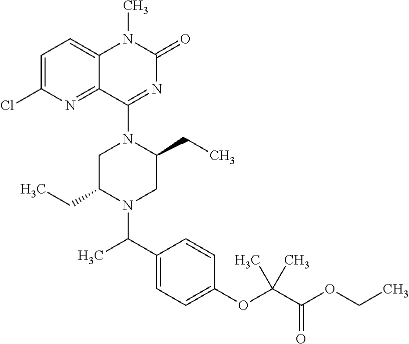 [SMILES: CCOC(=O)C(C)(C)Oc1ccc(C(C)N2C[C@H](CC)N(c3nc(=O)n(C)c4ccc(Cl)nc34)C[C@H]2CC)cc1]

To a stirred solution of 6-chloro-4-((2S,5R)-2,5-diethylpiperazin-1-yl)-1-methylpyrido[3,2-d]pyrimidin-2(1H)-one, HCl (500 mg, 1.34 mmol) in acetonitrile (20 mL) were added DIPEA (0.71 mL, 4.03 mmol) and ethyl 2-(4-(1-chloroethyl)phenoxy)-2-methylpropanoate (436 mg, 1.61 mmol). The reaction mixture was stirred at 80° C. overnight. The reaction mixture was diluted with ethyl acetate (100 mL) and washed with water, the organic layer was dried over anhydrous Na₂SO₄ and filtered. The volatiles were removed under reduced pressure to yield the crude product, which was purified by flash column chromatography on silica gel (Hexane:EtOAc=45:55) to give ethyl 2-(4-(1-((2R,5S)-4-(6-chloro-1-methyl-2-oxo-1,2-dihydropyrido[3,2-d]pyrimidin-4-yl)-2,5-diethylpiperazin-1-yl)ethyl)phenoxy)-2-methylpropanoate (0.35 g, 45% yield). LCMS: m/z=556.2 (M-Et+Me-+H); rt 2.38 min. LCMS Method: Column-Luna 3.0 C18 (2) 100 Å LC column (20×4.0 mm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm.

### Example 186

### 6-Chloro-4-((2S,5R)-2,5-diethyl-4-(1-(4-((1-hydroxy-2-methylpropan-2-yl)oxy)phenyl)ethyl)piperazin-1-yl)-1-methylpyrido[3,2-d]pyrimidin-2(1H)-one

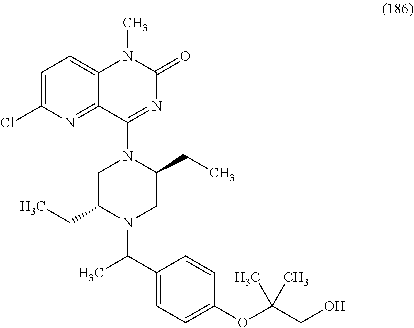 [SMILES: CC[C@H]1CN(C(C)c2ccc(OC(C)(C)CO)cc2)[C@H](CC)CN1c1nc(=O)n(C)c2ccc(Cl)nc12]

To a stirred solution of ethyl 2-(4-(1-((2R,5S)-4-(6-chloro-1-methyl-2-oxo-1,2-dihydropyrido[3,2-d]pyrimidin-4-yl)-2,5-diethylpiperazin-1-yl)ethyl)phenoxy)-2-methylpropanoate (180 mg, 0.32 mmol) in tetrahydrofuran (5 mL) was added LiBH₄ (2 M, 0.8 mL, 1.6 mmol) at 0° C. The reaction mixture was stirred at room temperature for 3 h. The reaction mixture was dissolved in ethyl acetate and washed with saturated aqueous NH₄Cl solution, the organic layer was dried over anhydrous Na₂SO₄ filtered and evaporated under reduced pressure to afford 6-chloro-4-((2S,5R)-2,5-diethyl-4-(1-(4-((1-hydroxy-2-methylpropan-2-yl)oxy)phenyl)ethyl)piperazin-1-yl)-1-methylpyrido[3,2-d] pyrimidin-2(1H)-one (0.16 g, 97% yield). LCMS: m/z=528.2 [M+H]⁺; rt 1.98 min. LCMS Method: Column-Luna C18 (20×4.0 mm, 100 Å); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm.

### Example 187

### 6-Chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one

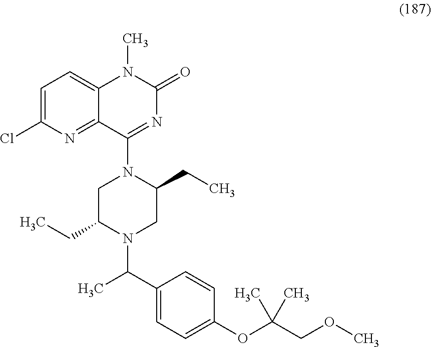 [SMILES: CC[C@H]1CN(C(C)c2ccc(OC(C)(C)COC)cc2)[C@H](CC)CN1c1nc(=O)n(C)c2ccc(Cl)nc12]

To a stirred solution of 6-chloro-4-((2S,5R)-2,5-diethyl-4-(1-(4-((1-hydroxy-2-methylpropan-2-yl)oxy)phenyl)ethyl)piperazin-1-yl)-1-methylpyrido[3,2-d]pyrimidin-2(1H)-one (200 mg, 0.379 mmol) in tetrahydrofuran (5 mL) was added NaH (22.72 mg, 0.57 mmol, 60% w/w) at 0° C. After 10 minutes, methyl iodide (0.03 mL, 0.45 mmol) was added dropwise and the reaction mixture was stirred at room temperature for 16 h. The reaction quenched with water (4 mL). The reaction mixture was extracted with EtOAc (25 mL), dried over anhydrous Na₂SO₄, filtered and evaporated under reduced pressure to afford 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one (0.16 g, 17% yield). LCMS: m/z=542.5 [M+H]⁺; rt 1.43 min, LCMS Method; Column: AQUITY UPLC BEH C18 (3.0×50 mm, 1.7 μm); mobile phase A: 2.5 mM NH₄OAc in water:acetonitrile (95:5); mobile phase B: 2.5 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Examples 188 and 189

### 6-Chloro-4-((2S,5R)-2,5-diethyl-4-(1-(4-((1-methoxy-2-methylpropan-2-yl)oxy)phenyl) ethyl)piperazin-1-yl)-1-methylpyrido[3,2-d]pyrimidin-2(1H)-one

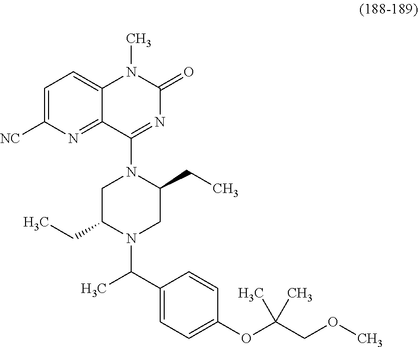 [SMILES: CC[C@H]1CN(C(C)c2ccc(OC(C)(C)COC)cc2)[C@H](CC)CN1c1nc(=O)n(C)c2ccc(C#N)nc12]

To a stirred solution of 6-chloro-4-((2S,5R)-2,5-diethyl-4-(1-(4-((1-methoxy-2-methylpropan-2-yl)oxy)phenyl)ethyl)piperazin-1-yl)-1-methylpyrido[3,2-d]pyrimidin-2(1H)-one (80 mg, 0.15 mmol) in DMF (1.5 mL) was added zinc (9.7 mg, 0.15 mmol), zinc cyanide (35 mg, 0.295 mmol) and triethylamine (0.062 mL, 0.443 mmol). Next, dichloro[9,9-dimethyl-4,5-bis(diphenylphosphino)xanthene] palladium(II) (11.16 mg, 0.015 mmol) was added under argon. The reaction mixture was heated to 90° C. for 16 h, cooled, diluted with ethyl acetate and filtered through the Celite® pad, and washed with ethyl acetate. The filtrate was removed under reduced pressure to yield the crude product which was purified by preparative HPLC method: Column: Sunfire OBD (250×30 mm, m), mobile phase A: 10 mM ammonium acetate in water, mobile phase B: acetonitrile, Flow 19 mL/min. to yield Examples 188 and 189.

EXAMPLE 188: Fraction 1 was concentrated under reduced pressure and the product was diluted with (EtOH/H₂O, 1:5) and lyophilized to yield Example 188 (6.1 mg, 7.14% yield); LCMS: m/z, 533.3 [M+H]⁺; rt 2.312 min; (LCMS method: Column: XBridge BEH C18 XP (50×2.1 mm, 2.5 μm); mobile phase A: 5:95 acetonitrile:water with 10 mM NH₄OAc in water; mobile phase B: 95:5 acetonitrile:water with 10 mM NH₄OAc in water; Temperature: 50° C.; Gradient: 0-100% B over 3 minutes. ¹H NMR (400 MHz, DMSO-d₆) δ (ppm)=8.29-8.16 (m, 1H), 7.98 (d, J=9.0 Hz, 1H), 7.28-7.21 (m, 2H), 6.91-6.96 (m, 2H), 5.82-5.41 (m, 1H), 5.00-4.76 (m, 1H), 3.43-3.66 (m, 1H), 3.42 (s, 3H), 3.31-3.29 (m, 4H), 3.10-3.03 (m, 1H), 2.93-2.86 (m, 1H), 2.74-2.67 (m, 1H), 2.15-2.33 (m, 1H), 1.95 (s, 2H), 1.22-1.28 (m, 11H), 0.96-0.84 (m, 3H), 0.71-0.51 (m, 3H).

EXAMPLE 189: Fraction 2 was concentrated under reduced pressure and the product was diluted with (EtOH/H₂O, 1:5) and lyophilized to yield Example 189 (6.0 mg, 6.95% yield); LCMS: m/z, 533.3 [M+H]⁺; rt 2.339 min; (LCMS Method: Column: XBridge BEH C18 XP (50×2.1 mm, 2.5 μm); mobile phase A: 5:95 acetonitrile:water with 10 mM NH₄OAc in water mobile phase B: 95:5 acetonitrile:water with 10 mM NH₄OAc in water; Temperature: 50° C.; Gradient: 0-100% B over 3 minutes. ¹H NMR (400 MHz, DMSO-d₆) δ (ppm)=8.28-8.16 (m, 1H), 8.02-7.91 (m, 1H), 7.28 (d, J=8.3 Hz, 2H), 6.92 (d, J=8.6 Hz, 2H), 5.21-5.05 (m, 1H), 4.70-4.64 (m, 1H), 3.63-3.47 (m, 1H), 3.43 (s, 3H), 3.31 (s, 5H), 3.16-3.04 (m, 1H), 2.45-2.41 (m, 1H), 2.30-2.20 (m, 1H), 2.13-2.05 (m, 1H), 1.73-1.39 (m, 3H), 1.23-1.25 (m, 3H), 1.20 (s, 7H), 1.03-0.90 (m, 3H), 0.62-0.53 (m, 3H).

### Intermediate 135

### 4-(Cyclopropylmethoxy)-2-fluorobenzaldehyde

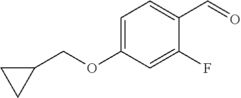 [SMILES: O=Cc1ccc(OCC2CC2)cc1F]

To a stirred solution of 2-fluoro-4-hydroxybenzaldehyde (5 g, 7.14 mmol) in acetonitrile (50 mL) were added K₂CO₃ (2.96 g, 21.41 mmol) and (bromomethyl) cyclopropane (0.832 mL, 8.56 mmol). The reaction mixture was heated to 60° C. for 16 h. The reaction mixture cooled to room temperature, the solvent was removed under reduced pressure and the residue was dissolved in water (150 mL) and extracted twice with ethyl acetate (2×40 mL). The organic layer was dried over Na₂SO₄, concentrated under reduced pressure to yield the crude product, which was purified by column chromatography (using 10-35% EtOAc in pet ether) to yield 4-(cyclopropylmethoxy)-2-fluorobenzaldehyde (4.2 g, 60.6% yield). ¹H NMR (DMSO-d₆, 400 MHz) δ (ppm) 10.07 (s, 1H), 7.77 (m, 1H), 6.81-7.08 (m, 2H), 3.96 (d, J=7.0 Hz, 2H), 1.21-1.29 (m, 1H), 0.53-0.69 (m, 2H), 0.23-0.44 (m, 2H).

### Intermediate 136

### 1-(4-(Cyclopropylmethoxy)-2-fluorophenyl)propan-1-ol

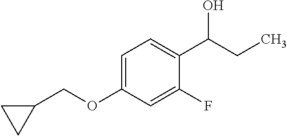 [SMILES: CCC(O)c1ccc(OCC2CC2)cc1F]

To a stirred solution of 4-(cyclopropylmethoxy)-2-fluorobenzaldehyde (4 g, 20.60 mmol) in THF (40 mL) was added ethylmagnesium bromide (13.73 mL, 41.2 mmol) at 0° C. The reaction mixture was stirred at room temperature for 4 h. The reaction was slowly quenched with the addition of saturated NH₄Cl solution (100 mL). The reaction mixture was extracted twice with ethyl acetate (2×30 mL). The organic layer was dried over Na₂SO₄, concentrated under reduced pressure to yield the crude product. The crude was purified by column chromatography (using 10-45% EtOAc in pet ether) to yield 1-(4-(cyclopropylmethoxy)-2-fluorophenyl)propan-1-ol (4.0 g, 85% yield). ¹H NMR (DMSO-d₆, 400 MHz) δ (ppm) 7.34 (m, 1H), 6.59-6.82 (m, 2H), 5.09 (d, J=4.5 Hz, 1H), 4.66 (m, 1H), 3.80 (d, J=7.0 Hz, 2H), 1.52-1.69 (m, 2H), 1.13-1.28 (m, 1H), 0.81 (t, J=7.5 Hz, 3H), 0.57 (m, 2H), 0.31 (m, 2H).

### Intermediate 137

### 1-(1-Chloropropyl)-4-(cyclopropylmethoxy)-2-fluorobenzene

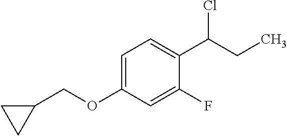 [SMILES: CCC(Cl)c1ccc(OCC2CC2)cc1F]

To a stirred solution of 1-(4-(cyclopropylmethoxy)-2-fluorophenyl)propan-1-ol (1 g, 4.46 mmol) in dichloromethane (10 mL) was added thionyl chloride (1.627 mL, 22.29 mmol). The reaction mixture was stirred at room temperature for 4 h. Evaporation the volatiles under reduced pressure gave the crude 1-(1-chloropropyl)-4-(cyclopropylmethoxy)-2-fluorobenzene (1 g, crude). ¹H NMR (DMSO-d₆, 400 MHz) δ (ppm) 7.44 (m, 1H), 6.73-6.89 (m, 2H), 5.17 (m, 1H), 3.84 (d, J=7.0 Hz, 2H), 1.99-2.24 (m, 2H), 1.14-1.28 (m, 1H), 0.93 (t, J=7.3 Hz, 3H), 0.51-0.63 (m, 2H), 0.24-0.37 (m, 2H).

The examples in Table 9 were prepared according to the general procedures described in Examples 1 to 4 and 185-186 (using the appropriate method A through D depending on the substrate used (benzhydryl/α-substituted benzyl/benzyl halides/benzaldehyde). When the reaction provided a mixture of diastereomers, the mixture was separated at the final stage using either preparative chromatography or preparative chiral chromatography. The absolute stereochemistry was not assigned at the newly formed carbon-nitrogen bond.

### Example 198 and 199

### 4-((2S,5R)-4-(1-(4-(cyclopropylmethoxy)-2-fluorophenyl)propyl)-2,5-diethylpiperazin-1-yl)-1-methyl-2-oxo-1,2-dihydropyrido[3,2-d]pyrimidine-6-carbonitrile

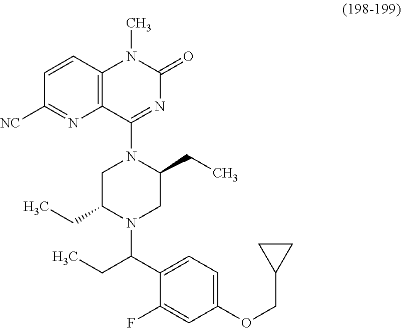 [SMILES: CCC(c1ccc(OCC2CC2)cc1F)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (200 mg, 0.55 mmol) in acetonitrile (5 mL) were added DIPEA (0.3 mL, 1.65 mmol), sodium iodide (83 mg, 0.55 mmol) and 1-(1-chloropropyl)-4-(cyclopropylmethoxy)-2-fluorobenzene (268 mg, 1.1 mmol). The reaction mixture was heated at 80° C. for 16 h. The reaction mixture was allowed to cool to room temperature. Another lot of 1-(1-chloropropyl)-4-(cyclopropylmethoxy)-2-fluorobenzene (268 mg, 1.102 mmol) was added and continued heating for another 16 h. The reaction mixture was cooled, the solvent was removed under reduced pressure and the residue was dissolved in ethyl acetate (10×20 mL). The organic layer was washed with brine, dried over Na₂SO₄, concentrated under reduced pressure to yield the crude product which was purified by preparative HPLC. HPLC method: Column: EXRS (20×250 mm, 5 μm), mobile phase A—10 mM ammonium acetate in water R, mobile phase A—B: acetonitrile, FLOW: 20 mL/min.

Fraction 1 was concentrated under reduced pressure and the product was diluted with (EtOH/H₂O, 1:5) and lyophilized to yield Example 198 (35 mg, 11.6% yield); LCMS: m/z, 533.4 [M+H]⁺, rt 1.57 min; (LCMS method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz) δ (ppm) 8.23 (d, J=9.0 Hz, 1H), 7.97 (d, J=9.0 Hz, 1H), 7.33 (m, 1H), 6.62-6.92 (m, 2H), 5.29-6.06 (m, 1H), 4.70-5.05 (m, 1H), 3.82 (m, 3H), 3.43 (s, 3H), 2.99-3.10 (m, 1H), 2.80-2.87 (m, 1H), 2.63-2.78 (m, 1H), 2.33 (s, 1H), 1.74-2.11 (m, 3H), 1.51-1.66 (m, 1H), 1.17-1.46 (m, 3H), 0.84-1.01 (m, 3H), 0.61-0.78 (m, 6H), 0.53-0.61 (m, 2H), 0.29-0.35 (m, 2H).

Fraction 2 was concentrated under reduced pressure and the product was diluted with (EtOH/H₂O, 1:5) and lyophilized to yield Example 199 (37 mg, 12.35% yield); LCMS: m/z, 533.4 [M+H]⁺, rt 2.72 min; [(LCMS Method: Column: KINETIX XB C18 (75×3 mm, 2.6 μm); mobile phase A: 10 mM ammonium acetate in water (pH 3.3), mobile phase B: acetonitrile. ¹H NMR (DMSO-d₆, 400 MHz): δ (ppm) 8.13-8.35 (m, 1H), 7.98 (m, 1H), 7.38 (m, 1H), 6.61-6.89 (m, 2H), 5.18-6.15 (m, 1H), 4.66-5.13 (m, 1H), 3.63-3.90 (m, 3H), 3.43 (s, 3H), 3.25 (m, 1H), 3.00-3.15 (m, 1H), 2.63-2.70 (m, 1H), 2.26-2.38 (m, 1H), 1.81 (m, 3H), 1.35-1.61 (m, 2H), 1.15-1.26 (m, 2H), 0.88-1.00 (m, 3H), 0.61-0.71 (m, 6H), 0.51-0.59 (m, 2H), 0.32 (m, 2H).

### Example 243 and 244

### 4-((2S,5R)-2,5-Diethyl-4-(1-(4-(trifluoromethyl)phenyl)butyl)piperazin-1-yl)-1-methyl-2-oxo-1,2-dihydropyrido[3,2-d]pyrimidine-6-carbonitrile

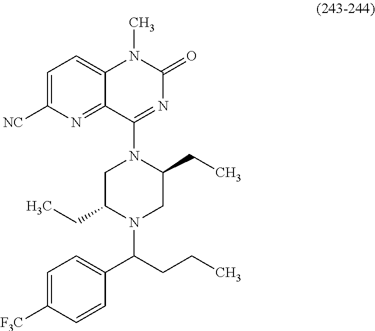 [SMILES: CCCC(c1ccc(C(F)(F)F)cc1)N1C[C@H](CC)N(c2nc(=O)n(C)c3ccc(C#N)nc23)C[C@H]1CC]

To a stirred solution of 4-((2S,5R)-2,5-diethylpiperazin-1-yl)-1-methyl-2-oxo-1,2-dihydropyrido[3,2-d]pyrimidine-6-carbonitrile, HCl (0.4 g, 1.1 mmol) in acetonitrile (10 mL) was added DIPEA (0.6 mL, 3.31 mmol), followed by 1-(1-chlorobutyl)-4-trifluoromethyl)benzene (0.783 g, 3.31 mmol) and sodium iodide (0.165 g, 1.102 mmol). The reaction mixture was heated at 85° C. for 16 h. The reaction mixture was filtered through a Celite pad, washed with ethyl acetate and the filtrate was concentrated under reduced pressure to give the crude compound, which was purified by preparative HPLC [HPLC Method: Column: YMC ExRS (250 mm×21.2 mm, 5 μm) Mobile phase A=10 mM ammonium acetate pH 4.5 in water. Mobile phase B=acetonitrile Gradient: 80% B over 2 minutes, then a 16 minute hold at 100% B; Flow: 19 mL/min) to yield Examples 243 and 244.

Example 243: (10 mg, 1.7% yield), LCMS: m/z=527.4 (M+H); rt 2.626 min; [LCMS Method: Column: XBridge BEH XP C18 (50×2.1 mm), 2.5 μm; Mobile phase A: 95% Water: 5% Acetonitrile; 10 mM NH₄OAC; Mobile phase B: 5% Water: 95% Acetonitrile; 10 mM NH₄OAC; Flow: 1.1 mL/min; Temp: 50° C.; Time (min)]. ¹H NMR (400 MHz, DMSO-d₆) δ 8.30-8.16 (m, 1H), 7.98 (d, J=9.0 Hz, 1H), 7.72 (d, J=8.3 Hz, 2H), 7.56 (br d, J=7.8 Hz, 2H), 5.86-5.44 (m, 1H), 5.01-4.77 (m, 1H), 3.730-3.718 (m, 1H), 3.46 (s, 3H), 3.43-3.35 (m, 1H) 3.13-3.01 (m, 1H), 2.93-2.75 (m, 2H), 2.38-2.26 (m, 1H), 2.17-1.74 (m, 3H), 1.63-1.22 (m, 3H), 1.01-0.86 (m, 4H), 0.84-0.75 (m, 3H), 0.73-0.54 (m, 3H).

Example 244: (7.2 mg, 1.23% yield), LCMS: m/z=527.3 (M+H); rt 2.654 min; [LCMS Method: Column: XBridge BEH XP C18 (50×2.1) mm, 2.5 μm; Mobile phase A: 95% Water: 5% Acetonitrile; 10 mM NH₄OAC; Mobile phase B: 5% Water: 95% Acetonitrile; 10 mM NH₄OAC; Flow: 1.1 mL/min; Temp: 50° C.; Time (min)]. ¹H NMR (400 MHz, DMSO-d₆) δ=8.29-8.15 (m, 1H), 7.96-8.02 (m, 1H), 7.70 (d, J=8.1 Hz, 2H), 7.58 (br d, J=8.1 Hz, 2H), 6.09-5.22 (m, 1H), 5.13-4.66 (m, 1H), 3.68-3.52 (m, 2H), 3.43 (s, 3H), 3.28-3.04 (m, 2H), 2.60-2.53 (m, 1H), 2.25-2.12 (m, 1H), 2.04-1.68 (m, 3H), 1.60-1.29 (m, 3H), 1.05-0.74 (m, 7H), 0.59 (t, J=7.5 Hz, 3H).

TABLE 9

| Ex. | | Stereo | LCMS | LCMS | | Coupling |
|---|---|---|---|---|---|---|
| No. | Structure | chem | Method | rt | [M + H]⁺ | Method |
| 190 191 | 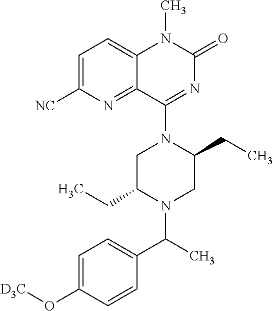 [SMILES: [2H]C([2H])([2H])Oc1ccc(C(C)N2C[C@H](CC)N(c3nc(=O)n(C)c4ccc(C#N)nc34)C[C@H]2CC)cc1] | H H | C C | 2.16 2.18 | 464.3 464.3 | B B |
| 192 193 | 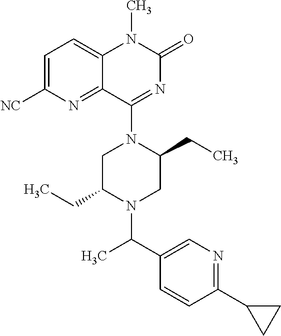 [SMILES: CC[C@H]1CN(C(C)c2ccc(C3CC3)nc2)[C@H](CC)CN1c1nc(=O)n(C)c2ccc(C#N)nc12] | H H | C C | 2.01 2.02 | 472.3 472.3 | B B |
| 194 195 | 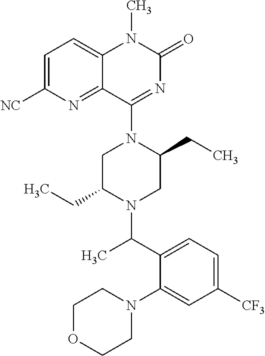 [SMILES: CC[C@H]1CN(C(C)c2ccc(C(F)(F)F)cc2N2CCOCC2)[C@H](CC)CN1c1nc(=O)n(C)c2ccc(C#N)nc12] | H H | C C | 2.41 2.40 | 584.4 584.3 | B B |
| 196 197 | 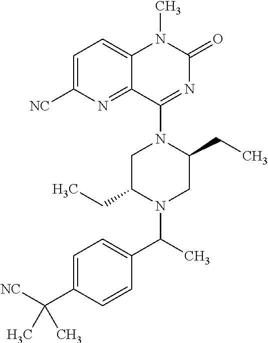 [SMILES: CC[C@H]1CN(C(C)c2ccc(C(C)(C)C#N)cc2)[C@H](CC)CN1c1nc(=O)n(C)c2ccc(C#N)nc12] | H H | C C | 2.13 2.15 | 498.4 498.3 | B B |
| 200 201 | 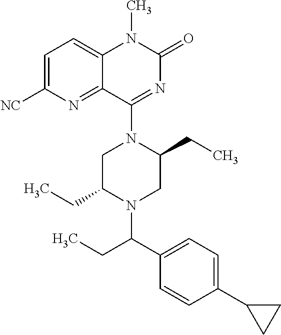 [SMILES: CCC(c1ccc(C2CC2)cc1)N1C[C@H](CC)N(c2nc(=O)n(C)c3ccc(C#N)nc23)C[C@H]1CC] | H H | C C | 2.59 2.62 | 485.4 485.3 | B B |
| 202 203 | 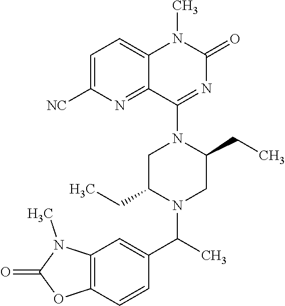 [SMILES: CC[C@H]1CN(C(C)c2ccc3oc(=O)n(C)c3c2)[C@H](CC)CN1c1nc(=O)n(C)c2ccc(C#N)nc12] | H H | C C | 1.90 1.91 | 502.3 502.3 | B B |
| 204 | 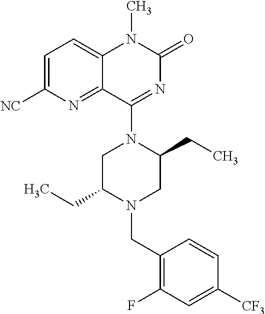 [SMILES: CC[C@@H]1CN(c2nc(=O)n(C)c3ccc(C#N)nc23)[C@@H](CC)CN1Cc1ccc(C(F)(F)F)cc1F] | H | C | 2.35 | 503.2 | A |
| 205 | 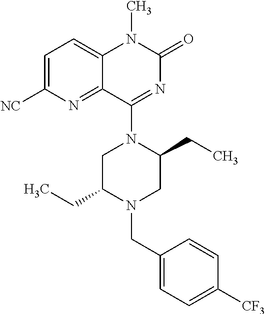 [SMILES: CC[C@@H]1CN(c2nc(=O)n(C)c3ccc(C#N)nc23)[C@@H](CC)CN1Cc1ccc(C(F)(F)F)cc1] | H | C | 2.33 | 485.3 | A |
| 206 207 | 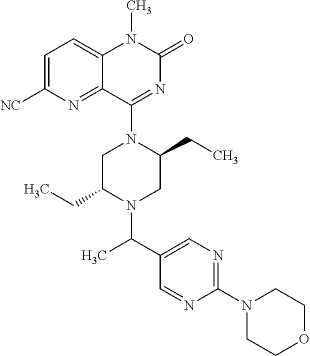 [SMILES: CC[C@H]1CN(C(C)c2cnc(N3CCOCC3)nc2)[C@H](CC)CN1c1nc(=O)n(C)c2ccc(C#N)nc12] | H H | C D | 1.83 1.11 | 518.3 518.5 | B B |
| 208 209 | 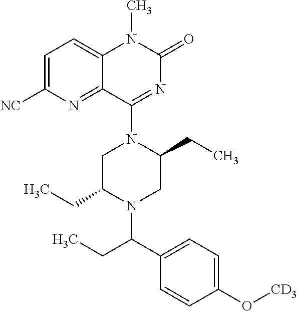 [SMILES: [2H]C([2H])([2H])Oc1ccc(C(CC)N2C[C@H](CC)N(c3nc(=O)n(C)c4ccc(C#N)nc34)C[C@H]2CC)cc1] | H H | C C | 2.29 2.32 | 478.3 478.4 | B B |
| 210 211 | 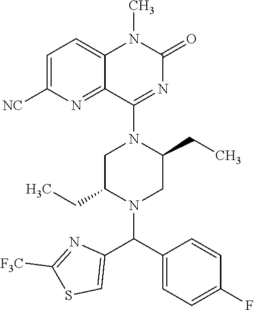 [SMILES: CC[C@H]1CN(C(c2ccc(F)cc2)c2csc(C(F)(F)F)n2)[C@H](CC)CN1c1nc(=O)n(C)c2ccc(C#N)nc12] | H H | C C | 2.35 2.36 | 586.3 586.3 | A A |
| 212 213 | 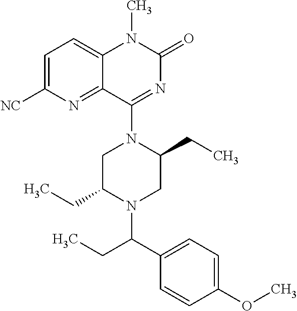 [SMILES: CCC(c1ccc(OC)cc1)N1C[C@H](CC)N(c2nc(=O)n(C)c3ccc(C#N)nc23)C[C@H]1CC] | H H | C C | 2.29 2.32 | 475.3 475.4 | B B |
| 214 215 | 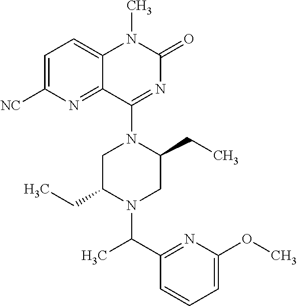 [SMILES: CC[C@H]1CN(C(C)c2cccc(OC)n2)[C@H](CC)CN1c1nc(=O)n(C)c2ccc(C#N)nc12] | H H | C C | 2.07 2.08 | 462.3 462.3 | B B |
| 216 217 | 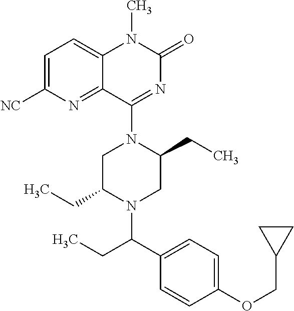 [SMILES: CCC(c1ccc(OCC2CC2)cc1)N1C[C@H](CC)N(c2nc(=O)n(C)c3ccc(C#N)nc23)C[C@H]1CC] | H H | D C | 1.57 2.57 | 515.4 515.3 | B B |
| 218 219 | 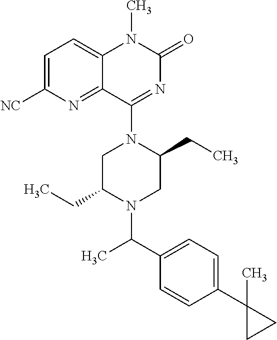 [SMILES: CC[C@H]1CN(C(C)c2ccc(C3(C)CC3)cc2)[C@H](CC)CN1c1nc(=O)n(C)c2ccc(C#N)nc12] | H H | C C | 2.62 2.64 | 485.3 485.3 | B B |
| 220 221 | 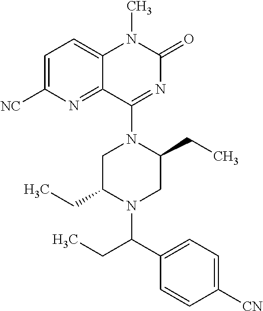 [SMILES: CCC(c1ccc(C#N)cc1)N1C[C@H](CC)N(c2nc(=O)n(C)c3ccc(C#N)nc23)C[C@H]1CC] | H H | C C | 2.13 2.15 | 470.3 470.3 | B B |
| 222 223 | 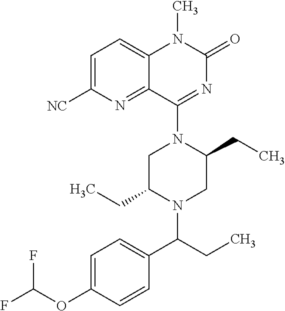 [SMILES: CCC(c1ccc(OC(F)F)cc1)N1C[C@H](CC)N(c2nc(=O)n(C)c3ccc(C#N)nc23)C[C@H]1CC] | H H | C C | 2.32 2.35 | 511.3 511.3 | B B |
| 224 225 | 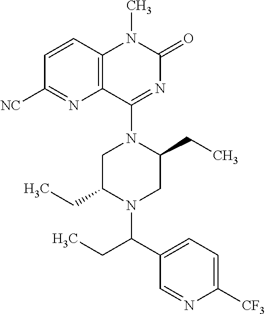 [SMILES: CCC(c1ccc(C(F)(F)F)nc1)N1C[C@H](CC)N(c2nc(=O)n(C)c3ccc(C#N)nc23)C[C@H]1CC] | H H | E E | 3.24 3.26 | 514.2 514.2 | B B |
| 226 227 | 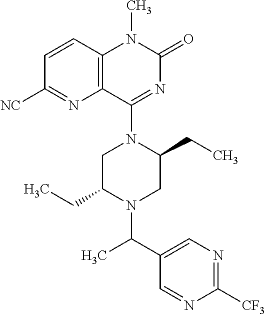 [SMILES: CC[C@H]1CN(C(C)c2cnc(C(F)(F)F)nc2)[C@H](CC)CN1c1nc(=O)n(C)c2ccc(C#N)nc12] | H H | E E | 2.87 2.86 | 501.2 501.2 | B B |
| 228 229 | 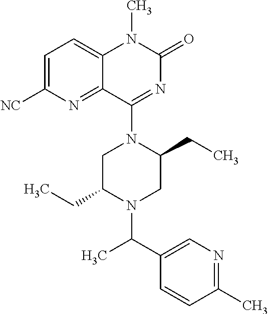 [SMILES: CC[C@H]1CN(C(C)c2ccc(C)nc2)[C@H](CC)CN1c1nc(=O)n(C)c2ccc(C#N)nc12] | H H | C C | 1.73 1.75 | 446.3 446.3 | B B |
| 230 231 | 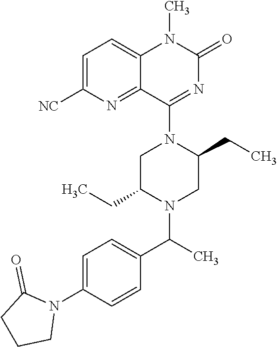 [SMILES: CC[C@H]1CN(C(C)c2ccc(N3CCCC3=O)cc2)[C@H](CC)CN1c1nc(=O)n(C)c2ccc(C#N)nc12] | H H | C C | 1.85 1.85 | 514.3 514.3 | B B |
| 232 233 | 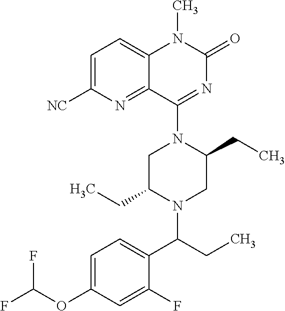 [SMILES: CCC(c1ccc(OC(F)F)cc1F)N1C[C@H](CC)N(c2nc(=O)n(C)c3ccc(C#N)nc23)C[C@H]1CC] | H H | C C | 2.34 2.37 | 529.3 529.3 | B B |
| 234 235 | 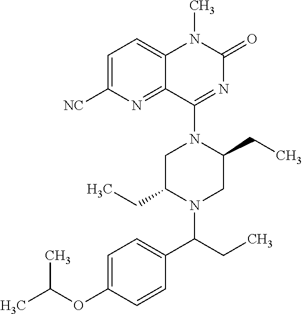 [SMILES: CCC(c1ccc(OC(C)C)cc1)N1C[C@H](CC)N(c2nc(=O)n(C)c3ccc(C#N)nc23)C[C@H]1CC] | H H | F F | 1.72 1.70 | 503.25 503.27 | B B |
| 236 237 | 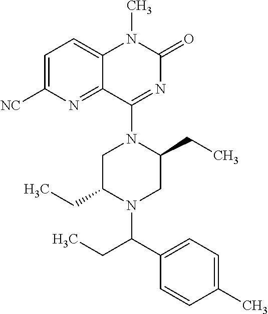 [SMILES: CCC(c1ccc(C)cc1)N1C[C@H](CC)N(c2nc(=O)n(C)c3ccc(C#N)nc23)C[C@H]1CC] | H H | E E | 3.86 3.95 | 459.4 459.4 | B B |
| 238 239 | 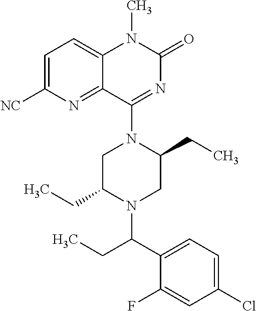 [SMILES: CCC(c1ccc(Cl)cc1F)N1C[C@H](CC)N(c2nc(=O)n(C)c3ccc(C#N)nc23)C[C@H]1CC] | H H | E E | 3.92 3.98 | 497.2 497.2 | B B |
| 240 241 | 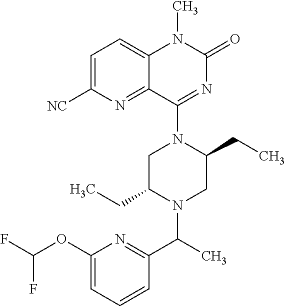 [SMILES: CC[C@H]1CN(C(C)c2cccc(OC(F)F)n2)[C@H](CC)CN1c1nc(=O)n(C)c2ccc(C#N)nc12] | H H | C C | 2.14 2.15 | 498.3 498.3 | B B |
| 242 | 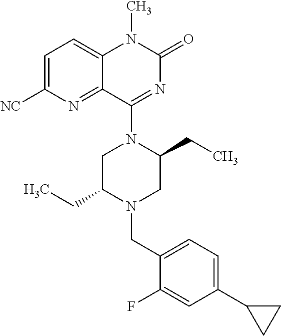 [SMILES: CC[C@@H]1CN(c2nc(=O)n(C)c3ccc(C#N)nc23)[C@@H](CC)CN1Cc1ccc(C2CC2)cc1F] | H | C | 2.37 | 475.3 | B |
| 245 246 | 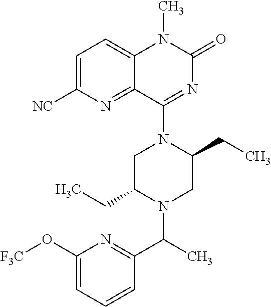 [SMILES: CC[C@H]1CN(C(C)c2cccc(OC(F)(F)F)n2)[C@H](CC)CN1c1nc(=O)n(C)c2ccc(C#N)nc12] | H H | C C | 2.24 2.24 | 516.3 516.3 | B B |
| 247 248 | 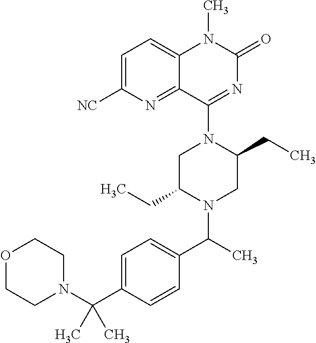 [SMILES: CC[C@H]1CN(C(C)c2ccc(C(C)(C)N3CCOCC3)cc2)[C@H](CC)CN1c1nc(=O)n(C)c2ccc(C#N)nc12] | H H | E E | 3.18 3.02 | 558.5 558.5 | B B |
| 249 250 | 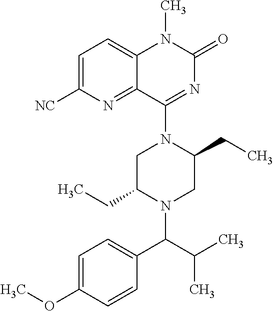 [SMILES: CC[C@H]1CN(C(c2ccc(OC)cc2)C(C)C)[C@H](CC)CN1c1nc(=O)n(C)c2ccc(C#N)nc12] | H H | C C | 2.40 2.45 | 489.3 489.3 | B B |
| 251 252 | 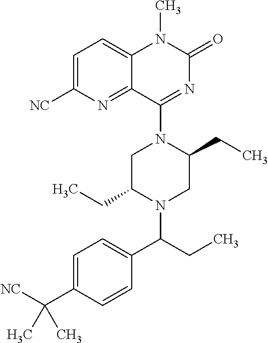 [SMILES: CCC(c1ccc(C(C)(C)C#N)cc1)N1C[C@H](CC)N(c2nc(=O)n(C)c3ccc(C#N)nc23)C[C@H]1CC] | H H | C C | 2.3 2.33 | 512.3 512.3 | B B |
| 253 254 | 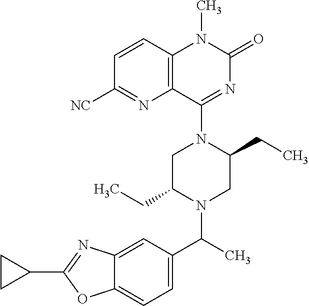 [SMILES: CC[C@H]1CN(C(C)c2ccc3oc(C4CC4)nc3c2)[C@H](CC)CN1c1nc(=O)n(C)c2ccc(C#N)nc12] | H H | E E | 1.88 1.96 | 512.4 513.4 | B B |
| 255 256 | 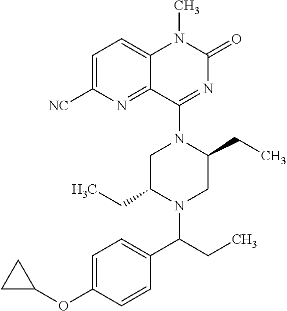 [SMILES: CCC(c1ccc(OC2CC2)cc1)N1C[C@H](CC)N(c2nc(=O)n(C)c3ccc(C#N)nc23)C[C@H]1CC] | H H | C C | 2.47 2.50 | 501.3 501.3 | B B |
| 257 258 | 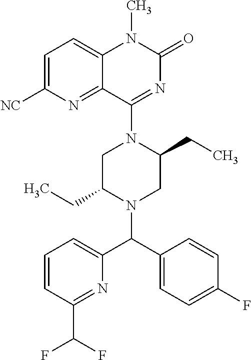 [SMILES: CC[C@H]1CN(C(c2ccc(F)cc2)c2cccc(C(F)F)n2)[C@H](CC)CN1c1nc(=O)n(C)c2ccc(C#N)nc12] | H H | C D | 2.21 1.72 | 562.3 562.3 | A A |
| 259 260 | 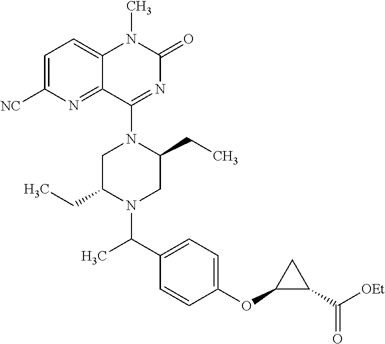 [SMILES: CCOC(=O)[C@H]1C[C@@H]1Oc1ccc(C(C)N2C[C@H](CC)N(c3nc(=O)n(C)c4ccc(C#N)nc34)C[C@H]2CC)cc1] | H H | C C | 1.87 1.87 | 559.3 559.3 | B B |
| 261 262 | 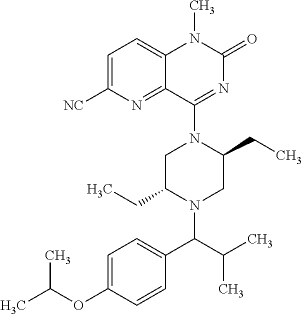 [SMILES: CC[C@H]1CN(C(c2ccc(OC(C)C)cc2)C(C)C)[C@H](CC)CN1c1nc(=O)n(C)c2ccc(C#N)nc12] | H H | C C | 2.72 2.76 | 517.4 517.4 | B B |
| 263 264 | 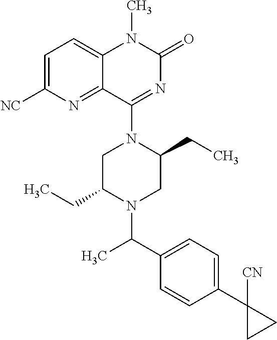 [SMILES: CC[C@H]1CN(C(C)c2ccc(C3(C#N)CC3)cc2)[C@H](CC)CN1c1nc(=O)n(C)c2ccc(C#N)nc12] | H H | D C | 1.29 2.23 | 496.4 496.4 | B B |
| 265 266 | 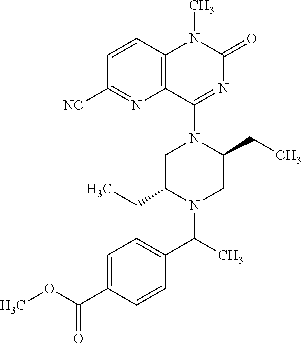 [SMILES: CC[C@H]1CN(C(C)c2ccc(C(=O)OC)cc2)[C@H](CC)CN1c1nc(=O)n(C)c2ccc(C#N)nc12] | H H | C C | 2.13 2.12 | 489.4 489.4 | B B |
| 267 268 | 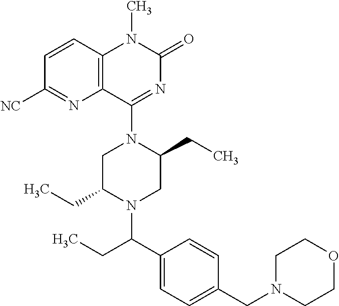 [SMILES: CCC(c1ccc(CN2CCOCC2)cc1)N1C[C@H](CC)N(c2nc(=O)n(C)c3ccc(C#N)nc23)C[C@H]1CC] | H H | C C | 2.13 2.18 | 544.4 544.4 | B B |
| 269 | 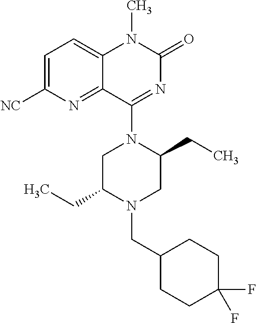 [SMILES: CC[C@@H]1CN(c2nc(=O)n(C)c3ccc(C#N)nc23)[C@@H](CC)CN1CC1CCC(F)(F)CC1] | H | C | 2.19 | 459.3 | C |

### Intermediate 29

### Methyl 4-(1-chloroethyl)benzoate

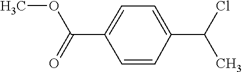

To a stirred solution of methyl 4-(1-hydroxyethyl)benzoate (2.3 g, 12.76 mmol) in DCM (20 mL) was added thionyl chloride (4.7 mL, 63.8 mmol) at 0° C. under nitrogen. The reaction mixture was allowed to warm to room temperature and stirring was continued for 2 h. The solvent was removed under reduced pressure, co-distilled with acetonitrile (2×10 mL) and dried to afford methyl 4-(1-chloroethyl)benzoate (2 g, 79% yield). ¹H NMR (300 MHz, DMSO-d₆) δ (ppm)=7.97 (d, J=8.3 Hz, 2H), 7.64 (d, J=8.3 Hz, 2H), 5.48-5.37 (m, 1H), 3.86 (s, 3H), 1.80 (d, J=6.8 Hz, 3H).

### Intermediate 30

### 4-(1-Chloroethyl)phenyl)methanol

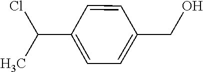

A stirred solution of methyl 4-(1-chloroethyl)benzoate (2.5 g, 12.6 mmol) in THF (25 mL) was cooled to 0° C. and lithium borohydride (2M in THF) (12.6 mL, 25.2 mmol) was added drop wise under nitrogen. The reaction mixture was allowed to warm room temperature and stirred for 12 h. The reaction mixture was cooled to 0° C. The reaction was quenched with the addition of saturated aqueous NH₄Cl solution. The reaction mixture was extracted with EtOAc (3×50 mL). The combined organic extract was washed with water, brine solution, dried over sodium sulphate and the solvent was removed under reduced pressure to afford (4-(1-chloroethyl)phenyl)methanol (1.5 g, 49% yield). LCMS: m/z, 135.1 [M-Cl]; rt 1.89 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.3 minute hold at 100% B, flow: 0.7 mL/min, temperature: 27° C.; detection: UV at 220 nm.

### Examples 270 and 271

### 4-((2S,5R)-2,5-Diethyl-4-(1-(4-(hydroxymethyl)phenyl)ethyl)piperazin-1-yl)-1-methyl-2-oxo-1,2-dihydropyrido[3,2-d]pyrimidine-6-carbonitrile

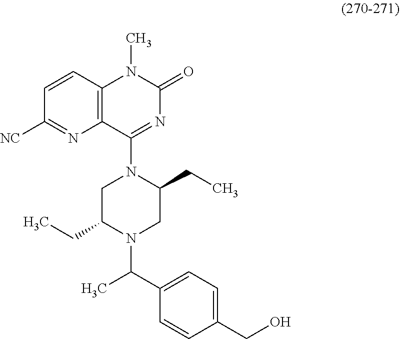 [SMILES: CC[C@H]1CN(C(C)c2ccc(CO)cc2)[C@H](CC)CN1c1nc(=O)n(C)c2ccc(C#N)nc12]

To a stirred solution of 4-((2S,5R)-2,5-diethylpiperazin-1-yl)-1-methyl-2-oxo-1,2-dihydropyrido[3,2-d]pyrimidine-6-carbonitrile hydrochloride (80 mg, 0.22 mmol) in acetonitrile (2 mL), DIPEA (0.12 mL, 0.66 mmol), (4-(1-chloroethyl)phenyl)methanol (56.4 mg, 0.33 mmol) and sodium iodide (33.0 mg, 0.22 mmol) were added sequentially at room temperature followed by heating at 80° C. for 12 h. The reaction mixture was cooled to room temperature and solvent was removed under reduced pressure to give the crude product, which was purified using preparative SFC. SFC Method: Column: Chiralpak IG (250×30 mm, 5 μm); mobile phase: 50% CO₂/50% of co-solvent: acetonitrile:MeOH; Flow: 95 g/min; Detector Wavelength: 220 nm; Temperature: 40° C.

EXAMPLE 270 (6.7 mg, 7% yield). LCMS: m/z=461.3 [M+H]⁺; rt 1.71 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.27-8.19 (m, 1H), 7.98 (d, J=9.0 Hz, 1H), 7.35-7.27 (m, 4H), 5.86-5.46 (m, 1H), 5.17-5.09 (m, 1H), 5.05-4.81 (m, 1H), 4.53-4.47 (m, 2H), 3.73-3.65 (m, 1H), 3.44 (s, 3H), 3.12-2.88 (m, 2H), 2.79-2.70 (m, 1H), 2.47-2.35 (m, 1H), 2.27-1.84 (m, 2H), 1.53-1.31 (m, 2H), 1.30-1.25 (m, 3H), 1.03-0.86 (m, 3H), 0.73-0.54 (m, 3H).

EXAMPLE 271 (8 mg, 8% yield). LCMS: m/z=461.3 [M+H]⁺; rt 1.73 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.29-8.13 (m, 1H), 8.05-7.89 (m, 1H), 7.39-7.16 (m, 4H), 6.10-5.19 (m, 1H), 5.14-4.71 (m, 2H), 4.47 (br d, J=5.6 Hz, 2H), 3.67-3.50 (m, 2H), 3.43, 3.41 (s, 3H), 3.18-3.04 (m, 1H), 2.45-2.24 (m, 2H), 2.10-1.66 (m, 2H), 1.54-1.38 (m, 2H), 1.25-1.21 (m, 3H), 1.00-0.85 (m, 3H), 0.69-0.55 (m, 3H).

### Example 272

### 4-((2S,5R)-4-(1-(4-(Bromomethyl)phenyl)ethyl)-2,5-diethylpiperazin-1-yl)-1-methyl-2-oxo-1,2-dihydropyrido[3,2-d]pyrimidine-6-carbonitrile (Diastereomeric Mixture)

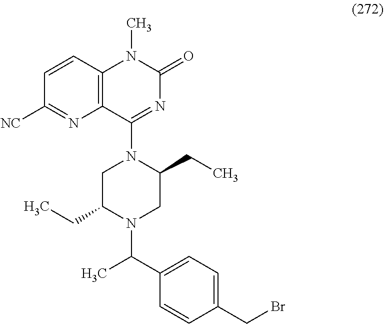 [SMILES: CC[C@H]1CN(C(C)c2ccc(CBr)cc2)[C@H](CC)CN1c1nc(=O)n(C)c2ccc(C#N)nc12]

A stirred solution of 4-((2S,5R)-2,5-diethyl-4-(1-(4-(hydroxymethyl)phenyl) ethyl)piperazin-1-yl)-1-methyl-2-oxo-1,2-dihydropyrido[3,2-d]pyrimidine-6-carbonitrile (1.2 g, 2.61 mmol) in DCM (20 mL) was cooled to 0° C. Triphenylphosphine polymer bound (1.3 mmol/g) (0.820 g, 3.13 mmol) was added followed by the addition of CBr₄ (1.73 g, 5.21 mmol) in DCM (10 mL). The reaction mixture was allowed to warm to room temperature and stirred for 2 h. The solvent was removed under reduced pressure to give the crude product, which was purified by silica gel column chromatography using 10-12% MeOH in CHCl₃ to afford 4-((2S,5R)-4-(1-(4-(bromomethyl)phenyl)ethyl)-2,5-diethylpiperazin-1-yl)-1-methyl-2-oxo-1,2-dihydropyrido[3,2-d]pyrimidine-6-carbonitrile (1 g, 0.955 mmol, 36.7% yield). LCMS: m/z, 525.1 [M+2]; rt 1.99 and 2.01 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Examples 273 and 274

### 4-((2S,5R)-2,5-Diethyl-4-(1-(4-((4-methoxypiperidin-1-yl)methyl)phenyl)ethyl)piperazin-1-yl)-1-methyl-2-oxo-1,2-dihydropyrido[3,2-d]pyrimidine-6-carbonitrile

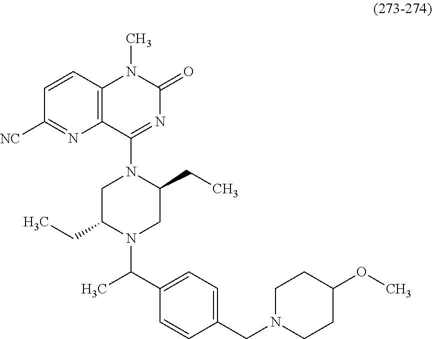 [SMILES: CC[C@H]1CN(C(C)c2ccc(CN3CCC(OC)CC3)cc2)[C@H](CC)CN1c1nc(=O)n(C)c2ccc(C#N)nc12]

To a stirred solution of 4-((2S,5R)-4-(1-(4-(bromomethyl)phenyl)ethyl)-2,5-diethylpiperazin-1-yl)-1-methyl-2-oxo-1,2-dihydropyrido[3,2-d]pyrimidine-6-carbonitrile (100 mg, 0.19 mmol) in acetonitrile (2 mL) were added DIPEA (0.1 mL, 0.57 mmol) and 4-methoxypiperidine (44.0 mg, 0.38 mmol) at room temperature. The reaction mixture was heated at 80° C. for 1 h. The reaction mixture was cooled to room temperature and the solvent was removed under reduced pressure to give the crude product, which was purified using preparative SFC. SFC Method: Column: Chiralpak IG (250×30 mm, 5 μm); mobile phase: 50% CO₂/50% of co-solvent: 0.2% ammonia in MeOH, Flow: 95 g/min; Detector Wavelength: 220 nm; Temperature: 40° C.

EXAMPLE 273: (5.4 mg, 5% yield). LCMS: m/z=558.2 [M+H]⁺; rt 1.91 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.24-8.18 (m, 1H), 7.96 (d, J=9.0 Hz, 1H), 7.32-7.21 (m, 4H), 5.82-5.44 (m, 1H), 5.03-4.78 (m, 1H), 3.66 (q, J=6.2 Hz, 1H), 3.42 (s, 5H), 3.20 (s, 3H), 3.19-3.03 (m, 2H), 2.93-2.86 (m, 1H), 2.76-2.68 (m, 1H), 2.65-2.58 (m, 2H), 2.45-2.33 (m, 1H), 2.24-2.01 (m, 3H), 1.85-1.75 (m, 2H), 1.48-1.18 (m, 8H), 0.98-0.86 (m, 3H), 0.71-0.51 (m, 3H).

EXAMPLE 274: (3.7 mg, 4% yield). LCMS: m/z=558.4 [M+H]⁺; rt 1.93 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.29-8.16 (m, 1H), 8.03-7.91 (m, 1H), 7.36-7.21 (m, 4H), 6.12-5.16 (m, 1H), 5.14-4.67 (m, 1H), 3.67-3.49 (m, 2H), 3.45-3.40 (m, 5H), 3.20 (s, 3H), 3.18-3.05 (m, 2H), 2.62-2.60 (m, 2H), 2.47-2.41 (m, 1H), 2.30-2.21 (m, 1H), 2.09-2.01 (m, 2H), 1.86-1.76 (m, 2H), 1.54-1.33 (m, 4H), 1.33-1.15 (m, 5H), 1.03-0.90 (m, 3H), 0.58 (t, J=7.3 Hz, 3H).

The examples in the Table 10 were prepared according to the general procedure described in Examples 273 and 274, using the appropriate amine and ((2S,5R)-4-(1-(4-(bromomethyl)phenyl)ethyl)-2,5-diethylpiperazin-1-yl)-1-methyl-2-oxo-1,2-dihydropyrido[3,2-d]pyrimidine-6-carbonitrile. When the reaction provided a mixture of diastereomers, the mixture was separated at the final stage using either preparative chromatography or preparative chiral chromatography. The absolute stereochemistry was not assigned at the benzylic position.

TABLE 10

| Ex. | | Stereo | LCMS | LCMS | |
|---|---|---|---|---|---|
| No. | Structure | Chem. | Method | rt | [M + H]⁺ |
| 275 276 | 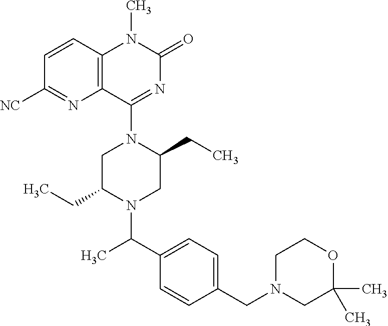 [SMILES: CC[C@H]1CN(C(C)c2ccc(CN3CCOC(C)(C)C3)cc2)[C@H](CC)CN1c1nc(=O)n(C)c2ccc(C#N)nc12] | H H | E E | 3.76 3.87 | 558.4 558.4 |
| 277 278 | 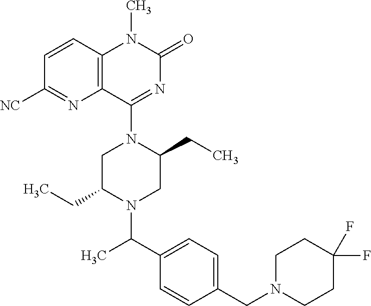 [SMILES: CC[C@H]1CN(C(C)c2ccc(CN3CCC(F)(F)CC3)cc2)[C@H](CC)CN1c1nc(=O)n(C)c2ccc(C#N)nc12] | H H | E E | 3.76 3.68 | 564.4 564.4 |
| 279 280 | 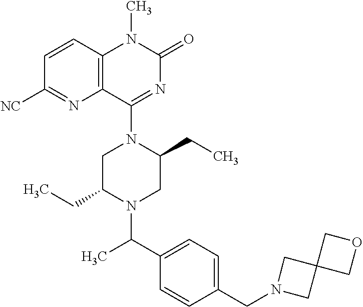 [SMILES: CC[C@H]1CN(C(C)c2ccc(CN3CC4(COC4)C3)cc2)[C@H](CC)CN1c1nc(=O)n(C)c2ccc(C#N)nc12] | H H | C C | 1.67 1.68 | 542.4 542.4 |
| 281 282 | 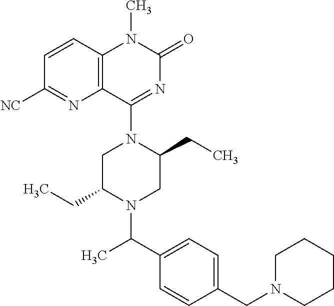 [SMILES: CC[C@H]1CN(C(C)c2ccc(CN3CCCCC3)cc2)[C@H](CC)CN1c1nc(=O)n(C)c2ccc(C#N)nc12] | H H | E E | 2.68 2.25 | 528.4 528.4 |
| 283 284 | 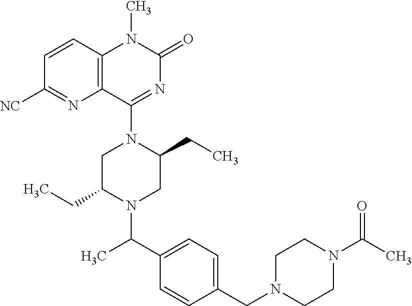 [SMILES: CC[C@H]1CN(C(C)c2ccc(CN3CCN(C(C)=O)CC3)cc2)[C@H](CC)CN1c1nc(=O)n(C)c2ccc(C#N)nc12] | H H | C C | 1.8 1.83 | 571.4 571.4 |
| 285 286 | 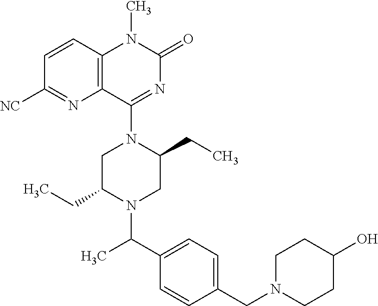 [SMILES: CC[C@H]1CN(C(C)c2ccc(CN3CCC(O)CC3)cc2)[C@H](CC)CN1c1nc(=O)n(C)c2ccc(C#N)nc12] | H H | C C | 1.52 1.54 | 544.4 544.4 |
| 287 288 | 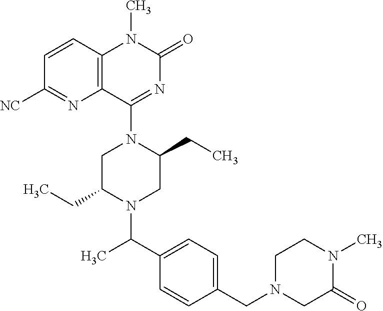 [SMILES: CC[C@H]1CN(C(C)c2ccc(CN3CCN(C)C(=O)C3)cc2)[C@H](CC)CN1c1nc(=O)n(C)c2ccc(C#N)nc12] | H H | C C | 1.76 1.77 | 557.4 557.3 |
| 289 290 | 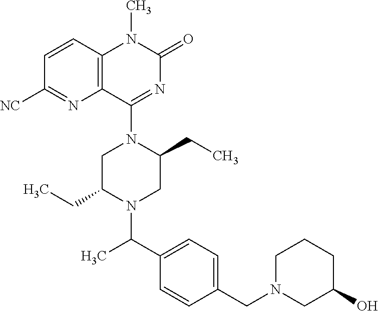 [SMILES: CC[C@H]1CN(C(C)c2ccc(CN3CCC[C@@H](O)C3)cc2)[C@H](CC)CN1c1nc(=O)n(C)c2ccc(C#N)nc12] | H H | C C | 1.68 1.69 | 544.4 544.4 |
| 291 292 | 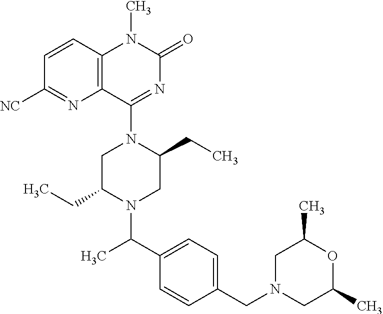 [SMILES: CC[C@H]1CN(C(C)c2ccc(CN3C[C@@H](C)O[C@@H](C)C3)cc2)[C@H](CC)CN1c1nc(=O)n(C)c2ccc(C#N)nc12] | H H | C C | 2.27 2.3 | 558.4 558.4 |

### Intermediate 31

### (3-Bromo-1-methyl-1H-1,2,4-triazol-5-yl)(4-fluorophenyl)methanol

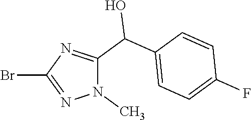 [SMILES: Cn1nc(Br)nc1C(O)c1ccc(F)cc1]

To a solution of 3,5-dibromo-1-methyl-1H-1,2,4-triazole (1.0 g, 4.15 mmol) in THF (15 mL) at −78° C. was added n-butyl lithium (2.5 M, 1.66 mL, 4.15 mmol). The reaction mixture was stirred for 20 minutes, 4-fluorobenzaldehyde (0.515 g, 4.15 mmol) was added at −78° C. and stirred for 30 minutes at room temperature. The reaction was quenched with the addition of saturated aqueous ammonium chloride solution (25 mL). The reaction mixture was extracted with ethyl acetate (2×50 mL) and the combined organic layer was dried over anhydrous sodium sulphate and filtered. Evaporation of the solvent under reduced pressure furnished the crude product, which was purified using silica gel chromatography (0-40% ethyl acetate/pet ether) to afford 3-bromo-1-methyl-1H-1,2,4-triazol-5-yl)(4-fluorophenyl)methanol (950 mg, 80% yield). LCMS: m/z, 286.0 [M+2]⁺; retention time 1.09 min; LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 4 min, then a 0.6 minute hold at 100% B, flow: 0.7 mL/min, temperature: 27° C.; detection: UV at 220 nm.

### Intermediate 32

### 3-Bromo-5-(chloro(4-fluorophenyl)methyl)-1-methyl-1H-1,2,4-triazole

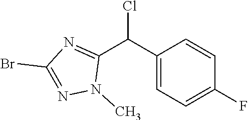 [SMILES: Cn1nc(Br)nc1C(Cl)c1ccc(F)cc1]

To a stirred solution of (3-bromo-1-methyl-1H-1,2,4-triazol-5-yl)(4-fluorophenyl) methanol (200 mg, 0.7 mmol) in dry DCM (3.0 mL) were added DIPEA (0.37 mL, 2.1 mmol) and mesyl chloride (0.07 mL, 0.84 mmol) at 0° C. The reaction mixture was stirred at room temperature for 2 h. The reaction was quenched with the addition of water (5 mL). The crude material was extracted with DCM (1×100 mL), washed with water, brine, and dried over sodium sulphate. Evaporation of the solvent under reduced pressure furnished 3-bromo-5-(chloro(4-fluorophenyl)methyl)-1-methyl-1H-1,2,4-triazole (200 mg, 94% yield) as a brown semi solid. LCMS: m/z, 304.0 [M+2]; retention time 1.56 min; LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 4 min, then a 0.6 minute hold at 100% B, flow: 0.7 mL/min, temperature: 27° C.; detection: UV at 220 nm.

### Example 293

### 4-((2S,5R)-4-((3-Bromo-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile

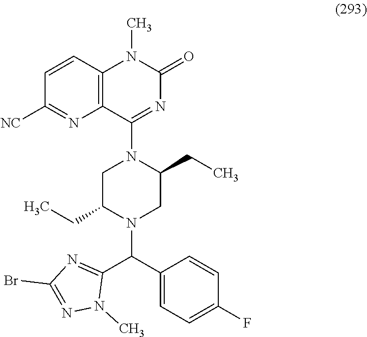 [SMILES: CC[C@H]1CN(C(c2ccc(F)cc2)c2nc(Br)nn2C)[C@H](CC)CN1c1nc(=O)n(C)c2ccc(C#N)nc12]

To a stirred solution of 3-bromo-5-(chloro(4-fluorophenyl)methyl)-1-methyl-1H-1,2,4-triazole (120 mg, 0.39 mmol), 4-((2S,5R)-2,5-diethylpiperazin-1-yl)-1-methyl-2-oxo-1,2-dihydropyrido[3,2-d]pyrimidine-6-carbonitrile, TFA (208 mg, 0.473 mmol) in acetonitrile (6.0 mL) was added DIPEA (0.21 mL, 1.18 mmol). The reaction mixture was heated at 85° C. for 3 h and then cooled to room temperature. The volatiles were removed under reduced pressure to obtain the crude product, which was purified by silica gel (12 g) chromatography by using 0-10% methanol in chloroform as eluent. The fractions were concentrated under reduced pressure to obtain 4-((2S,5R)-4-((3-bromo-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile (120 mg, 51% yield). LCMS: m/z, 594.3 [M+H]⁺; retention time 1.70 min; LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min, temperature: 27° C.; detection: UV at 220 nm.

### Examples 294 and 295

### 4-((2S,5R)-4-((3-Cyclopropyl-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile

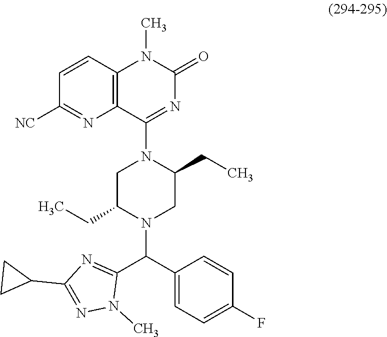 [SMILES: CC[C@H]1CN(C(c2ccc(F)cc2)c2nc(C3CC3)nn2C)[C@H](CC)CN1c1nc(=O)n(C)c2ccc(C#N)nc12]

A solution of 4-((2S,5R)-4-((3-bromo-1-methyl-1H-1,2,4-triazol-5-yl)(4-fluorophenyl)methyl)-2,5-diethylpiperazin-1-yl)-1-methyl-2-oxo-1,2-dihydropyrido[3,2-d]pyrimidine-6-carbonitrile (70 mg, 0.12 mmol), cyclopropylboronic acid (10.11 mg, 0.118 mmol) and Cs₂CO₃ (115 mg, 0.35 mmol) in dry dioxane (2.0 mL) was purged with argon for 10 minutes. PdCl₂(dppf)-CH₂Cl₂ adduct (9.62 mg, 0.01 mmol) was added. The reaction mixture was purged with argon for another 5 minutes and heated at 120° C. for 6 h. The reaction mixture was cooled to room temperature and the volatiles were removed under reduced pressure to obtain the crude product, which was purified using preparative chiral LCMS. HPLC Method: Column: XBridge C18 (19×150 mm, 5 μm); mobile phase A: water:acetonitrile (95:5); mobile phase B: water:acetonitrile (5:95); 15-47% over 25 minutes, then a 5 minute hold at 100% B; Flow: 15 mL/min; fractions were concentrated under reduced pressure and lyophilized from (EtOH/H₂O, 1:5) to yield Examples 294 and 295.

EXAMPLE 294: (13 mg, 19% yield); LCMS: m/z, 556.6 [M+H]⁺; rt 1.85 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.31-8.18 (m, 1H), 8.06-7.93 (m, 1H), 7.69-7.54 (m, 2H), 7.21 (t, J=8.8 Hz, 2H), 5.97-5.35 (m, 1H), 5.1 (s, 1H), 4.99-4.82 (m, 1H), 3.85 (s, 3H), 3.66-3.55 (m, 1H), 3.43 (s, 3H), 2.62 (ddd, J=1.3, 2.0, 14.2 Hz, 2H), 2.22-1.75 (m, 3H), 1.52-1.30 (m, 2H), 0.87 (dd, J=2.9, 8.3 Hz, 2H), 0.80-0.57 (m, 9H).

EXAMPLE 295: (21 mg, 30% yield); LCMS: m/z, 556.4 [M+H]⁺; rt 1.85 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.24 (d, J=8.8 Hz, 1H), 8.03-7.95 (m, 1H), 7.76-7.62 (m, 2H), 7.31-7.17 (m, 2H), 5.96-5.32 (m, 1H), 5.29-4.81 (m, 2H), 3.99-3.89 (m, 1H), 3.77 (br s, 3H), 3.69-3.58 (m, 1H), 3.32-3.24 (m, 1H), 2.90-2.73 (m, 1H), 2.62-2.55 (m, 1H), 2.31-2.00 (m, 3H), 1.95-1.69 (m, 2H), 1.62-1.33 (m, 2H), 0.94-0.58 (m, 10H).

### Intermediate 33

### methyl N-benzyl-N—((R)-2-((tert-butoxycarbonyl)amino)butanoyl)-L-alaninate

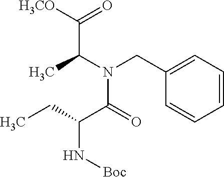 [SMILES: CC[C@@H](NC(=O)OC(C)(C)C)C(=O)N(Cc1ccccc1)[C@@H](C)C(=O)OC]

To a solution of (R)-2-((tert-butoxycarbonyl)amino)butanoic acid (6.31 g, 31.0 mmol) in DMF (2 mL) at room temperature was added HATU (8.85 g, 23.29 mmol), DIPEA (8.13 mL, 46.6 mmol), and methyl benzyl-L-alaninate (3 g, 15.52 mmol). The reaction mixture was stirred at room temperature for 2 h. The reaction was quenched with water. The reaction mixture was extracted with ethyl acetate (2×100 mL), the combined organic layer was washed with water, brine and dried over sodium sulfate. The solvent was evaporated under reduced pressure to obtain the crude product, which was purified by silica gel column chromatography using 30% EtOAc in pet ether. The fractions were concentrated under reduced pressure to obtain the purified methyl N-benzyl-N—((R)-2-((tert-butoxycarbonyl)amino)butanoyl)-L-alaninate (5 g, 13.21 mmol, 85% yield). LCMS: m/z=379.4 [M+H]⁺; retention time 1.80 min. LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Intermediate 34

### Methyl N—((R)-2-aminobutanoyl)-N-benzyl-L-alaninate

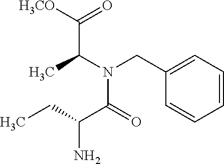 [SMILES: CC[C@@H](N)C(=O)N(Cc1ccccc1)[C@@H](C)C(=O)OC]

To a solution of methyl N-benzyl-N—((R)-2-((tert-butoxycarbonyl)amino) butanoyl)-L-alaninate (5 g, 13.21 mmol) in DCM (10 mL) at 0° C. was added TFA (15.27 mL, 198 mmol). The reaction mixture was stirred at room temperature for 3 h. The reaction mixture was concentrated under reduced pressure and the crude product was washed with 1:1 diethyl ether and pet ether and dried under high vacuum to yield the methyl N—((R)-2-aminobutanoyl)-N-benzyl-L-alaninate, TFA (5 g, 10.96 mmol, 83% yield)). LCMS: m/z=279.3 [M+H]⁺; retention time 0.96 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Intermediate 35

### (3R,6S)-1-Benzyl-3-ethyl-6-methylpiperazine-2,5-dione

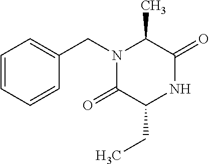 [SMILES: CC[C@H]1NC(=O)[C@H](C)N(Cc2ccccc2)C1=O]

A solution of methyl N—((R)-2-aminobutanoyl)-N-benzyl-L-alaninate, TFA (5 g, 12.74 mmol) in methanol (5 mL) was refluxed at 65° C. overnight. The reaction mixture was concentrated under reduced pressure and dried under high vacuum to afford the (3R,6S)-1-benzyl-3-ethyl-6-methylpiperazine-2,5-dione (2.5 g, 10.15 mmol, 80% yield). LCMS: m/z=247.3 [M+H]⁺; retention time 0.46 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Intermediate 36

### (2S,5R)-1-Benzyl-5-ethyl-2-methylpiperazine

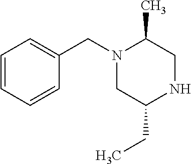 [SMILES: CC[C@@H]1CN(Cc2ccccc2)[C@@H](C)CN1]

To a stirred solution of (3R,6S)-1-benzyl-3-ethyl-6-methylpiperazine-2,5-dione (2.5 g, 10.15 mmol) in THF (10 mL) at 0° C. was added dropwise 1 M solution of borane tetrahydrofuran complex (50.7 mL, 50.7 mmol) in THF. The mixture was refluxed overnight and cooled. The reaction was quenched slowly with the addition of methanol. To this reaction mixture was added concentrated HCl (0.5 mL) and refluxed for 3 h, cooled, and concentrated under reduced pressure. The crude product was dissolved in DCM and washed with saturated aqueous NaHCO₃ solution, brine, dried over anhydrous Na₂SO₄, filtered and evaporated under reduced pressure to yield (2S,5R)-1-benzyl-5-ethyl-2-methylpiperazine (2.0 g, 7.33 mmol, 72.2% yield). LCMS: m/z=219.2 [M+H]⁺; retention time 0.33 min. Column: Kinetex XB-C18 (75×3 mm, 2.6 μm) mobile phase A: 10 mM NH₄OAc in water:acetonitrile (98:2) mobile phase B: 10 mM NH₄OAc in water:acetonitrile (2:98).

### Intermediate 37

### tert-Butyl (2R,5S)-4-benzyl-2-ethyl-5-methylpiperazine-1-carboxylate

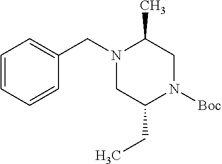 [SMILES: CC[C@@H]1CN(Cc2ccccc2)[C@@H](C)CN1C(=O)OC(C)(C)C]

To a solution of (2S,5R)-1-benzyl-5-ethyl-2-methylpiperazine (2.3 g, 10.53 mmol) in DCM (5 mL) was added TEA (2.202 mL, 15.80 mmol), Boc-anhydride (2.446 mL, 10.53 mmol) at room temperature. The reaction mixture was stirred for 3 h. The reaction was quenched with water. The reaction mixture was extracted with ethyl acetate (2×100 mL), the combined organic layer was washed with water, brine and dried over sodium sulfate. The solvent was evaporated under reduced pressure to obtain the crude product which was purified by silica gel column chromatography using 40% EtOAc in pet ether. The fractions were concentrated under reduced pressure to yield purified tert-butyl (2R,5S)-4-benzyl-2-ethyl-5-methylpiperazine-1-carboxylate (2 g, 5.53 mmol, 52.5% yield). LCMS: m/z=319.4 [M+H]⁺; retention time 1.69 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min, temperature: 27° C.; detection: UV at 220 nm.

### Intermediate 38

### tert-Butyl (2R,5S)-2-ethyl-5-methylpiperazine-1-carboxylate

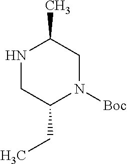 [SMILES: CC[C@@H]1CN[C@@H](C)CN1C(=O)OC(C)(C)C]

In a 100 mL autoclave, a solution of tert-butyl (2R,5S)-4-benzyl-2-ethyl-5-methylpiperazine-1-carboxylate (2.1 g, 6.59 mmol) in methanol (5 mL) and acetic acid (0.413 mL, 7.22 mmol) at room temperature was purged with nitrogen. Palladium on carbon (0.211 g, 1.978 mmol, 10% w/w) was added and the reaction mixture was evacuated. The reaction mixture was placed under hydrogen and stirred under hydrogen overnight. The reaction mixture was filtered through a Celite® bed, and the filtrate was concentrated under reduced pressure. The product was dried under high vacuum to yield the tert-butyl (2R,5S)-2-ethyl-5-methylpiperazine-1-carboxylate, AcOH (1.5 g, 5.20 mmol, 79% yield). LCMS: m/z=229.2 [M+H]⁺; retention time 0.90 min. Column: Kinetex XB-C18 (75×3 mm, 2.6 μm); mobile phase A: 10 mM NH₄OAc in water:acetonitrile (98:2) mobile phase B: 10 mM NH₄OAc in water:acetonitrile (2:98).

### Intermediate 39

### tert-Butyl (2R,5S)-4-(6-cyano-1-methyl-2-oxo-1,2-dihydro-1,5-naphthyridin-4-yl)-2-ethyl-5-methylpiperazine-1-carboxylate

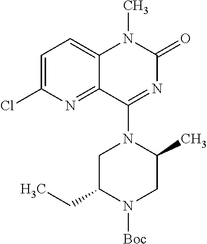 [SMILES: CC[C@@H]1CN(c2nc(=O)n(C)c3ccc(Cl)nc23)[C@@H](C)CN1C(=O)OC(C)(C)C]

To a stirred solution of tert-butyl (2R,5S)-2-ethyl-5-methylpiperazine-1-carboxylate (0.6 g, 2.63 mmol) in acetonitrile (2 mL) were added DIPEA (1.37 mL, 7.88 mmol) and 4,6-dichloro-1-methylpyrido[3,2-d]pyrimidin-2(1H)-one (0.907 g, 3.94 mmol) at room temperature. The reaction mixture was heated at 85° C. for 6 h. The reaction mixture was cooled to room temperature and concentrated under reduced pressure to obtain the crude product, which was purified using silica gel column chromatography (60-70% EtOAc/petroleum ether) to afford tert-butyl (2R,5S)-4-(6-cyano-1-methyl-2-oxo-1,2-dihydro-1,5-naphthyridin-4-yl)-2-ethyl-5-methylpiperazine-1-carboxylate (0.5 g, 0.797 mmol, 30.3% yield). LCMS: m/z=422.3 [M+H]⁺; rt 1.76 min. LCMS Method: Column: Acquity UPLC BEH C18 (2.1×50 mm, 1.7 μm); mobile phase A: 10 mM ammonium acetate:acetonitrile (95:5); mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 2200 nm.

### Intermediate 40

### tert-Butyl (2R,5S)-4-(6-cyano-1-methyl-2-oxo-1,2-dihydropyrido[3,2-d]pyrimidin-4-yl)-2-ethyl-5-methylpiperazine-1-carboxylate

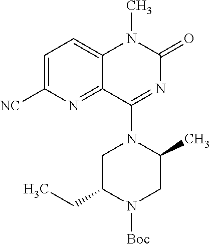 [SMILES: CC[C@@H]1CN(c2nc(=O)n(C)c3ccc(C#N)nc23)[C@@H](C)CN1C(=O)OC(C)(C)C]

To a stirred solution of tert-butyl (2R,5S)-4-(6-cyano-1-methyl-2-oxo-1,2-dihydro-1,5-naphthyridin-4-yl)-2-ethyl-5-methylpiperazine-1-carboxylate (0.55 g, 1.304 mmol) in NMP (2 mL) were added dppf (0.145 g, 0.261 mmol), zinc (0.085 g, 1.304 mmol) and zinc cyanide (0.306 g, 2.61 mmol). The reaction mixture was degassed for 5 min, followed by the addition of Pd₂(dba)₃ (0.119 g, 0.130 mmol) and heating at 90° C. for overnight. The reaction mixture was cooled to room temperature, diluted with ethyl acetate and filtered through a Celite® pad. The filtrate was washed with water, brine and the organic layer was dried over anhydrous Na₂SO₄, filtered and evaporated under reduced pressure to obtain the crude compound which was purified using silica gel chromatography (70-80% EtOAc/petroleum ether) to afford the tert-butyl (2R,5S)-4-(6-cyano-1-methyl-2-oxo-1,2-dihydropyrido[3,2-d]pyrimidin-4-yl)-2-ethyl-5-methylpiperazine-1-carboxylate (0.3 g, 0.473 mmol, 36.3% yield). LCMS: m/z=413.1 [M+H]⁺; rt 1.50 min. LCMS Method: Column: Acquity UPLC BEH C18 (2.1×50 mm, 1.7 μm); mobile phase A: 10 mM ammonium acetate:acetonitrile (95:5); mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

### Intermediate 41

### 4-((2S,5R)-5-Ethyl-2-methylpiperazin-1-yl)-1-methyl-2-oxo-1,2-dihydropyrido[3,2-d] pyrimidine-6-carbonitrile

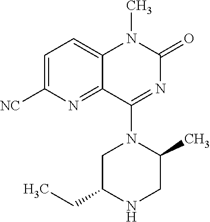 [SMILES: CC[C@@H]1CN(c2nc(=O)n(C)c3ccc(C#N)nc23)[C@@H](C)CN1]

To a stirred solution of tert-butyl (2R,5S)-4-(6-cyano-1-methyl-2-oxo-1,2-dihydropyrido[3,2-d]pyrimidin-4-yl)-2-ethyl-5-methylpiperazine-1-carboxylate (0.2 g, 0.485 mmol) in dry DCM (5 mL) was added TFA (0.560 mL, 7.27 mmol) at room temperature. The reaction mixture was stirred for 3 h. The solvent was removed under reduced pressure to afford 4-((2S,5R)-5-ethyl-2-methylpiperazin-1-yl)-1-methyl-2-oxo-1,2-dihydropyrido[3,2-d]pyrimidine-6-carbonitrile (0.15 g, 0.392 mmol, 81% yield) as a TFA salt. LCMS: m/z=313.1 [M+H]⁺; rt 0.63 min. LCMS Method: Column: Acquity UPLC BEH C18 (2.1×50 mm, 1.7 μm); mobile phase A: 10 mM ammonium acetate:acetonitrile (95:5); mobile phase B: 10 mM ammonium acetate:acetonitrile (5:95), Gradient=20% B over 1.1 minute, then a 2.2 minute hold at 100% B; Temperature: 50° C.; Flow rate: 0.7 mL/min; Detection: UV at 110 nm).

The examples in the Table 11 were prepared according to the general procedures described in Examples 152 and 153, using the appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 11

| Ex. | | Stereo | LCMS | LCMS | | Coupling |
|---|---|---|---|---|---|---|
| No. | Structure | chem. | Method | rt | [M + H]⁺ | Method |
| 296 297 | 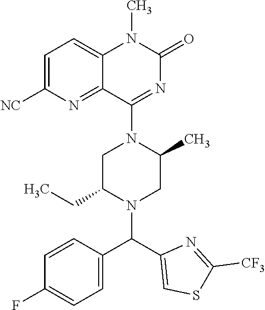 [SMILES: CC[C@@H]1CN(c2nc(=O)n(C)c3ccc(C#N)nc23)[C@@H](C)CN1C(c1ccc(F)cc1)c1csc(C(F)(F)F)n1] | H H | C C | 2.27 2.28 | 572.3 572.3 | A |
| 298 299 | 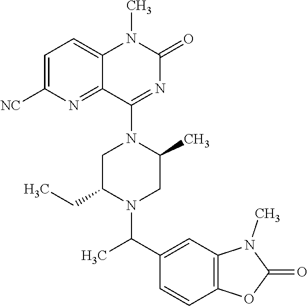 [SMILES: CC[C@@H]1CN(c2nc(=O)n(C)c3ccc(C#N)nc23)[C@@H](C)CN1C(C)c1ccc2oc(=O)n(C)c2c1] | H H | C C | 1.753 1.833 | 488.3 488.3 | B B |
| 300 301 | 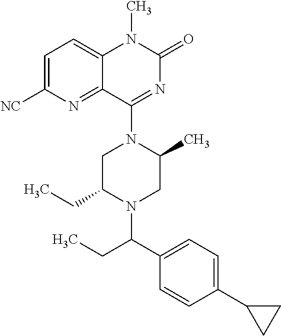 [SMILES: CCC(c1ccc(C2CC2)cc1)N1C[C@H](C)N(c2nc(=O)n(C)c3ccc(C#N)nc23)C[C@H]1CC] | H H | C C | 2.46 2.53 | 471.3 471.3 | B B |
| 302 303 | 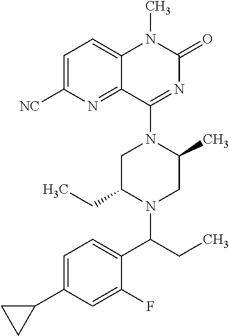 [SMILES: CCC(c1ccc(C2CC2)cc1F)N1C[C@H](C)N(c2nc(=O)n(C)c3ccc(C#N)nc23)C[C@H]1CC] | H H | C C | 2.45 2.52 | 489.3 489.3 | B B |
| 304 305 | 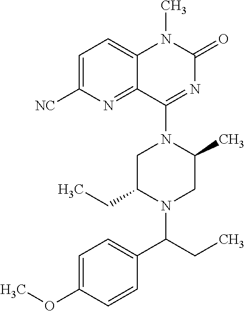 [SMILES: CCC(c1ccc(OC)cc1)N1C[C@H](C)N(c2nc(=O)n(C)c3ccc(C#N)nc23)C[C@H]1CC] | H H | C C | 2.146 2.23 | 461.3 461.3 | B B |
| 306 307 | 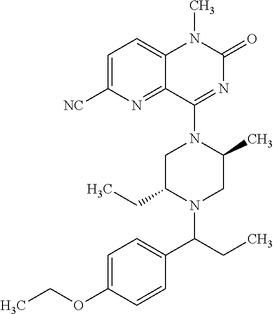 [SMILES: CCOc1ccc(C(CC)N2C[C@H](C)N(c3nc(=O)n(C)c4ccc(C#N)nc34)C[C@H]2CC)cc1] | H H | C C | 2.29 2.36 | 475.3 475.3 | B B |
| 308 309 | 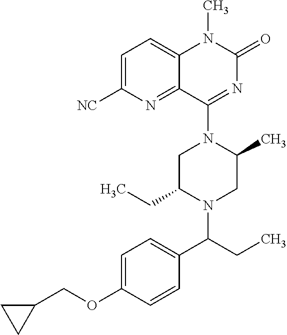 [SMILES: CCC(c1ccc(OCC2CC2)cc1)N1C[C@H](C)N(c2nc(=O)n(C)c3ccc(C#N)nc23)C[C@H]1CC] | H H | F F | 1.73 1.6 | 500.9 500.9 | B B |
| 310 | 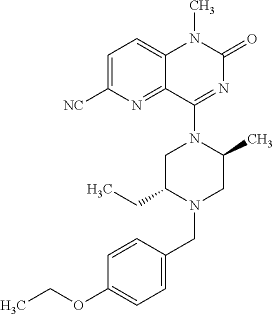 [SMILES: CCOc1ccc(CN2C[C@H](C)N(c3nc(=O)n(C)c4ccc(C#N)nc34)C[C@H]2CC)cc1] | H | C | 2.07 | 447.3 | B |
| 311 312 | 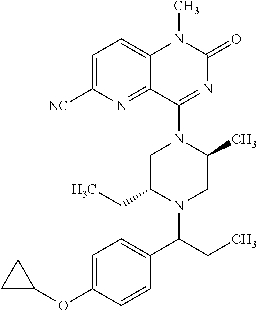 [SMILES: CCC(c1ccc(OC2CC2)cc1)N1C[C@H](C)N(c2nc(=O)n(C)c3ccc(C#N)nc23)C[C@H]1CC] | H H | C C | 2.33 2.40 | 487.2 487.2 | B B |
| 313 314 | 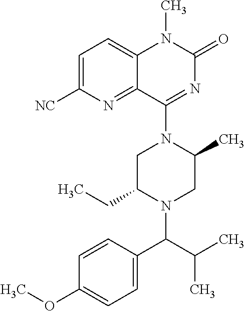 [SMILES: CC[C@@H]1CN(c2nc(=O)n(C)c3ccc(C#N)nc23)[C@@H](C)CN1C(c1ccc(OC)cc1)C(C)C] | H H | C C | 2.35 2.26 | 475.3 475.4 | B B |
| 315 316 | 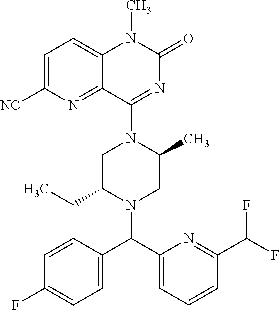 [SMILES: CC[C@@H]1CN(c2nc(=O)n(C)c3ccc(C#N)nc23)[C@@H](C)CN1C(c1ccc(F)cc1)c1cccc(C(F)F)n1] | H H | C C | 2.092 2.087 | 548.3 548.3 | A A |
| 317 318 | 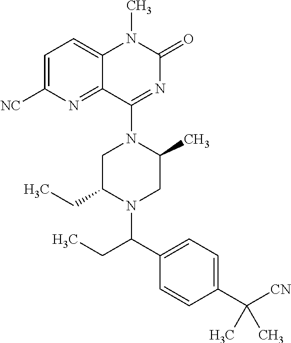 [SMILES: CCC(c1ccc(C(C)(C)C#N)cc1)N1C[C@H](C)N(c2nc(=O)n(C)c3ccc(C#N)nc23)C[C@H]1CC] | H H | C C | 2.145 2.22 | 498.3 498.3 | B B |
| 319 320 | 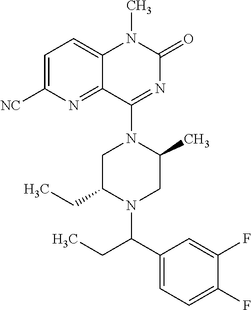 [SMILES: CCC(c1ccc(F)c(F)c1)N1C[C@H](C)N(c2nc(=O)n(C)c3ccc(C#N)nc23)C[C@H]1CC] | H H | C C | 2.28 2.34 | 467.3 467.3 | B B |
| 321 322 | 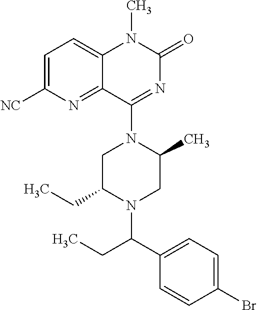 [SMILES: CCC(c1ccc(Br)cc1)N1C[C@H](C)N(c2nc(=O)n(C)c3ccc(C#N)nc23)C[C@H]1CC] | H H | C C | 2.464 2.521 | 511.2 511.2 | B B |
| 323 324 | 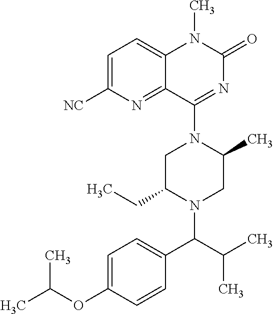 [SMILES: CC[C@@H]1CN(c2nc(=O)n(C)c3ccc(C#N)nc23)[C@@H](C)CN1C(c1ccc(OC(C)C)cc1)C(C)C] | H H | C C | 2.60 2.64 | 503.4 503.3 | B B |
| 325 326 | 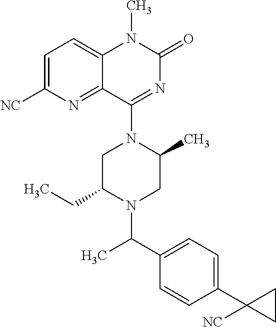 [SMILES: CC[C@@H]1CN(c2nc(=O)n(C)c3ccc(C#N)nc23)[C@@H](C)CN1C(C)c1ccc(C2(C#N)CC2)cc1] | H H | C C | 2.085 2.159 | 482.3 482.4 | B B |
| 327 328 | 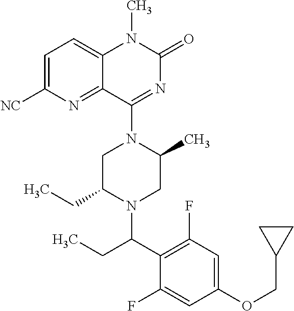 [SMILES: CCC(c1c(F)cc(OCC2CC2)cc1F)N1C[C@H](C)N(c2nc(=O)n(C)c3ccc(C#N)nc23)C[C@H]1CC] | H H | C C | 2.54 2.59 | 537.3 537.3 | B B |
| 329 330 | 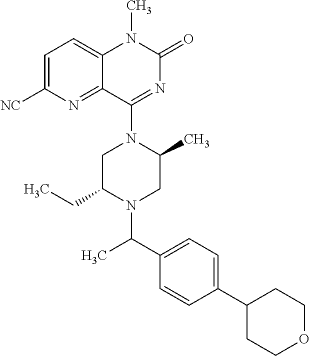 [SMILES: CC[C@@H]1CN(c2nc(=O)n(C)c3ccc(C#N)nc23)[C@@H](C)CN1C(C)c1ccc(C2CCOCC2)cc1] | H H | C C | 2.17 2.09 | 501.3 501.3 | B B |
| 331 332 | 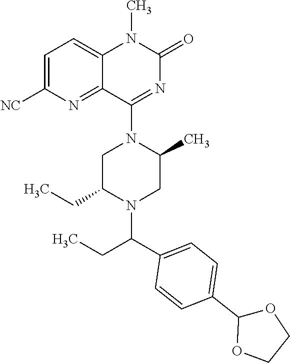 [SMILES: CCC(c1ccc(C2OCCO2)cc1)N1C[C@H](C)N(c2nc(=O)n(C)c3ccc(C#N)nc23)C[C@H]1CC] | H H | F F | 1.37 1.36 | 503.21 503.15 | B B |
| 333 334 | 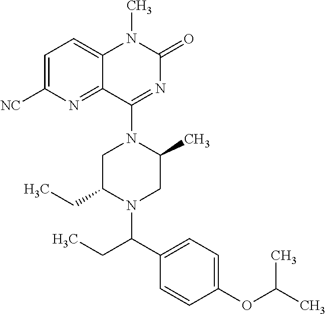 [SMILES: CCC(c1ccc(OC(C)C)cc1)N1C[C@H](C)N(c2nc(=O)n(C)c3ccc(C#N)nc23)C[C@H]1CC] | H H | F F | 1.65 1.57 | 489.16 489.22 | B B |

### Examples 335 and 336

### 4-((2S,5R)-5-Ethyl-2-methyl-4-(1-(4-(3,3,3-trifluoropropoxy)phenyl)propyl)piperazin-1-yl)-1-methyl-2-oxo-1,2-dihydropyrido[3,2-d]pyrimidine-6-carbonitrile

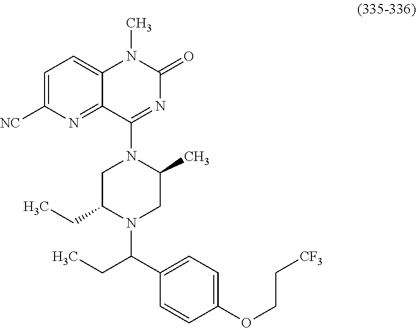 [SMILES: CCC(c1ccc(OCCC(F)(F)F)cc1)N1C[C@H](C)N(c2nc(=O)n(C)c3ccc(C#N)nc23)C[C@H]1CC]

To a stirred solution of 4-((2S,5R)-4-(1-(4-bromophenyl)propyl)-5-ethyl-2-methylpiperazin-1-yl)-1-methyl-2-oxo-1,2-dihydropyrido[3,2-d]pyrimidine-6-carbonitrile (100 mg, 0.20 mmol), 3,3,3-trifluoropropan-1-ol (45 mg, 0.39 mmol), Cs₂CO₃ (192 mg, 0.59 mmol), 2-di-tert-butylphosphino-3-methoxy-6-methyl-2′,4′,6′-triisopropyl-1,1′-biphenyl, 97% (9.20 mg, 0.02 mmol) in toluene (5 mL) was flushed with argon for 10 min. Then, Pd₂(dba)₃ (9 mg, 9.81 μmol) was added. The reaction vessel was sealed and heated at 110° C. overnight. The reaction mixture was cooled to room temperature, filtered through a Celite® pad, washed with excess 10% MeOH in DCM (20 mL) and the filtrate was concentrated under reduced pressure to give the crude product, which was purified by flash chromatography (3% MeOH in CHCl₃) to afford the diastereomeric mixture of 4-((2S,5R)-5-ethyl-2-methyl-4-(1-(4-(3,3,3-trifluoropropoxy)phenyl)propyl) piperazin-1-yl)-1-methyl-2-oxo-1,2-dihydropyrido[3,2-d]pyrimidine-6-carbonitrile. The diastereomeric mixture was further purified using preparative HPLC (Chiral Separation Method: Column: Gemini NX (250×21.2 mm, 5 μm); mobile phase A=10 mM ammonium acetate in MeOH; mobile phase B=acetonitrile; Flow 20 mL/min. Gradient: 10-90% B over 20 minutes). Fractions were concentrated under reduced pressure and lyophilized from (EtOH/H₂O, 1:5) to yield Examples 335 and 336.

EXAMPLE 335 (18 mg, 16% yield). LCMS: m/z, 543.3 [M+H]⁺; rt 2.03 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.26-8.16 (m, 1H), 7.98 (d, J=9.0 Hz, 1H), 7.28-7.15 (m, 2H), 6.98-6.91 (m, 2H), 5.95-5.55 (m, 1H), 5.04-4.81 (m, 1H), 4.26-4.13 (m, 2H), 3.51-3.41 (m, 5H), 2.91-2.69 (m, 3H), 1.91-1.20 (m, 9H), 0.76-0.56 (m, 6H).

EXAMPLE 336 (14 mg, 13% yield). LCMS: m/z, 543.3 [M+H]⁺; rt 2.09 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.31-8.17 (m, 1H), 8.08-7.91 (m, 1H), 7.26 (br d, J=8.5 Hz, 2H), 6.93 (d, J=8.8 Hz, 2H), 6.16-5.36 (m, 1H), 5.04-4.75 (m, 1H), 4.24-4.16 (m, 2H), 3.67-3.58 (m, 1H), 3.44 (s, 3H), 3.16-3.04 (m, 1H), 2.85-2.68 (m, 2H), 2.61-2.53 (m, 2H), 2.28-2.16 (m, 1H), 1.88-1.76 (m, 1H), 1.61-1.41 (m, 3H), 1.40-1.32 (m, 1H), 1.29-1.18 (m, 2H), 1.04-0.95 (m, 3H), 0.61 (t, J=7.3 Hz, 3H).

The examples in the Table 12 were prepared according to the general procedures described in Examples 335 and 336, using the appropriate alcohol and 4-((2S,5R)-4-(1-(4-bromophenyl)propyl)-5-ethyl-2-methylpiperazin-1-yl)-1-methyl-2-oxo-1,2-dihydropyrido[3,2-d]pyrimidine-6-carbonitrile. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 12

| Ex. | | Stereo- | LCMS | LCMS | |
|---|---|---|---|---|---|
| No. | Structure | chemistry | Method | rt | [M + H]⁺ |
| 337 338 | 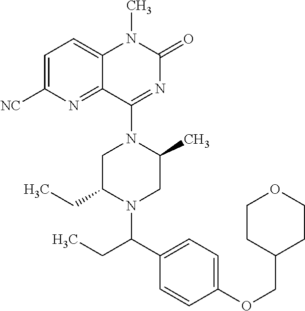 [SMILES: CCC(c1ccc(OCC2CCOCC2)cc1)N1C[C@H](C)N(c2nc(=O)n(C)c3ccc(C#N)nc23)C[C@H]1CC] | H H | E E | 1.74 1.83 | 545.4 545.4 |
| 339 340 | 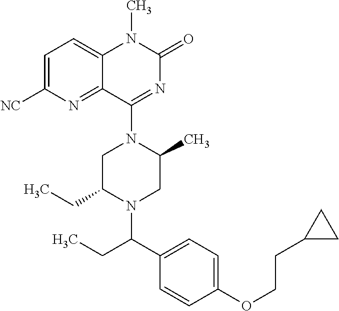 [SMILES: CCC(c1ccc(OCCC2CC2)cc1)N1C[C@H](C)N(c2nc(=O)n(C)c3ccc(C#N)nc23)C[C@H]1CC] | H H | E E | 2.22 2.28 | 515.4 515.4 |
| 341 342 | 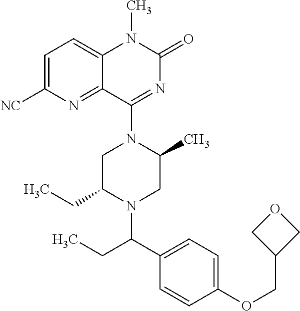 [SMILES: CCC(c1ccc(OCC2COC2)cc1)N1C[C@H](C)N(c2nc(=O)n(C)c3ccc(C#N)nc23)C[C@H]1CC] | H H | E E | 1.38 1.38 | 517.4 518.4 |
| 343 344 | 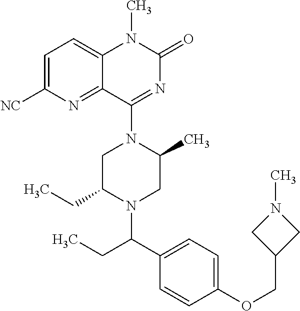 [SMILES: CCC(c1ccc(OCC2CN(C)C2)cc1)N1C[C@H](C)N(c2nc(=O)n(C)c3ccc(C#N)nc23)C[C@H]1CC] | H H | E E | 2.5 2.68 | 530.4 530.4 |
| 345 346 | 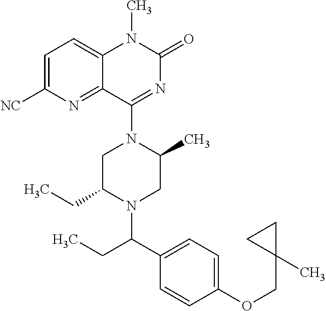 [SMILES: CCC(c1ccc(OCC2(C)CC2)cc1)N1C[C@H](C)N(c2nc(=O)n(C)c3ccc(C#N)nc23)C[C@H]1CC] | H H | E E | 2.27 2.27 | 515.4 515.3 |
| 347 348 | 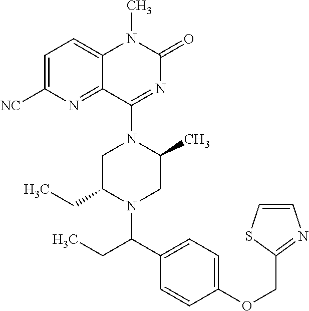 [SMILES: CCC(c1ccc(OCc2nccs2)cc1)N1C[C@H](C)N(c2nc(=O)n(C)c3ccc(C#N)nc23)C[C@H]1CC] | H H | E E | 1.65 1.61 | 544.3 544.3 |

### Intermediate 42

### 2-Bromo-4-(1-chloroethyl)-1-(trifluoromethyl)benzene

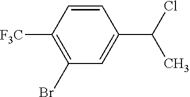

To a solution of 1-(3-bromo-4-(trifluoromethyl)phenyl)ethan-1-ol (600 mg, 2.23 mmol) in DCM (10 mL) was added thionyl chloride (0.16 mL, 2.23 mmol) slowly at 0° C. The reaction mixture was stirred for 10 minutes and then allowed it reach room temperature over 16 h. The reaction mixture was concentrated under reduced pressure to yield 2-bromo-4-(1-chloroethyl)-1-(trifluoromethyl)benzene (640 mg, 100% yield). ¹H NMR (400 MHz, CDCl₃) δ (ppm)=7.78 (s, 1H), 7.19-7.16 (m, 1H), 6.64-6.61 (m, 1H), 4.42-4.38 (m, 1H), 1.36 (d, J=6.4 Hz, 3H).

### Example 349

### 4-((2S,5R)-4-(1-(3-bromo-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile

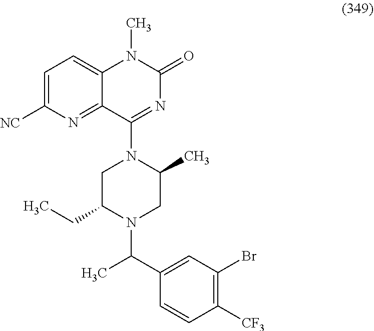 [SMILES: CC[C@@H]1CN(c2nc(=O)n(C)c3ccc(C#N)nc23)[C@@H](C)CN1C(C)c1ccc(C(F)(F)F)c(Br)c1]

To a stirred suspension of 4-((2S,5R)-5-ethyl-2-methylpiperazin-1-yl)-1-methyl-2-oxo-1,2-dihydropyrido[3,2-d]pyrimidine-6-carbonitrile, TFA (600 mg, 1.41 mmol), 2-bromo-4-(1-chloroethyl)-1-(trifluoromethyl)benzene (809 mg, 2.81 mmol), sodium iodide (211 mg, 1.41 mmol) in acetonitrile (6.0 mL) was added DIPEA (0.74 mL, 4.22 mmol). The reaction mixture was stirred for 5 minutes. The reaction mixture was heated to 85° C. and maintained for 3 h. The reaction mixture concentrated under reduced pressure to obtain the crude product, which was purified by silica gel (24 g) column chromatography by using 0-10% methanol in chloroform as eluent. The fractions were concentrated under reduced pressure to obtain 4-((2S,5R)-4-(1-(3-bromo-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile (750 mg, 80% yield). LCMS: m/z, 563.2 [M+H]⁺; retention time 2.12 min; LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Examples 350 and 351

### 4-((2S,5R)-5-ethyl-2-methyl-4-(1-(3-(morpholinomethyl)-4-(trifluoromethyl)phenyl) ethyl)piperazin-1-yl)-1-methyl-2-oxo-1,2-dihydropyrido[3,2-d]pyrimidine-6-carbonitrile

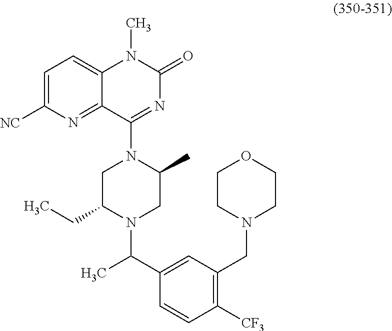

A suspension of 4-((2S,5R)-4-(1-(3-bromo-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile (50 mg, 0.09 mmol), trifluoro(morpholinomethyl)borate (17.88 mg, 0.106 mmol), Cs₂CO₃ (87 mg, 0.27 mmol) in THF (1.0 mL) and water (0.1 mL) was purged with argon gas for 10 minutes, followed by addition of XPhos (8.46 mg, 0.02 mmol) and Pd(OAc)₂ (1.992 mg, 8.87 μmol). The reaction mixture was purged argon for another 5 minutes. The reaction mixture was heated to 85° C. and was maintained for 16 h. The reaction mixture was concentrated under reduced pressure to obtain crude product, which was purified by preparative SFC. SFC Method: Column: ChiralCel OJ-H (250×21 mm, 5 μm); mobile phase: 90% CO₂/10% of co-solvent 0.2% DEA in methanol, Flow: 100 g/min; Detector Wavelength: 230 nm. Example 350: Isolate 1: First eluting peak, rt=2.69 min. Example 351: Isolate 2: Second eluting peak, rt=4.03 min.

EXAMPLE 350: (20.6 mg, 39% yield); LCMS: m/z, 584.2 [M+H]⁺; rt 2.32 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.38-8.13 (m, 2H), 7.98 (d, J=9.0 Hz, 1H), 7.82 (br d, J=0.7 Hz, 1H), 7.73-7.62 (m, 1H), 7.51-7.37 (m, 1H), 5.86-5.61 (m, 1H), 5.10-4.78 (m, 1H), 3.83 (ddd, J=1.7, 5.6, 6.9 Hz, 1H), 3.68-3.56 (m, 2H), 3.43 (s, 3H), 2.98-2.87 (m, 3H), 2.85-2.73 (m, 2H), 2.45-2.31 (m, 2H), 1.64-1.34 (m, 3H), 1.29 (br dd, J=1.8, 4.8 Hz, 3H), 1.16 (t, J=7.3 Hz, 7H), 0.69 (br t, J=5.5 Hz, 3H).

EXAMPLE 351: (18.4 mg, 35% yield); LCMS: m/z, 584.2 [M+H]⁺; rt 2.32 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.43-8.15 (m, 3H), 8.05-7.95 (m, 1H), 7.83 (br d, J=1.5 Hz, 1H), 7.70-7.61 (m, 1H), 7.52-7.42 (m, 1H), 6.08-5.52 (m, 1H), 5.08-4.83 (m, 1H), 3.75-3.55 (m, 2H), 3.44 (s, 3H), 3.18-3.03 (m, 1H), 2.99-2.88 (m, 4H), 2.63-2.55 (m, 1H), 2.46-2.13 (m, 3H), 1.62-1.36 (m, 3H), 1.32-1.21 (m, 3H), 1.16 (t, J=7.2 Hz, 6H), 1.07-0.91 (m, 3H).

The examples in the Table 13 were prepared according to the general procedure described in Examples 350 and 351, substituting ammonia with the appropriate trifluoroborate in the synthetic sequence. When the synthesis provided a mixture of diastereomer, the mixture was separated at the final stage using either preparative chromatography or preparative chiral chromatography.

TABLE 13

| Ex. | | Stereo- | LCMS | LCMS | |
|---|---|---|---|---|---|
| No. | Structure | chemistry | Method | rt | [M + H]⁺ |
| 352 353 | 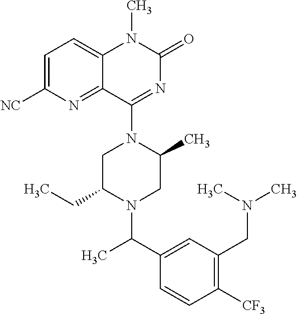 [SMILES: CC[C@@H]1CN(c2nc(=O)n(C)c3ccc(C#N)nc23)[C@@H](C)CN1C(C)c1ccc(C(F)(F)F)c(CN(C)C)c1] | H H | C C | 2.32 2.37 | 542.4 542.4 |
| 354 355 | 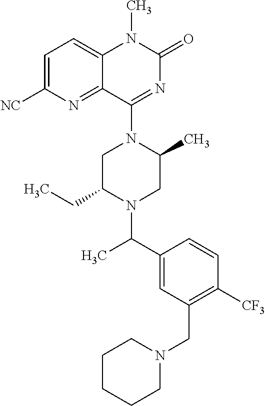 [SMILES: CC[C@@H]1CN(c2nc(=O)n(C)c3ccc(C#N)nc23)[C@@H](C)CN1C(C)c1ccc(C(F)(F)F)c(CN2CCCCC2)c1] | H H | C C | 2.73 2.77 | 582.3 582.3 |

### Examples 356 and 357

### 4-((2S,5R)-4-(1-(3-Cyano-4-(trifluoromethyl)phenyl)ethyl)-5-ethyl-2-methylpiperazin-1-yl)-1-methyl-2-oxo-1,2-dihydropyrido[3,2-d]pyrimidine-6-carbonitrile

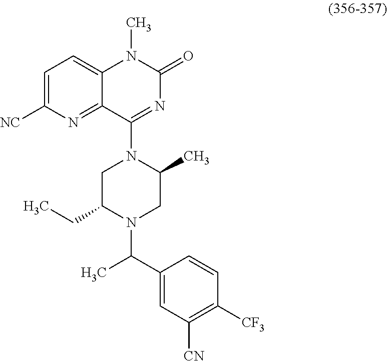 [SMILES: CC[C@@H]1CN(c2nc(=O)n(C)c3ccc(C#N)nc23)[C@@H](C)CN1C(C)c1ccc(C(F)(F)F)c(C#N)c1]

To a stirred solution of 4-((2S,5R)-4-(1-(3-bromo-4-(trifluoromethyl)phenyl) ethyl)-5-ethyl-2-methylpiperazin-1-yl)-1-methyl-2-oxo-1,2-dihydropyrido[3,2-d] pyrimidine-6-carbonitrile (50 mg, 0.089 mmol) in DMF (2.0 mL) was added copper (I) cyanide (15.90 mg, 0.177 mmol) at room temperature. The reaction mixture was heated at 130° C. for 16 h, cooled, and filtered through a Celite® pad, which was then washed with DCM. The filtrate was washed with water, brine, dried over sodium sulfate, and concentrated under reduced pressure to obtain the crude product, which was purified by using preparative SFC. SFC Method: Column: Chiralpak IG (250×30 mm, 5 μm); mobile phase: 65% CO₂/35% Methanol, 80 g/min; Detector Wavelength: 230 nm. Example 356: Isolate 1: First eluting peak, rt=7.5 min, Example 357: Isolate 2: Second eluting peak, rt=8.8 min.

EXAMPLE 356: (1.7 mg, 4% yield); LCMS: m/z, 510.3 [M+H]⁺; rt 2.14 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.26-8.20 (m, 1H), 8.18 (d, J=0.7 Hz, 1H), 8.03-7.93 (m, 3H), 6.54 (s, 1H), 3.97 (br d, J=5.9 Hz, 2H), 3.44 (s, 1H), 2.87-2.83 (m, 1H), 2.77-2.72 (m, 1H), 1.59-1.35 (m, 6H), 1.33-1.28 (m, 4H), 1.24 (m, 2H), 0.80-0.68 (m, 3H).

EXAMPLE 357: (2.0 mg, 4% yield); LCMS: m/z, 510.3 [M+H]⁺; rt 2.14 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.32-8.21 (m, 1H), 8.18 (d, J=0.7 Hz, 1H), 8.03-7.93 (m, 3H), 6.54 (s, 1H), 3.97 (br d, J=5.9 Hz, 2H), 3.44 (s, 1H), 2.87-2.83 (m, 1H), 2.77-2.72 (m, 1H), 1.59-1.35 (m, 6H), 1.33-1.28 (m, 4H), 1.24 (m, 2H), 01.00-0.98 (m, 3H).

### Intermediate 43

### 1-(4-(Azidomethyl)phenyl)ethan-1-one

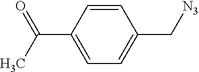

To a stirred solution of 1-(4-(bromomethyl)phenyl)ethan-1-one (2 g, 9.39 mmol) in DMF (20 mL) was added sodium azide (0.91 g, 14.08 mmol) at room temperature and the reaction mixture was heated at 80° C. for 12 h. The reaction mixture was cooled to room temperature, extracted with EtOAc (2×50 mL), washed with cold water (2×100 mL), brine, dried over sodium sulphate and concentrated under reduced pressure to give the crude product, which was purified by silica gel column chromatography using 20-30% EtOAc in n-hexane to afford 1-(4-(azidomethyl)phenyl)ethan-1-one (1.4 g, 51% yield). LCMS: m/z, 176.1 [M+H]⁺; rt 1.35 min. (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm). ¹H NMR (300 MHz, DMSO-d₆) δ (ppm)=7.99 (d, J=8.3 Hz, 2H), 7.52 (d, J=7.9 Hz, 2H), 4.57 (s, 2H), 2.59 (s, 3H).

### Intermediate 44

### 1-(4-(Azidomethyl)phenyl)ethan-1-ol (racemate)

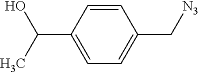

To a solution of 1-(4-(azidomethyl)phenyl)ethan-1-one (1.3 g, 7.42 mmol) in methanol (20 mL) was added NaBH₄ (0.56 g, 14.84 mmol) in two equal portions at 0° C. under nitrogen. The reaction mixture was allowed to warm to room temperature and stirred for 2 h. The reaction was quenched with the addition of saturated aqueous NH₄Cl solution, extracted with ethyl acetate (2×20 mL) and washed with water. The combined organic layer was dried over anhydrous Na₂SO₄, evaporated under reduced pressure to obtain 1-(4-(azidomethyl)phenyl)ethan-1-ol (1 g, 62% yield). ¹H NMR (300 MHz, DMSO-d₆) δ (ppm)=7.34 (d, J=10.6 Hz, 4H), 5.27-5.12 (m, 1H), 4.77-4.64 (m, 1H), 4.41 (s, 2H), 1.31 (d, J=6.4 Hz, 3H).

### Intermediate 45

### 1-(Azidomethyl)-4-(1-chloroethyl)benzene (Racemate)

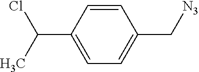

To a solution of 1-(4-(azidomethyl)phenyl)ethan-1-ol (0.6 g, 3.39 mmol) in dichloromethane (10.0 mL) was added SOCl₂ (1.25 mL, 16.93 mmol) at 0° C. The reaction mixture was stirred at room temperature for 2 h. The volatiles were removed from the reaction mixture under reduced pressure, co-distilled with acetonitrile (2×10 mL) and dried to obtain 1-(azidomethyl)-4-(1-chloroethyl)benzene (0.5 g, 75% yield). ¹H NMR (300 MHz, DMSO-d₆) δ (ppm)=7.52 (d, J=8.3 Hz, 2H), 7.41-7.35 (m, 2H), 5.46-5.29 (m, 1H), 4.46 (s, 2H), 1.79 (d, J=6.8 Hz, 3H).

### Intermediate 46

### 4-((2S,5R)-4-(1-(4-(Azidomethyl)phenyl)ethyl)-5-ethyl-2-methylpiperazin-1-yl)-1-methyl-2-oxo-1,2-dihydropyrido[3,2-d]pyrimidine-6-carbonitrile (Diastereomeric Mixture)

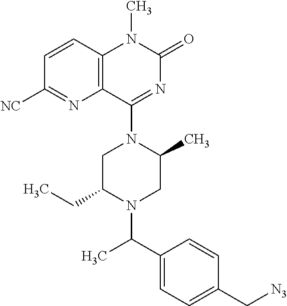 [SMILES: CC[C@@H]1CN(c2nc(=O)n(C)c3ccc(C#N)nc23)[C@@H](C)CN1C(C)c1ccc(CN=[N+]=[N-])cc1]

To a stirred solution of 4-((2S,5R)-5-ethyl-2-methylpiperazin-1-yl)-1-methyl-2-oxo-1,2-dihydropyrido[3,2-d]pyrimidine-6-carbonitrile (0.6 g, 1.921 mmol) in acetonitrile (10 mL), DIPEA (1 mL, 5.76 mmol), 1-(azidomethyl)-4-(1-chloroethyl)benzene (0.56 g, 2.88 mmol) and sodium iodide (0.29 g, 1.92 mmol) were added at room temperature. The reaction mixture was heated at 80° C. for 12 h. and then cooled to room temperature. The solvent was removed under reduced pressure to give the crude product, which was purified by silica gel column chromatography using 10-15% MeOH in DCM to afford 4-((2S,5R)-4-(1-(4-(azidomethyl)phenyl)ethyl)-5-ethyl-2-methylpiperazin-1-yl)-1-methyl-2-oxo-1,2-dihydropyrido[3,2-d]pyrimidine-6-carbonitrile (0.5 g, 0.944 mmol, 49.1% yield). LCMS: m/z, 472.2 [M+H]⁺; rt: 3.21 and 3.35 min. (LCMS Method: Column: Kinetex XB-C18 (3×75 mm) 2.6 μm; mobile phase A: 10 mM ammonium formate in 0.1% formic acid, mobile phase B: acetonitrile, Gradient=20-100% B over 4 minutes, then a 0.6 minute hold at 100% B; Temperature: 27° C.; Flow rate: 1.0 mL/min; Detection: UV at 220 nm).

### Example 358

### 4-((2S,5R)-4-(1-(4-(Aminomethyl)phenyl)ethyl)-5-ethyl-2-methylpiperazin-1-yl)-1-methyl-2-oxo-1,2-dihydropyrido[3,2-d]pyrimidine-6-carbonitrile (Diastereomeric Mixture)

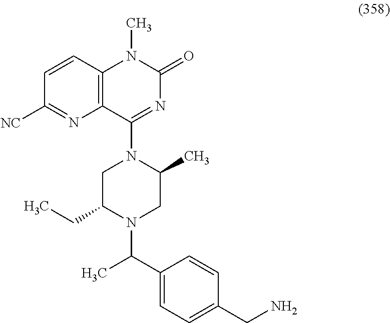 [SMILES: CC[C@@H]1CN(c2nc(=O)n(C)c3ccc(C#N)nc23)[C@@H](C)CN1C(C)c1ccc(CN)cc1]

To a stirred solution of 4-((2S,5R)-4-(1-(4-(azidomethyl)phenyl)ethyl)-5-ethyl-2-methylpiperazin-1-yl)-1-methyl-2-oxo-1,2-dihydropyrido[3,2-d]pyrimidine-6-carbonitrile (500 mg, 1.060 mmol) in THF (6 mL) and water (3 mL) was added triphenylphosphine (556 mg, 2.121 mmol) at room temperature. The reaction mixture was heated at 70° C. for 12 h. and then cooled to room temperature. The solvent was removed under reduced pressure to give the crude product which was purified by silica gel column chromatography using 8-10% MeOH in DCM to afford 4-((2S,5R)-4-(1-(4-(aminomethyl) phenyl)ethyl)-5-ethyl-2-methylpiperazin-1-yl)-1-methyl-2-oxo-1,2-dihydropyrido[3,2-d] pyrimidine-6-carbonitrile (0.3 g, 50% yield). LCMS: m/z, 446.4 [M+H]⁺; rt: 1.22 and 1.31 min. (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Examples 359 and 360

### Methyl (4-(1-((2R,5S)-4-(6-cyano-1-methyl-2-oxo-1,2-dihydropyrido[3,2-d]pyrimidin-4-yl)-2-ethyl-5-methylpiperazin-1-yl)ethyl)benzyl)carbamate

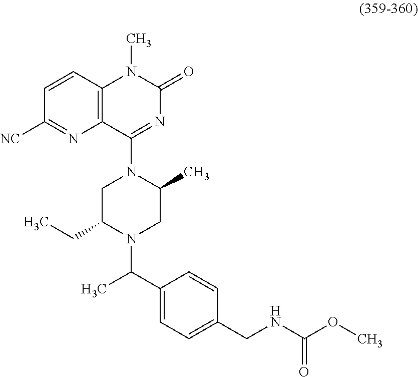 [SMILES: CC[C@@H]1CN(c2nc(=O)n(C)c3ccc(C#N)nc23)[C@@H](C)CN1C(C)c1ccc(CNC(=O)OC)cc1]

To a stirred solution of 4-((2S,5R)-4-(1-(4-(aminomethyl)phenyl)ethyl)-5-ethyl-2-methylpiperazin-1-yl)-1-methyl-2-oxo-1,2-dihydropyrido[3,2-d]pyrimidine-6-carbonitrile (60 mg, 0.14 mmol) in dichloromethane (5 mL) was cooled to 0° C. and added TEA (0.04 mL, 0.269 mmol) followed by methyl chloroformate (0.02 mL, 0.269 mmol) under nitrogen. The reaction mixture was allowed to warm to room temperature and stirred for 2 h. The volatiles were removed under reduced pressure to give the crude product, which was purified by prep-HPLC [Method: Column: Sunfire C18 (150×19 mm, 5 μm); mobile phase A=10 mM ammonium acetate in water pH ˜4.5; mobile phase B=MeOH; Gradient: 50-80% B over 22 minutes, then a 5 minute hold at 100% B; Flow rate: 19 mL/min] fractions were concentrated under reduced pressure and lyophilized from (EtOH/H₂O, 1:5) to yield Examples 359 and 360.

EXAMPLE 359: (5 mg, 7% yield), LCMS: m/z, 504.2 (([M+H]⁺; rt 1.14 min. (LCMS Method: Column: Kinetex XB-C18 (3×75 mm) 2.6 μm; mobile phase A: 10 mM ammonium formate in water (pH ˜3.3), mobile phase B: acetonitrile, Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.23 (d, J=8.5 Hz, 1H), 7.98 (d, J=9.0 Hz, 1H), 7.70-7.64 (m, 1H), 7.29 (s, 2H), 7.25-7.19 (m, 2H), 5.91-5.57 (m, 1H), 5.12-4.72 (m, 1H), 4.19 (d, J=6.0 Hz, 2H), 3.74-3.66 (m, 1H), 3.56 (s, 3H), 3.45-3.41 (m, 4H), 2.85-2.78 (m, 1H), 2.77-2.70 (m, 1H), 2.42-2.35 (m, 1H), 1.58-1.30 (m, 5H), 1.30-1.23 (m, 3H), 0.73-0.66 (m, 3H).

EXAMPLE 360: (5 mg, 7% yield), LCMS: m/z, 504.2 [M+H]⁺; rt 1.22 min. (LCMS Method: Column: Kinetex XB-C18 (3×75 mm, 2.6 μm); mobile phase A: 10 mM ammonium formate in water (pH ˜3.3), mobile phase B: acetonitrile, Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.31-8.17 (m, 1H), 8.04-7.92 (m, 1H), 7.64 (br s, 1H), 7.34 (br d, J=7.5 Hz, 2H), 7.20 (d, J=8.0 Hz, 2H), 6.13-5.35 (m, 1H), 5.16-4.75 (m, 1H), 4.17 (d, J=6.0 Hz, 2H), 3.74-3.53 (m, 5H), 3.46-3.39 (m, 4H), 3.14-3.03 (m, 1H), 2.27-2.17 (m, 1H), 1.59-1.42 (m, 2H), 1.41-1.32 (m, 1H), 1.29-1.18 (m, 5H), 0.99 (br t, J=7.3 Hz, 3H).

The examples in the Table 14 were prepared according to the general procedure described in Examples 110 and 111, using the appropriate piperazine. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 14

| Ex. | | Stereo | LCMS | LCMS | |
|---|---|---|---|---|---|
| No. | STRUCTURE | chemistry | Method | rt | [M + H]⁺ |
| 361 362 | 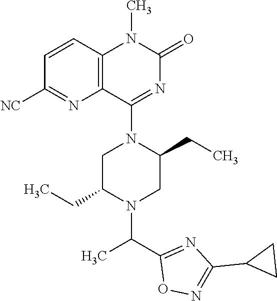 [SMILES: CC[C@H]1CN(C(C)c2nc(C3CC3)no2)[C@H](CC)CN1c1nc(=O)n(C)c2ccc(C#N)nc12] | H H | C C | 1.86 1.84 | 463.3 463.3 |
| 363 364 | 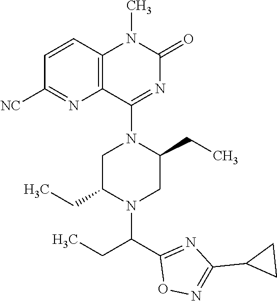 [SMILES: CCC(c1nc(C2CC2)no1)N1C[C@H](CC)N(c2nc(=O)n(C)c3ccc(C#N)nc23)C[C@H]1CC] | H H | C C | 2.01 2.02 | 477.3 477.3 |
| 365 366 | 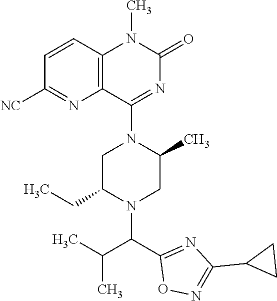 [SMILES: CC[C@@H]1CN(c2nc(=O)n(C)c3ccc(C#N)nc23)[C@@H](C)CN1C(c1nc(C2CC2)no1)C(C)C] | H H | C C | 2.02 2.06 | 477.3 477.3 |

### Intermediate 46

### N-(2-((2R,5S)-4-(6-Cyano-1-methyl-2-oxo-1,2-dihydropyrido[3,2-d]pyrimidin-4-yl)-2-ethyl-5-methylpiperazin-1-yl)-3-methylbutanoyl)cyclopropanecarbohydrazide

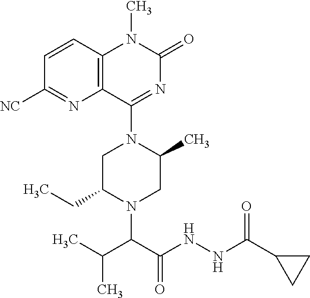 [SMILES: CC[C@@H]1CN(c2nc(=O)n(C)c3ccc(C#N)nc23)[C@@H](C)CN1C(C(=O)NNC(=O)C1CC1)C(C)C]

To a solution of 2-((2R,5S)-4-(6-cyano-1-methyl-2-oxo-1,2-dihydropyrido[3,2-d] pyrimidin-4-yl)-2-ethyl-5-methylpiperazin-1-yl)-3-methylbutanoic acid (500 mg, 1.21 mmol) in DMF (3 mL) were added HATU (922 mg, 2.42 mmol) and DIPEA (0.42 mL, 2.42 mmol). The reaction mixture was stirred for 10 minutes. Next, cyclopropanecarbohydrazide (182 mg, 1.82 mmol) was added and stirring was continued for 16 h. The reaction was quenched with cold water. The reaction mixture was diluted with EtOAc (100 mL), washed with water (20 mL), brine (20 mL), dried over Na₂SO₄ and concentrated under reduced pressure to obtain the crude compound, which was purified by silica gel chromatography (12 g) by using 0-10% MeOH/CHCl₃ as eluent. Pure fractions were collected and concentrated to obtain N-(2-((2R,5S)-4-(6-cyano-1-methyl-2-oxo-1,2-dihydropyrido[3,2-d]pyrimidin-4-yl)-2-ethyl-5-methylpiperazin-1-yl)-3-methylbutanoyl)cyclopropanecarbohydrazide (350 mg, 58% yield). LCMS: m/z=495.3 [M+H]⁺; retention time 1.05 and 1.11 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Examples 367 and 368

### 4-((2S,5R)-4-(1-(5-Cyclopropyl-1,3,4-oxadiazol-2-yl)-2-methylpropyl)-5-ethyl-2-methylpiperazin-1-yl)-1-methyl-2-oxo-1,2-dihydropyrido[3,2-d]pyrimidine-6-carbonitrile

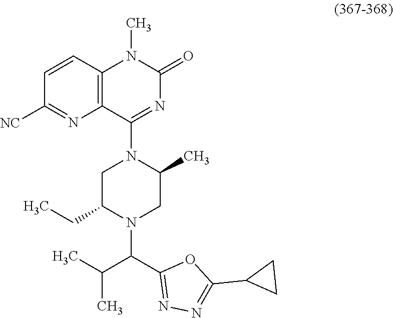 [SMILES: CC[C@@H]1CN(c2nc(=O)n(C)c3ccc(C#N)nc23)[C@@H](C)CN1C(c1nnc(C2CC2)o1)C(C)C]

To a solution of N′-(2-((2R,5S)-4-(6-cyano-1-methyl-2-oxo-1,2-dihydropyrido[3,2-d]pyrimidin-4-yl)-2-ethyl-5-methylpiperazin-1-yl)-3-ethylbutanoyl) cyclopropanecarbohydrazide (50 mg, 0.1 mmol) in acetonitrile (3 mL) was added POCl₃ (0.02 mL, 0.20 mmol). The reaction mixture was heated at 80° C. for 16 h, cooled, concentrated under reduced pressure. The reaction was quenched with saturated NaHCO₃ solution. The reaction mixture was diluted with EtOAc (100 mL), washed with water (20 mL), brine (20 mL), dried over Na₂SO₄ and concentrated under reduced pressure to obtain the crude compound, which was purified by preparative HPLC. HPLC Method: Column: XBridge C18 (19×50 mm, 5 μm); mobile phase A: 10 mM ammonium acetate in water; mobile phase B: acetonitrile; gradient 15-50% B over 23 minutes, then 5 minutes hold at 100 B, flow 20 mL/min.

EXAMPLE 367: (First eluting isomer, 2 mg, 2% yield); LCMS: m/z, 477.3 [M+H]⁺; rt 1.82 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.25 (d, J=9.0 Hz, 1H), 7.99 (d, J=9.0 Hz, 1H), 6.12-5.79 (m, 1H), 5.01-4.76 (m, 1H), 3.56 (d, J=10.5 Hz, 2H), 3.44 (s, 3H), 3.00 (dd, J=12.5, 6.0 Hz, 1H), 2.37 (br d, J=10.5 Hz, 1H), 2.30-2.20 (m, 2H), 1.44-1.33 (m, 1H), 1.28-1.10 (m, 6H), 1.06-0.88 (m, 6H), 0.84-0.70 (m, 6H).

EXAMPLE 368: (Second eluting isomer, 1.8 mg, 2% yield); LCMS: m/z, 477.3 [M+H]⁺; rt 2.02 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.24 (br d, J=9.0 Hz, 1H), 8.05-7.90 (m, 1H), 5.94-5.56 (m, 1H), 5.05-4.86 (m, 1H), 3.76-3.56 (m, 2H), 3.46-3.41 (m, 3H), 2.99-2.86 (m, 1H), 2.74 (dd, J=12.3, 3.8 Hz, 1H), 2.22 (br dd, J=4.5, 3.0 Hz, 1H), 2.14-2.04 (m, 1H), 1.58-1.25 (m, 4H), 1.20-1.02 (m, 6H), 1.00-0.81 (m, 6H), 0.72 (br d, J=5.5 Hz, 3H).

### Example 369

### 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)acetonitrile

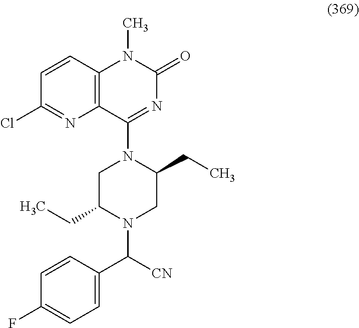 [SMILES: CC[C@H]1CN(C(C#N)c2ccc(F)cc2)[C@H](CC)CN1c1nc(=O)n(C)c2ccc(Cl)nc12]

To a solution of 6-chloro-4-((2S,5R)-2,5-diethylpiperazin-1-yl)-1-methylpyrido[3,2-d]pyrimidin-2(1H)-one (0.85 g, 2.53 mmol) in acetonitrile (2 mL) were added DIPEA (1.33 mL, 7.59 mmol) and 2-bromo-2-(4-fluorophenyl)acetonitrile (1.27 g, 5.06 mmol) at room temperature. The mixture was heated at 85° C. for 16 h. and then was allowed to cool to room temperature. The reaction mixture was concentrated under reduced pressure to obtain the crude product, which was purified by silica gel chromatography (0-100% ethyl acetate in pet ether) to obtain 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl) acetonitrile (550 mg, 29% yield). LCMS: m/z=469.4 [M+H]⁺; retention time 0.92 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Intermediate 47

### (Z)-2-((2R,5S)-4-(6-Chloro-1-methyl-2-oxo-1,2-dihydropyrido[3,2-d]pyrimidin-4-yl)-2,5-diethylpiperazin-1-yl)-2-(4-fluorophenyl)-N-hydroxyacetimidamide

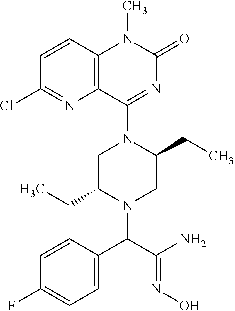 [SMILES: CC[C@H]1CN(C(/C(N)=N/O)c2ccc(F)cc2)[C@H](CC)CN1c1nc(=O)n(C)c2ccc(Cl)nc12]

To a solution of 2-((2R,5S)-4-(6-chloro-1-methyl-2-oxo-1,2-dihydropyrido[3,2-d] pyrimidin-4-yl)-2,5-diethylpiperazin-1-yl)-2-(4-fluorophenyl)acetonitrile (0.320 g, 0.68 mmol) in ethanol (5 mL), hydroxylamine solution (0.046 mL, 0.751 mmol) was added at room temperature and was stirred for 16 h. The reaction mixture was concentrated under reduced pressure, suspended in water (10 mL) and extracted with EtOAc (2×10 mL). The combined organic layer was dried over Na₂SO₄ and concentrated to obtain the crude product, which was purified by silica gel chromatography (0-10% MeOH in DCM) to afford 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)-N-hydroxyacetimidamide (200 mg, 43% yield). LCMS: m/z=502.2 [M+H]⁺; retention time 1.41 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 48

### (Z)-2-((2R,5S)-4-(6-Chloro-1-methyl-2-oxo-1,2-dihydropyrido[3,2-d]pyrimidin-4-yl)-2,5-diethylpiperazin-1-yl)-N′-((cyclopropanecarbonyl)oxy)-2-(4-fluorophenyl)acetimidamide

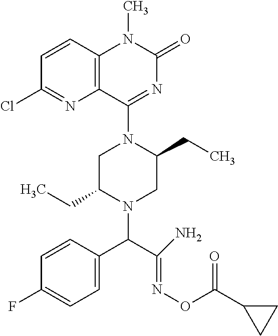 [SMILES: CC[C@H]1CN(C(/C(N)=N/OC(=O)C2CC2)c2ccc(F)cc2)[C@H](CC)CN1c1nc(=O)n(C)c2ccc(Cl)nc12]

To a stirred solution of 2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)-N-hydroxyacetimidamide (0.200 g, 0.32 mmol) in DMF (2 mL), triethylamine (0.14 mL, 0.97 mmol), BOP (0.36 g, 0.81 mmol) and cyclopropanecarboxylic acid (0.034 g, 0.39 mmol) were added at room temperature. After stirring for 2 h, the reaction mixture was washed with saturated ammonium chloride (5 mL) and extracted with ethyl acetate (3×10 mL). The combined organic layer was dried over anhydrous sodium sulphate and then concentrated under reduced pressure to obtain the crude compound N-((E)-2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)-1-(hydroxyimino)ethyl)cyclopropanecarboxamide (180 mg, 69% yield). LCMS: m/z=570.2 [M+H]⁺; retention time 1.59 and 1.61 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Example 370

### 6-Chloro-4-((2S,5R)-4-((5-cyclopropyl-1,2,4-oxadiazol-3-yl)(4-fluorophenyl)methyl)-2,5-diethylpiperazin-1-yl)-1-methylpyrido[3,2-d]pyrimidin-2(1H)-one

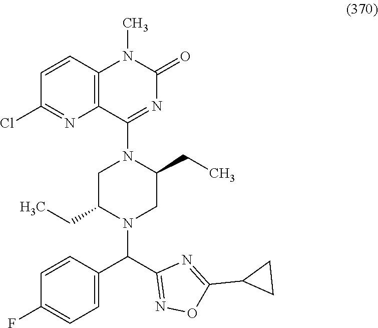 [SMILES: CC[C@H]1CN(C(c2ccc(F)cc2)c2noc(C3CC3)n2)[C@H](CC)CN1c1nc(=O)n(C)c2ccc(Cl)nc12]

To a stirred solution of N-((E)-2-((2R,5S)-4-(6-chloro-1-methyl-2-oxo-1,2-dihydropyrido[3,2-d]pyrimidin-4-yl)-2,5-diethylpiperazin-1-yl)-2-(4-fluorophenyl)-1-(hydroxyimino)ethyl)cyclopropanecarboxamide, TFA (0.180 g, 0.263 mmol) in THF (3 mL), TBAF (1.316 mL, 1.316 mmol) was added at room temperature. The reaction mixture was heated at 65° C. for 16 h. The reaction mixture was allowed to reach room temperature, washed with water (10 mL) and extracted with ethyl acetate (2×10 mL). The organic layer was washed with brine solution, dried over anhydrous sodium sulphate and then concentrated under reduced pressure to give the crude product, which was purified by silica gel chromatography (10% MeOH in DCM as eluent) to obtain 6-chloro-4-((2S,5R)-4-((5-cyclopropyl-1,2,4-oxadiazol-3-yl)(4-fluorophenyl)methyl)-2,5-diethylpiperazin-1-yl)-1-methylpyrido[3,2-d]pyrimidin-2(1H)-one (130 mg, 58% yield) LCMS: m/z=552.2 [M+H]⁺; retention time 2.04 and 2.06 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Examples 371 and 372

### 4-((2S,5R)-4-((5-Cyclopropyl-1,2,4-oxadiazol-3-yl)(4-fluorophenyl)methyl)-2,5-diethylpiperazin-1-yl)-1-methyl-2-oxo-1,2-dihydropyrido[3,2-d]pyrimidine-6-carbonitrile

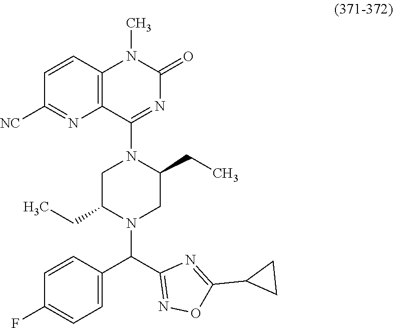 [SMILES: CC[C@H]1CN(C(c2ccc(F)cc2)c2noc(C3CC3)n2)[C@H](CC)CN1c1nc(=O)n(C)c2ccc(C#N)nc12]

To a solution of 6-chloro-4-((2S,5R)-4-((5-cyclopropyl-1,2,4-oxadiazol-3-yl)(4-fluorophenyl)methyl)-2,5-diethylpiperazin-1-yl)-1-methylpyrido[3,2-d]pyrimidin-2(1H)-one (0.06 g, 0.11 mmol) in THF (3 mL) and water (3 mL) were added zinc cyanide (0.03 g, 0.22 mmol), t-BuXPhos (4.62 mg, 10.87 mmol). The reaction mixture was flushed with nitrogen followed by the addition of t-BuXPhos-Pd-G1 (0.043 mg, 1.087 μmol) and again flushed with nitrogen for 1 min. The reaction mixture was heated at 40° C. for 16 h. The reaction mixture was treated with saturated aqueous NaHCO₃ (2 mL) and EtOAc (3 mL). The biphasic mixture was stirred for 5 min. The aqueous layer was further extracted with EtOAc (3×3 mL). The combined organic layer was dried over MgSO₄, filtered, and concentrated in vacuo to obtain the crude product, which was purified by preparative HPLC. HPLC Method: Column: Cellulose-2 (250×21 mm, 5 μm); mobile phase 0.1% DEA in acetonitrile; Flow: 19 mL/min). Example 371: Isolate 1: First eluting peak, rt=9.54 min. Example 372: Isolate 2: Second eluting peak, rt=12.04 min.

EXAMPLE 371: (2.3 mg, 4% yield); LCMS: m/z, 543.3 [M+H]⁺; rt 2.13 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.29-8.16 (m, 1H), 8.04-7.92 (m, 1H), 7.70-7.56 (m, 2H), 7.20 (t, J=8.9 Hz, 2H), 5.99-5.25 (m, 1H), 5.02-4.80 (m, 2H), 3.64-3.53 (m, 1H), 3.43 (s, 3H), 2.65-2.60 (m, 1H), 2.41-2.35 (m, 1H), 2.12-1.40 (m, 3H), 1.23 (br s, 3H), 1.12-1.05 (m, 2H), 0.81-0.64 (m, 6H), (2H are buried under solvent peak).

EXAMPLE 372: (2.2 mg, 4% yield); LCMS: m/z, 543.3 [M+H]⁺; rt 2.15 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.23 (d, J=9.0 Hz, 1H), 8.05-7.91 (m, 1H), 7.66 (br dd, J=5.8, 8.3 Hz, 2H), 7.22 (t, J=9.0 Hz, 2H), 5.92-5.31 (m, 1H), 5.01 (s, 1H), 4.97-4.81 (m, 1H), 3.43 (s, 3H), 3.20-3.16 (m, 1H), 2.80-2.71 (m, 1H), 2.46-2.31 (m, 2H), 2.08-1.70 (m, 1H), 1.57-1.35 (m, 2H), 1.31-1.18 (m, 2H), 1.17-0.98 (m, 3H), 0.92-0.76 (m, 4H), 0.74-0.55 (m, 3H).

The examples in the Table 15 were prepared from general procedure described in Examples 371 and 372, using appropriate benzhydryl/α-substituted benzyl/benzyl halide. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 15

| | | | | | | Synthetic |
|---|---|---|---|---|---|---|
| Ex. | | Stereo | LCMS | LCMS | [M + | Route (A |
| No. | STRUCTURE | chem. | Method | rt | H]⁺ | or B) |
| 373 | 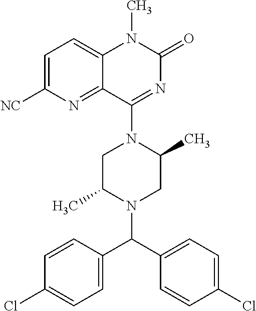 [SMILES: C[C@@H]1CN(c2nc(=O)n(C)c3ccc(C#N)nc23)[C@@H](C)CN1C(c1ccc(Cl)cc1)c1ccc(Cl)cc1] | H | D | 1.53 | 533.25 | D |
| 374 375 | 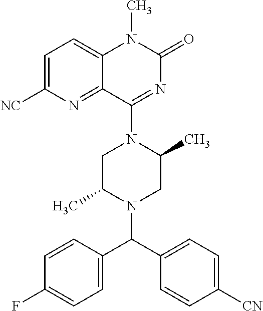 [SMILES: C[C@@H]1CN(c2nc(=O)n(C)c3ccc(C#N)nc23)[C@@H](C)CN1C(c1ccc(F)cc1)c1ccc(C#N)cc1] | H H | F F | 1.89 1.87 | 508.33 508.08 | B B |
| 376 377 | 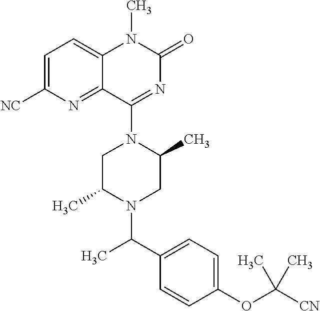 [SMILES: CC(c1ccc(OC(C)(C)C#N)cc1)N1C[C@H](C)N(c2nc(=O)n(C)c3ccc(C#N)nc23)C[C@H]1C] | H H | E E | 2.83 3.02 | 486.2 486.2 | D D |
| 378 379 | 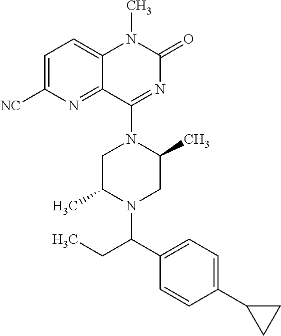 [SMILES: CCC(c1ccc(C2CC2)cc1)N1C[C@H](C)N(c2nc(=O)n(C)c3ccc(C#N)nc23)C[C@H]1C] | H H | C C | 2.38 2.4 | 457.3 457.3 | B B |
| 380 381 | 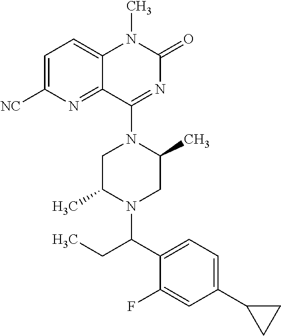 [SMILES: CCC(c1ccc(C2CC2)cc1F)N1C[C@H](C)N(c2nc(=O)n(C)c3ccc(C#N)nc23)C[C@H]1C] | H H | C C | 2.39 2.4 | 475.3 475.3 | B B |

### Intermediate 49

### tert-Butyl (2S,5R)-4-(1-(4-(methoxycarbonyl)phenyl)ethyl)-2,5-dimethylpiperazine-1-carboxylate

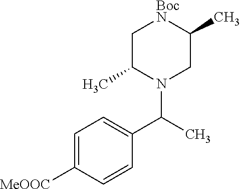 [SMILES: COC(=O)c1ccc(C(C)N2C[C@H](C)N(C(=O)OC(C)(C)C)C[C@H]2C)cc1]

To a stirred solution of tert-butyl (2S,5R)-2,5-dimethylpiperazine-1-carboxylate (2.5 g, 11.67 mmol) in acetonitrile (30 mL), DIPEA (6.1 mL, 35.0 mmol), potassium iodide (1.94 g, 11.67 mmol) and methyl 4-(1-chloroethyl)benzoate (2.32 g, 11.67 mmol) were added sequentially at room temperature. The reaction mixture was heated at 80° C. for 24 h, then it was cooled to room temperature and the solvent was removed under reduced pressure to give the crude product, which was purified by silica gel column chromatography using 20-30% EtOAc in n-hexane to afford tert-butyl (2S,5R)-4-(1-(4-(methoxycarbonyl)phenyl)ethyl)-2,5-dimethylpiperazine-1-carboxylate (3.5 g, 62% yield). LCMS: m/z, 377.3 [M+H]⁺; rt 1.39 min rt (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Intermediate 50

### tert-Butyl (2S,5R)-4-(1-(4-(hydroxymethyl)phenyl)ethyl)-2,5-dimethylpiperazine-1-carboxylate

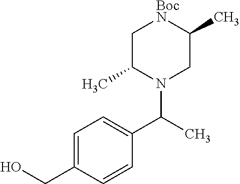 [SMILES: CC(c1ccc(CO)cc1)N1C[C@H](C)N(C(=O)OC(C)(C)C)C[C@H]1C]

To a stirred solution of tert-butyl (2S,5R)-4-(1-(4-(methoxycarbonyl)phenyl) ethyl)-2,5-dimethylpiperazine-1-carboxylate (1.8 g, 4.78 mmol) in THF (20 mL) was cooled to 0° C. and lithium borohydride (2 M in THF) (24 mL, 47.8 mmol) was added drop wise under nitrogen. The reaction mixture was allowed to warm room temperature and stirred for 2 h. The reaction mixture was cooled to 0° C. The reaction was quenched with the addition of saturated aqueous NH₄Cl solution. The reaction mixture was extracted with EtOAc (3×50 mL). The combined organic extract was washed with water, brine, dried over sodium sulphate and the solvent was removed under reduced pressure to afford tert-butyl (2S,5R)-4-(1-(4-(hydroxymethyl)phenyl)ethyl)-2,5-dimethylpiperazine-1-carboxylate (1.3 g, 76% yield). LCMS: m/z, 349.3 [M+H]⁺; rt 3.05 min. (LCMS Method: Column-Kinetex XB-C18 (75×3 mm-2.6 μm); Mobile phase A: 10 mM ammonium formate in water:acetonitrile (98:2); Mobile phase B: 10 mM ammonium formate in water:acetonitrile (2:98); Flow: 1 mL/min; Gradient: 20-100% B over 4 minutes then 0.6 min hold at 100% B, Detection: UV at 220 nm).

### Intermediate 51

### (4-(1-((2R,5S)-2,5-Dimethylpiperazin-1-yl)ethyl)phenyl)methanol, HCl Salt (Diastereomeric Mixture)

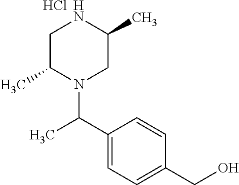 [SMILES: Cl ; CC(c1ccc(CO)cc1)N1C[C@H](C)NC[C@H]1C]

To a stirred solution of tert-butyl (2S,5R)-4-(1-(4-(hydroxymethyl)phenyl)ethyl)-2,5-dimethylpiperazine-1-carboxylate (0.8 g, 2.30 mmol) in DCM (15 mL), HCl (4 N in dioxane) (2.9 mL, 11.5 mmol) was added drop wise at room temperature. The reaction mixture was stirred for 3 h, solvent was removed under reduced pressure, the solids were co-distilled with acetonitrile (3×10 mL), and dried to afford (4-(1-((2R,5S)-2,5-dimethylpiperazin-1-yl)ethyl)phenyl)methanol HCl salt (0.4 g, 45% yield) as an off-white solid. LCMS: m/z, 249.2 [M+H]⁺; rt 0.51 and 0.55 min. (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Example 382

### 6-Chloro-4-((2S,5R)-4-(1-(4-(hydroxymethyl)phenyl)ethyl)-2,5-dimethylpiperazin-1-yl)-1-methylpyrido[3,2-d]pyrimidin-2(1H)-one (Diastereomeric Mixture)

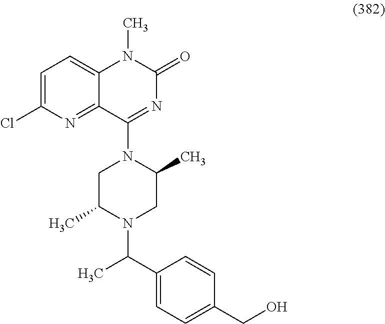 [SMILES: CC(c1ccc(CO)cc1)N1C[C@H](C)N(c2nc(=O)n(C)c3ccc(Cl)nc23)C[C@H]1C]

To a stirred solution of (4-(1-((2R,5S)-2,5-dimethylpiperazin-1-yl)ethyl)phenyl) methanol HCl salt (600 mg, 2.416 mmol) in acetonitrile (10 mL), DIPEA (2.1 mL, 12.1 mmol) and 4,6-dichloro-1-methylpyrido[3,2-d]pyrimidin-2(1H)-one (560 mg, 2.42 mmol) were added sequentially at room temperature. The reaction mixture was heated at 80° C. for 12 h. The reaction mixture was cooled to room temperature and the solvent was removed under reduced pressure to give the crude product, which was purified by silica gel column chromatography using 0-10% MeOH in CHCl₃ to afford 6-chloro-4-((2S,5R)-4-(1-(4-(hydroxymethyl)phenyl)ethyl)-2,5-dimethylpiperazin-1-yl)-1-methylpyrido[3,2-d] pyrimidin-2(1H)-one (0.6 g, 51% yield). LCMS: m/z, 442.3 [M+H]⁺; rt 1.48 min. (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Example 383

### 4-((2S,5R)-4-(1-(4-(Hydroxymethyl)phenyl)ethyl)-2,5-dimethylpiperazin-1-yl)-1-methyl-2-oxo-1,2-dihydropyrido[3,2-d]pyrimidine-6-carbonitrile (Diastereomeric Mixture)

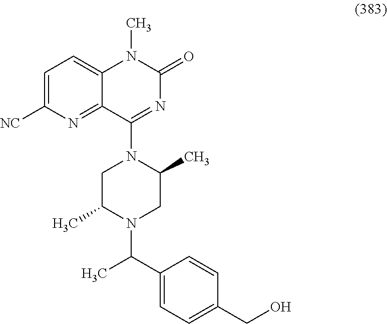 [SMILES: CC(c1ccc(CO)cc1)N1C[C@H](C)N(c2nc(=O)n(C)c3ccc(C#N)nc23)C[C@H]1C]

To a stirred solution of 6-chloro-4-((2S,5R)-4-(1-(4-(hydroxymethyl)phenyl) ethyl)-2,5-dimethylpiperazin-1-yl)-1-methylpyrido[3,2-d]pyrimidin-2(1H)-one (400 mg, 0.905 mmol) in DMF (10.0 mL) were added zinc (89 mg, 1.36 mmol) and TEA (0.5 mL, 3.62 mmol). The reaction mixture was flushed with argon for 5 min followed by the addition of zinc cyanide (320 mg, 2.72 mmol) and dichloro[9,9-dimethyl-4,5-bis(diphenylphosphino)xanthene]palladium(II) (68.4 mg, 0.091 mmol). The reaction mixture heated at 90° C. for 16 h, then cooled to room temperature, filtered through Celite® pad, washed with excess EtOAc (40 mL) and the filtrate was concentrated under reduced pressure to give the crude product, which was purified by silica gel column chromatography using 0-10% MeOH in CHCl₃ to afford 4-((2S,5R)-4-(1-(4-(hydroxymethyl)phenyl)ethyl)-2,5-dimethylpiperazin-1-yl)-1-methyl-2-oxo-1,2-dihydropyrido[3,2-d]pyrimidine-6-carbonitrile (260 mg, 57% yield). LCMS: m/z, 433.2 [M+H]⁺; rt 1.22 and 1.23 min. (LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm).

### Example 384

### 4-((2S,5R)-4-(1-(4-(Bromomethyl)phenyl)ethyl)-2,5-dimethylpiperazin-1-yl)-1-methyl-2-oxo-1,2-dihydropyrido[3,2-d]pyrimidine-6-carbonitrile (Diastereomeric Mixture)

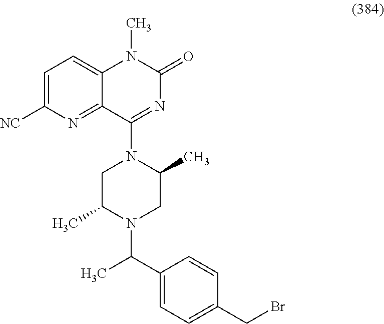 [SMILES: CC(c1ccc(CBr)cc1)N1C[C@H](C)N(c2nc(=O)n(C)c3ccc(C#N)nc23)C[C@H]1C]

To a stirred solution of 4-((2S,5R)-4-(1-(4-(hydroxymethyl)phenyl)ethyl)-2,5-dimethylpiperazin-1-yl)-1-methyl-2-oxo-1,2-dihydropyrido[3,2-d]pyrimidine-6-carbonitrile (300 mg, 0.69 mmol) in DCM (10 mL) was cooled 0° C., added triphenylphosphine polymer bound (1.3 mmol/g) (1.5 g, 5.55 mmol) followed by CBr₄ (460 mg, 1.40 mmol) in DCM (5 mL). The reaction mixture was allowed to warm to room temperature and stirred for 2 h. The solvent was removed under reduced pressure to give the crude product, which was purified by silica gel column chromatography using 80-100% EtOAc in n-hexane to afford 4-((2S,5R)-4-(1-(4-(bromomethyl)phenyl) ethyl)-2,5-dimethylpiperazin-1-yl)-1-methyl-2-oxo-1,2-dihydropyrido[3,2-d]pyrimidine-6-carbonitrile (360 mg, 49% yield). LCMS: m/z, 495.2 [M+H]⁺; rt 1.89 min. LCMS Method: Column: Acquity UPLC BEH C18 (3.0×50 mm, 1.7 μm); mobile phase A: 10 mM ammonium acetate:acetonitrile (95:5); mobile phase B: 10 mM ammonium acetate:acetonitrile (5:95), Gradient=20-100% B over 2 minute, then a 0.3 minute hold at 100% B; Temperature: 50° C.; Flow rate: 0.7 mL/min; Detection: UV at 220 nm.

### Examples 385 and 386

### 4-((2S,5R)-4-(1-(4-((2,2-Dimethylmorpholino)methyl)phenyl)ethyl)-2,5-dimethylpiperazin-1-yl)-1-methyl-2-oxo-1,2-dihydropyrido[3,2-d]pyrimidine-6-carbonitrile

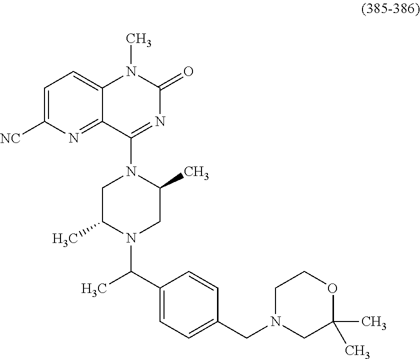 [SMILES: CC(c1ccc(CN2CCOC(C)(C)C2)cc1)N1C[C@H](C)N(c2nc(=O)n(C)c3ccc(C#N)nc23)C[C@H]1C]

To a stirred solution of 4-((2S,5R)-4-(1-(4-(bromomethyl)phenyl)ethyl)-2,5-dimethylpiperazin-1-yl)-1-methyl-2-oxo-1,2-dihydropyrido[3,2-d]pyrimidine-6-carbonitrile (80 mg, 0.16 mmol) in acetonitrile (2 mL), potassium carbonate (89 mg, 0.65 mmol) and 2,2-dimethylmorpholine (37.2 mg, 0.32 mmol) were added sequentially at room temperature and heated at 60° C. for 1 h. The reaction mixture cooled to room temperature, filtered through a Celite® pad, washed with excess acetonitrile (10 mL) and the filtrate was concentrated under reduced pressure to give the crude product, which was purified using preparative HPLC (Chiral Separation Method: Column: Cellulose-5 (250×21.2 mm, 5 μm); mobile phase: 0.3% DEA in MeOH; Flow: 20 mL/min; UV detection: 215 nm).

EXAMPLE 385: (2 mg, 2% yield). LCMS: m/z=530.4 [M+H]⁺; rt 2.19 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.22 (br d, J=9.0 Hz, 1H), 7.98 (d, J=9.0 Hz, 1H), 7.36-7.26 (m, 4H), 5.81-5.32 (m, 1H), 5.19-4.53 (m, 1H), 3.63-3.57 (m, 3H), 3.45-3.40 (m, 5H), 2.88-2.75 (m, 3H), 2.28 (br s, 2H), 2.16 (s, 2H), 1.57-1.36 (m, 3H), 1.24 (br d, J=6.4 Hz, 3H), 1.16 (s, 6H), 0.93-0.86 (m, 3H), (1H might be obscured with solvent peak).

EXAMPLE 386: (2.0 mg, 2% yield). LCMS: m/z=530.4 [M+H]⁺; rt 2.15 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.23 (br d, J=8.8 Hz, 1H), 7.98 (d, J=9.0 Hz, 1H), 7.36-7.22 (m, 4H), 5.78-5.46 (m, 1H), 4.97-4.72 (m, 1H), 3.75-3.53 (m, 4H), 3.48-3.39 (m, 6H), 2.61-2.55 (m, 1H), 2.28 (br d, J=3.4 Hz, 2H), 2.21-2.12 (m, 3H), 1.35-1.19 (m, 6H), 1.15 (s, 6H), 1.10-0.99 (m, 3H).

The examples in the Table 16 were prepared from general procedure described in Examples 385 and 386, using appropriate amine and 4-((2S,5R)-4-(1-(4-(bromomethyl) phenyl)ethyl)-2,5-dimethylpiperazin-1-yl)-1-methyl-2-oxo-1,2-dihydropyrido[3,2-d] pyrimidine-6-carbonitrile (diastereomeric mixture). Diastereomers was separated at the final stage using either preparative chromatography or preparative chiral chromatography. The absolute stereochemistry was not assigned at the newly formed carbon-nitrogen bond.

TABLE 16

| Ex. | | Stereo | LCMS | LCMS | |
|---|---|---|---|---|---|
| No. | Structure | chemistry | Method | rt | [M + H]⁺ |
| 387 388 | 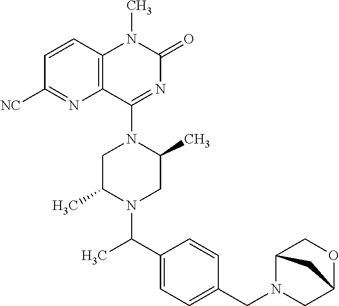 [SMILES: CC(c1ccc(CN2C[C@@H]3C[C@H]2CO3)cc1)N1C[C@H](C)N(c2nc(=O)n(C)c3ccc(C#N)nc23)C[C@H]1C] | H H | C C | 1.53 1.55 | 514.3 514.3 |
| 389 | 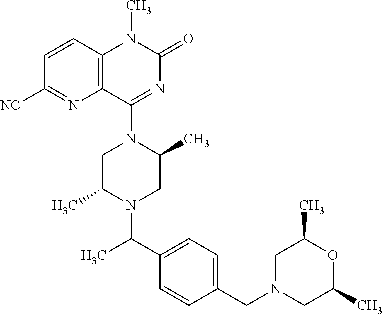 [SMILES: CC(c1ccc(CN2C[C@@H](C)O[C@@H](C)C2)cc1)N1C[C@H](C)N(c2nc(=O)n(C)c3ccc(C#N)nc23)C[C@H]1C] | H | C | 2.04 | 530.4 |
| 390 391 | 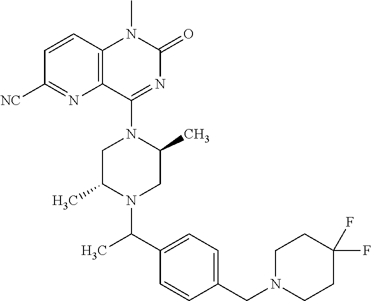 [SMILES: CC(c1ccc(CN2CCC(F)(F)CC2)cc1)N1C[C@H](C)N(c2nc(=O)n(C)c3ccc(C#N)nc23)C[C@H]1C] | H H | C C | 2.18 2.2 | 536.3 536.3 |
| 392 393 | 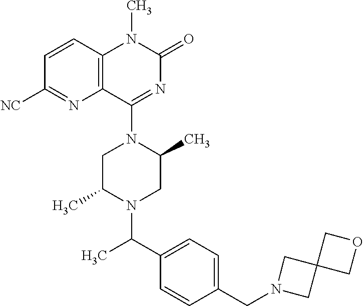 [SMILES: CC(c1ccc(CN2CC3(COC3)C2)cc1)N1C[C@H](C)N(c2nc(=O)n(C)c3ccc(C#N)nc23)C[C@H]1C] | H H | C C | 1.43 1.43 | 514.3 514.4 |

### Intermediate 52

### Methyl (S)-2-((R)—N-benzyl-2-((tert-butoxycarbonyl)amino)propanamido)butanoate

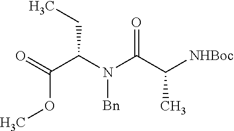 [SMILES: CC[C@@H](C(=O)OC)N(Cc1ccccc1)C(=O)[C@@H](C)NC(=O)OC(C)(C)C]

To a solution of methyl (S)-2-(benzylamino) butanoate (3.0 g, 14.47 mmol) and (tert-butoxycarbonyl)-D-alanine (4.11 g, 21.71 mmol) in DMF (30 mL) were added DIPEA (7.58 mL, 43.4 mmol) and HATU (11.01 g, 28.9 mmol) at room temperature. The reaction mixture was stirred at room temperature for 16 h. The reaction was quenched with the addition of water. The reaction mixture was extracted with ethyl acetate (2×100 mL), the combined organic layer was washed 0.5 N aqueous HCl, water, brine and dried over sodium sulfate. Evaporation of the solvent yielded methyl (S)-2-((R)—N-benzyl-2-((tert-butoxycarbonyl)amino)propanamido)butanoate (4.0 g, 10.57 mmol, 73.0% yield). LCMS: m/z=379.2 [M+H]⁺; retention time 2.95 min. (Column: Kinetex XB-C18 (75×3 mm, 2.6 μm); mobile phase A: 10 mM NH₄OAc in water:acetonitrile (98:2) mobile phase B: 10 mM NH₄OAc in water:acetonitrile (2:98). Gradient=20-100% B over 4 minutes, then a 0.6 minute hold at 100% B; Flow rate: 1.5 mL/min; Detection: UV at 220 nm.

### Intermediate 53

### Methyl (S)-2-((R)-2-amino-n-benzylpropanamido)butanoate, TFA

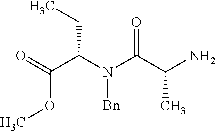 [SMILES: CC[C@@H](C(=O)OC)N(Cc1ccccc1)C(=O)[C@@H](C)N]

To a solution of methyl (S)-2-((R)—N-benzyl-2-((tert-butoxycarbonyl)amino) propanamido)butanoate (4.0 g, 10.57 mmol) in DCM (30 mL) was added TFA (10 mL, 130 mmol) at 0° C. The reaction mixture was stirred at room temperature for 4 h. The reaction mixture was concentrated under reduced pressure to afford methyl (S)-2-((R)-2-amino-N-benzylpropanamido)butanoate, TFA (4.15 g, 10.58 mmol, 100% yield). LCMS: m/z=279.3 [M+H]⁺; retention time 0.75 min, LCMS Method: Column-Luna 3.0 C18 (2) 100 Å LC column (20×4.0 mm); mobile phase A: 0.1% TFA in water, mobile phase B: 0.1% TFA in acetonitrile. Gradient=20-100% B over 2.5 minutes, then a 0.8 minute hold at 100% B; Flow rate: 1.5 mL/min; Detection: UV at 220 nm.

### Intermediate 54

### (3R,6S)-1-Benzyl-6-ethyl-3-methylpiperazine-2,5-dione

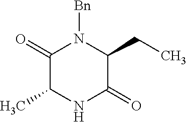 [SMILES: CC[C@H]1C(=O)N[C@H](C)C(=O)N1Cc1ccccc1]

A solution of methyl (S)-2-((R)-2-amino-N-benzylpropanamido)butanoate, TFA (4.0 g, 10.19 mmol) in methanol (5 mL) was refluxed at 65° C. for 24 h. The reaction mixture was concentrated under reduced pressure and dried under high vacuum to afford (3R,6S)-1-benzyl-6-ethyl-3-methylpiperazine-2,5-dione (2.2 g, 8.93 mmol, 88% yield). LCMS: m/z=247.2 [M+H]⁺; retention time 1.41 min. Column: Kinetex XB-C18 (3×75 mm, 2.6 μm); mobile phase A: 10 mM ammonium acetate:acetonitrile (98:2), mobile phase B: 10 mM ammonium acetate:acetonitrile (2:98), Gradient=20-100% B over 4 minutes, then a 0.6 minute hold at 100% B; Flow rate: 1.0 mL/min; Detection: UV at 220 nm.

### Intermediate 55

### (2S,5R)-1-Benzyl-2-ethyl-5-methylpiperazine

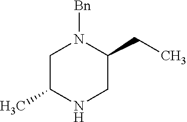 [SMILES: CC[C@H]1CN[C@H](C)CN1Cc1ccccc1]

To a solution of (3R,6S)-1-benzyl-6-ethyl-3-methylpiperazine-2,5-dione (2.2 g, 8.93 mmol) in THF (30 mL) at 0° C. was added 1 M solution of borane tetrahydrofuran complex (26.8 mL, 26.8 mmol) in THF. The reaction mixture was stirred at reflux for 24 h. The reaction mixture was cooled. The reaction was quenched with the addition of methanol. To the mixture was added concentrated HCl (5.0 mL). The mixture was refluxed for 3 h and cooled to room temperature. The mixture was concentrated under reduced pressure and the resultant residue was dissolved in DCM and washed with water, brine and dried over sodium sulphate and concentrated under reduced pressure to afford (2S,5R)-1-benzyl-2-ethyl-5-methylpiperazine (1.90 g, 8.70 mmol, 97% yield). LCMS: m/z=219.2 [M+H]⁺; retention time 0.43 min, LCMS Method: Column-Luna 3.0 C18 (2) 100 Å LC column (20×4.0 mm); mobile phase A: 0.1% TFA in water mobile phase B: 0.1% TFA in acetonitrile. Gradient=20-100% B over 2.5 minutes, then a 0.3 minute hold at 100% B; Flow rate: 1.5 mL/min; Detection: UV at 220 nm.

### Intermediate 56

### tert-Butyl (2R,5S)-4-benzyl-5-ethyl-2-methylpiperazine-1-carboxylate

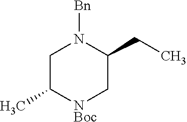 [SMILES: CC[C@H]1CN(C(=O)OC(C)(C)C)[C@H](C)CN1Cc1ccccc1]

To a solution of (2S,5R)-1-benzyl-2-ethyl-5-methylpiperazine (1.9 g, 8.70 mmol) in DCM (20 mL) was added TEA (2.43 mL, 17.40 mmol) and Boc-anhydride (4.04 mL, 17.40 mmol). The reaction mixture was stirred at room temperature for 2 h. The reaction mixture was concentrated under reduced pressure to obtain the crude product which was purified by silica gel column chromatography using 20% EtOAc in pet ether. The fractions were concentrated under reduced pressure to yield the pure product tert-butyl (2R,5S)-4-benzyl-5-ethyl-2-methylpiperazine-1-carboxylate (2.0 g, 6.28 mmol, 72.2% yield). LCMS: m/z=319.2 [M+H]⁺; retention time 4.2 min. Column: Kinetex XB-C18 (3×75 mm, 2.6 μm); mobile phase A: 10 mM ammonium acetate:acetonitrile (98:2), mobile phase B: 10 mM ammonium acetate:acetonitrile (2:98), Gradient=20-100% B over 4 minutes, then a 0.6 minute hold at 100% B; Flow rate: 1.5 mL/min; Detection: UV at 220 nm.

### Intermediate 57

### tert-Butyl (2R,5S)-5-ethyl-2-methylpiperazine-1-carboxylate

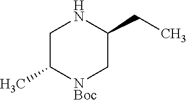 [SMILES: CC[C@H]1CN(C(=O)OC(C)(C)C)[C@H](C)CN1]

In a 100 mL autoclave, to a solution of tert-butyl (2R,5S)-4-benzyl-5-ethyl-2-methylpiperazine-1-carboxylate (2.0 g, 6.28 mmol) in methanol (40 mL) was added acetic acid (0.360 mL, 6.28 mmol) at room temperature. The reaction mixture was purged with N₂ and 10% palladium on carbon (200 mg, 0.188 mmol) was added. The reaction mixture was evacuated and stirred overnight under hydrogen (70 psi) atmosphere. The reaction mixture was filtered through a Celite® pad. The filtrate was concentrated under reduced pressure and dried under high vacuum to yield tert-butyl (2R,5S)-5-ethyl-2-methylpiperazine-1-carboxylate, AcOH (1.4 g, 4.85 mmol, 77% yield). ¹H NMR (400 MHz, DMSO-d₆) δ (ppm)=0.79-0.91 (m, 3H) 1.13 (d, J=6.53 Hz, 3H) 1.23-1.35 (m, 9H) 1.37-1.40 (m, 2H), 2.28-2.45 (m, 2H) 2.90 (m, 1H), 3.07 (m, 1H), 3.52 (m, 1H), 3.59 (m, 1H) 3.91-4.13 (m, 1H).

### Intermediate 58

### Methyl N-benzyl-N—((R)-2-((tert-butoxycarbonyl)amino)pentanoyl)-L-alaninate

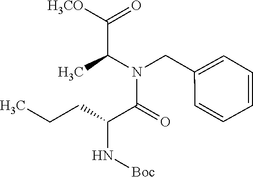 [SMILES: CCC[C@@H](NC(=O)OC(C)(C)C)C(=O)N(Cc1ccccc1)[C@@H](C)C(=O)OC]

To a solution of (R)-2-((tert-butoxycarbonyl)amino)pentanoic acid (13.49 g, 62.1 mmol) in DMF (50 mL) at room temperature was added HATU (18.89 g, 49.7 mmol) and DIPEA (21.7 mL, 124 mmol), followed by methyl benzyl-L-alaninate (8 g, 41.4 mmol). The reaction mixture was stirred at room temperature for 16 h. Water was added to quench the reaction. The mixture was extracted with ethyl acetate (2×100 mL), the combined organic layer was washed with water, brine and dried over sodium sulfate. The solvent was evaporated under reduced pressure to yield the crude product, which was purified by silica gel column chromatography using 30% EtOAc in pet ether as eluent. The fractions were concentrated under reduced pressure to yield methyl N-benzyl-N—((R)-2-((tert-butoxycarbonyl)amino)pentanoyl)-L-alaninate (11 g, 68% yield). LCMS: m/z=393.2 [M+H]⁺; retention time 1.84 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 59

### Methyl N—((R)-2-aminopentanoyl)-N-benzyl-L-alaninate

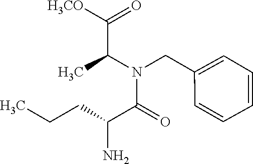 [SMILES: CCC[C@@H](N)C(=O)N(Cc1ccccc1)[C@@H](C)C(=O)OC]

To a solution of methyl N-benzyl-N—((R)-2-((tert-butoxycarbonyl)amino) pentanoyl)-L-alaninate (3.5 g, 8.92 mmol) in DCM (15 mL) at 0° C. was added TFA (0.7 mL, 8.92 mmol). The reaction mixture was stirred at room temperature for 16 h. The reaction mixture was concentrated under reduced pressure and washed with 1:1 diethyl ether:pet ether and dried under high vacuum to yield methyl N—((R)-2-aminopentanoyl)-N-benzyl-L-alaninate, TFA (3 g, 7.38 mmol, 83% yield). LCMS: m/z=293.2 [M+H]⁺; retention time 1.08 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 60

### (3R,6S)-1-Benzyl-6-methyl-3-propylpiperazine-2, 5-Dione

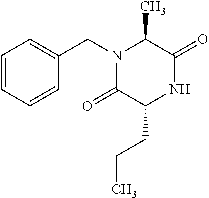 [SMILES: CCC[C@H]1NC(=O)[C@H](C)N(Cc2ccccc2)C1=O]

A stirred solution of methyl N—((R)-2-aminopentanoyl)-N-benzyl-L-alaninate, TFA (3 g, 10.26 mmol) in methanol (15 mL) was heated at 70° C. for 16 h. The reaction mixture was concentrated and dried under reduced pressure to afford (3R,6S)-1-benzyl-6-methyl-3-propylpiperazine-2,5-dione (2 g, 23% yield). LCMS: m/z=261.2 [M+H]⁺; retention time 1.06 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 61

### (2S, 5R)-1-Benzyl-2-methyl-5-propylpiperazine

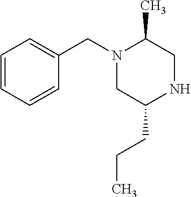 [SMILES: CCC[C@@H]1CN(Cc2ccccc2)[C@@H](C)CN1]

To a stirred solution of (3R,6S)-1-benzyl-6-methyl-3-propylpiperazine-2,5-dione (0.5 g, 1.92 mmol) in THF (25 mL) at 0° C. was added borane tetrahydrofuran complex (9.6 mL, 9.6 mmol). The reaction mixture was heated at reflux overnight. The reaction was quenched with the addition of methanol. To the reaction mixture was added aqueous concentrated HCl (0.5 mL). The reaction mixture was refluxed for 3 h. Then the reaction mixture was concentrated under reduced pressure and the residue was dissolved in DCM, washed with saturated aqueous NaHCO₃ solution, water, brine and dried with anhydrous sodium sulphate and concentrated under reduced pressure to yield (2S,5R)-1-benzyl-2-methyl-5-propylpiperazine (0.3 g, 59% yield). LCMS: m/z=233.2 [M+H]⁺; retention time 1.7 min. LCMS Method: Column-Kinetex XB-C18 (75×3 mm, 2.6 μm); mobile phase A: 0.1% TFA in water, mobile phase B: 0.1% TFA in acetonitrile.

### Intermediate 62

### tert-Butyl (2R,5S)-4-benzyl-5-methyl-2-propylpiperazine-1-carboxylate

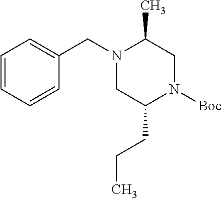 [SMILES: CCC[C@@H]1CN(Cc2ccccc2)[C@@H](C)CN1C(=O)OC(C)(C)C]

To a solution of (2S,5R)-1-benzyl-2-methyl-5-propylpiperazine (0.35 g, 1.51 mmol) in DCM (10 mL) was added triethylamine (0.6 mL, 4.52 mmol), followed by Boc-anhydride (0.5 mL, 2.26 mmol) at room temperature. The reaction mixture was stirred for 3 h. The reaction was quenched with the addition of water. The mixture was extracted with ethyl acetate (2×100 mL). The combined organic layer was washed with water, brine and dried over sodium sulfate. The solvent was evaporated under reduced pressure to yield the crude product, which was purified by silica gel column chromatography using 40% EtOAc in pet ether as eluent. The homogenous fractions were concentrated under reduced pressure to yield the product tert-butyl (2R,5S)-4-benzyl-5-methyl-2-propylpiperazine-1-carboxylate (0.3 g, 60% yield). LCMS: m/z=333.2 [M+H]⁺; retention time 4.26 min LCMS Method: Column-Kinetex XB-C18 (75×3 mm, 2.6 μm); mobile phase A: 10 mM NH₄OAc in water:acetonitrile (98:2) Mobile phase B: 10 mM NH₄OAc in water:acetonitrile (2:98).

### Intermediate 63

### tert-Butyl (2R,5S)-5-methyl-2-propylpiperazine-1-carboxylate

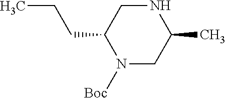 [SMILES: CCC[C@@H]1CN[C@@H](C)CN1C(=O)OC(C)(C)C]

To a solution of tert-butyl (2R,5S)-4-benzyl-5-methyl-2-propylpiperazine-1-carboxylate (0.4 g, 1.2 mmol) in methanol (5 mL) was added acetic acid (0.07 mL, 1.2 mmol) and 10% Pd on carbon (0.333 g, 1.564 mmol) at room temperature. The reaction mixture was stirred overnight under a hydrogen atmosphere. The reaction mixture was filtered through a Celite® pad, and the filtrate was concentrated and dried under reduced pressure to yield tert-butyl (2R,5S)-5-methyl-2-propylpiperazine-1-carboxylate AcO— (0.2 g, 0.584 mmol, 48.5% yield). LCMS: m/z=243.2 [M+H]⁺; retention time 2.50 min. (Column: Kinetex XB-C18 (75×3 mm, 2.6 μm); mobile phase A: 10 mM NH₄OAc in water:acetonitrile (98:2) mobile phase B: 10 mM NH₄OAc in water:acetonitrile (2:98).

### Intermediate 64

### tert-Butyl (2R,5S)-4-(6-chloro-1-methyl-2-oxo-1,2-dihydropyrido[3,2-d]pyrimidin-4-yl)-5-methyl-2-propylpiperazine-1-carboxylate

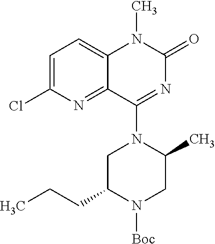 [SMILES: CCC[C@@H]1CN(c2nc(=O)n(C)c3ccc(Cl)nc23)[C@@H](C)CN1C(=O)OC(C)(C)C]

To a stirred solution of tert-butyl (2R,5S)-5-methyl-2-propylpiperazine-1-carboxylate AcO— (0.45 g, 1.493 mmol) in acetonitrile (10 mL) were added DIPEA (0.97 mL, 5.6 mmol) and 4,6-dichloro-1-methylpyrido[3,2-d]pyrimidin-2(1H)-one (0.427 g, 1.86 mmol) sequentially at room temperature. The reaction mixture was heated at 85° C. for 16 h. The reaction mixture was concentrated under reduced pressure to yield the crude product, which was purified by silica gel column chromatography (eluted with 50% EtOAc in pet ether) to yield tert-butyl (2R,5S)-4-(6-chloro-1-methyl-2-oxo-1,2-dihydropyrido[3,2-d]pyrimidin-4-yl)-5-methyl-2-propylpiperazine-1-carboxylate (0.3 g, 25% yield). LCMS: m/z=436.2 [M+H]⁺; retention time 1.87 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min.

### Intermediate 65

### 6-Chloro-1-methyl-4-((2S,5R)-2-methyl-5-propylpiperazin-1-yl)pyrido[3,2-d]pyrimidin-2(1H)-one

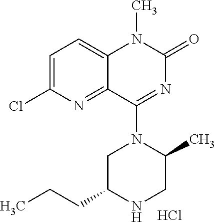 [SMILES: CCC[C@@H]1CN(c2nc(=O)n(C)c3ccc(Cl)nc23)[C@@H](C)CN1 ; Cl]

To a solution of tert-butyl (2R,5S)-4-(6-chloro-1-methyl-2-oxo-1,2-dihydropyrido[3,2-d]pyrimidin-4-yl)-5-methyl-2-propylpiperazine-1-carboxylate (0.15 g, 0.34 mmol) in dioxane (5 mL) was added 4 N HCl in 1,4-dioxane (4, M, 0.9 mL, 3.4 mmol) at 0° C. The reaction mixture was stirred at room temperature for 3 h. The reaction mixture was concentrated under reduced pressure yield 6-chloro-1-methyl-4-((2S,5R)-2-methyl-5-propylpiperazin-1-yl)pyrido[3,2-d]pyrimidin-2(1H)-one HCl salt (yield: 0.12 g, 79%). LCMS: m/z=336.2 [M+H]⁺; retention time 0.88 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min.

### Example 394

### 6-Chloro-1-methyl-4-((2S,5R)-2-methyl-5-propyl-4-(1-(4-(trifluoromethyl)phenyl)ethyl)piperazin-1-yl)pyrido[3,2-d]pyrimidin-2(1H)-one

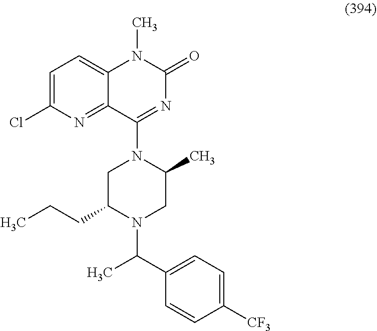 [SMILES: CCC[C@@H]1CN(c2nc(=O)n(C)c3ccc(Cl)nc23)[C@@H](C)CN1C(C)c1ccc(C(F)(F)F)cc1]

To a stirred solution of 6-chloro-1-methyl-4-((2S,5R)-2-methyl-5-propylpiperazin-1-yl)pyrido[3,2-d]pyrimidin-2(1H)-one HCl salt (0.15 g, 0.333 mmol) in acetonitrile (5 mL) were added DIPEA (0.2 mL, 1.0 mmol), 1-(1-chloroethyl)-4-(trifluoromethyl) benzene (0.139 g, 0.67 mmol) and sodium iodide (0.05 g, 0.33 mmol) at room temperature. The reaction mixture was heated at 85° C. for 16 h. The reaction was quenched with the addition of water. The reaction mixture was extracted with ethyl acetate (2×100 mL), the combined organic layer was washed with water, brine and dried over sodium sulfate. The solvent was evaporated under reduced pressure to yield the crude product, which was purified by silica gel column chromatography (eluted with 30% EtOAc in pet ether). The fractions were concentrated under reduced pressure to yield 6-chloro-1-methyl-4-((2S,5R)-2-methyl-5-propyl-4-(1-(4-(trifluoromethyl)phenyl)ethyl) piperazin-1-yl)pyrido[3,2-d]pyrimidin-2(1H)-one (0.1 g, 22% yield). LCMS: m/z=508.3 [M+H]⁺; retention time 1.59 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min.

### Examples 395 and 396

### 1-Methyl-4-((2S,5R)-2-methyl-5-propyl-4-(1-(4-(trifluoromethyl)phenyl)ethyl)piperazin-1-yl)-2-oxo-1,2-dihydropyrido[3,2-d]pyrimidine-6-carbonitrile

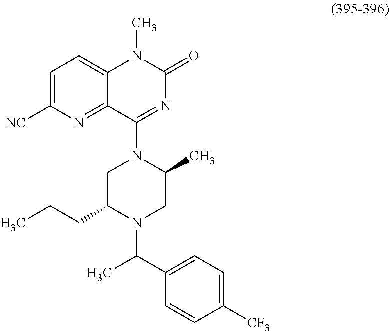 [SMILES: CCC[C@@H]1CN(c2nc(=O)n(C)c3ccc(C#N)nc23)[C@@H](C)CN1C(C)c1ccc(C(F)(F)F)cc1]

To a solution of 6-chloro-1-methyl-4-((2S,5R)-2-methyl-5-propyl-4-(1-(4-(trifluoromethyl)phenyl)ethyl)piperazin-1-yl)pyrido[3,2-d]pyrimidin-2(1H)-one (0.1 g, 0.19 mmol) in DMF (2 mL) were added zinc cyanide (0.046 g, 0.39 mmol), zinc (0.7 mg, 9.8 μmol) and triethylamine (0.1 mL, 0.59 mmol) followed by dichloro[9,9-dimethyl-4,5-bis(diphenylphosphino)xanthene]palladium(II) (0.015 g, 0.02 mmol) at room temperature under argon atmosphere. The reaction mixture was heated at 90° C. overnight. The reaction mixture was diluted with EtOAc (50 mL) and filtered through Celite® pad, washed with additional ethyl acetate (2×50 mL). The filtrate was washed with water (50 mL), brine, dried over Na₂SO₄ and concentrated under reduced pressure to yield the crude product, which was purified by preparative HPLC (HPLC method: Column: YMC EXRS (250×19 mm, 5 μm); mobile phase A: 10 mM ammonium acetate in water pH ˜4.5; mobile phase B: acetonitrile Flow: 20 mL/min) to yield Example 395 and Example 396.

EXAMPLE 395: (13 mg, 14% yield). LCMS: m/z=499.3 [M+H]⁺; rt 2.376 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50° C.). ¹H NMR (400 MHz, DMSO-d₆) δ (ppm)=8.22 (br d, J=8.8 Hz, 1H), 7.98 (d, J=8.8 Hz, 1H), 7.70-7.72 (m, 2H), 7.59-7.61 (m, 2H), 5.84-5.59 (m, 1H), 5.10-4.67 (m, 1H), 3.91-3.75 (m, 1H), 3.38-3.43 (m, 4H), 2.86-2.70 (m, 2H), 2.47-2.36 (m, 1H), 1.63-1.51 (m, 1H), 1.47-1.18 (m, 8H), 0.9-0.99 (m, 1H), 0.75-0.59 (m, 3H).

EXAMPLE 396: (13 mg, 13% yield); LCMS: m/z=499.3 [M+H]⁺; rt 2.436 min; (LCMS Method: Column: XBridge BEH XP C18 (50×2.1 mm, 2.5 μm); mobile phase A: 95% water: 5% acetonitrile; 10 mM NH₄OAc; mobile phase B: 5% water: 95% acetonitrile; 10 mM NH₄OAC; Flow: 1.1 mL/min; Temp: 50° C.). ¹H NMR (400 MHz, DMSO-d₆) δ (ppm)=8.25 (br d, J=2.4 Hz, 1H), 8.06-7.92 (m, 1H), 7.77-7.65 (m, 2H), 7.65-7.54 (m, 2H), 6.09-5.44 (m, 1H), 5.04-4.68 (m, 1H), 3.81-3.59 (m, 2H), 3.44 (s, 3H), 3.28-3.13 (m, 1H), 2.52-2.61 (m, 1H), 2.24-2.05 (m, 1H), 1.72-1.48 (m, 2H), 1.47-1.15 (m, 8H), 0.98-0.75 (m, 3H).

### Intermediate 66

### tert-Butyl (2S,5S)-5-(hydroxymethyl)-2-methyl-4-(1-(4-(trifluoromethyl)phenyl)propyl)piperazine-1-carboxylate (Diasteromeric Mixture)

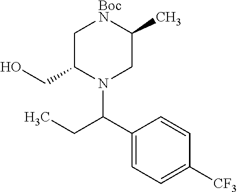 [SMILES: CCC(c1ccc(C(F)(F)F)cc1)N1C[C@H](C)N(C(=O)OC(C)(C)C)C[C@H]1CO]

To a stirred solution of tert-butyl (2S,5S)-5-(hydroxymethyl)-2-methylpiperazine-1-carboxylate (1.0 g, 4.34 mmol) in acetonitrile (8 mL), 2,2,6,6-tetramethylpiperidine (3.7 mL, 21.7 mmol), 1-(4-(trifluoromethyl)phenyl)propyl methanesulfonate (1.84 g, 6.51 mmol) and sodium iodide (0.65 g, 4.34 mmol) were added sequentially at room temperature. The reaction mixture was heated at 60° C. for 14 h. and cooled to room temperature. The solvent was removed under reduced pressure to give the crude product, which was purified by silica gel flash column chromatography (30-50% EtOAc in n-hexane) to afford tert-butyl (2S,5S)-5-(hydroxymethyl)-2-methyl-4-(1-(4-(trifluoromethyl)phenyl)propyl)piperazine-1-carboxylate (800 mg, 44% yield). LCMS: m/z, 417.3 [M+H]⁺; rt 3.68 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Intermediate 67

### tert-Butyl (2S,5S)-5-(methoxymethyl)-2-methyl-4-(1-(4-(trifluoromethyl)phenyl)propyl)piperazine-1-carboxylate (Diasteromeric Mixture)

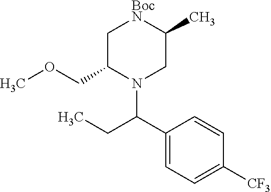 [SMILES: CCC(c1ccc(C(F)(F)F)cc1)N1C[C@H](C)N(C(=O)OC(C)(C)C)C[C@H]1COC]

To a stirred solution of tert-butyl (2S,5S)-5-(hydroxymethyl)-2-methyl-4-(1-(4-(trifluoromethyl)phenyl)propyl)piperazine-1-carboxylate (200 mg, 0.480 mmol) in THF (5 mL) was added NaH (58 mg, 1.44 mmol, 60% w/w) at 0° C. under nitrogen. The reaction mixture was stirred for 10 min. Iodomethane (0.15 mL, 2.40 mmol) was added. The reaction mixture was allowed to warm to room temperature and stirred for 2 h. The reaction mixture was then cooled to 0° C. The reaction was quenched with ice-cold water. The reaction mixture was extracted with EtOAc (2×50 mL). The combined organic extract was washed with brine, dried over Na₂SO₄ and concentrated to give the crude product, which was purified by silica gel flash column chromatography (15-20% EtOAc in n-hexane) to afford a tert-butyl (2S,5S)-5-(methoxymethyl)-2-methyl-4-(1-(4-(trifluoromethyl)phenyl)propyl) piperazine-1-carboxylate (130 mg, 63% yield). LCMS: m/z, 431.3 [M+H]⁺; rt 1.55 and 1.59 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 68

### (2S,5S)-2-(Methoxymethyl)-5-methyl-1-(1-(4-(trifluoromethyl)phenyl)propyl)piperazine, TFA (Diastereomeric Mixture)

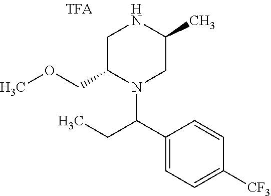 [SMILES: CCC(c1ccc(C(F)(F)F)cc1)N1C[C@H](C)NC[C@H]1COC]

To a stirred solution of tert-butyl (2S,5S)-5-(methoxymethyl)-2-methyl-4-(1-(4-(trifluoromethyl)phenyl)propyl)piperazine-1-carboxylate (150 mg, 0.35 mmol) in dry DCM (4 mL) was added TFA (0.3 mL, 3.50 mmol) at 0° C. The reaction mixture was allowed to warm to room temperature and stirred for 2 h. The solvent was removed under reduced pressure to afford (2S,5S)-2-(methoxymethyl)-5-methyl-1-(1-(4-(trifluoromethyl) phenyl)propyl)piperazine, TFA (120 mg, 77% yield). LCMS: m/z=331.3 [M+H]⁺; rt 1.36 and 1.38 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Example 397

### 6-Chloro-4-((2S,5S)-5-(methoxymethyl)-2-methyl-4-(1-(4-(trifluoromethyl)phenyl) propyl)piperazin-1-yl)-1-methylpyrido[3,2-d]pyrimidin-2(1H)-one (Diastereomeric Mixture)

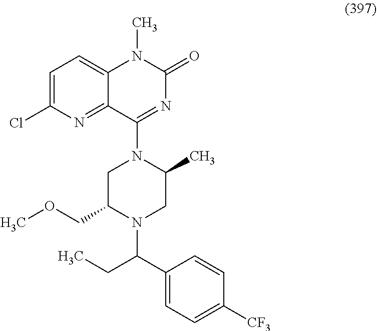 [SMILES: CCC(c1ccc(C(F)(F)F)cc1)N1C[C@H](C)N(c2nc(=O)n(C)c3ccc(Cl)nc23)C[C@H]1COC]

To a stirred solution of (2S,5S)-2-(methoxymethyl)-5-methyl-1-(1-(4-(trifluoromethyl)phenyl)propyl)piperazine, TFA (130 mg, 0.29 mmol) in acetonitrile (5 mL), DIPEA (0.5 mL, 2.93 mmol) and 4,6-dichloro-1-methylpyrido[3,2-d]pyrimidin-2(1H)-one (101 mg, 0.44 mmol) were added sequentially at room temperature followed by heating the mixture at 80° C. for 12 h. The reaction mixture was then cooled to room temperature and the solvent was removed under reduced pressure to give the crude product, which was purified by silica gel column chromatography using 0-10% MeOH in CHCl₃ to afford 6-chloro-4-((2S,5S)-5-(methoxymethyl)-2-methyl-4-(1-(4-(trifluoromethyl)phenyl)propyl)piperazin-1-yl)-1-methylpyrido[3,2-d]pyrimidin-2(1H)-one (120 mg, 78% yield). LCMS: m/z, 524.3 [M+H]⁺; rt 1.28 and 1.34 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.3 minute hold at 100% B, flow: 0.7 mL/min.

### Examples 398 and 399

### 4-((2S,5S)-5-(Methoxymethyl)-2-methyl-4-(1-(4-(trifluoromethyl)phenyl)propyl) piperazin-1-yl)-1-methyl-2-oxo-1,2-dihydropyrido[3,2-d]pyrimidine-6-carbonitrile

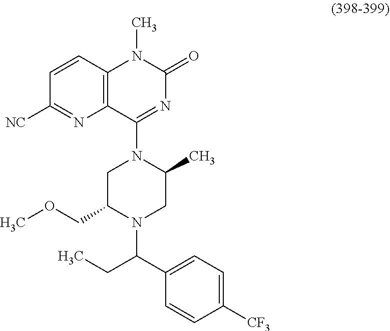 [SMILES: CCC(c1ccc(C(F)(F)F)cc1)N1C[C@H](C)N(c2nc(=O)n(C)c3ccc(C#N)nc23)C[C@H]1COC]

To a stirred solution of 6-chloro-4-((2S,5S)-5-(methoxymethyl)-2-methyl-4-(1-(4-(trifluoromethyl)phenyl)propyl)piperazin-1-yl)-1-methylpyrido[3,2-d]pyrimidin-2(1H)-one (120 mg, 0.23 mmol) in DMF (5 mL) were added zinc (20 mg, 0.30 mmol) and TEA (0.16 mL, 1.14 mmol). The reaction mixture was then flushed with argon for 5 minutes, followed by the addition of zinc cyanide (81 mg, 0.69 mmol) and dichloro[9,9-dimethyl-4,5-bis(diphenylphosphino)xanthene]palladium(II) (17.3 mg, 0.02 mmol). The reaction mixture was heated at 90° C. for 12 h, cooled to room temperature and concentrated under reduced pressure to afford the product, which was purified using preparative HPLC (HPLC Method: Column: Sunfire C18 (150×19 mm, 5 μm); mobile phase A: 10 mM ammonium acetate in water; mobile phase B: acetonitrile; Gradient: 0-100% B over 18 minutes, then a 5 minute hold at 100% B; Flow: 19 mL/min).

EXAMPLE 398: (11.6 mg, 10% yield): LCMS: m/z, 515.3 [M+H]⁺; rt 2.22 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.23 (d, J=9.0 Hz, 1H), 7.98 (d, J=9.0 Hz, 1H), 7.74 (d, J=8.1 Hz, 2H), 7.53 (d, J=7.8 Hz, 2H), 6.10-5.37 (m, 1H), 5.01-4.75 (m, 1H), 3.86-3.70 (m, 1H), 3.58-3.38 (m, 5H), 3.23-2.86 (m, 6H), 2.82-2.72 (m, 1H), 1.98-1.82 (m, 1H), 1.74-1.59 (m, 1H), 1.44-1.11 (m, 3H), 0.83-0.61 (m, 3H).

EXAMPLE 399: (10.5 mg, 9% yield): LCMS: m/z, 515.3 [M+H]⁺; rt 2.26 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=8.24 (d, J=8.8 Hz, 1H), 7.99 (d, J=8.8 Hz, 1H), 7.70 (d, J=8.1 Hz, 2H), 7.56 (d, J=8.1 Hz, 2H), 6.05-5.40 (m, 1H), 5.05-4.74 (m, 1H), 3.79-3.49 (m, 3H), 3.44 (s, 3H), 3.39-3.35 (m, 2H), 3.28-3.18 (m, 1H), 3.15-3.00 (m, 2H), 2.71-2.58 (m, 1H), 2.40-2.16 (m, 1H), 2.06-1.89 (m, 1H), 1.78-1.60 (m, 1H), 1.40-1.17 (m, 3H), 0.66-0.63 (m, 3H).

The examples in the Table 17 were prepared according to the general procedure described in Examples 398 and 399, using ethyl iodide instead of methyl iodide in the synthetic sequence. Mixture of diastereomers were separated at the final stage using either preparative chromatography or preparative chiral chromatography. The absolute stereochemistry was not assigned at the carbon-nitrogen bond.

TABLE 17

| Ex. | | Stereo | LCMS | LCMS | |
|---|---|---|---|---|---|
| No. | Structure | Chem. | Method | rt | [M + H]⁺ |
| 400 401 | 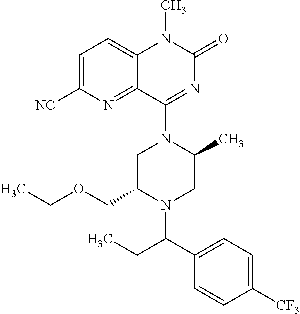 [SMILES: CCOC[C@@H]1CN(c2nc(=O)n(C)c3ccc(C#N)nc23)[C@@H](C)CN1C(CC)c1ccc(C(F)(F)F)cc1] | H H | C C | 2.32 2.36 | 529.3 529.3 |

### Intermediate 69

### tert-Butyl (2S,5S)-5-(chloromethyl)-2-methyl-4-(1-(4-(trifluoromethyl)phenyl)ethyl)piperazine-1-carboxylate

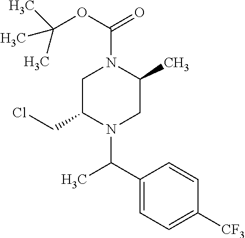 [SMILES: CC(c1ccc(C(F)(F)F)cc1)N1C[C@H](C)N(C(=O)OC(C)(C)C)C[C@H]1CCl]

To a stirred solution of tert-butyl (2S,5S)-5-(hydroxymethyl)-2-methyl-4-(1-(4-(trifluoromethyl)phenyl)ethyl)piperazine-1-carboxylate (2.3 g, 5.71 mmol) in DCM (25 mL) were added Et₃N (2.4 mL, 17.14 mmol) and DMAP (0.035 g, 0.286 mmol) followed by methanesulfonyl chloride (0.9 mL, 11.43 mmol) at 0° C. The reaction mixture was stirred at room temperature for 16 h. Water was added to quench the reaction. The mixture was extracted with DCM (2×50 mL), washed with water, brine and dried over anhydrous sodium sulphate. Evaporation of the solvent under reduced pressure furnished tert-butyl (2S,5S)-5-(chloromethyl)-2-methyl-4-(1-(4-(trifluoromethyl)phenyl)ethyl) piperazine-1-carboxylate (2 g, 81% yield). LCMS: m/z, 421.2 [M+H]⁺; retention time 4.17 and 4.26 min. [LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm].

### Intermediate 70

### tert-Butyl (2S,5S)-5-(azidomethyl)-2-methyl-4-(1-(4-(trifluoromethyl)phenyl)ethyl)piperazine-1-carboxylate

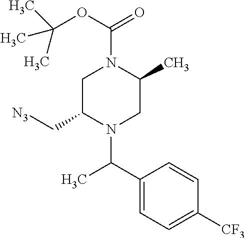 [SMILES: CC(c1ccc(C(F)(F)F)cc1)N1C[C@H](C)N(C(=O)OC(C)(C)C)C[C@H]1CN=[N+]=[N-]]

To a stirred solution of tert-butyl (2S,5S)-5-(chloromethyl)-2-methyl-4-(1-(4-(trifluoromethyl)phenyl)ethyl)piperazine-1-carboxylate (1.5 g, 3.56 mmol) in DMF (40 mL) were added tetrabutylammonium iodide (0.13 g, 0.36 mmol) and sodium azide (0.7 g, 10.7 mmol) at room temperature. The reaction mixture was heated at 80° C. for 16 h, cooled to room temperature, diluted with water, extracted with EtOAc (2×50 mL), washed with water and brine solution. The combined organic extract was dried over sodium sulphate and the solvent was concentrated under reduced pressure to obtain the crude product, which was purified by silica gel flash column chromatography (50-60% EtOAc in n-hexane) to afford tert-butyl (2S,5S)-5-(azidomethyl)-2-methyl-4-(1-(4-(trifluoromethyl)phenyl)ethyl) piperazine-1-carboxylate (1.2 g, 73% yield). LCMS: m/z, 428.2 [M+H]⁺; rt 4.16 & 4.23 min. [LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm].

### Intermediate 71

### (2S,5S)-2-(Azidomethyl)-5-methyl-1-(1-(4-(trifluoromethyl)phenyl)ethyl)piperazine·HCl Salt (Diastereomeric Mixture)

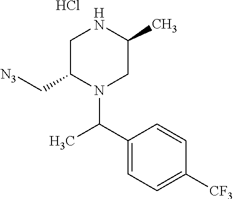 [SMILES: Cl ; CC(c1ccc(C(F)(F)F)cc1)N1C[C@H](C)NC[C@H]1CN=[N+]=[N-]]

To a stirred solution of tert-butyl (2S,5S)-5-(azidomethyl)-2-methyl-4-(1-(4-(trifluoromethyl)phenyl)ethyl) piperazine-1-carboxylate (0.6 g, 1.40 mmol) in DCM (15 mL), HCl (4 N in dioxane) (0.43 mL, 14.0 mmol) was added drop wise at room temperature. The reaction mixture was stirred for 3 h. The solvent was removed under reduced pressure, co-distilled with acetonitrile (3×10 mL), and dried to afford (2S,5S)-2-(azidomethyl)-5-methyl-1-(1-(4-(trifluoromethyl)phenyl)ethyl) piperazine, HCl salt (0.42 g, 81% yield) as an off-white solid. LCMS: m/z, 328.2 [M+H]⁺; rt 2.07 and 2.19 min. (LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Example 402

### 4-((2S,5S)-5-(Azidomethyl)-2-methyl-4-(1-(4-(trifluoromethyl)phenyl)ethyl)piperazin-1-yl)-6-chloro-1-methylpyrido[3,2-d]pyrimidin-2(1H)-one (Diastereomeric Mixture)

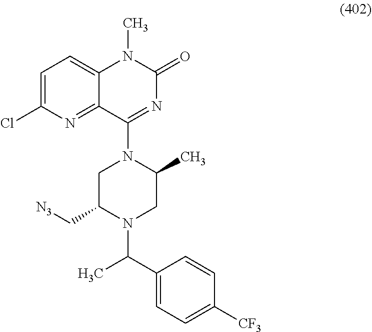 [SMILES: CC(c1ccc(C(F)(F)F)cc1)N1C[C@H](C)N(c2nc(=O)n(C)c3ccc(Cl)nc23)C[C@H]1CN=[N+]=[N-]]

To a stirred solution of (2S,5S)-2-(azidomethyl)-5-methyl-1-(1-(4-(trifluoromethyl)phenyl)ethyl) piperazine·HCl salt (400 mg, 1.22 mmol) in acetonitrile (10 mL), DIPEA (1.1 mL, 6.11 mmol) and 4,6-dichloro-1-methylpyrido[3,2-d]pyrimidin-2(1H)-one (281 mg, 1.22 mmol) were added sequentially at room temperature followed by heating at 80° C. for 12 h. The reaction mixture was cooled to room temperature and the solvent was removed under reduced pressure to give the crude product, which was purified by silica gel column chromatography using 0-10% MeOH in CHCl₃ to afford 4-((2S,5S)-5-(azidomethyl)-2-methyl-4-(1-(4-(trifluoromethyl)phenyl)ethyl)piperazin-1-yl)-6-chloro-1-methylpyrido[3,2-d]pyrimidin-2(1H)-one (420 mg, 43% yield). LCMS: m/z, 521.3 [M+H]⁺; rt 3.59 and 3.66 min. (LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Example 403

### 4-((2S,5R)-5-(Aminomethyl)-2-methyl-4-(1-(4-(trifluoromethyl)phenyl)ethyl)piperazin-1-yl)-6-chloro-1-methylpyrido[3,2-d]pyrimidin-2(1H)-one (Diastereomeric Mixture)

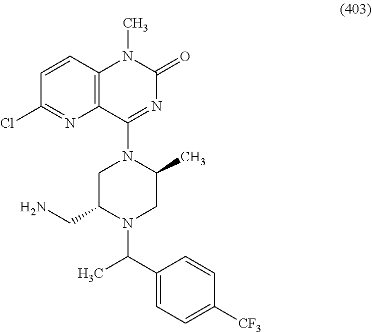 [SMILES: CC(c1ccc(C(F)(F)F)cc1)N1C[C@H](C)N(c2nc(=O)n(C)c3ccc(Cl)nc23)C[C@H]1CN]

To a stirred solution of 4-((2S,5S)-5-(azidomethyl)-2-methyl-4-(1-(4-(trifluoromethyl)phenyl)ethyl)piperazin-1-yl)-6-chloro-1-methylpyrido[3,2-d]pyrimidin-2(1H)-one (200 mg, 0.38 mmol) in THF (7 mL) and water (3 mL) was added polymer bound triphenylphosphine (1.4 mmol to 2 mmol per gram) (302 mg, 1.15 mmol) at room temperature. The reaction mixture was heated at 65° C. for 16 h. The reaction mixture was cooled to room temperature, filtered through Celite® pad and washed with excess EtOAc (20 mL). The filtrate was dried over sodium sulphate and concentrated under reduced pressure to give 4-((2S,5R)-5-(aminomethyl)-2-methyl-4-(1-(4-(trifluoromethyl) phenyl)ethyl)piperazin-1-yl)-6-chloro-1-methylpyrido[3,2-d]pyrimidin-2(1H)-one (150 mg, 41% yield). LCMS: m/z, 495.1 [M+H]⁺; rt 1.38 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.3 minute hold at 100% B, flow: 0.7 mL/min.

### Examples 404 and 405

### 4-((2S,5S)-5-(Methoxymethyl)-2-methyl-4-(1-(4-(trifluoromethyl)phenyl)ethyl)piperazin-1-yl)-1-methyl-2-oxo-1,2-dihydropyrido[3,2-d]pyrimidine-6-carbonitrile

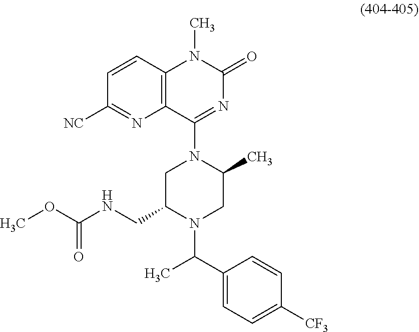 [SMILES: COC(=O)NC[C@@H]1CN(c2nc(=O)n(C)c3ccc(C#N)nc23)[C@@H](C)CN1C(C)c1ccc(C(F)(F)F)cc1]

To a stirred solution of 4-((2S,5R)-5-(aminomethyl)-2-methyl-4-(1-(4-(trifluoromethyl)phenyl)ethyl)piperazin-1-yl)-6-chloro-1-methylpyrido[3,2-d]pyrimidin-2(1H)-one (200 mg, 0.40 mmol) in DCM (5 mL) were added DIPEA (0.11 mL, 0.61 mmol) and methyl chloroformate (0.1 mL, 1.21 mmol) at room temperature. The reaction mixture was stirred for 3 h. The solvent was removed under reduced pressure and co-distilled with acetonitrile (2×5 mL) to afford methyl (((2R,5S)-4-(6-chloro-1-methyl-2-oxo-1,2-dihydropyrido[3,2-d]pyrimidin-4-yl)-5-methyl-1-(1-(4-(trifluoromethyl)phenyl) ethyl)piperazin-2-yl)methyl)carbamate (170 mg, 76% yield).

To a stirred solution of methyl (((2R,5S)-4-(6-chloro-1-methyl-2-oxo-1,2-dihydropyrido[3,2-d]pyrimidin-4-yl)-5-methyl-1-(1-(4-(trifluoromethyl)phenyl)ethyl) piperazin-2-yl)methyl)carbamate (140 mg, 0.25 mmol) in DMF (5 mL) were added zinc (25 mg, 0.38 mmol) and TEA (0.15 mL, 1.08 mmol). The reaction mixture was flushed with argon for 5 minutes, followed by the addition of zinc cyanide (89 mg, 0.76 mmol) and dichloro[9,9-dimethyl-4,5-bis(diphenylphosphino)xanthene] palladium(II) (19 mg, 0.025 mmol). The reaction mixture was heated at 90° C. for 12 h, cooled to room temperature and concentrated under reduced pressure to afford the crude product, which was purified using preparative HPLC. (Column: Sunfire C18 (150 mm×19 mm, 5 μm); mobile phase A: 10 mM NH₄OAc in water; mobile phase B: acetonitrile; Gradient: 50-75% B over 20 minutes, then a 5 minute hold at 100% B; Flow: 19 mL/min; UV detection: 220 nm).

EXAMPLE 404: (40 mg, 28% yield): LCMS: m/z, 544.3 [M+H]⁺; rt 3.08 min; (LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.25 (d, J=8.5 Hz, 1H), 8.00 (br d, J=9.0 Hz, 1H), 7.75-7.68 (m, J=8.0 Hz, 2H), 7.62 (br d, J=8.0 Hz, 2H), 7.01-6.76 (m, 1H), 5.96-5.27 (m, 1H), 5.12-4.65 (m, 1H), 3.91-3.51 (m, 3H), 3.49-3.36 (m, 4H), 3.30-3.20 (m, 3H), 3.04-2.92 (m, 1H), 2.65-2.53 (m, 1H), 2.46-2.35 (m, 1H), 1.43-1.33 (m, 4H), 1.27-1.17 (m, 2H).

EXAMPLE 405: (50 mg, 35% yield): LCMS: m/z, 544.4 [M+H]⁺; rt 3.01 min; (LCMS Method: Column: Kinetex −18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.30-8.19 (m, 1H), 8.00 (br s, 1H), 7.72 (d, J=8.0 Hz, 2H), 7.61 (d, J=8.0 Hz, 2H), 6.80 (br s, 1H), 5.72-5.46 (m, 1H), 5.13-4.54 (m, 1H), 4.03-3.90 (m, 1H), 3.73-3.58 (m, 1H), 3.52-3.41 (m, 4H), 3.24-3.13 (m, 2H), 3.08-2.83 (m, 3H), 2.83-2.77 (m, 1H), 2.75-2.69 (m, 1H), 1.56-1.26 (m, 6H).

### Intermediate 72

### tert-Butyl (2R,5R)-4-(6-chloro-1-methyl-2-oxo-1,2-dihydropyrido[3,2-d]pyrimidin-4-yl)-5-(hydroxymethyl)-2-methylpiperazine-1-carboxylate (Homochiral)

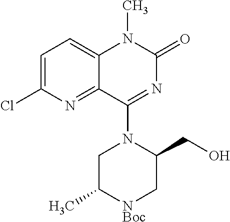 [SMILES: C[C@@H]1CN(c2nc(=O)n(C)c3ccc(Cl)nc23)[C@@H](CO)CN1C(=O)OC(C)(C)C]

To a stirred solution of tert-butyl (2R,5R)-5-(hydroxymethyl)-2-methylpiperazine-1-carboxylate (0.60 g, 2.61 mmol) in acetonitrile (8 mL), DIPEA (3.8 mL, 21.7 mmol) and 4,6-dichloro-1-methylpyrido[3,2-d]pyrimidin-2(1H)-one (0.5 g, 2.17 mmol) were added at room temperature followed by heating at 80° C. for 12 h. The reaction mixture was cooled to room temperature. Solvent was removed under reduced pressure to give the crude product, which was purified by silica gel column chromatography using 7-10% MeOH in CHCl₃ to afford tert-butyl (2R,5R)-4-(6-chloro-1-methyl-2-oxo-1,2-dihydropyrido[3,2-d]pyrimidin-4-yl)-5-(hydroxymethyl)-2-methylpiperazine-1-carboxylate (0.7 g, 76% yield). LCMS: m/z, 424.2 [M+H]⁺; rt 2.08 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Intermediate 73

### tert-Butyl (2R,5R)-4-(6-cyano-1-methyl-2-oxo-1,2-dihydropyrido[3,2-d]pyrimidin-4-yl)-5-(hydroxymethyl)-2-methylpiperazine-1-carboxylate (Homochiral)

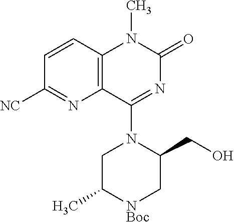 [SMILES: C[C@@H]1CN(c2nc(=O)n(C)c3ccc(C#N)nc23)[C@@H](CO)CN1C(=O)OC(C)(C)C]

To a stirred solution of tert-butyl (2R,5R)-4-(6-chloro-1-methyl-2-oxo-1,2-dihydropyrido[3,2-d]pyrimidin-4-yl)-5-(hydroxymethyl)-2-methylpiperazine-1-carboxylate (300 mg, 0.71 mmol) in DMF (10 mL) were added zinc (5 mg, 0.07 mmol) and TEA (0.5 mL, 3.54 mmol). The reaction mixture was flushed with argon for 5 minutes, followed by the addition of zinc cyanide (250 mg, 2.12 mmol) and dichloro[9,9-dimethyl-4,5-bis(diphenylphosphino)xanthene]palladium(II) (53.5 mg, 0.07 mmol). The reaction mixture was heated at 95° C. for 6 h, cooled to room temperature, filtered through a Celite® pad and washed with DCM (50 mL). The filtrate was concentrated under reduced pressure to afford the crude product, which was purified by silica gel column chromatography using 0-10% MeOH in CHCl₃ to afford tert-butyl (2R,5R)-4-(6-cyano-1-methyl-2-oxo-1,2-dihydropyrido[3,2-d]pyrimidin-4-yl)-5-(hydroxymethyl)-2-methylpiperazine-1-carboxylate (220 mg, 75% yield). LCMS: m/z, 415.2 [M+H]⁺; rt 1.84 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Intermediate 74

### 4-((2R,5R)-2-(Hydroxymethyl)-5-methylpiperazin-1-yl)-1-methyl-2-oxo-1,2-dihydropyrido[3,2-d]pyrimidine-6-carbonitrile, TFA

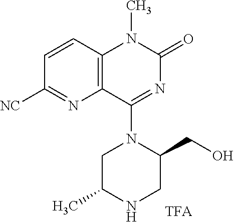 [SMILES: C[C@@H]1CN(c2nc(=O)n(C)c3ccc(C#N)nc23)[C@@H](CO)CN1]

To a stirred solution of tert-butyl (2R,5R)-4-(6-cyano-1-methyl-2-oxo-1,2-dihydropyrido[3,2-d]pyrimidin-4-yl)-5-(hydroxymethyl)-2-methylpiperazine-1-carboxylate (250 mg, 0.60 mmol) in dry DCM (8 mL) was added TFA (0.2 mL, 3.02 mmol) at 0° C. The reaction mixture was allowed to warm to room temperature and stirred for 2 h. The solvent was removed under reduced pressure to afford 4-((2R,5R)-2-(hydroxymethyl)-5-methylpiperazin-1-yl)-1-methyl-2-oxo-1,2-dihydropyrido[3,2-d] pyrimidine-6-carbonitrile, TFA (200 mg, 77% yield). LCMS: m/z=315.2 [M+H]⁺; rt 0.47 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 2.2 minute hold at 100% B, flow: 0.7 mL/min.

### Examples 406 and 407

### 4-((2R,5R)-2-(Hydroxymethyl)-5-methyl-4-(1-(4-(trifluoromethyl)phenyl)ethyl) piperazin-1-yl)-1-methyl-2-oxo-1,2-dihydropyrido[3,2-d]pyrimidine-6-carbonitrile

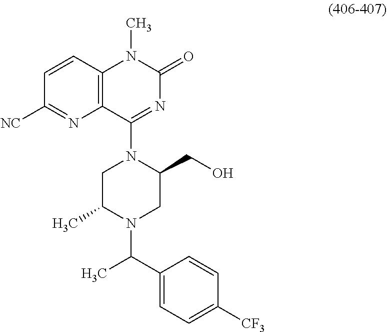 [SMILES: CC(c1ccc(C(F)(F)F)cc1)N1C[C@H](CO)N(c2nc(=O)n(C)c3ccc(C#N)nc23)C[C@H]1C]

To a stirred solution of 4-((2R,5R)-2-(hydroxymethyl)-5-methylpiperazin-1-yl)-1-methyl-2-oxo-1,2-dihydropyrido[3,2-d]pyrimidine-6-carbonitrile, TFA (80 mg, 0.19 mmol) in acetonitrile (4 mL) were added DIPEA (0.33 mL, 1.87 mmol) and potassium iodide (31 mg, 0.19 mmol) followed by 1-(1-chloroethyl)-4-(trifluoromethyl)benzene (78 mg, 0.37 mmol). The reaction mixture was heated at 85° C. for 4 h. and then cooled to room temperature. The solvent was removed under reduced pressure to yield the crude product, which was purified by preparative HPLC (HPLC Method: Sunfire OBD (250×30 mm, 5 μm); mobile phase A: 10 mM ammonium acetate in water; mobile phase B: acetonitrile; Gradient: 65-100% B over 16 minutes, then a 5 minute hold at 100% B; Flow: 25 mL/min).

EXAMPLE 406 (18.6 mg, 20% yield); LCMS: m/z=487.2 [M+H]⁺; rt 2.76 min; (LCMS Method: Column: Kinetex XB-C18 (3×75 mm) 2.6 μm; mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.29-8.19 (m, 1H), 7.99 (d, J=9.0 Hz, 1H), 7.72 (d, J=8.5 Hz, 2H), 7.62 (d, J=8.0 Hz, 2H), 6.06-5.28 (m, 1H), 5.14-4.87 (m, 1H), 4.03-3.90 (m, 1H), 3.78-3.70 (m, 2H), 3.57-3.42 (m, 5H), 3.20-3.08 (m, 1H), 2.99-2.71 (m, 2H), 1.27 (br d, J=6.5 Hz, 3H), 0.90 (br d, J=6.0 Hz, 3H).

EXAMPLE 407 (23 mg, 25% yield); LCMS: m/z=487.2 [M+H]⁺; rt 2.81 min; (LCMS Method: Column: Kinetex XB-C18 (3×75 mm) 2.6 μm; mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.28-8.19 (m, 1H), 8.00 (br d, J=9.0 Hz, 1H), 7.69 (d, J=8.0 Hz, 2H), 7.63-7.57 (m, 2H), 5.85-5.60 (m, 1H), 4.93-4.78 (m, 1H), 4.70-4.45 (m, 2H), 3.80-3.67 (m, 2H), 3.65-3.58 (m, 2H), 3.45 (s, 3H), 3.06-2.70 (m, 1H), 2.61-2.55 (m, 1H), 1.27 (d, J=6.5 Hz, 3H), 1.04 (d, J=6.5 Hz, 3H).

### Intermediate 75

### tert-Butyl (2R,5R)-4-(6-chloro-1-methyl-2-oxo-1,2-dihydropyrido[3,2-d]pyrimidin-4-yl)-5-(methoxymethyl)-2-methylpiperazine-1-carboxylate (Homochiral)

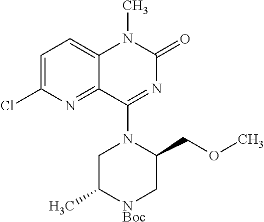 [SMILES: COC[C@H]1CN(C(=O)OC(C)(C)C)[C@H](C)CN1c1nc(=O)n(C)c2ccc(Cl)nc12]

To a stirred solution of tert-butyl (2R,5R)-4-(6-chloro-1-methyl-2-oxo-1,2-dihydropyrido[3,2-d]pyrimidin-4-yl)-5-(hydroxymethyl)-2-methylpiperazine-1-carboxylate (200 mg, 0.47 mmol) in THF (5 mL) was added NaH (57 mg, 1.42 mmol, 60% w/w) at 0° C. under nitrogen. The reaction mixture was stirred for 10 min. Next, iodomethane (0.06 mL, 0.94 mmol) was added. The reaction mixture was allowed to warm to room temperature and stirred for 2 h. The reaction mixture was cooled to 0° C. The reaction was quenched with the addition of ice cold water. The mixture was extracted with EtOAc (2×50 mL). The combined organic extract was washed with brine, dried over Na₂SO₄ and concentrated to give the crude product, which was purified by silica gel flash column chromatography (5-10% MeOH in DCM) to afford a tert-butyl (2R,5R)-4-(6-chloro-1-methyl-2-oxo-1,2-dihydropyrido[3,2-d]pyrimidin-4-yl)-5-(methoxymethyl)-2-methylpiperazine-1-carboxylate (140 mg, 68% yield). LCMS: m/z, 438.2 [M+H]⁺; rt 2.56 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm).

### Intermediate 76

### 6-Chloro-4-((2R,5R)-2-(methoxymethyl)-5-methylpiperazin-1-yl)-1-methylpyrido[3,2-d] pyrimidin-2(1H)-one, TFA

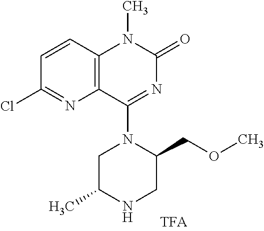 [SMILES: COC[C@H]1CN[C@H](C)CN1c1nc(=O)n(C)c2ccc(Cl)nc12]

To a stirred solution of tert-butyl (2R,5R)-4-(6-chloro-1-methyl-2-oxo-1,2-dihydropyrido[3,2-d]pyrimidin-4-yl)-5-(methoxymethyl)-2-methylpiperazine-1-carboxylate (190 mg, 0.43 mmol) in dry DCM (4 mL) was added TFA (0.17 mL, 2.17 mmol) at 0° C. The reaction mixture was allowed to warm to room temperature and stirred for 2 h. The solvent was removed under reduced pressure to afford 6-chloro-4-((2R,5R)-2-(methoxymethyl)-5-methylpiperazin-1-yl)-1-methylpyrido[3,2-d]pyrimidin-2(1H)-one, TFA (150 mg, 77% yield). LCMS: m/z=338.2 [M+H]⁺; rt 0.57 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 2.2 minute hold at 100% B, flow: 0.7 mL/min.

### Example 408

### 6-Chloro-4-((2R,5R)-2-(methoxymethyl)-5-methyl-4-(1-(4-(trifluoromethyl)phenyl)ethyl) piperazin-1-yl)-1-methylpyrido[3,2-d]pyrimidin-2(1H)-one

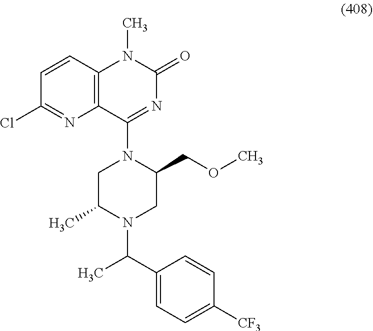 [SMILES: COC[C@H]1CN(C(C)c2ccc(C(F)(F)F)cc2)[C@H](C)CN1c1nc(=O)n(C)c2ccc(Cl)nc12]

To a stirred solution of 6-chloro-4-((2R,5R)-2-(methoxymethyl)-5-methylpiperazin-1-yl)-1-methylpyrido[3,2-d]pyrimidin-2(1H)-one, TFA (100 mg, 0.22 mmol) in acetonitrile (4 mL) was added DIPEA (0.4 mL, 2.21 mmol), followed by 1-(1-chloroethyl)-4-(trifluoromethyl)benzene (92 mg, 0.44 mmol) and NaI (33.2 mg, 0.21 mmol). The reaction mixture was heated at 85° C. for 4 h. The reaction mixture was cooled to room temperature and the solvent was removed under reduced pressure to yield the crude product, which was purified by silica gel flash column chromatography (5-10% MeOH in DCM) to afford a 6-chloro-4-((2R,5R)-2-(methoxymethyl)-5-methyl-4-(1-(4-(trifluoromethyl)phenyl)ethyl)piperazin-1-yl)-1-methylpyrido[3,2-d]pyrimidin-2(1H)-one (65 mg, 58% yield). LCMS: m/z=510.2 [M+H]⁺; rt 2.11 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 2.2 minute hold at 100% B, flow: 0.7 mL/min.

### Examples 409 and 410

### 4-((2R,5R)-2-(Methoxymethyl)-5-methyl-4-(1-(4-(trifluoromethyl)phenyl)ethyl) piperazin-1-yl)-1-methyl-2-oxo-1,2-dihydropyrido[3,2-d]pyrimidine-6-carbonitrile

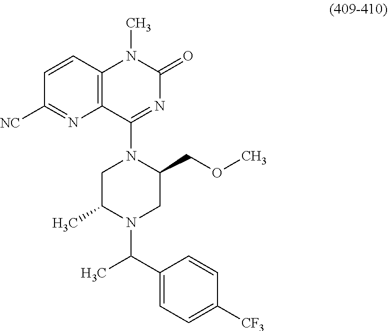 [SMILES: COC[C@H]1CN(C(C)c2ccc(C(F)(F)F)cc2)[C@H](C)CN1c1nc(=O)n(C)c2ccc(C#N)nc12]

To a stirred solution of 6-chloro-4-((2R,5R)-2-(methoxymethyl)-5-methyl-4-(1-(4-(trifluoromethyl)phenyl)ethyl)piperazin-1-yl)-1-methylpyrido[3,2-d]pyrimidin-2(1H)-one (80 mg, 0.16 mmol) in DMF (10 mL) were added zinc (10 mg, 0.16 mmol) and TEA (0.11 mL, 0.78 mmol). The reaction mixture was degassed with argon gas for 5 minutes, followed by the addition of zinc cyanide (92 mg, 0.78 mmol) and dichloro[9,9-dimethyl-4,5-bis(diphenylphosphino)xanthene]palladium(II) (12 mg, 0.02 mmol). The reaction mixture was heated at 95° C. for 6 h. The reaction mixture was cooled to room temperature, filtered through Celite® pad and washed with excess DCM (50 mL). The filtrate was concentrated under reduced pressure to afford the crude product, which was purified by SFC. SFC Method: Column: Chiralpak IC (250×4.6 mm, 5 μm); mobile phase: % CO₂=80%; co-solvent: 20% of NH₄OAc in acetonitrile:methanol (50:50), total flow: 80.0 g/min. back pressure: 100 bar; temperature: 30° C.; UV detection: 215 nm. Example 409; Isolate 1: First eluting peak, rt=3.09 min; Example 410 Isolate 2: Second eluting peak, rt=3.62 min.

EXAMPLE 409: (10.2 mg, 13% yield); LCMS: m/z=501.3 [M+H]⁺; rt 3.28 min; (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.29-8.20 (m, 1H), 8.01 (d, J=8.5 Hz, 1H), 7.74-7.70 (m, 2H), 7.65-7.61 (m, 2H), 5.61-5.10 (m, 1H), 4.56-4.39 (m, 1H), 3.92-3.86 (m, 2H), 3.78-3.71 (m, 3H), 3.13-3.05 (m, 3H), 2.88-2.77 (m, 1H), 1.26 (br d, J=6.5 Hz, 3H), 0.90 (br d, J=6.5 Hz, 3H).

EXAMPLE 410: (9.1 mg, 12% yield); LCMS: m/z=501.3 [M+H]⁺; rt 3.26 min; (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.29-8.21 (m, 1H), 8.01 (d, J=8.5 Hz, 1H), 7.70 (d, J=8.0 Hz, 2H), 7.60 (d, J=8.0 Hz, 2H), 5.85-5.60 (m, 1H), 5.05-4.64 (m, 1H), 3.77-3.57 (m, 5H), 3.45 (s, 3H), 3.21-3.17 (m, 1H), 3.13-3.06 (m, 2H), 2.62-2.58 (m, 1H), 2.39 (br dd, J=3.3, 8.3 Hz, 1H), 1.28 (d, J=6.5 Hz, 3H), 1.04 (br d, J=6.0 Hz, 3H).

### Intermediate 77

### Methyl (R)-2-((S)-2-(((benzyloxy)carbonyl)amino)-3-hydroxypropanamido)butanoate

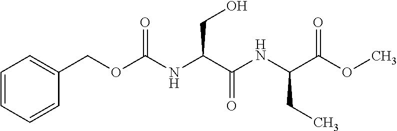 [SMILES: CC[C@@H](NC(=O)[C@H](CO)NC(=O)OCc1ccccc1)C(=O)OC]

To a stirred solution of (R)-2-aminobutanoate hydrochloride (4.82 g, 31.4 mmol), EDC (4.81 g, 25.1 mmol) and ((benzyloxy)carbonyl)-L-serine (5.0 g, 21.0 mmol) in dichloromethane (50 mL) was added DIPEA (11 mL, 62.7 mmol) dropwise. The resulting mixture was stirred under nitrogen at ambient temperature for 16 h. After removing solvent in vacuo at 40° C., the crude product was diluted with saturated sodium carbonate (20 mL), water (20 mL) and extracted with EtOAc (2×100 mL). The combined organic phase was washed with 1.5 M hydrochloric acid, brine, dried over sodium sulfate and concentrated under reduced pressure to yield methyl (R)-2-((S)-2-(((benzyloxy)carbonyl)amino)-3-hydroxypropanamido)butanoate (5.0 g, 71% yield) as an off-white solid. LCMS: m/z, 339.2 [M+H]⁺; rt 1.12 min. (LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm). ¹H NMR (400 MHz, DMSO-d₆) δ (ppm)=8.20 (br d, J=7.5 Hz, 1H), 7.41-7.27 (m, 5H), 7.13 (br d, J=8.5 Hz, 1H), 5.03 (s, 2H), 4.90-4.79 (m, 1H), 4.24-4.07 (m, 2H), 3.63-3.49 (m, 5H), 1.76-1.55 (m, 2H), 0.83 (t, J=7.3 Hz, 3H).

### Intermediate 78

### Methyl (R)-2-((S)-2-amino-3-hydroxypropanamido)butanoate

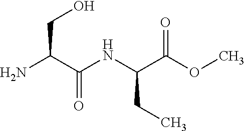

To a stirred solution of methyl (R)-2-((S)-2-(((benzyloxy)carbonyl)amino)-3-hydroxypropanamido)butanoate (4.0 g, 11.8 mmol) in MeOH (40 mL) was added 10% palladium on carbon (1.26 g, 1.18 mmol). The reaction mixture was stirred under a hydrogen atmosphere at 1 atm for 16 h. The reaction mixture was filtered through Celite® pad, washed with excess MeOH (50 mL) and the filtrate was removed under reduced pressure to give methyl (R)-2-((S)-2-amino-3-hydroxypropanamido)butanoate (2.3 g, 95% yield). LCMS: m/z, 205.2 [M+H]⁺; rt 1.19 min. LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: ELSD detector.

### Intermediate 79

### (3R,6S)-3-Ethyl-6-(hydroxymethyl)piperazine-2,5-dione

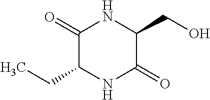

Methyl (R)-2-((S)-2-amino-3-hydroxypropanamido)butanoate (1.0 g, 4.90 mmol) was added to MeOH (8 mL). The reaction mixture was heated at 70° C. for 14 h. The reaction mixture was cooled to room temperature and concentrated under reduced pressure to give (3R,6S)-3-ethyl-6-(hydroxymethyl)piperazine-2,5-dione (0.7 g, 83% yield). ¹H NMR (400 MHz, DMSO-d₆) δ (ppm)=8.05 (s, 1H), 7.93 (s, 1H), 5.08 (br s, 1H), 3.86 (t, J=4.5 Hz, 1H), 3.77-3.67 (m, 2H), 3.56-3.49 (m, 1H), 1.85-1.56 (m, 2H), 0.81 (t, J=7.3 Hz, 3H).

### Intermediate 80

### ((2R,5R)-5-Ethylpiperazin-2-yl)methanol, 2 HCl

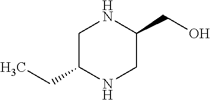

To (3R,6S)-3-ethyl-6-(hydroxymethyl)piperazine-2,5-dione (0.8 g, 4.65 mmol) was added a solution of 1 M borane in THF (35 mL, 34.8 mmol). The mixture was heated at 70° C. for 18 h. and then cooled to 0° C. Next, MeOH (10 mL) was gradually added, followed by the addition of 5 M hydrochloric acid (5 mL). The mixture was refluxed for 2 h at 70° C. and then cooled to ambient temperature. The resulting solid was filtered, triturated with THF (20 mL) and dried to give ((2R,5R)-5-ethylpiperazin-2-yl) methanol, 2 HCl (700 mg, 69% yield). ¹H NMR (400 MHz, DMSO-d₆) δ (ppm)=10.19-9.87 (m, 1H), 9.84-9.60 (m, 1H), 3.76-3.55 (m, 3H), 3.52-3.45 (m, 2H), 3.42-3.35 (m, 2H), 3.17-3.04 (m, 2H), 1.83-1.60 (m, 2H), 0.97 (t, J=7.5 Hz, 3H).

### Intermediate 81

### tert-Butyl (2R,5R)-2-ethyl-5-(hydroxymethyl)piperazine-1-carboxylate

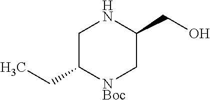 [SMILES: CC[C@@H]1CN[C@@H](CO)CN1C(=O)OC(C)(C)C]

To an ice cooled stirred solution of ((2R,5R)-5-ethylpiperazin-2-yl)methanol, 2 HCl (2.0 g, 9.21 mmol) in MeOH (10 mL), TEA (12.8 mL, 92 mmol) was added. The reaction mixture was stirred for 5 min. and Boc-anhydride (8.6 mL, 36.8 mmol) in MeOH (10 mL) was added dropwise over a period of 15 min. The reaction mixture was allowed to reach to room temperature, stirred for 1 h followed by heating at 50° C. for 16 h. The reaction mixture was concentrated and dissolved in EtOH (10 mL). A solution of NaOH (1.0 g, 25.0 mmol) in water (5 mL) was added and the reaction mixture was heated at 100° C. for 16 h, then cooled to room temperature, pH was adjusted to ˜9 by aqueous 1.5 N HCl and extracted with chloroform (3×80 mL). The combined organic extract was dried over sodium sulfate, filtered and concentrated to give tert-butyl (2R,5R)-2-ethyl-5-(hydroxymethyl)piperazine-1-carboxylate (1.8 g, 80% yield). ¹H NMR (400 MHz, METHANOL-d₄) δ (ppm)=3.97-3.90 (m, 1H), 3.89-3.82 (m, 1H), 3.71-3.63 (m, 1H), 3.58-3.52 (m, 1H), 3.15 (dd, J=4.5, 14.1 Hz, 1H), 3.02 (dd, J=5.0, 13.1 Hz, 1H), 2.92-2.85 (m, 1H), 2.60 (dd, J=2.8, 12.8 Hz, 1H), 1.97-1.82 (m, 1H), 1.68-1.57 (m, 1H), 1.48 (s, 9H), 0.90 (t, J=7.5 Hz, 3H).

### Intermediate 82

### tert-Butyl (2R,5R)-4-(6-chloro-1-methyl-2-oxo-1,2-dihydropyrido[3,2-d]pyrimidin-4-yl)-2-ethyl-5-(hydroxymethyl)piperazine-1-carboxylate (Homochiral)

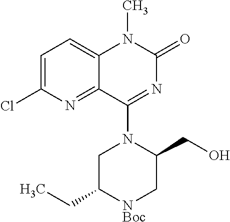 [SMILES: CC[C@@H]1CN(c2nc(=O)n(C)c3ccc(Cl)nc23)[C@@H](CO)CN1C(=O)OC(C)(C)C]

To a stirred solution of 4,6-dichloro-1-methylpyrido[3,2-d]pyrimidin-2(1H)-one (300 mg, 1.30 mmol) in acetonitrile (8 mL), DIPEA (2.3 mL, 13.04 mmol) and 4 tert-butyl (2R,5R)-2-ethyl-5-(hydroxymethyl)piperazine-1-carboxylate (382 mg, 1.57 mmol) were added sequentially at room temperature followed by heating at 80° C. for 12 h. The reaction mixture was cooled to room temperature and the solvent was removed under reduced pressure to give the crude product, which was purified by silica gel column chromatography using 7-10% MeOH in CHCl₃ to afford tert-butyl (2R,5R)-4-(6-chloro-1-methyl-2-oxo-1,2-dihydropyrido[3,2-d]pyrimidin-4-yl)-2-ethyl-5-(hydroxymethyl) piperazine-1-carboxylate (200 mg, 35% yield). LCMS: m/z, 438.3 [M+H]⁺; rt 1.43 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 83

### tert-Butyl (2R,5R)-4-(6-cyano-1-methyl-2-oxo-1,2-dihydropyrido[3,2-d]pyrimidin-4-yl)-2-ethyl-5-(hydroxymethyl)piperazine-1-carboxylate (Homochiral)

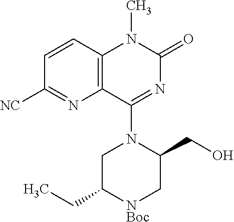 [SMILES: CC[C@@H]1CN(c2nc(=O)n(C)c3ccc(C#N)nc23)[C@@H](CO)CN1C(=O)OC(C)(C)C]

To a stirred solution of tert-butyl (2R,5R)-4-(6-chloro-1-methyl-2-oxo-1,2-dihydropyrido[3,2-d]pyrimidin-4-yl)-2-ethyl-5-(hydroxymethyl)piperazine-1-carboxylate (200 mg, 0.46 mmol) in DMF (10 mL) were added zinc (30 mg, 0.46 mmol) and TEA (0.32 mL, 2.28 mmol). The reaction mixture was flushed with argon for 5 minutes, followed by the addition of zinc cyanide (161 mg, 1.37 mmol) and dichloro[9,9-dimethyl-4,5-bis(diphenylphosphino)xanthene]palladium(II) (35 mg, 0.05 mmol). The reaction mixture was heated at 95° C. for 6 h, cooled to room temperature, filtered through Celite® pad and washed with excess DCM (50 mL). The filtrate was concentrated under reduced pressure to afford the crude product, which was purified by silica gel column chromatography using 7-10% MeOH in CHCl₃ to afford a tert-butyl (2R,5R)-4-(6-cyano-1-methyl-2-oxo-1,2-dihydropyrido[3,2-d]pyrimidin-4-yl)-2-ethyl-5-(hydroxymethyl) piperazine-1-carboxylate (150 mg, 77% yield). LCMS: m/z, 429.3 [M+H]⁺; rt 1.25 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 84

### 4-((2R,5R)-5-ethyl-2-(hydroxymethyl)piperazin-1-yl)-1-methyl-2-oxo-1,2-dihydropyrido[3,2-d]pyrimidine-6-carbonitrile, TFA

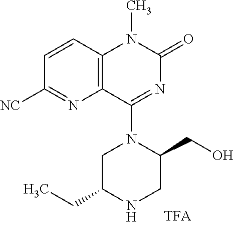 [SMILES: CC[C@@H]1CN(c2nc(=O)n(C)c3ccc(C#N)nc23)[C@@H](CO)CN1]

To a stirred solution of tert-butyl (2R,5R)-4-(6-cyano-1-methyl-2-oxo-1,2-dihydropyrido[3,2-d]pyrimidin-4-yl)-2-ethyl-5-(hydroxymethyl)piperazine-1-carboxylate (150 mg, 0.35 mmol) in dry DCM (4 mL) was added TFA (0.3 mL, 3.50 mmol) at 0° C. The reaction mixture was allowed to warm to room temperature and stirred for 2 h. The solvent was removed under reduced pressure to afford 4-((2R,5R)-5-ethyl-2-(hydroxymethyl)piperazin-1-yl)-1-methyl-2-oxo-1,2-dihydropyrido[3,2-d]pyrimidine-6-carbonitrile, TFA (120 mg, 77% yield). LCMS: m/z=329.2 [M+H]⁺; rt 0.56 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 2.2 minute hold at 100% B, flow: 0.7 mL/min.

### Examples 411 and 412

### 4-((2R,5R)-5-Ethyl-2-(hydroxymethyl)-4-(1-(4-(trifluoromethyl)phenyl)ethyl)piperazin-1-yl)-1-methyl-2-oxo-1,2-dihydropyrido[3,2-d]pyrimidine-6-carbonitrile

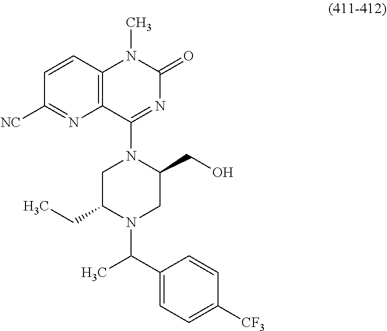 [SMILES: CC[C@@H]1CN(c2nc(=O)n(C)c3ccc(C#N)nc23)[C@@H](CO)CN1C(C)c1ccc(C(F)(F)F)cc1]

To a stirred solution of 4-((2R,5R)-5-ethyl-2-(hydroxymethyl)piperazin-1-yl)-1-methyl-2-oxo-1,2-dihydropyrido[3,2-d]pyrimidine-6-carbonitrile, TFA (80 mg, 0.18 mmol) in acetonitrile (4 mL) were added DIPEA (0.32 mL, 1.81 mmol) and sodium iodide (27.1 mg, 0.18 mmol) followed by the addition of 1-(1-chloroethyl)-4-(trifluoromethyl) benzene (45 mg, 0.22 mmol). The reaction mixture was heated at 85° C. for 4 h, cooled to room temperature and the solvent was removed under reduced pressure to yield the crude product, which was purified by preparative HPLC (HPLC Method: Column: GEMINI NX C18 (250×21.2 mm, 5 μm); mobile phase A: 10 mM ammonium acetate in water, pH 4.5; mobile phase B: acetonitrile; Gradient: 40-58% B over 20 minutes, then a 5 minute hold at 100% B; Flow: 20 mL/min.

EXAMPLE 411: (4.1 mg, 5% yield); LCMS: m/z=501.4 [M+H]⁺; rt 1.98 min; (LCMS Method: Column: Kinetex XB-C18 (3×75 mm, 2.6 μm); mobile phase A: 10 mM ammonium formate in water (pH ˜3.3), mobile phase B: acetonitrile, Gradient=20-100% B over 4 minutes, then a 0.6 minute hold at 100% B; Temperature: 27° C.; Flow rate: 1.0 mL/min; Detection: UV at 220 nm).

EXAMPLE 412: (4.8 mg, 5% yield); LCMS: m/z=501.3 [M+H]⁺; rt 2.13 min; LCMS Method: Column: Kinetex XB-C18 (3×75 mm, 2.6 μm); mobile phase A: 10 mM ammonium formate in water (pH ˜3.3), mobile phase B: acetonitrile, Gradient=20-100% B over 4 minutes, then a 0.6 minute hold at 100% B; Temperature: 27° C.; Flow rate: 1.0 mL/min; Detection: UV at 220 nm.

### Intermediate 85

### 6-Chloro-3-((4-methoxybenzyl)amino)picolinonitrile

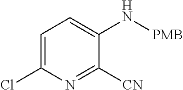

To a solution of 3-amino-6-chloropicolinonitrile (3 g, 19.54 mmol) in ethyl acetate (60 mL) at room temperature were added 4-methoxybenzaldehyde (2.66 g, 19.54 mmol) and TFA (1.505 mL, 19.54 mmol). The reaction mixture was stirred at room temperature for 1 h. Sodium triacetoxyborohydride (6.21 g, 29.3 mmol) was added and the reaction mixture was stirred at room temperature for 3 h. The reaction was quenched with the addition of water. The reaction mixture was extracted with ethyl acetate (2×100 mL), the combined organic layer was washed with water, brine and dried over sodium sulfate. The solvent was evaporated under reduced pressure to yield the crude product, which was purified by silica gel column chromatography using 30% EtOAc in pet ether. The fractions were concentrated under reduced pressure to yield the product 6-chloro-3-((4-methoxybenzyl)amino) picolinonitrile (2 g, 30% yield). LCMS: m/z=274.2 [M+H]⁺; rt 2.69 min; LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Intermediate 86

### 6-Chloro-3-((4-methoxybenzyl)amino)picolinamide

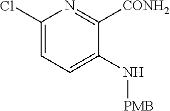

To a solution of 6-chloro-3-((4-methoxybenzyl)amino)picolinonitrile (2 g, 7.31 mmol) in DMSO (10 mL) and water (5 mL) were added K₂C₀₃ (2.020 g, 14.61 mmol) and H₂O₂ (1.493 mL, 14.61 mmol, 30% water) at room temperature. The reaction mixture was stirred for 4 h. The reaction was quenched with the addition of water. The reaction mixture was extracted with ethyl acetate (2×100 mL), the combined organic layer was washed with water, brine and dried over sodium sulfate. The solvent was evaporated under reduced pressure to yield the crude product, which was purified by silica gel column chromatography using 30% EtOAc in pet ether. The fractions were concentrated under reduced pressure to yield 6-chloro-3-((4-methoxybenzyl)amino)picolinamide (1.5 g, 42. % yield). LCMS: m/z=292.1 [M+H]⁺; rt 1.58 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 100% B, flow: 0.7 mL/min.

### Intermediate 87

### 6-Chloro-1-(4-methoxybenzyl)pyrido[3,2-d]pyrimidine-2,4(1H,3H)-dione

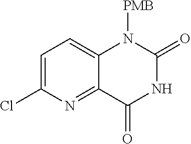

To a solution of 6-chloro-3-((4-methoxybenzyl)amino)picolinamide (1.3 g, 4.46 mmol) in DMF (15 mL) was added sodium hydride (0.356 g, 8.91 mmol, 60% w/w) at 0° C. The reaction mixture was stirred for 1 h. and CDI (1.084 g, 6.68 mmol) was added. The reaction mixture was heated at 70° C. for 2 h. Cold water was added to quench the reaction. The reaction mixture was acidified with 1 N HCl, filtered the yellow solid formed, dried under reduced pressure to yield 6-chloro-1-(4-methoxybenzyl)pyrido[3,2-d] pyrimidine-2,4(1H,3H)-dione (0.8 g, 57% yield). LCMS: m/z=318.1 [M+H]⁺; rt 1.06 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 0.6 minute hold at 90% B, flow: 0.7 mL/min.

### Intermediate 88

### 4,6-Dichloro-1-(4-methoxybenzyl)pyrido[3,2-d]pyrimidin-2(1H)-one

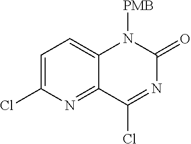

To a solution of 6-chloro-1-(4-methoxybenzyl)pyrido[3,2-d]pyrimidine-2,4(1H,3H)-dione (1.5 g, 4.72 mmol) in toluene (50 mL) at room temperature were added DIPEA (2.061 mL, 11.80 mmol) and phosphoryl chloride (2.2 mL, 23.61 mmol). The reaction mixture was heated at 110° C. for 16 h. The reaction mixture was concentrated under reduced pressure to yield 4,6-dichloro-1-(4-methoxybenzyl)pyrido[3,2-d] pyrimidin-2(1H)-one (1.5 g, 94% yield). LCMS: m/z=336.0 [M+H]⁺; rt 2.2 min; LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Intermediate 89

### tert-Butyl (2R,5S)-4-(6-chloro-1-(4-methoxybenzyl)-2-oxo-1,2-dihydropyrido[3,2-d] pyrimidin-4-yl)-2-ethyl-5-methylpiperazine-1-carboxylate

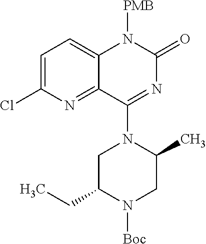

To a solution of tert-butyl (2R,5S)-2-ethyl-5-methylpiperazine-1-carboxylate (0.5 g, 1.74 mmol) in acetonitrile (20 mL) was added DIPEA (3.04 mL, 17.40 mmol) at room temperature. The reaction mixture was stirred for 15 mins. and 4,6-dichloro-1-(4-methoxybenzyl)pyrido[3,2-d]pyrimidin-2(1H)-one (0.585 g, 1.74 mmol) was added. The reaction mixture was heated at 85° C. for 16 h. The reaction was quenched with the addition of water. The mixture was extracted with ethyl acetate (2×100 mL). The combined organic layer was washed with water, brine and dried over sodium sulfate, concentrated under reduced pressure to yield the crude product, which was purified by silica gel column chromatography using 30% EtOAc in pet ether. The fractions were concentrated under reduced pressure to yield tert-butyl (2R,5S)-4-(6-chloro-1-(4-methoxybenzyl)-2-oxo-1,2-dihydropyrido[3,2-d]pyrimidin-4-yl)-2-ethyl-5-methylpiperazine-1-carboxylate (0.55 g, 51% yield). LCMS: m/z=528.2 [M+H]⁺; rt 3.4 min; LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Intermediate 90

### tert-Butyl (2R,5S)-4-(6-cyano-1-(4-methoxybenzyl)-2-oxo-1,2-dihydropyrido[3,2-d]pyrimidin-4-yl)-2-ethyl-5-methylpiperazine-1-carboxylate

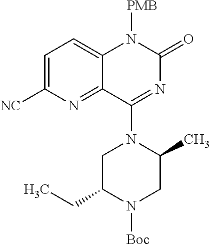

To a stirred solution of tert-butyl (2R,5S)-4-(6-chloro-1-(4-methoxybenzyl)-2-oxo-1,2-dihydropyrido[3,2-d]pyrimidin-4-yl)-2-ethyl-5-methylpiperazine-1-carboxylate (0.5 g, 0.95 mmol) in NMP (2 mL) at room temperature were added zinc cyanide (0.222 g, 1.89 mmol), zinc (0.062 g, 0.95 mmol), and Pd₂(dba)₃ (0.087 g, 0.1 mmol), dppf (0.105 g, 0.19 mmol). The reaction mixture was heated at 90° C. for overnight. The reaction mixture was diluted with EtOAc, filtered through Celite® pad and the filtrate was concentrated under reduced pressure to yield the crude product, which was purified by silica gel column chromatography using 80% EtOAc in pet ether. The fractions were concentrated under reduced pressure to yield tert-butyl (2R,5S)-4-(6-cyano-1-(4-methoxybenzyl)-2-oxo-1,2-dihydropyrido[3,2-d]pyrimidin-4-yl)-2-ethyl-5-methylpiperazine-1-carboxylate (0.43 g, 28% yield). LCMS: m/z=519.4 [M+H]⁺; rt 1.83 min LCMS Method: Column: Acquity UPLC BEH C18 (2.1×50 mm, 1.7 μm); mobile phase A: 10 mM NH₄OAc in water:acetonitrile (95:5); mobile phase B: 10 mM NH₄OAc in water:acetonitrile (5:95), Gradient=20-90% B over 1.1 minute, then a 0.6 minute hold at 90% B; Temperature: 50° C.; Flow rate: 0.7 mL/min; Detection: UV at 220 nm.

### Intermediate 91

### 4-((2S,5R)-5-Ethyl-2-methylpiperazin-1-yl)-1-(4-methoxybenzyl)-2-oxo-1,2-dihydropyrido[3,2-d]pyrimidine-6-carbonitrile·HCl

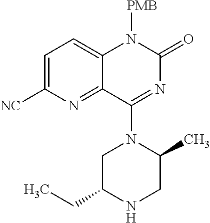

To a solution of tert-butyl (2R,5S)-4-(6-cyano-1-(4-methoxybenzyl)-2-oxo-1,2-dihydropyrido[3,2-d]pyrimidin-4-yl)-2-ethyl-5-methylpiperazine-1-carboxylate (400 mg, 0.77 mmol) in ethyl acetate (5 mL) was added HCl in 1,4-Dioxane (4M, 1.9 mL, 7.71 mmol) at 0° C. The reaction mixture was stirred at room temperature for 6 h. The reaction mixture was evaporated under reduced pressure to yield 4-((2S,5R)-5-ethyl-2-methylpiperazin-1-yl)-1-(4-methoxybenzyl)-2-oxo-1,2-dihydropyrido[3,2-d]pyrimidine-6-carbonitrile, HCl (250 mg, yield 77%). LCMS: m/z=419.2 [M+H]⁺; rt 1.76 min; LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Intermediate 92

### 4-((2S,5R)-5-Ethyl-2-methyl-4-(1-(4-(trifluoromethyl)phenyl)ethyl)piperazin-1-yl)-1-(4-methoxybenzyl)-2-oxo-1,2-dihydropyrido[3,2-d]pyrimidine-6-carbonitrile

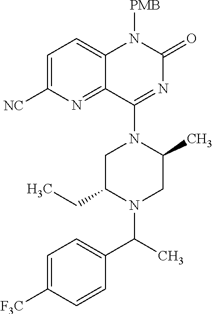

To a solution of 4-((2S,5R)-5-ethyl-2-methylpiperazin-1-yl)-1-(4-methoxybenzyl)-2-oxo-1,2-dihydropyrido[3,2-d]pyrimidine-6-carbonitrile, HCl (0.4 g, 0.879 mmol) in acetonitrile (20 mL) were added DIPEA (0.768 mL, 4.4 mmol), 1-(1-chloroethyl)-4 (trifluoromethyl)benzene (0.55 g, 2.64 mmol) and sodium iodide (0.132 g, 0.879 mmol) at room temperature. The reaction mixture was heated at 85° C. for 16 h. The reaction was quenched with saturated aqueous NH₄Cl solution. The reaction mixture was extracted with ethyl acetate (2×100 mL), the combined organic layer was washed with water, brine and dried over sodium sulfate, and concentrated under reduced pressure to yield the crude product, which was purified by silica gel column chromatography using 30% EtOAc in pet ether. The fractions were concentrated under reduced pressure to yield 4-((2S,5R)-5-ethyl-2-methyl-4-(1-(4-(trifluoromethyl)phenyl)ethyl) piperazin-1-yl)-1-(4-methoxybenzyl)-2-oxo-1,2-dihydropyrido[3,2-d]pyrimidine-6-carbonitrile (0.2 g, 35% yield). LCMS: m/z=591.2 [M+H]⁺; rt 3.77 min; LCMS Method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Examples 413 and 414

### 4-((2S,5R)-5-Ethyl-2-methyl-4-(1-(4-(trifluoromethyl)phenyl)ethyl)piperazin-1-yl)-2-oxo-1,2-dihydropyrido[3,2-d]pyrimidine-6-carbonitrile

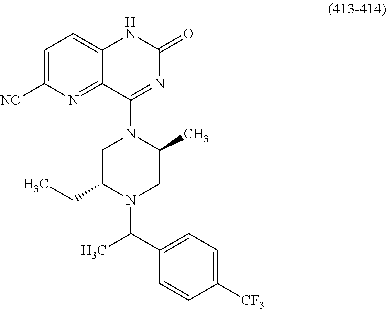 [SMILES: CC[C@@H]1CN(c2nc(=O)[nH]c3ccc(C#N)nc23)[C@@H](C)CN1C(C)c1ccc(C(F)(F)F)cc1]

To a solution of 4-((2S,5R)-5-ethyl-2-methyl-4-(1-(4-(trifluoromethyl)phenyl) ethyl)piperazin-1-yl)-1-(4-methoxybenzyl)-2-oxo-1,2-dihydropyrido[3,2-d]pyrimidine-6-carbonitrile (150 mg, 0.25 mmol) in TFA (1.5 mL, 19.47 mmol) at 0° C. was added trifluoromethane sulfonic acid (0.75 mL, 8.45 mmol). The reaction mixture was stirred at room temperature for 4 h. The reaction mixture was basified with NaHCO₃ solution and extracted with EtOAc. The organic layer was washed with water, brine and dried over sodium sulfate. The solvent was evaporated under reduced pressure to yield crude product, which was purified by preparative HPLC Method: Column: YMC TRIART C18 EXRS (250×20 mm, 5 μm); mobile phase A: 10 mM ammonium acetate in water; mobile phase B: acetonitrile; Flow: 20 mL/min] to yield Examples 413 and 414.

EXAMPLE 413: (30 mg, 25% yield), LCMS: m/z=469.2 [M−H]; rt 3.19 min; LCMS Method: Column: Kinetex XB-C18 (3×75 mm, 2.6 μm); mobile phase A: 10 mM ammonium formate in water (pH ˜3.3); mobile phase B: acetonitrile, Gradient=20-100% B over 4 minutes, then a 0.6 minute hold at 100% B; Temperature: 27° C.; Flow rate: 1.0 mL/min; Detection: UV at 220 nm. ¹H NMR (DMSO-d₆, 400 MHz) δ (ppm)=10.9-11.2 (m, 1H), 8.06 (d, J=8.5 Hz, 1H), 7.71-7.73 (m, 2H), 7.6-7.7 (m, 3H), 5.62-5.97 (m, 1H), 4.86-5.29 (m, 1H), 3.82-3.85 (m, 1H), 2.7-2.9 (m, 2H), 2.6-2.7 (m, 1H), 2.2-2.5 (m, 1H), 1.4-1.6 (m, 5H), 1.29 (d, J=6.5 Hz, 3H), 0.70 (t, J=7.3 Hz, 3H).

EXAMPLE 414: (30 mg, 25% yield), LCMS: m/z=471.2 [M+H]⁺; rt 3.33 min; LCMS Method: Column: Kinetex XB-C18 (3×75 mm, 2.6 μm); mobile phase A: 10 mM ammonium formate in water (pH 3.3); mobile phase B: acetonitrile, Gradient=20-100% B over 4 minutes, then a 0.6 minute hold at 100% B; Temperature: 27° C.; Flow rate: 1.0 mL/min; Detection: UV at 220 nm. ¹H NMR (DMSO-d₆, 400 MHz) δ (ppm)=10.8-11.7 (m, 1H), (8.08 (d, 1H, J=8.8 Hz), 7.7-7.7 (m, 2H), 7.6-7.7 (m, 3H), 4.6-6.3 (m, 2H), 3.70-3.76 (m, 1H), 3.09-3.11 (m, 1H), 3.1-3.1 (m, 1H), 2.58-2.62 (m, 1H), 2.15-2.18 (m, 1H), 1.4-1.6 (m, 2H), 1.2-1.3 (m, 6H), 1.01 (t, 3H, J=7.4 Hz).

The example in the Table 18 were prepared according to the general procedure described in Examples 413 and 414. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 18

| Ex. | | Stereo | LCMS | | |
|---|---|---|---|---|---|
| No. | Structure | chem. | Method | LCMS rt | [M + H]⁺ |
| 415 416 | 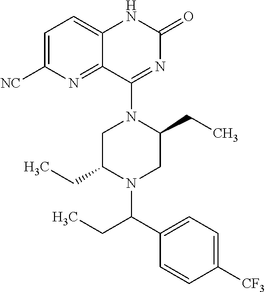 [SMILES: CCC(c1ccc(C(F)(F)F)cc1)N1C[C@H](CC)N(c2nc(=O)[nH]c3ccc(C#N)nc23)C[C@H]1CC] | H H | E E | 3.19 2.70 | 499.3 499.2 |

### Intermediate 93

### 6-Chloro-3-((methyl-d₃)amino)picolinonitrile

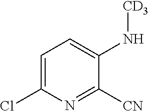 [SMILES: [2H]C([2H])([2H])Nc1ccc(Cl)nc1C#N]

To a solution of N-(6-chloro-2-cyanopyridin-3-yl)-2,2,2-trifluoroacetamide (3 g, 12.02 mmol) in DMF (15 mL) at room temperature was added potassium carbonate (4.15 g, 30.1 mmol), followed by iodomethane-d₃ (3.9 mL, 60.1 mmol). The reaction mixture was stirred for 16 h. The reaction was quenched with the addition of water. The mixture was extracted with ethyl acetate (2×100 mL), the combined organic layer was washed with water, brine and dried over sodium sulfate. The solvent was evaporated under reduced pressure to yield the crude product, which was purified by silica gel column chromatography using 30% EtOAc in pet ether. The fractions were concentrated under reduced pressure to yield N-(6-chloro-2-cyanopyridin-3-yl)-2,2,2-trifluoro-N-(methyl-d₃) acetamide (1.5 g, 47% yield). LCMS: m/z=171.1 [M+H]⁺; rt 1.19 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 1.1 min, then a 2.2 minute hold at 100% B, flow: 0.7 mL/min.

### Intermediate 94

### 6-Chloro-3-((methyl-d₃)amino)picolinamide

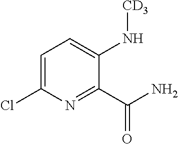 [SMILES: [2H]C([2H])([2H])Nc1ccc(Cl)nc1C(N)=O]

To a solution of 6-chloro-3-((methyl-d₃)amino)picolinonitrile (2 g, 11.72 mmol) in DMSO (100 mL) and water (50 mL) were added potassium carbonate (3.24 g, 23.44 mmol) and hydrogen peroxide (2.4 mL, 23.44 mmol, 30% in water) at 0° C. The reaction mixture was stirred at room temperature for 3 h. The reaction mixture was diluted with water and the precipitate formed was filtered, dried under reduced pressure to yield 6-chloro-3-((methyl-d₃)amino)picolinamide (1 g, 45% yield). LCMS: m/z=189.1 [M+H]⁺; rt 1.06 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 95

### 6-Chloro-4-hydroxy-1-(methyl-d₃)pyrido[3,2-d]pyrimidin-2(1H)-one

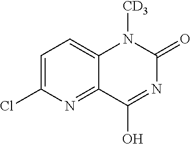 [SMILES: [2H]C([2H])([2H])n1c(=O)nc(O)c2nc(Cl)ccc21]

To a solution of 6-chloro-3-((methyl-d₃)amino)picolinamide (1.4 g, 7.42 mmol) in DMF (10 mL) at 0° C. were added sodium hydride (0.594 g, 14.84 mmol, 60% w/w) and CDI (1.805 g, 11.13 mmol). The reaction mixture was stirred at room temperature for 3 h. The reaction was quenched with the addition of ice water. The mixture was acidified with 1.5 N HCl. The mixture was filtered to separate a yellow solid, which was dried under reduced pressure to yield 6-chloro-1-(methyl-d₃) pyrido[3,2-d]pyrimidine-2,4(1H,3H)-dione (1.0 g, 62% yield). LCMS: m/z=215.1 [M+H]⁺; rt 0.50 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 96

### 4,6-Dichloro-1-(methyl-d₃)pyrido[3,2-d]pyrimidin-2(1H)-one

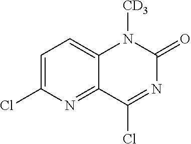 [SMILES: [2H]C([2H])([2H])n1c(=O)nc(Cl)c2nc(Cl)ccc21]

To a solution of 6-chloro-4-hydroxy-1-(methyl-d₃)pyrido[3,2-d]pyrimidin-2(1H)-one (0.5 g, 2.33 mmol) in toluene (30 mL) at room temperature were added DIPEA (1.0 mL, 5.82 mmol) and phosphoryl chloride (1.1 mL, 11.65 mmol). The reaction mixture was heated at 110° C. for 16 h. The reaction mixture was concentrated under reduced pressure to yield 4,6-dichloro-1-(methyl-d₃)pyrido[3,2-d]pyrimidin-2(1H)-one (0.4 g, 74% yield). LCMS: m/z=232.9 (M+H); retention time 0.84 min. (Column: AQUITY UPLC BEH C18 (3.0×50 mm, 1.7 μm), mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 97

### tert-Butyl (2R,5S)-4-(6-chloro-1-(methyl-d₃)-2-oxo-1,2-dihydropyrido[3,2-d]pyrimidin-4-yl)-2-ethyl-5-methylpiperazine-1-carboxylate

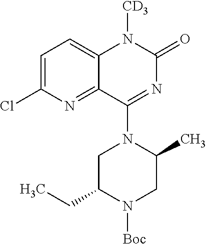 [SMILES: [2H]C([2H])([2H])n1c(=O)nc(N2C[C@@H](CC)N(C(=O)OC(C)(C)C)C[C@@H]2C)c2nc(Cl)ccc21]

To a solution of tert-butyl (2R,5S)-2-ethyl-5-methylpiperazine-1-carboxylate (0.5 g, 2.190 mmol) in acetonitrile (20 mL) at room temperature was added DIPEA (3.8 mL, 21.90 mmol), followed by 4,6-dichloro-1-(methyl-d₃)pyrido[3,2-d]pyrimidin-2(1H)-one (0.510 g, 2.19 mmol). The reaction mixture was heated at 85° C. for 16 h. The reaction was quenched with the addition of water. The mixture was extracted with ethyl acetate (2×100 mL), the combined organic layer was washed with water, brine and dried over sodium sulfate. The solvent was evaporated under reduced pressure to yield the crude product, which was purified by silica gel column chromatography using 90% EtOAc in pet ether. The fractions were concentrated under reduced pressure to yield tert-butyl (2R,5S)-4-(6-chloro-1-(methyl-d₃)-2-oxo-1,2-dihydropyrido[3,2-d]pyrimidin-4-yl)-2-ethyl-5-methylpiperazine-1-carboxylate (0.4 g, 41% yield). LCMS: m/z=425.2 [M+H]⁺; rt 2.77 min; LCMS method: Column: Kinetex XB-C18 (3×75 mm, 2.6 μm); mobile phase A: 10 mM ammonium formate:acetonitrile (98:2), mobile phase B: 10 mM ammonium formate:acetonitrile (2:98), Gradient=20-100% B over 4 minutes, then a 0.6 minute hold at 100% B; Temperature: 27° C.; Flow rate: 1.0 mL/min; Detection: UV at 220 nm.

### Intermediate 98

### 6-Chloro-4-((2S,5R)-5-ethyl-2-methylpiperazin-1-yl)-1-(methyl-d₃)pyrido[3,2-d]pyrimidin-2(1H)-one·TFA

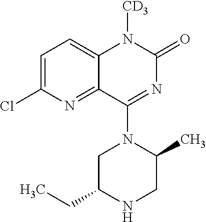 [SMILES: [2H]C([2H])([2H])n1c(=O)nc(N2C[C@@H](CC)NC[C@@H]2C)c2nc(Cl)ccc21]

To a solution of tert-butyl (2R,5S)-4-(6-chloro-1-(methyl-d₃)-2-oxo-1,2-dihydropyrido[3,2-d]pyrimidin-4-yl)-2-ethyl-5-methylpiperazine-1-carboxylate (0.42 g, 0.988 mmol) in DCM (5 mL) at 0° C. was added TFA (1.2 mL, 14.8 mmol). The reaction mixture was stirred at room temperature for 3 h. The volatiles were removed under reduced pressure to yield 6-chloro-4-((2S,5R)-5-ethyl-2-methylpiperazin-1-yl)-1-(methyl-d₃)pyrido[3,2-d]pyrimidin-2(1H)-one, TFA (0.4 g, 76% yield). LCMS: m/z=325.2 [M+H]⁺; rt 0.67 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Examples 417 and 418

### 6-Chloro-4-((2S,5R)-5-ethyl-2-methyl-4-(1-(4-(trifluoromethyl)phenyl)ethyl)piperazin-1-yl)-1-(methyl-d₃)pyrido[3,2-d]pyrimidin-2(1H)-one

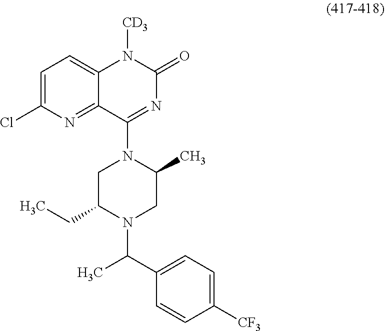 [SMILES: [2H]C([2H])([2H])n1c(=O)nc(N2C[C@@H](CC)N(C(C)c3ccc(C(F)(F)F)cc3)C[C@@H]2C)c2nc(Cl)ccc21]

To a solution of 6-chloro-4-((2S,5R)-5-ethyl-2-methylpiperazin-1-yl)-1-(methyl-d₃)pyrido[3,2-d]pyrimidin-2(1H)-one, TFA (0.4 g, 0.911 mmol) in acetonitrile (5 mL) at room temperature was added DIPEA (0.5 mL, 2.73 mmol), followed by 1-(1-chloroethyl)-4-(trifluoromethyl)benzene (0.570 g, 2.73 mmol), sodium iodide (0.137 g, 0.911 mmol). The reaction mixture was heated at 85° C. for 16 h. The reaction mixture was diluted with EtOAc (50 mL) and filtered through Celite® pad, washed with additional ethyl acetate (2×50 mL). The filtrate was washed with water (50 mL), brine (50 mL), dried over Na₂SO₄ and concentrated under reduced pressure to yield the crude product, which was purified by preparative HPLC to yield Examples 414 and 415. HPLC method: column: HPLC Method: Column: YMC TRIART C18 EXRS (250×4.6 mm, 5 μm); mobile phase A=10 mM ammonium acetate in water; mobile phase B=acetonitrile, Gradient: 0-20% B over 20 minutes: 20.0 mL/min.

Example 417: (13 mg, 3% yield), LCMS: m/z=497.3 [M+H]⁺; rt 2.52 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min. ¹H NMR (400 MHz, DMSO-d₆) δ (ppm)=7.91 (d, J=9.0 Hz, 1H), 7.78 (d, J=9.0 Hz, 1H), 7.72-7.74 (m, 2H), 7.60-7.62 (m, 2H), 5.99-5.73 (m, 1H), 5.17-4.70 (m, 1H), 3.84 (q, J=6.4 Hz, 1H), 3.53-3.36 (m, 1H), 2.89-2.68 (m, 2H), 2.37-2.25 (m, 1H), 1.61-1.22 (m, 8H), 0.69 (t, J=6.8 Hz, 3H).

EXAMPLE 418: (13 mg, 3% yield), LCMS: m/z=497.3 [M+H]⁺; rt 2.58 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min. ¹H NMR (400 MHz, DMSO-d₆) δ (ppm)=7.93 (d, J=9.0 Hz, 1H), 7.79 (br d, J=9.0 Hz, 1H), 7.73-7.66 (m, 2H), 7.66-7.54 (m, 2H), 6.23-5.47 (m, 1H), 5.06-4.73 (m, 1H), 3.77-3.54 (m, 2H), 3.19-2.95 (m, 1H), 2.64-2.54 (m, 1H), 2.24-2.01 (m, 1H), 1.60-1.40 (m, 2H), 1.36-1.08 (m, 5H), 0.99 (t, J=7.3 Hz 3H).

### Examples 419 and 420

### 4-((2S,5R)-5-Ethyl-2-methyl-4-(1-(4-(trifluoromethyl)phenyl)ethyl)piperazin-1-yl)-1-(methyl-d₃)-2-oxo-1,2-dihydropyrido[3,2-d]pyrimidine-6-carbonitrile

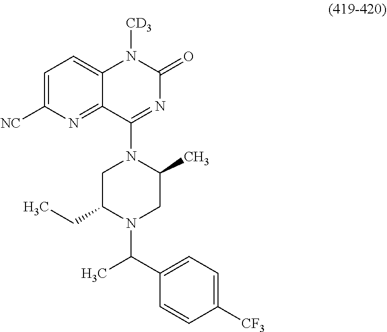 [SMILES: [2H]C([2H])([2H])n1c(=O)nc(N2C[C@@H](CC)N(C(C)c3ccc(C(F)(F)F)cc3)C[C@@H]2C)c2nc(C#N)ccc21]

To a stirred solution of 6-chloro-4-((2S,5R)-5-ethyl-2-methyl-4-(1-(4-(trifluoromethyl)phenyl)ethyl)piperazin-1-yl)-1-(methyl-d₃)pyrido[3,2-d]pyrimidin-2(1H)-one (150 mg, 0.30 mmol) in NMP (2 mL) at room temperature were added zinc cyanide (70.9 mg, 0.604 mmol), zinc (19.73 mg, 0.30 mmol), Pd₂(dba)₃ (27.6 mg, 0.030 mmol) and dppf (33.5 mg, 0.060 mmol) under an argon atmosphere. The reaction mixture was heated at 90° C. for overnight. The reaction mixture was diluted with EtOAc (50 mL) and filtered through a Celite® pad, washed with additional ethyl acetate (2×50 mL). The filtrate was washed with water (50 mL), brine (50 mL), dried over Na₂SO₄ and concentrated under reduced pressure to yield the crude product, which was purified by preparative HPLC method: Column: Sunfire C18 (150×19 mm, 5 μm); mobile phase A=10 mM ammonium acetate in water; mobile phase B=acetonitrile; Flow 19 mL/min; Gradient: 10-85% B, over 0-22 min] to yield Examples 419 and 420.

EXAMPLE 419: (25 mg, 17% yield), LCMS: m/z=488.3 [M+H]⁺; rt 2.31 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min. ¹H NMR (400 MHz, DMSO-d₆) δ (ppm)=8.22 (d, J=9.0 Hz, 1H), 7.97 (d, J=8.8 Hz, 1H), 7.72-7.74 (m, 2H), 7.60-7.62 (m, 2H), 5.85-5.63 (m, 1H), 5.10-4.77 (m, 1H), 3.83-3.88 (m, 1H), 3.51-3.39 (m, 1H), 2.89-2.71 (m, 2H), 2.41-2.32 (m, 1H), 1.68-1.00 (m, 8H), 0.79-0.59 (m, 3H).

EXAMPLE 420: (30 mg, 20% yield), LCMS: m/z=488.3 [M+H]⁺; rt 2.36 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min. ¹H NMR (400 MHz, DMSO-d₆) δ (ppm)=8.33-8.15 (m, 1H), 8.06-7.90 (m, 1H), 7.77-7.51 (m, 4H), 6.06-5.43 (m, 1H), 5.10-4.71 (m, 1H), 3.79-3.55 (m, 2H), 3.17-2.99 (m, 1H), 2.64-2.55 (m, 1H), 2.26-2.02 (m, 1H), 1.61-1.11 (m, 8H), 1.00 (br t, J=7.2 Hz, 3H).

### Examples 421 and 422

### 6-Chloro-4-((2S,5R)-2,5-diethyl-4-(1-(4-(trifluoromethyl)phenyl)propyl)piperazin-1-yl)-1-methylpyrido[3,2-d]pyrimidin-2(1H)-one

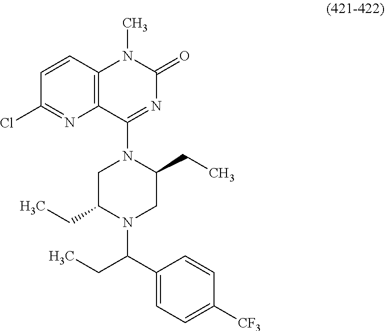 [SMILES: CCC(c1ccc(C(F)(F)F)cc1)N1C[C@H](CC)N(c2nc(=O)n(C)c3ccc(Cl)nc23)C[C@H]1CC]

To a stirred suspension of 6-chloro-4-((2S,5R)-2,5-diethylpiperazin-1-yl)-1-methylpyrido[3,2-d]pyrimidin-2(1H)-one, TFA (500 mg, 1.111 mmol), 1-(1-chloropropyl)-4-(trifluoromethyl)benzene (297 mg, 1.334 mmol) in acetonitrile (15 mL) were added sodium iodide (333 mg, 2.223 mmol) and DIPEA (1.165 mL, 6.67 mmol). The reaction mixture was heated at 85° C. and stirred for 16 h. The reaction mixture was cooled to room temperature and was concentrated under reduced pressure to obtain the crude product, which was purified using silica gel (24 g) chromatography by using 0-10% methanol in chloroform as eluent. The fractions were concentrated under reduced pressure to obtain 6-chloro-4-((2S,5R)-2,5-diethyl-4-(1-(4-(trifluoromethyl)phenyl) propyl)piperazin-1-yl)-1-methylpyrido[3,2-d]pyrimidin-2(1H)-one (255 mg, 38% yield) as diasteromeric mixture. LCMS: m/z=522.3 [M+H]⁺; rt 1.59 and 1.62 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

The diasteromeric mixture was purified by preparative SFC. SFC Method: Column: Chiralpak IG (250×30 mm, 5 μm); mobile phase: 80% CO₂/20% of 4 M methanolic ammonia; Flow: 90 g/min; Detector Wavelength: 260 nm; Temperature: 40° C. Example 421; Isolate 1: First eluting peak, rt=3.09 min; Example 422 Isolate 2: Second eluting peak, rt=3.62 min.

EXAMPLE 421: LCMS: m/z, 522.3 [M+H]⁺; rt 2.77 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.92 (d, J=9.0 Hz, 1H), 7.78 (br d, J=8.6 Hz, 1H), 7.72 (d, J=8.3 Hz, 2H), 7.55 (br d, J=8.3 Hz, 2H), 5.99-5.56 (s, 1H), 5.06-4.78 (m, 1H), 3.67-3.60 (m, 1H), 3.42 (s, 3H), 3.08-2.74 (m, 3H), 2.37-2.27 (m, 1H), 1.96-1.80 (m, 2H), 1.67-1.53 (m, 2H), 1.50-1.18 (m, 2H), 1.03-0.86 (m, 3H), 0.71-0.47 (m, 6H).

EXAMPLE 422: LCMS: m/z, 522.3 [M+H]⁺; rt 2.78 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.98-7.86 (m, 1H), 7.85-7.65 (m, 3H), 7.57 (d, J=8.1 Hz, 2H), 6.20-5.32 (m, 1H), 5.11-4.66 (m, 1H), 3.66-3.46 (m, 2H), 3.42 (s, 3H), 3.28-3.00 (m, 2H), 2.25-2.11 (m, 1H), 1.99-1.69 (m, 3H), 1.59-1.35 (m, 3H), 1.02-0.86 (m, 3H), 0.61-0.57 (m, 6H).

### Intermediate 99

### 4-((2S,5R)-2,5-Diethyl-4-(1-(4-(trifluoromethyl)phenyl)ethyl)piperazin-1-yl)-1-methyl-2-oxo-1,2-dihydropyrido[3,2-d]pyrimidine-6-carboxylic Acid

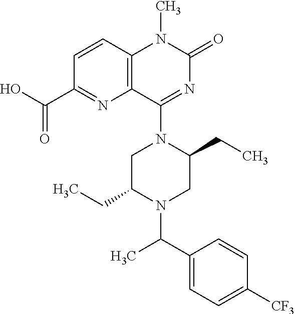 [SMILES: CC[C@H]1CN(C(C)c2ccc(C(F)(F)F)cc2)[C@H](CC)CN1c1nc(=O)n(C)c2ccc(C(=O)O)nc12]

To a solution of 4-((2S,5R)-2,5-diethyl-4-(1-(4-(trifluoromethyl)phenyl)ethyl) piperazin-1-yl)-1-methyl-2-oxo-1,2-dihydropyrido[3,2-d]pyrimidine-6-carbonitrile (350 mg, 0.70 mmol) in ethanol (2.5 mL) was added NaOH (281 mg, 7.02 mmol) in H₂O (2.5 mL). The reaction mixture was heated at 90° C. and stirred overnight. The reaction mixture was cooled to room temperature and concentrated under reduced pressure. The residue was acidified with 1.5 N HCl to pH ˜3 and was extracted with EtOAc (100 mL). The organic layer was washed with water (20 mL), brine (20 mL), dried over Na₂SO₄ and concentrated under reduced pressure to yield 4-((2S,5R)-2,5-diethyl-4-(1-(4-(trifluoromethyl)phenyl)ethyl) piperazin-1-yl)-1-methyl-2-oxo-1,2-dihydropyrido[3,2-d] pyrimidine-6-carboxylic acid (300 mg, 83% yield). LCMS: m/z=518.3 [M+H]⁺; retention time 1.13 and 1.14 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 2.2 minute hold at 90% B, flow: 0.7 mL/min.

### Example 423

### 4-((2S,5R)-2,5-Diethyl-4-(1-(4-(trifluoromethyl)phenyl)ethyl)piperazin-1-yl)-6-(hydroxymethyl)-1-methylpyrido[3,2-d]pyrimidin-2(1H)-one

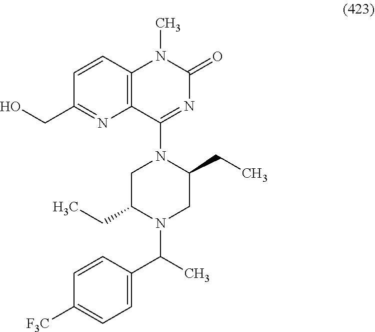 [SMILES: CC[C@H]1CN(C(C)c2ccc(C(F)(F)F)cc2)[C@H](CC)CN1c1nc(=O)n(C)c2ccc(CO)nc12]

To a solution of 4-((2S,5R)-2,5-diethyl-4-(1-(4-(trifluoromethyl)phenyl)ethyl) piperazin-1-yl)-1-methyl-2-oxo-1,2-dihydropyrido[3,2-d]pyrimidine-6-carboxylic acid (150 mg, 0.29 mmol) in THF (5 mL) was added drop wise BH₃·dimethyl sulfide (0.08 mL, 0.87 mmol) at 0° C. After warming-up to ambient temperature, stirring was continued for additional 24 h. The solution was cooled again to 0° C. The reaction was quenched with MeOH. The reaction mixture was refluxed for 1 h. The volatiles removed under the reduced pressure and reaction mixture was diluted with water. The aqueous phase was extracted with ethyl acetate (50 mL) and the organic layer was washed with water, brine, dried, filtered and concentrated under reduced pressure to afford 4-((2S,5R)-2,5-diethyl-4-(1-(4-(trifluoromethyl)phenyl)ethyl)piperazin-1-yl)-6-(hydroxymethyl)-1-methylpyrido[3,2-d]pyrimidin-2(1H)-one (100 mg, 69% yield). LCMS: m/z=504.3 [M+H]⁺; retention time 1.78 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 2.2 minute hold at 90% B, flow: 0.7 mL/min.

### Examples 424 and 425

### 4-((2S,5R)-2,5-Diethyl-4-(1-(4-(trifluoromethyl)phenyl)ethyl)piperazin-1-yl)-6-(methoxymethyl)-1-methylpyrido[3,2-d]pyrimidin-2(1H)-one

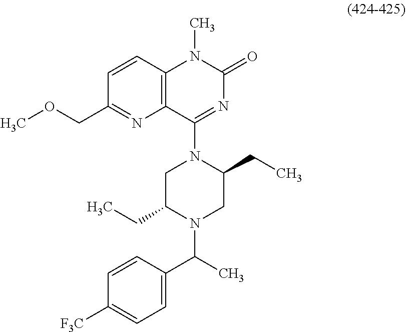 [SMILES: CC[C@H]1CN(C(C)c2ccc(C(F)(F)F)cc2)[C@H](CC)CN1c1nc(=O)n(C)c2ccc(COC)nc12]

To a stirred suspension of NaH (17 mg, 0.42 mmol, 60% w/w) in DMF (3 mL) was added 4-((2S,5R)-2,5-diethyl-4-(1-(4-(trifluoromethyl)phenyl)ethyl) piperazin-1-yl)-6-(hydroxymethyl)-1-methylpyrido[3,2-d]pyrimidin-2(1H)-one (70 mg, 0.14 mmol) at 0° C. After 5 minutes, a solution of methyl iodide (0.02 mL, 0.28 mmol) in DMF (1 mL) was added and stirred for 2 h at room temperature. The reaction mixture was cooled to 0° C. The reaction was quenched with the addition of ice cold water. The reaction mixture was extracted with EtOAc (2×50 mL). The combined organic extract was washed with brine, dried over Na₂SO₄ and concentrated under reduced pressure to give the crude product, which was purified using preparative SFC. SFC Method: Column: Chiralpak IG (250×30 mm, 5 μm); mobile phase: 70% CO₂/30% of MeOH; Flow: 80 g/min; Detector Wavelength: 220 nm; Temperature: 35° C. Example 424: Isolate 1: First eluting peak, rt=2.96 min. Example 425: Isolate 2: Second eluting peak, rt=4.01 min.

EXAMPLE 424: (5.1 mg, 7% yield): LCMS: m/z, 518.3 [M+H]⁺; retention time 2.48 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.86 (d, J=8.8 Hz, 1H), 7.77-7.64 (m, 3H), 7.60 (br d, J=8.3 Hz, 2H), 6.28-5.87 (m, 1H), 5.18-4.80 (m, 1H), 4.60-4.34 (m, 2H), 3.91-3.68 (m, 1H), 3.42 (s, 3H), 3.28 (br s, 2H), 3.11-2.85 (m, 2H), 2.79-2.69 (m, 1H), 2.40-2.18 (m, 1H), 2.14-1.83 (m, 2H), 1.32-1.18 (m, 3H), 0.89 (br t, J=7.2 Hz, 3H), 0.75-0.36 (m, 3H).

EXAMPLE 425: (2.2 mg, 3% yield)): LCMS: m/z, 518.3 [M+H]⁺; retention time 2.49 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.92-7.83 (m, 1H), 7.75-7.64 (m, 3H), 7.64-7.56 (m, 2H), 5.91-5.73 (m, 1H), 5.15-4.84 (m, 1H), 4.60-4.35 (m, 2H), 3.72-3.60 (m, 1H), 3.42 (s, 3H), 3.19-2.96 (m, 2H), 2.26-2.08 (m, 2H), 1.94-1.74 (m, 2H), 1.52-1.38 (m, 2H), 1.25 (br d, J=6.1 Hz, 3H), 1.03-0.73 (m, 3H), 0.67-0.47 (m, 3H).

### Example 426

### 6-Chloro-4-((2S,5R)-2,5-diethyl-4-(1-(4-(trifluoromethyl)phenyl)ethyl)piperazin-1-yl)-1-methylpyrido[3,2-d]pyrimidin-2(1H)-one

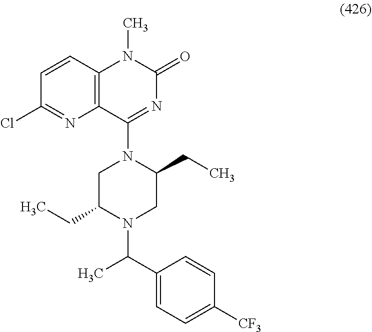 [SMILES: CC[C@H]1CN(C(C)c2ccc(C(F)(F)F)cc2)[C@H](CC)CN1c1nc(=O)n(C)c2ccc(Cl)nc12]

To a stirred solution of 6-chloro-4-((2S,5R)-2,5-diethylpiperazin-1-yl)-1-methylpyrido[3,2-d]pyrimidin-2(1H)-one, TFA (1.0 g, 2.22 mmol) in acetonitrile (15 mL) was added DIPEA (1.165 mL, 6.67 mmol). The reaction mixture was stirred for 5 min and then 1-(1-chloroethyl)-4-(trifluoromethyl)benzene (0.556 g, 2.67 mmol) was added at room temperature. The reaction mixture was heated at 85° C. and was stirred for 20 h. The reaction mixture was cooled to room temperature and concentrated under reduced pressure to obtain the crude product, which was purified using silica gel (24 g) chromatography by using 0-10% methanol in chloroform as eluent. The fractions were concentrated under reduced pressure to obtain 6-chloro-4-((2S,5R)-2,5-diethyl-4-(1-(4-(trifluoromethyl)phenyl)ethyl)piperazin-1-yl)-1-methylpyrido[3,2-d]pyrimidin-2(1H)-one (750 mg, 58% yield) as a diasteromeric mixture. LCMS: m/z=508.3 [M+H]⁺; rt 1.50 and 1.53 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Examples 427 and 428

### 4-((2S,5R)-2,5-Diethyl-4-(1-(4-(trifluoromethyl)phenyl)ethyl)piperazin-1-yl)-6-methoxy-1-methylpyrido[3,2-d]pyrimidin-2(1H)-one

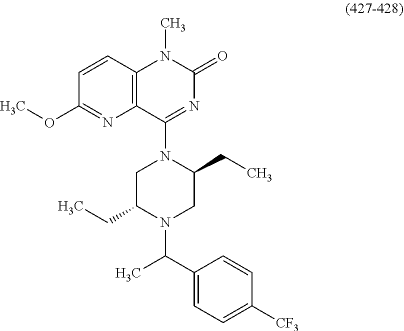 [SMILES: CC[C@H]1CN(C(C)c2ccc(C(F)(F)F)cc2)[C@H](CC)CN1c1nc(=O)n(C)c2ccc(OC)nc12]

To a solution of 6-chloro-4-((2S,5R)-2,5-diethyl-4-(1-(4-(trifluoromethyl)phenyl) ethyl)piperazin-1-yl)-1-methylpyrido[3,2-d]pyrimidin-2(1H)-one (100 mg, 0.2 mmol) in MeOH (2.0 mL) was added sodium methoxide (0.23 mL, 0.98 mmol). The reaction mixture was stirred at 60° C. for 48 h. The reaction mixture was concentrated under reduced pressure, dissolved in ethyl acetate (50 mL), washed with water, brine, dried over Na₂SO₄, concentrated under reduced pressure to give the crude product which was purified by chiral preparative HPLC. HPLC Method: Column: Cellulose-5 (150×19 mm, 5 μm); mobile phase: 10 mM ammonium acetate in MeOH, Flow: 22 mL/min). Peak 1 (Diastereomer 1): rt=10.04 min and Peak 2 (Diastereomer 2), rt=11.53 min.

EXAMPLE 427: (17 mg, 23% yield); LCMS: m/z, 504.3 [M+H]⁺; rt 2.48 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.88 (d, J=9.3 Hz, 1H), 7.71 (d, J=8.3 Hz, 2H), 7.61 (d, J=7.8 Hz, 2H), 7.24 (br d, J=8.8 Hz, 1H), 6.31-5.91 (m, 1H), 5.23-4.69 (m, 1H), 3.87-3.75 (m, 3H), 3.42 (s, 3H), 3.13-2.71 (m, 3H), 2.98-2.73 (m, 2H), 2.39-2.29 (m, 1H), 2.16-1.88 (m, 2H), 1.54-1.34 (m, 1H), 1.49-1.16 (m, 3H), 0.93-0.71 (m, 3H), 0.68-0.36 (m, 3H).

EXAMPLE 428: (14 mg, 18% yield); LCMS: m/z, 504.3 [M+H]⁺; rt 2.49 min; LCMS method: Column: XBridge BEH XP C18 (50×2.1 mm, 2.5 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR (400 MHz, DMSO-d₆) δ (ppm)=7.88 (d, J=9.3 Hz, 1H), 7.76-7.67 (m, 2H), 7.64-7.53 (m, 2H), 7.31-7.14 (m, 1H), 6.32-6.01 (m, 1H), 5.00 (br s, 1H), 3.97-3.62 (m, 4H), 3.43 (s, 3H), 3.20-2.97 (m, 2H), 2.76-2.57 (m, 1H), 2.27-2.15 (m, 1H), 2.05-1.83 (m, 1H), 1.76-1.63 (m, 1H), 1.58-1.32 (m, 2H), 1.24 (d, J=6.6 Hz, 3H), 1.00-0.43 (m, 6H).

The examples in the Table 19 were prepared according to the general procedure described in Examples 427 and 428, using the appropriate alcohol and chloro derivative in the synthetic sequence. When the synthesis provided a mixture of diastereomers, the mixture was separated at the final stage using either preparative chromatography or preparative chiral chromatography.

TABLE 19

| Ex. | | Stereo- | LCMS | LCMS | |
|---|---|---|---|---|---|
| No. | Structure | chemistry | Method | rt | [M + H]⁺ |
| 429 430 | 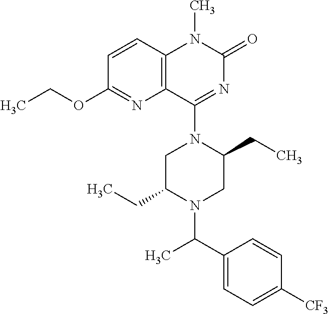 [SMILES: CCOc1ccc2c(n1)c(N1C[C@@H](CC)N(C(C)c3ccc(C(F)(F)F)cc3)C[C@@H]1CC)nc(=O)n2C] | H H | C C | 2.60 2.61 | 518.3 518.3 |
| 431 432 | 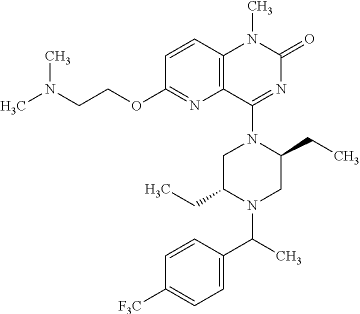 [SMILES: CC[C@H]1CN(C(C)c2ccc(C(F)(F)F)cc2)[C@H](CC)CN1c1nc(=O)n(C)c2ccc(OCCN(C)C)nc12] | H H | C C | 2.08 2.10 | 561.4 561.4 |
| 433 434 | 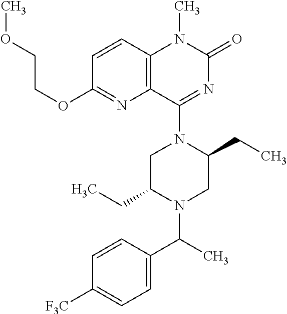 [SMILES: CC[C@H]1CN(C(C)c2ccc(C(F)(F)F)cc2)[C@H](CC)CN1c1nc(=O)n(C)c2ccc(OCCOC)nc12] | H H | C C | 2.37 2.38 | 548.4 548.4 |
| 435 436 | 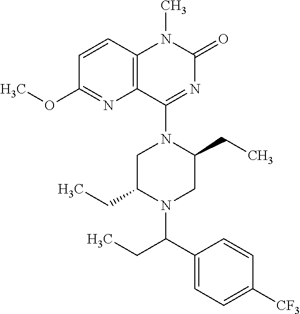 [SMILES: CCC(c1ccc(C(F)(F)F)cc1)N1C[C@H](CC)N(c2nc(=O)n(C)c3ccc(OC)nc23)C[C@H]1CC] | H H | C C | 2.61 2.61 | 518.3 518.3 |
| 437 438 | 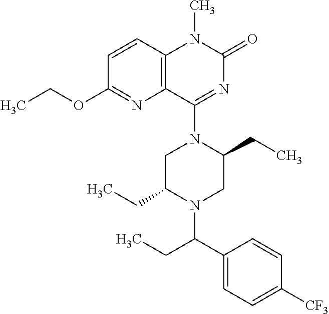 [SMILES: CCOc1ccc2c(n1)c(N1C[C@@H](CC)N(C(CC)c3ccc(C(F)(F)F)cc3)C[C@@H]1CC)nc(=O)n2C] | H H | C C | 2.72 2.72 | 532.4 532.4 |

### Examples 439 and 440

### 4-((2S,5R)-2,5-Diethyl-4-(1-(4-(trifluoromethyl)phenyl)ethyl)piperazin-1-yl)-6-(difluoromethyl)-1-methylpyrido[3,2-d]pyrimidin-2(1H)-one

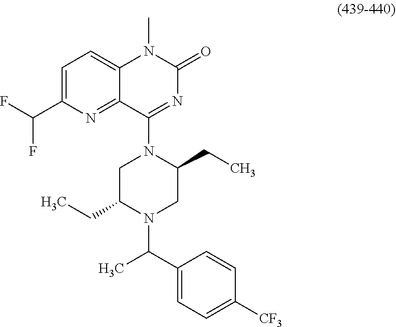 [SMILES: CC[C@H]1CN(C(C)c2ccc(C(F)(F)F)cc2)[C@H](CC)CN1c1nc(=O)n(C)c2ccc(C(F)F)nc12]

To a stirred solution of 6-chloro-4-((2S,5R)-2,5-diethyl-4-(1-(4-(trifluoromethyl) phenyl)ethyl)piperazin-1-yl)-1-methylpyrido[3,2-d]pyrimidin-2(1H)-one (80 mg, 0.16 mmol) in toluene (5 mL) were added (1,3-bis(2,6-diisopropylphenyl)imidazolidin-2-ylidene)(difluoromethyl)silver (87 mg, 0.16 mmol), bis(2-diphenylphosphinophenyl) ether (9.0 mg, 0.016 mmol) and bis(dibenzylideneacetone)palladium(0) (4.53 mg, 7.87 μmol). The reaction mixture was purged with argon over 5 min and was heated at 80° C. for 16 h. The reaction was quenched with the addition of water (20 mL). The reaction mixture was extracted with ethyl acetate (3×20 mL). The combined organic layer was dried over anhydrous sodium sulphate, filtered and evaporated under reduced pressure to yield crude product, which was purified by preparative HPLC. HPLC Method: Column: Gemini NX (250×21.2 mm, 5 μm); mobile phase A=10 mM ammonium acetate in water, pH 4.5, mobile phase B=acetonitrile, Flow 20 mL/min to yield Example 439 and Example 440.

EXAMPLE 439: (2 mg, 3.0% yield). LCMS: m/z=524.3 [M+H]⁺; rt 2.67 min; LCMS Method: Column: Ascentis Express C18 (50×2.1 mm, 2.7 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR: (400 MHz, DMSO-d₆) δ (ppm)=8.03-7.92 (m, 2H), 7.71-7.71 (m, 2H), 7.60-7.62 (m, 2H), 7.19-6.80 (m, 1H), 6.18-5.87 (m, 1H), 5.21-4.80 (m, 1H), 3.90-3.79 (m, 1H), 3.51-3.40 (m, 3H), 3.14-3.04 (m, 1H), 2.99-2.87 (m, 1H), 2.84-2.70 (m, 1H), 2.34-2.38 (m, 1H), 2.29-1.84, (m, 2H), 1.52-1.36 (m, 2H), 1.29 (d, J=6.5 Hz, 3H), 0.99-0.82 (m, 3H), 0.77-0.31 (m, 3H).

EXAMPLE 440: (2.3 mg, 4.39 μmol, 2.79% yield). LCMS: m/z=524.3 [M+H]⁺; rt 2.68 min; LCMS Method: Column: Ascentis Express C18 (50×2.1 mm, 2.7 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR: (400 MHz, DMSO-d₆) δ (ppm)=7.95-7.95 (m, 1H), 8.09-7.86 (m, 1H), 7.78-7.65 (m, 2H), 7.65-7.51 (m, 2H), 7.25-6.75 (m, 1H), 6.42-5.72 (m, 1H), 5.21-4.80 (m, 1H), 3.66-3.70 (m, 2H), 3.45 (s, 3H), 3.29-2.92 (m, 1H), 2.60-2.54 (m, 1H), 2.14-2.18 (m, 1H), 1.94-1.72 (m, 2H), 1.61-1.34 (m, 2H), 1.26 (d, J=6.5 Hz, 3H), 1.04-0.71 (m, 3H), 0.68-0.45 (m, 3H).

TABLE 20

| Ex. | | Stereo- | LCMS | LCMS | |
|---|---|---|---|---|---|
| No. | Structure | chemistry | Method | rt | [M + H]+ |
| 441 442 | 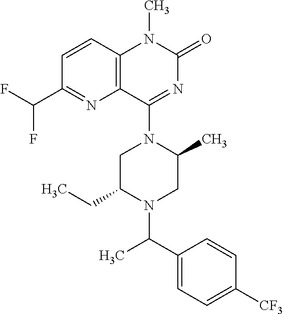 [SMILES: CC[C@@H]1CN(c2nc(=O)n(C)c3ccc(C(F)F)nc23)[C@@H](C)CN1C(C)c1ccc(C(F)(F)F)cc1] | H H | C C | 2.46 2.42 | 510.3 510.3 |

### Example 443

### 4-((2S,5R)-2,5-Diethyl-4-(1-(4-(trifluoromethyl)phenyl)ethyl)piperazin-1-yl)-6-hydroxy-1-methylpyrido[3,2-d]pyrimidin-2(1H)-one

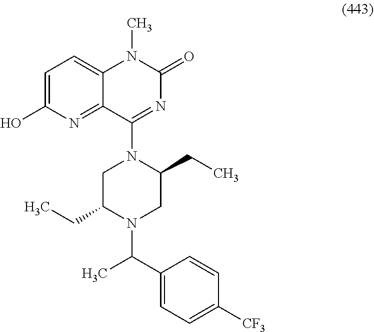 [SMILES: CC[C@H]1CN(C(C)c2ccc(C(F)(F)F)cc2)[C@H](CC)CN1c1nc(=O)n(C)c2ccc(O)nc12]

To a solution of 4-((2S,5R)-2,5-diethyl-4-(1-(4-(trifluoromethyl)phenyl)ethyl) piperazin-1-yl)-6-methoxy-1-methylpyrido[3,2-d]pyrimidin-2(1H)-one (150 mg, 0.3 mmol) in DMF (4 mL) was added lithium chloride (63.1 mg, 1.49 mmol). The reaction mixture was heated at 180° C. for 6 h. Water (50 mL) was added to quench the reaction. The reaction mixture was extracted with ethyl acetate (3×50 mL). The combined organic layer was dried over anhydrous sodium sulphate, filtered and evaporated under reduced pressure to yield 4-((2S,5R)-2,5-diethyl-4-(1-(4-(trifluoromethyl)phenyl)ethyl) piperazin-1-yl)-6-hydroxy-1-methylpyrido[3,2-d]pyrimidin-2(1H)-one (90 mg, 61.7% yield). LCMS: m/z=490.2 [M+H]⁺; retention time 0.79 min; LCMS Method; Column: AQUITY BEH C18 (2.1×50 mm, 1.7 μm); mobile phase A: 10 mM NH₄OAc/HCO₂H (pH: 5) in water:acetonitrile (95:5); mobile phase B: 10 mM NH₄OAc/HCO₂H (pH: 5) in water:acetonitrile (5:95); gradient=0-5% B over 1.1 min, then a 1.7 minute hold at 95% B, flow: 0.8 mL/min, temperature: 27° C.; detection: UV at 220 nm.

### Examples 444 and 445

### 4-((2S,5R)-2,5-Diethyl-4-(1-(4-(trifluoromethyl)phenyl)ethyl)piperazin-1-yl)-6-(difluoromethoxy)-1-methylpyrido[3,2-d]pyrimidin-2(1H)-one

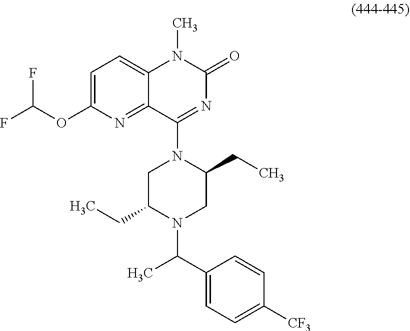 [SMILES: CC[C@H]1CN(C(C)c2ccc(C(F)(F)F)cc2)[C@H](CC)CN1c1nc(=O)n(C)c2ccc(OC(F)F)nc12]

To a stirred solution of 4-((2S,5R)-2,5-diethyl-4-(1-(4-(trifluoromethyl)phenyl) ethyl)piperazin-1-yl)-6-hydroxy-1-methylpyrido[3,2-d]pyrimidin-2(1H)-one (90 mg, 0.18 mmol) in acetonitrile (5 mL) was added NaH (18 mg, 0.46 mmol, 60% w/w) at 0° C. The reaction mixture was stirred at room temperature for 20 min and 2 2-difluoro-2-(fluorosulfonyl)acetic acid (0.03 mL, 0.31 mmol) was added. The reaction mixture was stirred at room temperature for another 3 h. Water was added to quench the reaction. The mixture was concentrated under reduced pressure to obtain the crude product, which was suspended with water (20 mL) and was extracted with ethyl acetate (3×20 mL). The combined organic layer was dried over anhydrous sodium sulphate, filtered and evaporated under reduced pressure to obtain the crude product, which was purified by preparative HPLC [Method Info: Preparative Column: ACE C18 (250×21.2 mm, 5 μm), mobile phase A=10 mM ammonium acetate in water, mobile phase B=acetonitrile:MeOH (1:1), Flow 19 mL/min, Gradient: 70-100% B over 25 minutes, then a 5 minute hold at 100% B].

EXAMPLE 444: (1.0 mg, 1.853 μmol, 1.0% yield). LCMS: m/z=540.3 [M+H]⁺; rt 2.48 min; LCMS Method: Column: Ascentis Express C18 (50×2.1 mm, 2.7 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR: (400 MHz, DMSO-d₆) δ (ppm)=8.03 (d, J=9.0 Hz, 1H), 7.71 (d, J=8.1 Hz, 2H), 7.60-7.62 (m, 2H), 7.36-7.55 (m, 2H), 6.00-5.34 (m, 1H), 5.25-4.64 (m, 1H), 3.91-3.75 (m, 1H), 3.62-3.41 (m, 1H), 3.38-3.44 (m, 4H), 3.16-2.98 (m, 1H), 2.98-2.72 (m, 2H), 2.39-2.28 (m, 1H), 2.14-1.84 (m, 2H), 1.32-1.20, (m, 3H), 1.54-1.11 (m, 2H), 0.92-0.71 (m, 3H), 0.94-0.25 (m, 1H).

EXAMPLE 445: (1.0 mg, 1.86 μmol, 1% yield). LCMS: m/z=540.3 [M+H]⁺; rt 2.49 min; LCMS Method: Column: Ascentis Express C18 (50×2.1 mm, 2.7 μm); mobile phase A: 10 mM NH₄OAc in water:acetonitrile (95:5); mobile phase B: 10 mM NH₄OAc in water:acetonitrile (5:95); Gradient=0-100% B over 3 minutes; Temperature: 50° C.; Flow rate: 1.1 mL/min; Detection: UV at 220 nm. ¹H NMR: (400 MHz, DMSO-d₆) δ (ppm)=8.04 (d, J=9.3 Hz, 1H), 7.68-7.76 (m, 2H), 7.61-7.63 (m, 2H), 7.43-7.50 (m, 2H), 5.80-5.59 (m, 1H), 5.04-4.89 (m, 1H), 3.80-3.71 (m, 1H), 3.67-3.70 (m, 1H), 3.44 (s, 3H), 3.27-3.22 (m, 1H), 3.04-2.98 (m, 1H), 2.17-2.20 (m, 1H), 2.04-1.87 (m, 1H), 1.63-1.70 (m, 1H), 1.42-1.46 (m, 2H), 1.24 (d, J=6.4 Hz, 3H), 1.01-0.78 (m, 2H), 0.73-0.39 (m, 4H).

### Intermediate 100

### (5-Cyclopropylisoxazol-3-yl)(4-(trifluoromethoxy)phenyl)methanone

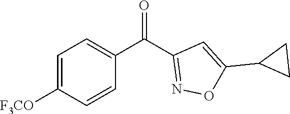 [SMILES: O=C(c1ccc(OC(F)(F)F)cc1)c1cc(C2CC2)on1]

To a stirred solution of 5-cyclopropyl-N-methoxy-N-methylisoxazole-3-carboxamide (250 mg, 1.27 mmol) in tetrahydrofuran (4 mL) was added (4-(trifluoromethoxy)phenyl)magnesium bromide (0.5 M, 12.7 mL, 6.37 mmol) at 0° C. The reaction mixture was stirred at room temperature for 16 h. The reaction was quenched with the addition of a saturated aqueous NH₄Cl solution. The mixture was extracted with ethyl acetate (2×20 mL). The organic layer was separated, dried over Na₂SO₄ and evaporated under reduced pressure to yield (5-cyclopropylisoxazol-3-yl)(4-(trifluoromethoxy)phenyl)methanone (250 mg, 66% yield). ¹H NMR (CDCl₃, 400 MHz): δ (ppm)=8.38-8.43 (m, 2H), 7.33-7.38 (m, 2H), 6.46 (s, 1H), 1.67-1.78 (m, 1H), 1.15-1.21 (m, 2H), 1.04-1.10 (m, 2H).

### Intermediate 101

### (5-Cyclopropylisoxazol-3-yl)(4-fluorophenyl)methanol

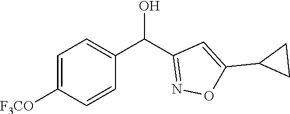 [SMILES: OC(c1ccc(OC(F)(F)F)cc1)c1cc(C2CC2)on1]

To a stirred solution of (5-cyclopropylisoxazol-3-yl)(4-(trifluoromethoxy)phenyl) methanone (800 mg, 2.69 mmol) in MeOH (10 mL) was added NaBH₄ (204 mg, 5.38 mmol) at 0° C. The reaction mixture was stirred at room temperature for 3 h. The reaction was quenched with the addition of saturated aqueous NH₄Cl. The mixture was diluted with water and extracted with ethyl acetate (20 mL). The organic layer was separated, dried over Na₂SO₄ and evaporated under reduced pressure to yield (5-cyclopropylisoxazol-3-yl)(4-fluorophenyl)methanol (710 mg, 88% yield). ¹H NMR (CDCl₃, 400 MHz): δ (ppm)=7.44-7.49 (m, 2H), 7.19-7.23 (m, 2H), 5.92 (d, J=4.0 Hz, 1H), 5.78 (s, 1H), 2.80 (d, J=4.0 Hz, 1H), 1.98 (tt, J=8.5, 5.0 Hz, 1H), 1.01-1.05 (m, 2H), 0.91-0.97 (m, 2H).

### Intermediate 102

### 3-(Bromo(4-(trifluoromethoxy)phenyl)methyl)-5-cyclopropylisoxazole

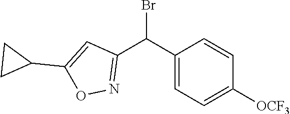 [SMILES: FC(F)(F)Oc1ccc(C(Br)c2cc(C3CC3)on2)cc1]

To a stirred solution of (5-cyclopropylisoxazol-3-yl)(4-(trifluoromethoxy)phenyl) methanol (300 mg, 1.00 mmol) in dichloromethane (5 mL) was added BBr₃ (2.005 mL, 2.005 mmol) at 0° C. and stirred at room temperature for 2 h. The reaction mixture evaporated under reduced pressure to yield 3-(bromo(4-(trifluoromethoxy)phenyl) methyl)-5-cyclopropylisoxazole (240 mg, 66% yield). ¹H NMR (CDCl₃, 400 MHz): δ (ppm)=7.54-7.59 (m, 2H), 7.23 (dd, J=8.8, 1.0 Hz, 2H), 6.15 (s, 1H), 6.10 (s, 1H), 2.05 (tt, J=8.5, 5.2 Hz, 1H), 1.07-1.13 (m, 2H), 0.99-1.05 (m, 2H).

### Intermediate 103

### 2-Cyclopropyl-N-methoxy-N-methylthiazole-5-carboxamide

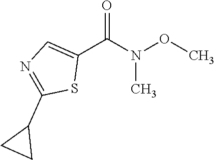

To a solution of 2-cyclopropylthiazole-5-carboxylic acid (0.4 g, 2.36 mmol) in DMF (5 mL) at room temperature was added HATU (1.348 g, 3.55 mmol), DIPEA (1.2 mL, 7.09 mmol) and N,O-dimethylhydroxylamine hydrochloride (0.346 g, 3.55 mmol). The reaction mixture was stirred at room temperature for 5 h. The reaction was quenched with the addition of water. The mixture was extracted with ethyl acetate (2×100 mL), the combined organic layer was washed with water, brine and dried over sodium sulfate. The solvent was evaporated under reduced pressure to yield the crude product (2-cyclopropylthiazol-5-yl)(4-fluorophenyl)methanol (0.15 g, 46% yield. LCMS: m/z=213.2 [M+H]⁺; rt 0.91 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min.

### Intermediate 104

### (2-Cyclopropylthiazol-5-yl)(4-fluorophenyl)methanone

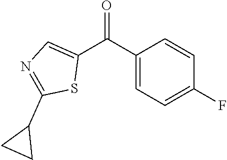 [SMILES: O=C(c1ccc(F)cc1)c1cnc(C2CC2)s1]

To a solution of 2-cyclopropyl-N-methoxy-N-methylthiazole-5-carboxamide (0.2 g, 0.94 mmol) in THF (10 mL) at 0° C. was added (4-fluorophenyl)magnesium bromide (1 M, 1.8 mL, 1.8 mmol). The reaction mixture was and stirred at 25° C. for 16 h. The reaction mixture was quenched with saturated ammonium chloride solution and extracted with ethylacetate (2×100 mL), the combined organic layer was washed with water, brine and dried over sodium sulfate. The solvent was evaporated under reduced pressure to yield the crude desired product (2-cyclopropylthiazol-5-yl)(4-fluorophenyl)methanol (0.15 g, 48% yield). LCMS: m/z=248.1 [M+H]⁺; rt 1.56 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 2 min, then a 1 minute hold at 90% B, flow: 0.7 mL/min.

### Intermediate 105

### (2-Cyclopropylthiazol-5-yl)(4-fluorophenyl)methanol

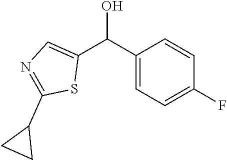 [SMILES: OC(c1ccc(F)cc1)c1cnc(C2CC2)s1]

To a solution of (2-cyclopropylthiazol-5-yl)(4-fluorophenyl)methanone (0.2 g, 0.81 mmol) in methanol (5 mL) at 0° C. was added sodium borohydride (0.046 g, 1.21 mmol). The reaction mixture was stirred at room temperature for 3 h. The reaction was quenched with the addition of saturated aqueous ammonium chloride solution. The mixture was extracted with ethyl acetate (2×100 mL), the combined organic layer was washed with water, brine and dried over sodium sulfate. The solvent was evaporated under reduced pressure to yield (2-cyclopropylthiazol-5-yl)(4-fluorophenyl)methanol (0.15 g, 48% yield). LCMS: m/z=250.2 [M+H]⁺; rt 1.22 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 106

### 5-(Bromo(4-fluorophenyl)methyl)-2-cyclopropylthiazoles

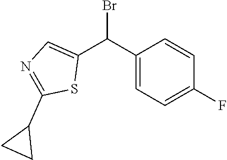 [SMILES: Fc1ccc(C(Br)c2cnc(C3CC3)s2)cc1]

To a solution of (2-cyclopropylthiazol-5-yl)(4-fluorophenyl)methanol (0.2 g, 0.8 mmol) in DCM (10 mL) at 0° C. was added boron tribromide (1.2 mL, 1.2 mmol). The reaction mixture was stirred at room temperature for 3 h. The reaction was quenched with the addition of water. The mixture was extracted with ethyl acetate (2×100 mL), the combined organic layer was washed with water, brine and dried over sodium sulfate. The solvent was evaporated under reduced pressure to yield 5-(bromo(4-fluorophenyl) methyl)-2-cyclopropylthiazole (0.18 g, 0.577 mmol, 71.9% yield).

### Intermediate 107

### Ethyl (1R,2R)-2-(4-formylphenoxy)cyclopropane-1-carboxylate

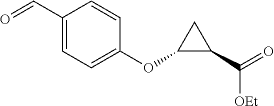 [SMILES: CCOC(=O)[C@@H]1C[C@H]1Oc1ccc(C=O)cc1]

To a stirred suspension of KOH (1.378 g, 24.57 mmol) in DMSO (12 mL) were added 4-hydroxybenzaldehyde (0.75 g, 6.14 mmol) and ethyl (1S,2R)-2-iodocyclopropane-1-carboxylate (1.769 g, 7.37 mmol). The reaction mixture was stirred at room temperature for 16 h. The reaction mixture was quenched with water (100 mL) and was extracted with ethyl acetate (3×100 mL). The combined organic layer was dried over anhydrous sodium sulfate, filtered and evaporated under reduced pressure to yield the crude product (350 mg, 24%). LCMS: m/z=235.1 [M+H]⁺; retention time 1.49 & 1.51 min (diastereomeric mixture), LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min.

### Intermediate 108

### Ethyl (1R,2R)-2-(4-(1-hydroxyethyl)phenoxy)cyclopropane-1-carboxylate

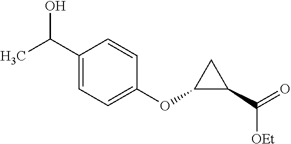 [SMILES: CCOC(=O)[C@@H]1C[C@H]1Oc1ccc(C(C)O)cc1]

To a stirred solution of ethyl (1R,2R)-2-(4-formylphenoxy)cyclopropane-1-carboxylate (50 mg, 0.21 mmol) in tetrahydrofuran (3 mL) at 0° C. was added methylmagnesium bromide in diethyl ether (3 M, 0.1 mL, 0.3 mmol) drop wise. The reaction mixture stirred at room temperature for 2 h. The reaction was quenched with the addition of saturated aqueous ammonium chloride solution (10 mL). The mixture was extracted with ethyl acetate (3×10 mL). The combined organic layer was dried over anhydrous sodium sulfate, filtered and evaporated under reduced pressure to yield crude product. ¹H NMR (300 MHz, CDCl₃) δ (ppm)=7.25-7.19 (m, 2H), 6.88 (d, J=9.1 Hz, 2H), 4.80 (q, J=6.4 Hz, 1H), 4.17-4.08 (m, 2H), 4.02-3.95 (m, 1H), 3.51-3.54 (m, 1H), 1.87-1.83 (m, 1H), 1.65-1.70 (m, 1H), 1.42-1.36 (m, 3H), 1.23-1.19 (m, 3H).

### Intermediate 109

### Ethyl (1S,2R)-2-(4-(1-chloroethyl)phenoxy)cyclopropane-1-carboxylate

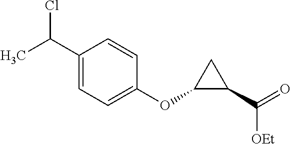 [SMILES: CCOC(=O)[C@@H]1C[C@H]1Oc1ccc(C(C)Cl)cc1]

To a stirred solution of ethyl (1R,2R)-2-(4-(1-hydroxyethyl)phenoxy) cyclopropane-1-carboxylate (70 mg, 0.28 mmol) in DCM (2 mL) was added SOCl₂ (0.16 mL, 2.24 mmol). The reaction mixture was heated at 40° C. for 5 h. The reaction mixture was concentrated under reduced pressure to yield ethyl (1R,2R)-2-(4-(1-chloroethyl) phenoxy)cyclopropane-1-carboxylate (68 mg, 0.25 mmol, 90% yield). ¹H NMR (300 MHz, CDCl₃) δ (ppm)=7.40-7.20 (m, 2H), 6.97-6.74 (m, 2H), 5.17-4.89 (m, 1H), 4.21-4.03 (m, 2H), 2.00-1.77, (m, 2H), 1.57-1.34 (m, 4H), 1.34-1.14 (m, 4H).

### Intermediate 110

### 4-(2-(4-Bromophenyl)propan-2-yl)morpholine

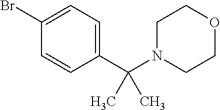 [SMILES: CC(C)(c1ccc(Br)cc1)N1CCOCC1]

To a stirred solution of 2-(4-bromophenyl)propan-2-amine (1 g, 4.67 mmol) in acetonitrile (8 mL) were added K₂CO₃ (1.94 g, 14.01 mmol) and 2-bromoethyl ether (1.63 g, 7.01 mmol) at room temperature. The reaction mixture was stirred under microwave irradiation at 100° C. for 2 h. The reaction mixture was cooled to room temperature and solvent was removed under reduced pressure to give the crude product, which was purified by silica gel column chromatography using 70-80% EtOAc in n-hexane to afford 4-(2-(4-bromophenyl)propan-2-yl)morpholine (1 g, 62% yield). LCMS: m/z, 285.1 [M+2]; rt 2.05 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 2 min, then a 0.3 minute hold at 90% B, flow: 0.7 mL/min.

### Intermediate 111

### 1-(4-(2-Morpholinopropan-2-yl)phenyl)ethan-1-one

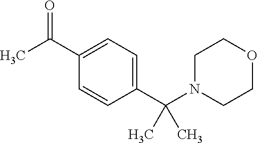 [SMILES: CC(=O)c1ccc(C(C)(C)N2CCOCC2)cc1]

To a stirred solution of 4-(2-(4-bromophenyl)propan-2-yl)morpholine (0.5 g, 1.759 mmol) in DMF (5 mL) were added tributyl(1-ethoxyvinyl)tin (0.83 g, 2.29 mmol), bis(triphenylphosphine)palladium(II) dichloride (0.12 g, 0.176 mmol) at room temperature. The reaction mixture was flushed with nitrogen and heated at 70° C. for 16 h. The reaction mixture was cooled to room temperature and the volatiles were removed under reduced pressure to obtain the crude product. The residue was dissolved in 1 mL of aqueous 5 N HCl and stirred for 15 min. The solvent was removed under reduced pressure to yield the product, which was purified by silica gel column chromatography (eluting with 40-50% EtOAc in n-hexane) to afford 1-(4-(2-morpholinopropan-2-yl) phenyl)ethan-1-one (0.3 g, 69% yield). ¹H NMR (300 MHz, DMSO-d₆) δ (ppm)=8.05 (d, J=8.3 Hz, 2H), 7.97-7.90 (m, 2H), 3.89 (br d, J=6.8 Hz, 4H), 3.20-3.10 (m, 2H), 3.05-2.91 (m, 2H), 2.62 (s, 3H), 1.84 (s, 6H).

### Intermediate 112

### 1-(4-(2-Morpholinopropan-2-yl)phenyl)ethan-1-ol (racemate)

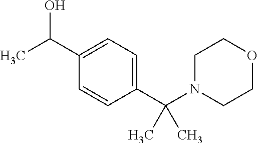 [SMILES: CC(O)c1ccc(C(C)(C)N2CCOCC2)cc1]

To a solution of 1-(4-(2-morpholinopropan-2-yl)phenyl)ethan-1-one (0.3 g, 1.21 mmol) in methanol (10 mL) was added NaBH₄ (0.09 g, 2.43 mmol) at 0° C. under a nitrogen atmosphere. The reaction mixture was stirred at room temperature for 2 h. The reaction was quenched with water (5 mL). The reaction mixture was extracted with ethyl acetate (2×20 mL). The combined organic layer was dried over anhydrous Na₂SO₄, evaporated under reduced pressure to obtain 1-(4-(2-morpholinopropan-2-yl)phenyl) ethan-1-ol (250 mg, 50% yield). LCMS: m/z, 250.2 [M+H]⁺; rt 1.07 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 113

### 4-(2-(4-(1-chloroethyl)phenyl)propan-2-yl)morpholine (racemate)

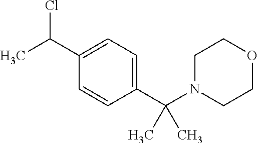 [SMILES: CC(Cl)c1ccc(C(C)(C)N2CCOCC2)cc1]

To a solution of 1-(4-(2-morpholinopropan-2-yl)phenyl)ethan-1-ol (0.1 g, 0.40 mmol) in dichloromethane (5.0 mL) was added SOCl₂ (0.15 mL, 2.00 mmol)) at 0° C. The reaction mixture was stirred at room temperature for 2 h. The volatiles were removed from the reaction mixture under reduced pressure to obtain 4-(2-(4-(1-chloroethyl)phenyl)propan-2-yl)morpholine (0.1 g, 0.273 mmol, 68% yield). LCMS: m/z, 268.1 [M+H]⁺; rt 1.89 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 114

### 1-(4-(1-Hydroxyethyl)phenyl)pyrrolidin-2-one

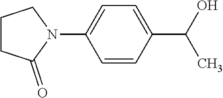

To a solution of 1-(4-acetylphenyl)pyrrolidin-2-one (250 mg, 1.23 mmol) in dry MeOH (5.0 mL) at 0° C., sodium borohydride (140 mg, 3.69 mmol) was added. The reaction mixture stirred for 2 h at room temperature. The reaction was quenched with saturated sodium bicarbonate solution. The reaction mixture was stirred for 10 minutes and was extracted with ethyl acetate (2×25 mL). The organic layer was separated, washed with water, brine, dried over sodium sulfate and concentrated under reduced pressure to yield 1-(4-(1-hydroxyethyl)phenyl)pyrrolidin-2-one (250 mg, 99% yield) as an off-white solid. LCMS: m/z=206.1 [M+H]⁺; retention time 0.76 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.2 minute hold at 100% B, flow: 0.7 mL/min.

### Intermediate 115

### 1-(4-(1-Chloroethyl)phenyl)pyrrolidin-2-one

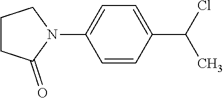

To a solution of 1-(4-(1-hydroxyethyl)phenyl)pyrrolidin-2-one (100 mg, 0.49 mmol) in DCM (2.0 mL), thionyl chloride (0.053 mL, 0.731 mmol) was added slowly at 0° C. The mixture was stirred for 1 h. and then diluted with DCM (50 mL). The mixture was washed with water, brine, dried over sodium sulfate and concentrated under reduced pressure to yield 1-(4-(1-chloroethyl)phenyl)pyrrolidin-2-one (100 mg, 92% yield). LCMS: m/z=242.1 [M+NH₄⁺]; retention time 1.11 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 116

### 5-(1-Chloroethyl)-3-methylbenzo[d]oxazole-2(3H)-one

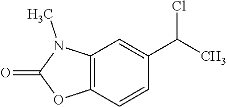

To a solution of 5-(1-hydroxyethyl)-3-methylbenzo[d]oxazol-2(3H)-one (0.2 g, 1.04 mmol) in DCM (5 mL) was added thionyl chloride (0.1 mL, 1.55 mmol) at 0° C. The reaction mixture was stirred at room temperature for 16 h. The volatiles were evaporated under reduced pressure to afford 5-(1-chloroethyl)-3-methylbenzo[d]oxazol-2(3H)-one (0.2 g, 59% yield). LCMS: m/z=212.1 [M+H]⁺; retention time 0.60 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-90% B over 1.1 min, then a 0.6 minute hold at 90% B, flow: 0.7 mL/min.

### Intermediate 117

### 1-(4-(Methoxy-d₃)phenyl)propan-1-one

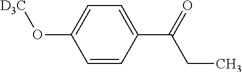

To a stirred solution of 1-(4-hydroxyphenyl)propan-1-one (0.5 g, 3.33 mmol) in DMF (10 mL) were added K₂CO₃ (0.92 g, 6.66 mmol) and iodomethane-d₃ (0.579 g, 4.00 mmol) at room temperature. The reaction mixture was stirred for 16 h. The reaction mixture was diluted with ethyl acetate (200 mL) and washed with water. The organic layer was dried over anhydrous Na₂SO₄, filtered and evaporated under reduced pressure to afford 1-(4-(methoxy-d₃)phenyl)propan-1-one (0.52 g, 92% yield). LCMS: m/z=168.2 [M+H]⁺; rt 2.129 min. LCMS Method: (LCMS method: Column: Column-Kinetex XB-C18 (75×3 mm-2.6 μm), mobile phase A: 98% water: 2% acetonitrile; 10 mM ammonium formate; mobile phase B: 2% water: 98% acetonitrile; 10 mM ammonium Formate; Flow: 1.0 mL/min; Temp: 50° C.; Time (min): 0-4; % B: 0-100%).

### Intermediate 118

### 1-(4-(Methoxy-d₃)phenyl)propan-1-ol

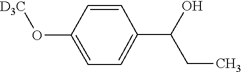

To a stirred solution of 1-(4-(methoxy-d₃)phenyl)propan-1-one (0.5 g, 2.99 mmol) in MeOH (1 mL) and THF (10 mL) was added NaBH₄ (0.283 g, 7.48 mmol) at 0° C. The reaction mixture was stirred at room temperature for 3 h. The solvent was removed under reduced pressure to yield the crude product, which was dissolved in ethyl acetate and washed with saturated aqueous NH₄Cl solution. The organic layer was dried over anhydrous Na₂SO₄, filtered and evaporated under reduced pressure to afford 1-(4-(methoxy-d₃)phenyl)propan-1-ol (0.38 g, 75% yield). ¹H NMR (400 MHz, DMSO-d₆) δ (ppm)=7.21 (d, J=8.80 Hz, 2H) 6.86 (d, J=7.83 Hz, 2H) 4.98 (d, J=4.40 Hz, 1H) 4.34-4.40 (m, 1H) 1.49-1.66 (m, 2H) 0.79 (t, J=7.34 Hz, 3H).

### Intermediate 119

### 1-(1-Chloropropyl)-4-(methoxy-d₃)benzene

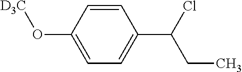

To a stirred solution of 1-(4-(methoxy-d₃)phenyl)propan-1-ol (0.35 g, 2.07 mmol) in dry DCM (10 mL) was added SOCl₂ (1 mL, 13.70 mmol) at room temperature. The reaction mixture was stirred for 2 h. The solvent was removed under reduced pressure to yield 1-(1-chloropropyl)-4-(methoxy-d₃)benzene (0.380 g, 98% yield). ¹H NMR (400 MHz, DMSO-d₆) δ (ppm)=7.24-7.39 (m, 2H) 6.84-6.98 (m, 2H) 5.03 (t, J=7.28 Hz, 1H) 1.96-2.14 (m, 2H) 0.91 (t, J=7.28 Hz, 3H).

### Intermediate 120

### 1-(2-Morpholino-4-(trifluoromethyl)phenyl)ethan-1-one

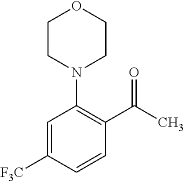

To a stirred solution of 1-(2-fluoro-4-(trifluoromethyl)phenyl)ethan-1-one (0.5 g, 2.43 mmol) in DMF (3 mL) was added morpholine (0.528 g, 6.06 mmol) and the reaction mixture was heated at 100° C. for 16 h. The reaction mixture was cooled to room temperature and diluted with ethyl acetate and washed with water, the organic layer was dried over anhydrous Na₂SO₄, filtered and evaporated under reduced pressure to yield the crude compound which was purified by column chromatography (eluted with 20-10% ethyl acetate/pet ether) to yield 1-(2-morpholino-4-(trifluoromethyl)phenyl) ethan-1-one (0.55 g, 82% yield)). LCMS: m/z=274.2 [M+H]⁺; rt 2.502 min. LCMS Method: Method info: Column-Kinetex XB-C18 (75×3 mm-2.6 μm), Mobile phase A: 10 mM NH₄OAc in water:acetonitrile (98:02); mobile phase B: 10 mM NH₄OAc in water:acetonitrile (02:98), Gradient: 20-100% B over 4 minutes, then a 16 minute hold at 100% B; Flow: 1.5 mL/min).

### Intermediate 121

### 1-(2-Morpholino-4-(trifluoromethyl)phenyl)ethan-1-ol

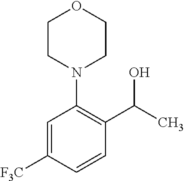

To a stirred solution of 1-(2-morpholino-4-(trifluoromethyl)phenyl)ethan-1-one (0.3 g, 1.1 mmol) in dry methanol (10 mL) was added NaBH₄ (0.104 g, 2.74 mmol) at 0° C. The reaction mixture was stirred at room temperature for 4 h. The solvent was removed under reduced pressure to yield the crude product, which was dissolved in ethyl acetate and washed with saturated aqueous ammonium chloride solution. The organic layer was dried over anhydrous Na₂SO₄, filtered and evaporated under reduced pressure to yield crude compound, which was purified by column chromatography (eluted with 40-30% ethyl acetate/pet ether) to afford 1-(2-morpholino-4-(trifluoromethyl)phenyl)ethan-1-ol (0.3 g, 98% yield. LCMS: m/z=276.2 [M+H]⁺; rt 2.174 min. LCMS Method: Method info: Column-Kinetex XB-C18 (75×3 mm-2.6 μm), Mobile phase A: 10 mM NH₄OAc in water:acetonitrile (98:02); mobile phase B: 10 mM NH₄OAc in water:acetonitrile (02:98), Gradient: 20-100% B over 4 minutes, then a 16 minute hold at 100% B; Flow: 1.5 mL/min).

### Intermediate 122

### 4-(2-(1-Chloroethyl)-5-(trifluoromethyl)phenyl)morpholine

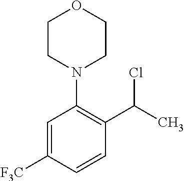

To a stirred solution of 1-(2-morpholino-4-(trifluoromethyl)phenyl)ethan-1-ol (0.3 g, 1.09 mmol) in dry DCM (5 mL) was added SOCl₂ (1 mL, 13.70 mmol) at room temperature. The reaction mixture was stirred at room temperature for 16 h. The solvent was removed under reduced pressure to yield 4-(2-(1-chloroethyl)-5-(trifluoromethyl) phenyl)morpholine (0.32 g, 100% yield).

### Intermediate 123

### 1-(4-(3,6-Dihydro-2H-pyran-4-yl)phenyl)ethan-1-one

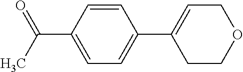

To a stirred solution of 2-(3,6-dihydro-2H-pyran-4-yl)-4,4,5,5-tetramethyl-1,3,2-dioxaborolane (1.15 g, 5.53 mmol) in 1,4-dioxane (15 mL) and H₂O (1 mL) were added 1-(4-bromophenyl)ethan-1-one (1.0 g, 5.02 mmol) and K₂CO₃ (1.4 g, 10.05 mmol). The reaction mixture was degassed with argon for 10 min. and PdCl₂(dppf) (0.37 g, 0.502 mmol) was added. The reaction mixture was heated at 100° C. for 16 h. The reaction mixture was cooled to room temperature, extracted with EtOAc (2×100 mL), washed with water, brine, dried over Na₂SO₄ and concentrated to give crude product, which was purified by flash chromatography using with 30%-40% EtOAc in pet. ether to afford 1-(4-(3,6-dihydro-2H-pyran-4-yl)phenyl)ethan-1-one (800 mg, 79% yield). ¹H NMR (300 MHz, CDCl₃) δ (ppm)=7.96-7.73 (m, 2H), 7.41 (d, J=8.3 Hz, 2H), 6.26-6.11 (m, 1H), 4.37-4.22 (m, 2H), 3.94-3.81 (m, 2H), 2.51 (s, 3H), 2.50-2.44 (m, 2H).

### Intermediate 124

### 1-(4-(Tetrahydro-2H-pyran-4-yl)phenyl)ethan-1-one

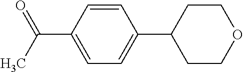

To a solution of 1-(4-(3,6-dihydro-2H-pyran-4-yl)phenyl)ethan-1-one (500 mg, 2.47 mmol) in EtOAc (50 mL) was added platinum (IV) oxide (56.1 mg, 0.247 mmol). The solution was stirred under H₂ for 16 h. The reaction mixture was filtered through a Celite® pad and washed with excess EtOAc (50 mL). The filtrate was concentrated under reduced pressure to give 1-(4-(tetrahydro-2H-pyran-4-yl)phenyl)ethan-1-one (400 mg, 79% yield). ¹H NMR (300 MHz, CDCl₃) δ (ppm)=7.97-7.75 (m, 2H), 7.37-7.24 (m, 2H), 4.15-3.81 (m, 2H), 3.59-3.35 (m, 2H), 2.87-2.68 (m, 1H), 2.50 (s, 3H), 1.86-1.66 (m, 4H).

### Intermediate 125

### 1-(4-(Tetrahydro-2H-pyran-4-yl)phenyl)ethan-1-one

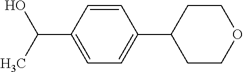

To a solution of 1-(4-(2-morpholinopropan-2-yl)phenyl)ethan-1-one (0.3 g, 1.21 mmol) in methanol (10 mL) was added NaBH₄ (0.09 g, 2.43 mmol) at 0° C. under nitrogen. The reaction mixture was allowed to warm to room temperature and stirred for 2 h. The reaction was quenched with the addition of water (5 mL). The reaction mixture was extracted with ethyl acetate (2×20 mL). The combined organic layer was dried over anhydrous Na₂SO₄, evaporated under reduced pressure to obtain 1-(4-(2-morpholinopropan-2-yl)phenyl)ethan-1-ol (250 mg, 50% yield). LCMS: m/z, 189.1 [M-OH]; rt 1.31 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min. ¹H NMR (300 MHz, CDCl₃) δ (ppm)=7.30-7.24 (m, 2H), 7.17-7.12 (m, 2H), 4.90-4.75 (m, 1H), 4.08-3.94 (m, 2H), 3.50-3.40 (m, 2H), 2.75-2.60 (m, 1H), 1.78-1.67 (m, 4H), 1.43 (d, J=6.4 Hz, 3H).

### Intermediate 126

### 4-(4-(1-Chloroethyl)phenyl)tetrahydro-2H-pyran

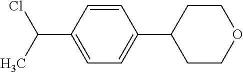

To a solution of 1-(4-(tetrahydro-2H-pyran-4-yl)phenyl)ethan-1-ol (100 mg, 0.49 mmol) in dichloromethane (5.0 mL) was added SOCl₂ (0.14 mL, 1.94 mmol)) at 0° C. The reaction mixture was stirred at room temperature for 3 h. The volatiles were removed from the reaction mixture under reduced pressure to give crude product, which was extracted with DCM (2×50 mL) and washed with saturate NaHCO₃ solution, water, brine, dried over sodium sulphate and concentrated under reduced pressure to obtain 4-(4-(1-chloroethyl)phenyl)tetrahydro-2H-pyran (90 mg, 83% yield). LCMS: m/z, 189.1 [M-Cl]; rt 1.96 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 0.3 minute hold at 100% B, flow: 0.7 mL/min. ¹H NMR (300 MHz, CDCl₃) δ (ppm)=7.31 (d, J=8.3 Hz, 2H), 7.18-7.12 (m, 2H), 5.03 (q, J=6.9 Hz, 1H), 4.08-3.94 (m, 2H), 3.54-3.37 (m, 2H), 2.79-2.60 (m, 1H), 1.84-1.75 (m, 4H), 1.74-1.67 (m, 3H).

### Intermediate 127

### Ethyl 2-(4-acetylphenoxy)-2-methylpropanoate

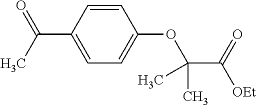 [SMILES: CCOC(=O)C(C)(C)Oc1ccc(C(C)=O)cc1]

To a stirred solution of 1-(4-hydroxyphenyl)ethan-1-one (5 g, 36.7 mmol) in acetonitrile (20 mL) was added K₂CO₃ (25.4 g, 184 mmol) and ethyl 2-bromo-2-methylpropanoate (10.9 mL, 73.4 mmol) at room temperature. The reaction mixture was heated at 80° C. for 16 h. The reaction mixture was diluted with ethyl acetate (100 mL) and washed with water, the organic layer was dried over anhydrous Na₂SO₄ and filtered. The volatiles were removed under reduced pressure to yield ethyl 2-(4-acetylphenoxy)-2-methylpropanoate (5.1 g, 56% yield). LCMS: m/z=251.3 [M+H]⁺; rt 1.52 min, LCMS Method; Column: AQUITY UPLC BEH C18 (3.0×50 mm, 1.7 μm); mobile phase A: 10 mM NH₄OAc in water:acetonitrile (95:5); mobile phase B: 10 mM NH₄OAc in water:acetonitrile (5:95); gradient=20-100% B over 2 min, then a 1 minute hold at 100% B, flow: 0.7 mL/min.

### Intermediate 128

### Ethyl 2-(4-(1-hydroxyethyl)phenoxy)-2-methylpropanoate

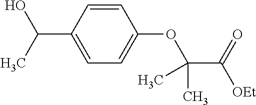 [SMILES: CCOC(=O)C(C)(C)Oc1ccc(C(C)O)cc1]

To a stirred solution of ethyl 2-(4-acetylphenoxy)-2-methylpropanoate (4 g, 15.98 mmol) in MeOH (1 mL) was added sodium borohydride (1.21 g, 32.0 mmol) at 0° C. The reaction mixture was stirred at room temperature for 3 h. The solvent was removed under reduced pressure to obtain the crude product, which was dissolved in ethyl acetate, washed with saturated aqueous NH₄Cl solution. The organic layer was dried over anhydrous, filtered, and evaporated under reduced pressure to yield ethyl 2-(4-(1-hydroxyethyl)phenoxy)-2-methylpropanoate (2.8 g, 69% yield). ¹H NMR (CDCl₃, 400 MHz): δ (ppm)=7.20-7.31 (m, 2H), 6.74-6.86 (m, 2H), 4.81-4.87 (m, 1H), 4.01-4.37 (m, 2H), 3.77 (s, 3H), 1.59 (s, 6H), 1.47 (d, J=6.5 Hz, 3H).

### Intermediate 129

### Ethyl 2-(4-(1-chloroethyl)phenoxy)-2-methylpropanoate

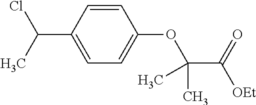 [SMILES: CCOC(=O)C(C)(C)Oc1ccc(C(C)Cl)cc1]

To a stirred solution of ethyl 2-(4-(1-hydroxyethyl)phenoxy)-2-methylpropanoate (1 g, 3.96 mmol) in dry DCM (10 mL) was added SOCl₂ (1.45 mL, 19.82 mmol) at room temperature. The reaction mixture was stirred for 2 h. The solvent was removed under reduced pressure to yield ethyl 2-(4-(1-chloroethyl)phenoxy)-2-methylpropanoate (0.92 g, 86% yield). ¹H NMR (CDCl₃, 300 MHz): δ (ppm)=7.24-7.28 (m, 2H), 6.79-6.84 (m, 2H), 4.21-4.27 (m, 1H), 3.74-3.80 (m, 2H), 1.58 (s, 3H), 1.59 (s, 3H), 1.45-1.49 (m, 3H), 1.21-1.28 (m, 3H).

### Intermediate 130

### 1-(4-(trifluoromethyl)phenyl)ethan-1-ol

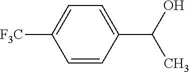

To a solution of 4-(trifluoromethyl)benzaldehyde (4.2 g, 24.12 mmol) in THF (10 mL) at 0° C. was added methylmagnesium bromide (3 M in Et₂O, 12.06 mL, 36.2 mmol). The reaction mixture was stirred at room temperature for 2 h. The reaction was quenched with the addition of saturated aqueous NH₄Cl solution. The reaction was extracted with EtOAc (2×100 mL), the combined organic layer was washed with water, brine and dried over sodium sulfate. The solvent was evaporated under reduced pressure to obtain the crude product, which was purified by silica gel column chromatography using 20% EtOAc in pet ether. The fractions were concentrated under reduced pressure to obtain 1-(4-(trifluoromethyl)phenyl)ethan-1-ol (4 g, 21.03 mmol, 87% yield). ¹H NMR (400 MHz, CDCl₃) δ (ppm)=7.67-7.57 (m, 2H), 7.50 (d, J=8.0 Hz, 2H), 4.97 (dd, J=3.8, 6.3 Hz, 1H), 1.88 (d, J=3.5 Hz, 1H), 1.51 (d, J=6.5 Hz, 3H).

### Intermediate 131

### 1-(1-Chloroethyl)-4-(trifluoromethyl)benzene

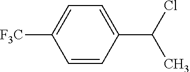

To a solution of 1-(4-(trifluoromethyl)phenyl)ethan-1-ol (1.1 g, 5.78 mmol) in DCM (10 mL) at 0° C. was added SOCl₂ (0.633 mL, 8.68 mmol). The reaction mixture was stirred at room temperature for 16 h. The volatiles were evaporated under reduced pressure to afford 1-(1-chloroethyl)-4-(trifluoromethyl)benzene (1 g, 4.79 mmol, 83% yield). ¹H NMR (400 MHz, CDCl₃) δ (ppm)=7.65-7.60 (m, 2H), 7.56-7.52 (m, 2H), 5.11 (q, J=6.8 Hz, 1H), 1.86 (d, J=7.0 Hz, 3H).

### Intermediate 132

### tert-butyl (2S,5R)-2,5-dimethyl-4-(3-(trifluoromethyl)bicyclo[1.1.1]pentane-1-carbonyl)piperazine-1-carboxylate

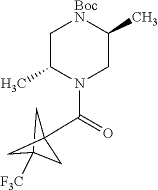 [SMILES: C[C@@H]1CN(C(=O)OC(C)(C)C)[C@@H](C)CN1C(=O)C12CC(C(F)(F)F)(C1)C2]

To a solution of tert-butyl (2S,5R)-2,5-dimethylpiperazine-1-carboxylate (74.4 mg, 0.347 mmol) and 3-(trifluoromethyl)bicyclo[1.1.1]pentane-1-carboxylic acid (50 mg, 0.278 mmol) in anhydrous DMF (2.776 mL), 1-methylimidazole (0.055 mL, 0.694 mmol) and chloro-N,N,N′,N′-tetramethylformamidinium hexafluorophosphate (117 mg, 0.416 mmol) were added sequentially and the reaction mixture was stirred at room temperature for 17 h. The reaction was quenched with the addition of water (30 mL) and the mixture extracted with EtOAc (3×). The combined extracts were washed with brine, dried over Na₂SO₄, and the solvent was removed under reduced pressure, and the crude material was purified by silica gel column chromatography using 0-60% EtOAc in n-hexane to afford tert-butyl (2S,5R)-2,5-dimethyl-4-(3-(trifluoromethyl)bicyclo[1.1.1]pentane-1-carbonyl)piperazine-1-carboxylate (92 mg, 88% yield).

LCMS: m/z, 377.4 [M+H]⁺; rt 1.45 mins (LCMS Method: Column: Acquity BEH C18 (2.1×50 mm, 1.7 μm); mobile phase A: 0.05% TFA in acetonitrile:water (5:95); mobile phase B: 0.05% TFA in acetonitrile:water (95:5), Gradient=0-100% B over 1.8 minute, then a 0.2 minute hold at 100% B; Temperature: 50° C.; Flow rate: 1.0 mL/min; Detection: UV at 220 nm). ¹H NMR (500 MHz, CDCl₃) δ 4.79-4.67 (m, 1.2H), 4.54-4.40 (m, 1.2H), 4.34-4.21 (m, 1.5H), 4.17 (br dd, J=13.4, 3.0 Hz, 1.4H), 3.83 (br d, J=13.4 Hz, 0.5H), 3.74 (br dd, J=13.2, 9.5 Hz, 0.9H), 3.65 (br d, J=14.1 Hz, 0.7H), 3.60-3.54 (m, 1.3H), 3.53-3.42 (m, 1.3H), 3.26-3.15 (m, 1.2H), 3.10 (br d, J=13.5 Hz, 0.9H), 3.03 (dd, J=13.6, 4.4 Hz, 1.0H), 2.36-2.32 (m, J=3.1 Hz, 5.6H), 2.30 (s, 6.7H), 1.47 (br s, 7.8H), 1.46 (s, 10.1H), 1.28-1.23 (m, 3.0H), 1.15 (br t, J=6.3 Hz, 7.4H), 1.10 (t, J=7.0 Hz, 2.8H). Proton NMR shows characteristics of restricted rotation (rotamers).

### Intermediate 133

### tert-butyl (2S,5R)-2,5-dimethyl-4-(1-(3-(trifluoromethyl)bicyclo[1.1.1]pentan-1-yl)propyl)piperazine-1-carboxylate (Diastereomeric Mixture)

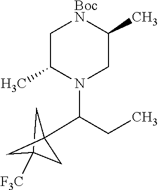 [SMILES: CCC(N1C[C@H](C)N(C(=O)OC(C)(C)C)C[C@H]1C)C12CC(C(F)(F)F)(C1)C2]

An oven-dried vial with stir bar was charged with tert-butyl (2S,5R)-2,5-dimethyl-4-(3-(trifluoromethyl)bicyclo[1.1.1]pentane-1-carbonyl)piperazine-1-carboxylate (90 mg, 0.239 mmol) and chlorocarbonylbis(triphenylphosphine)iridium(I) (Vaska's complex) (5.60 mg, 7.17 μmol) and purged with nitrogen for 5 mins. Anhydrous THF (2.657 mL), under nitrogen atmosphere was added and the solvent level marked on the vial. Additional anhydrous THF (3.5 mL), under nitrogen, was added, and the mixture continued to sparge with nitrogen for 15 mins at room temperature. The solvent level decreased during this time, approximately arriving at the marked 2.7 mL line once sparging was complete. Subsequently, 1,1,3,3-tetramethyldisiloxane (0.085 mL, 0.478 mmol) was added at room temperature and the mixture sparged with nitrogen for another 3 mins. The nitrogen sparge was stopped and the mixture further stirred at room temperature under nitrogen atmosphere for 1 h and 15 mins. Gradually, the bright yellow solution became colorless. The reaction mixture was cooled to −78° C. Ethylmagnesium bromide (0.159 mL, 0.478 mmol, 3 M in diethyl ether) was then added dropwise and the mixture stirred at −78° C. for 5 mins, and then warmed to room temperature and stirred for 17 h. The reaction was cooled to 0° C. and quenched with saturated aqueous NH₄Cl (10 mL). The mixture was stirred vigorously, warmed to room temperature and EtOAc and water were added. The aqueous layer was extracted with EtOAc (4×). The combined organic layers were washed with brine (1×) and the brine layer back-extracted with EtOAc (1×). The combined organic layers were dried over Na₂SO₄, filtered, and concentrated under reduced pressure to afford a yellow oil. The crude material was purified by silica gel column chromatography using 2-60% EtOAc in n-hexane to afford tert-butyl (2S,5R)-2,5-dimethyl-4-(1-(3-(trifluoromethyl)bicyclo[1.1.1]pentan-1-yl)propyl)piperazine-1-carboxylate (93 mg, 100% yield) as a diastereomeric mixture (1.3:1 dr).

LCMS: m/z, 391.0 [M+H]+; rt 1.16 mins (LCMS Method: Column: Acquity BEH C18 (2.1×50 mm, 1.7 μm); mobile phase A: 0.05% TFA in acetonitrile:water (5:95); mobile phase B: 0.05% TFA in acetonitrile:water (95:5), Gradient=0-100% B over 1.8 minute, then a 0.2 minute hold at 100% B; Temperature: 50° C.; Flow rate: 1.0 mL/min; Detection: UV at 220 nm, although product has no chromophore; peak detected by MS TIC). ¹H NMR (500 MHz, CDCl₃) δ 4.10-3.98 (m, 1.9H), 3.59 (dd, J=13.2, 1.0 Hz, 1.9H), 3.26 (td, J=13.4, 4.2 Hz, 1.9H), 2.96-2.86 (m, 1.5H), 2.86-2.79 (m, 2.3H), 2.44-2.33 (m, 3.8H), 1.96-1.90 (m, 5.7H), 1.86 (td, J=9.6, 1.5 Hz, 5.9H), 1.46 (s, 9.2H), 1.46 (s, 7.6H), 1.44-1.38 (m, 1.3H), 1.38-1.34 (m, 1.9H), 1.34-1.26 (m, 1.6H), 1.17 (d, J=6.6 Hz, 2.5H), 1.14 (d, J=6.6 Hz, 3.3H), 1.02-0.94 (m, 8.9H), 0.90 (t, J=7.5 Hz, 3.0H). The proton signals of this 1.3:1 diastereomeric mixture are reported with fractional numbers to be reflective of the mixture.

### Intermediate 134

### (2R,5S)-2,5-dimethyl-1-(1-(3-(trifluoromethyl)bicyclo[1.1.1]pentan-1-yl)propyl)piperazine (Diastereomeric Mixture)

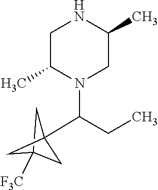 [SMILES: CCC(N1C[C@H](C)NC[C@H]1C)C12CC(C(F)(F)F)(C1)C2]

To a stirred solution of tert-butyl (2S,5R)-2,5-dimethyl-4-(1-(3-(trifluoromethyl) bicyclo[1.1.1]pentan-1-yl)propyl)piperazine-1-carboxylate (93 mg, 0.238 mmol, diastereomeric mixture) in anhydrous CH₂Cl₂ (2.382 mL) at room temperature was added TFA (0.183 mL, 2.382 mmol). The reaction mixture was stirred for 19 h, after which the solvent was removed under reduced pressure to afford the TFA salt of (2R,5S)-2,5-dimethyl-1-(1-(3-(trifluoromethyl)bicyclo[1.1.1]pentan-1-yl)propyl)piperazine (123 mg, 100% yield) after drying under high vacuum.

LCMS: m/z, 291.0 and 290.9 [M+H]⁺; rt 0.97 and 0.99 min. (LCMS Method: Column: Acquity BEH C18 (2.1×50 mm, 1.7 μm); mobile phase A: 0.05% TFA in acetonitrile:water (5:95); mobile phase B: 0.05% TFA in acetonitrile:water (95:5), Gradient=0-100% B over 1.8 minute, then a 0.2 minute hold at 100% B; Temperature: 50° C.; Flow rate: 1.0 mL/min; Detection: UV at 220 nm, although product has no chromophore; peak detected by MS TIC). ¹H NMR (500 MHz, methanol-d₄) δ 3.59-3.43 (m, 2.0H), 3.43-3.33 (m, 4.9H), 3.23 (dd, J=13.4, 2.9 Hz, 1.0H), 3.05 (t, J=11.9 Hz, 1.0H), 2.98-2.90 (m, 0.8H), 2.86-2.71 (m, 1.8H), 2.22-2.09 (m, 8.1H), 2.03 (dd, J=9.4, 1.5 Hz, 3.0H), 1.81-1.68 (m, 1.1H), 1.64-1.58 (m, 1.7H), 1.56-1.50 (m, 0.7H), 1.35 (d, J=2.3 Hz, 2.6H), 1.33 (d, J=2.4 Hz, 2.5H), 1.31-1.25 (m, 7.0H), 1.05 (t, J=7.5 Hz, 3.0H), 0.98 (t, J=7.4 Hz, 2.4H). The proton signals of this diastereomeric mixture are reported with fractional numbers to be reflective of the mixture.

### Example 446

### 6-chloro-4-((2S,5R)-2,5-dimethyl-4-(1-(3-(trifluoromethyl)bicyclo[1.1.1]pentan-1-yl) propyl)piperazin-1-yl)-1-methylpyrido[3,2-d]pyrimidin-2(1H)-one (Diastereomeric Mixture)

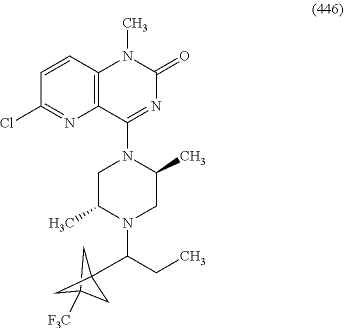 [SMILES: CCC(N1C[C@H](C)N(c2nc(=O)n(C)c3ccc(Cl)nc23)C[C@H]1C)C12CC(C(F)(F)F)(C1)C2]

To a stirred solution of (2R,5S)-2,5-dimethyl-1-(1-(3-(trifluoromethyl) bicyclo[1.1.1]pentan-1-yl)propyl)piperazine TFA salt (96 mg, 0.238 mmol) in acetonitrile (2 mL), DIPEA (0.416 mL, 2.380 mmol) and 4,6-dichloro-1-methylpyrido[3,2-d] pyrimidin-2(1H)-one (65.7 mg, 0.286 mmol) were added sequentially at room temperature. The reaction mixture was heated at 80° C. for 2 h. The reaction mixture was cooled to room temperature and the solvent removed under reduced pressure. The crude material was dry-loaded onto Celite and purified by silica gel column chromatography using 0-15% MeOH in CH₂Cl₂ to afford 6-chloro-4-((2S,5R)-2,5-dimethyl-4-(1-(3-(trifluoromethyl)bicyclo[1.1.1]pentan-1-yl)propyl)piperazin-1-yl)-1-methylpyrido[3,2-d] pyrimidin-2(1H)-one (101.9 mg, 88% yield). LCMS: m/z, 484.05 [M+H]⁺; rt 1.11 and 1.13 min. (LCMS Method: Column: Acquity BEH C18 (2.1×50 mm, 1.7 μm); mobile phase A: 0.05% TFA in acetonitrile:water (5:95); mobile phase B: 0.05% TFA in acetonitrile:water (95:5), Gradient=0-100% B over 1.8 minute, then a 0.2 minute hold at 100% B; Temperature: 50° C.; Flow rate: 1.0 mL/min; Detection: UV at 220 nm). ¹H NMR (400 MHz, CDCl₃) δ 7.50 (s, 4.0H), 5.91-5.14 (bm, 2.2H), 3.69-3.59 (m, 1.5H), 3.56 (s, 3.2H), 3.55 (s, 3.0H), 3.25-3.07 (m, 2.2H), 3.03-2.87 (m, 2.0H), 2.65 (dt, J=12.1, 6.1 Hz, 2.0H), 2.52-2.36 (m, 2.2H), 2.05-1.96 (m, 2.9H), 1.96-1.88 (m, 6.9H), 1.87-1.78 (m, 3.9H), 1.52-1.38 (m, 3.6H), 1.37-1.28 (m, 8.3H), 1.04 (d, J=6.2 Hz, 6.1H), 1.02-0.96 (m, 4.2H), 0.88 (t, J=7.3 Hz, 3.4H). The proton signals of this diastereomeric mixture were reported with fractional numbers to be reflective of the mixture.

### Examples 447-449

### 4-((2S,5R)-2,5-dimethyl-4-(1-(3-(trifluoromethyl)bicyclo[1.1.1]pentan-1-yl)propyl) piperazin-1-yl)-1-methyl-2-oxo-1,2-dihydropyrido[3,2-d]pyrimidine-6-carbonitrile (Diastereomeric Mixture and Resolved Homochiral Samples)

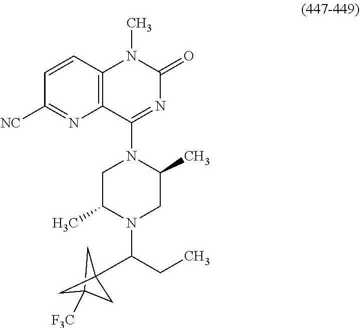 [SMILES: CCC(N1C[C@H](C)N(c2nc(=O)n(C)c3ccc(C#N)nc23)C[C@H]1C)C12CC(C(F)(F)F)(C1)C2]

A solution of 6-chloro-4-((2S,5R)-2,5-dimethyl-4-(1-(3-(trifluoromethyl) bicyclo[1.1.1]pentan-1-yl)propyl)piperazin-1-yl)-1-methylpyrido[3,2-d]pyrimidin-2(1H)-one (101.9 mg, 0.211 mmol), PdCl₂(dppf)-CH₂Cl₂ adduct (17.2 mg, 0.021 mmol), zinc (11.0 mg, 0.168 mmol), and zinc cyanide (33 mg, 0.281 mmol) in anhydrous NMP (2.376 mL) was sparged with nitrogen for 15 min. The reaction mixture was heated at 80° C. for 19 h. The reaction was quenched with saturated aqueous sodium bicarbonate. EtOAc was added and the mixture stirred vigorously. The aqueous layer was extracted with EtOAc (4×). The combined organic layers were washed with brine (2×), dried over Na₂SO₄, filtered, and concentrated under reduced pressure to afford a red-brown oil. The crude material was purified via preparative LCMS with the following conditions: Column: XBridge C18, 200 mm×19 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 42% B, 42-82% B over 20 minutes, then a 0-minute hold at 100% B; Flow Rate: 20 mL/min; Column Temperature: 25° C. Fraction collection was triggered by MS and UV signals. Fractions containing the product were combined and dried via centrifugal evaporation. The yield of the product was 60.5 mg (60.6% yield), and its estimated purity by LCMS analysis was 100%. Analytical LCMS was used to determine the final purity. Injection 1 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 475.07, 475.07; Retention Time: 1.39, 1.42 min. Injection 2 conditions: Column: Waters Xbridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 2 results: Purity: 100%; Observed Mass: 475.06; Retention Time: 2.35 min. ¹H NMR (500 MHz, DMSO-d₆) δ 8.22 (d, J=8.9 Hz, 2H), 7.97 (br d, J=9.2 Hz, 2H), 5.83-4.65 (m, 2H), 3.44 (s, 6H), 3.37-3.30 (m, 1H), 3.26-3.06 (m, 1H), 2.97-2.86 (m, 1H), 2.85-2.73 (m, 1H), 2.72-2.60 (m, 1H), 2.49-2.41 (m, 1H), 2.04 (br d, J=9.5 Hz, 2H), 1.98-1.82 (m, 10H), 1.48-1.30 (m, 4H), 1.30-1.20 (m, 6H), 1.01-0.89 (m, 9H), 0.82 (br t, J=7.2 Hz, 3H). Some peaks are obscured due to the water suppression technique employed. The multiplet between 2.49-2.41 ppm is obscured due to overlap with the DMSO-d6 NMR solvent peak.

The diastereomeric mixture of 4-((2S,5R)-2,5-dimethyl-4-(1-(3-(trifluoromethyl)bicyclo[1.1.1]pentan-1-yl)propyl)piperazin-1-yl)-1-methyl-2-oxo-1,2-dihydropyrido[3,2-d]pyrimidine-6-carbonitrile was resolved by the following chiral SFC separation method: Approximately 29 mg of sample were resolved into two peaks collected in IPA w/ 0.10% DEA. The chiral purity for the isolates were determined using the analytical chromatogram below.

### Isolate Chiral Purity

1st Eluting Peak >95%
2nd Eluting Peak >95%

### Preparative Chromatographic Conditions:

Instrument: Waters 100 Prep SFC
Column: Chiral OD, 30×250 mm. 5 micron
Mobile Phase: 80% CO₂/20% IPA w/ 0.1% DEA
Flow Conditions: 100 mL/min
Detector Wavelength: 220 nm
Injection Details: 1000 μL 29 mg dissolved in 3 mL MeOH

### Analytical Chromatographic Conditions (Before Prep):

Instrument: Shimadzu Nexera UC SFC
Column: Chiral OD, 4.6×100 mm, 5 micron
Mobile Phase: 80% CO₂/20% IPA w/ 0.1% DEA
Flow Conditions: 2 mL/min
Detector Wavelength: 220 nm
Example 448: Isolate 1: First eluting peak
Example 449: Isolate 2: Second eluting peak

EXAMPLE 448: The yield of the product was 6.7 mg (22.3%), and its purity was 100%. Analytical LCMS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 475.09; Retention Time: 2.34 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 100%; Observed Mass: 474.90; Retention Time: 1.51 min. ¹H NMR (500 MHz, DMSO-d6) δ 8.22 (d, J=8.9 Hz, 1H), 7.96 (d, J=8.9 Hz, 1H), 5.88-4.58 (m, 1H), 4.36-4.28 (m, 1H), 3.43 (s, 3H), 3.39-3.28 (m, 1H), 3.25-3.13 (m, 1H), 2.94-2.84 (m, 1H), 2.83-2.70 (m, 1H), 2.49-2.45 (m, 1H), 2.11-1.90 (m, 6H), 1.46-1.30 (m, 2H), 1.26 (br s, 3H), 0.92 (br d, J=5.8 Hz, 3H), 0.82 (br t, J=7.2 Hz, 3H). Some peaks are obscured due to the water suppression technique employed. The multiplet between 2.49-2.45 ppm is obscured due to overlap with the DMSO-d₆ NMR solvent peak.

EXAMPLE 449: The yield of the product was 10.0 mg (33.3%), and its purity was 100%. Analytical LCMS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 475.30; Retention Time: 2.36 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 100%; Observed Mass: 474.92; Retention Time: 1.49 min. ¹H NMR (500 MHz, DMSO-d₆) Shift 8.21 (d, J=8.9 Hz, 1H), 7.96 (d, J=8.9 Hz, 1H), 5.86-4.52 (m, 1H), 3.43 (s, 3H), 3.40-3.31 (m, 1H), 3.16-3.06 (m, 1H), 2.99-2.85 (m, 1H), 2.74-2.61 (m, 1H), 2.45 (br t, J=6.7 Hz, 1H), 1.95-1.79 (m, 6H), 1.48-1.32 (m, 2H), 1.26 (br d, J=2.4 Hz, 3H), 1.02-0.88 (m, 6H). Some peaks are obscured due to the water suppression technique employed.

The examples in the Table 21 were prepared from general procedure described in Examples 447-449, using appropriate carboxylic acid and Grignard reagent in the first and second steps, respectively. When the reaction provided a mixture of diastereomers, the mixture was separated at the final stage using either preparative chromatography or preparative chiral chromatography. The absolute stereochemistry was not assigned at the newly formed carbon-nitrogen bond.

TABLE 21

| Ex. | | Stereo | LCMS | LCMS | |
|---|---|---|---|---|---|
| No. | STRUCTURE | chem. | Method | rt | [M + H]⁺ |
| 450 451 452 | 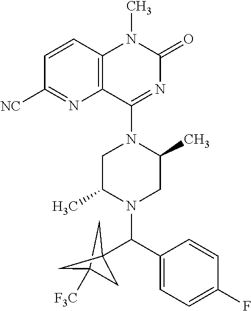 [SMILES: C[C@@H]1CN(c2nc(=O)n(C)c3ccc(C#N)nc23)[C@@H](C)CN1C(c1ccc(F)cc1)C12CC(C(F)(F)F)(C1)C2] | D H H | F F F | 1.80 1.76 1.79 | 540.9 541.2 541.0 |
| 453 454 455 | 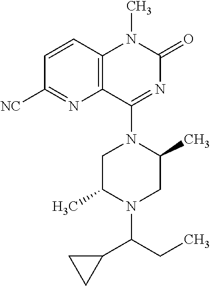 [SMILES: CCC(C1CC1)N1C[C@H](C)N(c2nc(=O)n(C)c3ccc(C#N)nc23)C[C@H]1C] | D H H | F F F | 1.04 1.21 1.20 | 381.0 381.3 381.3 |
| 456 457 458 | 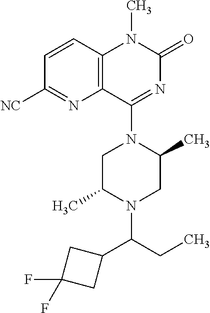 [SMILES: CCC(C1CC(F)(F)C1)N1C[C@H](C)N(c2nc(=O)n(C)c3ccc(C#N)nc23)C[C@H]1C] | D H H | F F F | 1.07 1.23 1.23 | 431.0 431.2 431.2 |
| 459 460 | 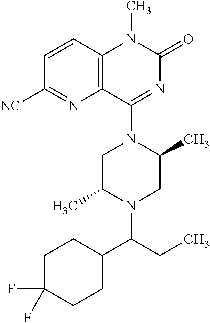 [SMILES: CCC(C1CCC(F)(F)CC1)N1C[C@H](C)N(c2nc(=O)n(C)c3ccc(C#N)nc23)C[C@H]1C] | H H | F F | 1.20 1.23 | 459.1 459.1 |

### Biological Assays

The pharmacological properties of the compounds of this invention may be confirmed by a number of biological assays. The exemplified biological assays, which follow, have been carried out with compounds of the invention.
DGK alpha LIPGLO IC₅₀ (uM)
DGK alpha ADPGLO (Truncated) IC₅₀ (uM)

### 1. In Vitro DGK Inhibition Assays

The DGKα and DGKζ reactions were performed using either extruded liposome (DGKα and DGKζ LIPGLO assays) or detergent/lipid micelle substrate (DGKα and DGKζ assays). The reactions were carried out in 50 mM MOPS pH 7.5, 100 mM NaCl, 10 mM MgCl₂, 1 μM CaCl₂, and 1 mM DTT (assay buffer). The reactions using a detergent/lipid micelle substrate also contained 50 mM octyl B-D-glucopyranoside. The lipid substrate concentrations were 11 mM PS and 1 mM DAG for the detergent/lipid micelle reactions. The lipid substrate concentrations were 2 mM PS, 0.25 mM DAG, and 2.75 mM PC for the extruded liposome reactions (5 mM total lipid). The reactions were carried out in 150 μM ATP. The enzyme concentrations for the DGKα and DGKζ were 5 nM

The compound inhibition studies were carried out as follows: 25 nL (ADPGLO assay) or 50 nL (LIPGLO assay) droplets of each test compound (top concentration 10 mM with 11 point, 3-fold dilution series for each compound) solubilized in DMSO were transferred to wells of a white 1536 well plate (Corning 3725). A 5 mL enzyme/lipid substrate solution at 2× final reaction concentration was prepared by combining 2.5 mL 4× enzyme solution (20 nM DGKα or DGKζ (prepared as described below) in assay buffer) and 2.5 mL of either 4× liposome or 4× detergent/lipid micelle solution (compositions described below) and incubated at room temperature for 10 minutes. Next, 1 μL 2× enzyme/lipid substrate solution was added to wells containing the test compound and reactions were initiated with the addition of 1 μL 300 uM ATP. The reactions were allowed to proceed for 2 hr (ADPGLO assay) or 1 hr (LIPGLO assay), after which 2 μL Glo Reagent (Promega V9101) was added and incubated for 40 minutes. Next, 4 μL Kinase Detection Reagent was added and incubated for 30 minutes. Luminescence was recorded using an EnVision microplate reader. The percent inhibition was calculated from the ATP conversion generated by no enzyme control reactions for 100% inhibition and vehicle-only reactions for 0% inhibition. The compounds were evaluated at 11 concentrations to determine IC₅₀.

### 4× Detergent/Lipid Micelle Preparation

The detergent/lipid micelle was prepared by combining 15 g phosphatidylserine (Avanti 840035P) and 1 g diacylglycerol (8008110) and dissolving into 150 mL chloroform in a 2 L round bottom flask. Chloroform was removed under high vacuum by rotary evaporation. The resulting colorless, tacky oil was resuspended in 400 mL 50 mM MOPS pH 7.5, 100 mM NaCl, 20 mM NaF, 10 mM MgCl₂, 1 μM CaCl₂, 1 mM DTT, and 200 mM octyl glucoside by vigorous mixing. The lipid/detergent solution was split into 5 mL aliquots and stored at −80° C.

### 2× Liposome Preparation

The lipid composition was 5 mol % DAG (Avanti 8008110), 40 mol % PS (Avanti 840035P), and 55 mol % PC (Avanti 850457) at a total lipid concentration of 7-8 mg/mL for the liposome solution. The PC, DAG, and PS were dissolved in chloroform, combined, and dried in vacuo to a thin film. The lipids were hydrated to 20 mM in 50 mM MOPS pH 7.5, 100 mM NaCl, 5 mM MgCl₂, and were freeze-thawed five times. The lipid suspension was extruded through a 100 nm polycarbonate filter 10-12 times. Dynamic light scattering was carried out to confirm liposome size (50-60 nm radius). The liposome preparation was stored at 4° C. for as long as four weeks.

### 4× Liposome Preparation

The lipid composition was 5 mol % DAG (Avanti 8008110), 40 mol % PS (Avanti 840035P), and 55 mol % PC (Avanti 850457) at a total lipid concentration of 15.2 mg/mL for the 4× liposome solution. The PC, DAG, and PS were dissolved in chloroform, combined, and dried in vacuo to a thin film. The lipids were hydrated to 20 mM in 50 mM MOPS pH 7.5, 100 mM NaCl, 5 mM MgCl₂, and were freeze-thawed five times. The lipid suspension was extruded through a 100 nm polycarbonate filter eleven times. Dynamic light scattering was carried out to confirm liposome size (50-60 nm radius). The liposome preparation was stored at 4° C. for as long as four weeks.

### Baculovirus Expression of Human DGKα and DGKζ

Human DGK-alpha-TVMV-His-pFBgate and human DGK-zeta-transcript variant-2-TVMV-His-pFBgate baculovirus samples were generated using the Bac-to-Bac baculovirus expression system (Invitrogen) according to the manufacturer's protocol. The DNA used for expression of DGK-alpha and DGK-zeta have SEQ ID NOs: 1 and 3, respectively. Baculovirus amplification was achieved using infected Sf9 cells at 1:1500 virus/cell ratios, and grown for 65 hours at 27° C. post-transfection.

The expression scale up for each protein was carried out in the Cellbag 50L WAVE-Bioreactor System 20/50 from GE Healthcare Bioscience. 12 L of 2×10⁶ cells/mL Sf9 cells (Expression System, Davis, CA) grown in ESF921 insect medium (Expression System) were infected with virus stock at 1:200 virus/cell ratios, and grown for 66-68 hours at 27° C. post-infection. The infected cell culture was harvested by centrifugation at 2000 rpm for 20 min 4° C. in a SORVALL® RC12BP centrifuge. The cell pellets were stored at −70° C. until purification.

### Purification of Human DGK-Alpha and DGK-Zeta

Full length human DGKα and DGKζ, each expressed containing a TVMV-cleavable C-terminal Hexa-His tag sequence (SEQ ID NOs: 2 and 4, respectively) and produced as described above, were purified from Sf9 baculovirus-infected insect cell paste. The cells were lysed using nitrogen cavitation method with a nitrogen bomb (Parr Instruments), and the lysates were clarified by centrifugation. The clarified lysates were purified to ˜90% homogeneity, using three successive column chromatography steps on an AKTA Purifier Plus system. The three steps column chromatography included nickel affinity resin capture (i.e. HisTrap FF crude, GE Healthcare), followed by size exclusion chromatography (i.e. HiLoad 26/600 Superdex 200 prep grade, GE Healthcare for DGK-alpha, and HiPrep 26/600 Sephacryl S 300_HR, GE Healthcare for DGK-zeta). The third step was ion exchange chromatography, and differed for the two isoforms. DGKα was polished using Q-Sepharose anion exchange chromatography (GE Healthcare). DGKζ was polished using SP Sepharose cation exchange chromatography (GE Healthcare). The proteins were delivered at concentrations of ≥2 mg/mL. The formulation buffers were identical for both proteins: 50 mM Hepes, pH 7.2, 500 mM NaCl, 10% v/v glycerol, 1 mM TCEP, and 0.5 mM EDTA.

### 2. Raji CD4 T Cell IL2 Assay

A 1536-well IL-2 assay was performed in 4 μL volume using pre-activated CD4 T cells and Raji cells. Prior to the assay, CD4 T cells were pre-activated by treatment with α-CD3, α-CD28 and PHA at 1.5 μg/mL, 1 μg/mL, and 10 μg/mL, respectively. Raji cells were treated with Staphylococcal enterotoxin B (SEB) at 10,000 ng/mL. Serially diluted compounds were first transferred to 1536-well assay plate (Corning, #3727), followed by addition of 2 μL of pre-activated CD4 T cells (final density at 6000 cells/well) and 2 μL of SEB-treated Raji cells (2000 cells/well). After 24 hours incubation at a 37° C./5% CO₂ incubator, 4 μl of IL-2 detection reagents were added to the assay plate (Cisbio, #64IL2PEC). The assay plates were read on an Envision reader. To assess compound cytotoxicity, either Raji or CD4 T cells were incubated with the serially diluted compounds. After 24 hours incubation, 4 μL of Cell Titer Glo (Promega, #G7572) were added, and the plates were read on an Envision reader. The 50% effective concentration (IC₅₀) was calculated using the four-parameter logistic formula y=A+((B−A)/(1+((C/x)\hat{ }D))), where A and B denote minimal and maximal % activation or inhibition, respectively, C is the IC₅₀, D is hill slope and x represent compound concentration.

### 3. CellTiter-Glo CD8 T Cell Proliferation Assay

Frozen naïve human CD8 T cells were thawed in RPMI+10% FBS, incubated for 2 h in 37° C., and counted. The 384-well tissue culture plate was coated overnight at 4° C. with 20 μl anti-human CD3 at 0.1 μg/mL in plain RPMI, which was removed off the plate before 20 k/40 μL CD8 T cells with 0.5 μg/ml soluble anti-human CD28 were added to each well. The compounds were echoed to the cell plate immediately after the cells were plated. After 72 h incubation at 37° C. incubator, 10 μL CellTiter-glo reagent (Promega catalog number G7570) was added to each well. The plate was vigorously shaken for 5 mins, incubated at room temperature for another 15 mins and read on Envision for CD8 T cell proliferation. In analysis, 0.1 μg/mL anti-CD3 and 0.5 μg/mL anti-CD28 stimulated CD8 T cell signal was background. The reference compound, 8-(4-(bis(4-fluorophenyl)methyl) piperazin-1-yl)-5-methyl-7-nitro-6-oxo-5,6-dihydro-1,5-naphthyridine-2-carbonitrile, at 3 μM was used to set the 100% range and EC₅₀ was at absolute 50% to normalize the data.

### 4. DGK AP1-Reporter Assay

The Jurkat AP1-luciferase Reporter was generated using the Cignal Lenti AP1 Reporter (luc) Kit from SABiosciences (CLS-011L).

The compounds were transferred from an Echo LDV plate to individual wells of a 384-well plate (white, solid-bottom, opaque PE CulturPlate 6007768) using an Echo550 instrument. The sample size was 30 nL per well; and one destination plate per source plate. The cell suspensions were prepared by transferring 40 mL cells (2×20 mL) to clean 50 mL conical tubes. The cells were concentrated by centrifugation (1200 rpm; 5 mins; ambient temperature). The supernatant was removed and all cells were suspended in RPMI (Gibco 11875)+10% FBS to make a 1.35×10⁶ cells/ml concentration. The cells were added manually using a multi-channel pipette, 30 μL/well of cell suspension to a 384-well TC plate containing the compounds, 4.0×10⁴ cells per well. The cell plates were incubated for 20 minutes at 37° C. and 5% CO₂.

During the incubation, anti-CD3 antibody (αCD3) solutions were prepared by mixing 3 μL aCD3 (1.3 mg/mL) with 10 mL medium [final conc=0.4 μg/mL]. Next, 1.5 μl aCD3 (1.3 mg/mL) was mixed with 0.5 mL medium [final conc=4 μg/ml]. After 20 minutes, 10 μL medium was added to all wells in column 1, wells A to M, and 10 μL αCD3 (4 ug/mL) per well was added in column 1, rows N to P for reference. Then using a multi-channel pipette, 10 μL αCD3 (0.4 ug/mL) per well was added. The αCD3 stimulated+/−compound-treated cells were incubated at 37° C., 5% CO₂ for 6 hours.

During this incubation period, Steady-Glo (Promega E2520) reagent was slowly thawed to ambient temperature. Next, 20 μL Steady-Glo reagent per well was added using a multi-drop Combi-dispenser. Bubbles were removed by centrifugation (2000 rpm, ambient temperature, 10 secs). The cells were incubated at room temperature for 5 minutes. Samples were characterized by measuring the Relative Light Units (RLU) with an using Envision Plate Reader Instrument on a luminescence protocol. The data was analyzed using the reference compound, 8-(4-(bis(4-fluorophenyl)methyl)piperazin-1-yl)-5-methyl-7-nitro-6-oxo-5,6-dihydro-1,5-naphthyridine-2-carbonitrile, to normalize 100% inhibition.

### 5. Murine Cytotoxic T Lymphocyte Assay

An antigen-specific cytolytic T-cell (CTL) assay was developed to evaluate functionally the ability of DGKα and DGKζ inhibitors to enhance effector T cell mediated tumor cell killing activity. CD8+ T-cells isolated from the OT-1 transgenic mouse recognize antigen presenting cells, MC38, that present the ovalbumin derived peptide SIINFEKL. Recognition of the cognate antigen initiates the cytolytic activity of the OT-1 antigen-specific CD8+ T cells.

Functional CTL cells were generated as follows: OT-1 splenocytes from 8-12 week old mice were isolated and expanded in the presence of the SIINFEKL peptide at 1 μg/mL and mIL2 at 10 U/mL. After three days, fresh media with mIL2 U/ml was added. On day 5 of the expansion, the CD8+ T cells were isolated and ready for use. Activated CTL cells may be stored frozen for 6 months. Separately, one million MC38 tumor cells were pulsed with 1 μg/mL of SIINFEKL-OVA peptide for 3 hours at 37° C. The cells were washed (3×) with fresh media to remove excess peptide. Finally, CTL cells that were pretreated with DGK inhibitors for 1 hour in a 96-well U bottom plate were combined with the antigen loaded MC38 tumor cells at a 1:10 ratio. The cells were then spun at 700 rpm for 5 min and placed in an incubator overnight at 37° C. After 24 hours, the supernatant was collected for analysis of IFN-γ cytokine levels by AlphaLisa purchased from Perkin Elmer.

### 6. PHA Proliferation Assay

Phytohaemagglutinin (PHA)-stimulated blast cells from frozen stocks were incubated in RPMI medium (Gibco, ThermoFisher Scientific, Waltham, MA) supplemented with 10% fetal bovine serum (Sigma Aldrich, St. Louis, MO) for one hour prior to adding to individual wells of a 384-well plate (10,000 cells per well). The compounds were transferred to individual wells of a 384-well plate and the treated cells are maintained at 37° C., 5% CO₂ for 72 h in culture medium containing human IL2 (20 ng/mL) prior to measuring growth using MTS reagent [3-(4,5-dimethyl-2-yl)-5-(3-carboxymethoxyphenyl)-2-(4-sulfophenyl)-2H-tetrazolium] following manufacturer's instructions (Promega, Madison, WI). Percent inhibition was calculated comparing values between IL2 stimulated (0% inhibition) and unstimulated control (100% inhibition). Inhibition concentration (IC₅₀) determinations were calculated based on 50% inhibition on the fold-induction between IL2 stimulated and unstimulated treatments.

### 7. Human CD8 T Cells IFN-γ Assay

Frozen naïve human CD8 T cells were thawed in AIM-V media, incubated for 2 h in 37° C., and counted. The 384-well tissue culture plate was coated overnight at 4° C. with 20 μL anti-human CD3 at 0.05 μg/mL in PBS, which was removed off the plate before 40,000 cells per 40 microliters CD8 T cells with 0.1 μg/mL soluble anti-human CD28 were added to each well. The compounds were transferred using an Echo liquid handler to the cell plate immediately after the cells were plated. After 20 h incubation at 37° C. incubator, 3 microliters per well supernatants transferred into anew 384-well white assay plate for cytokine measurement.

Interferon-γ (IFN-γ) was quantitated using the AlphLISA kit (Cat #AL217) as described by the manufacturer manual (Perkin Elmer). The counts from each well were converted to IFN-γ concentration (pg/mL). The compound EC₅₀ values were determined by setting 0.05 μg/mL anti-CD3 plus 0.1 μg/mL anti-CD28 as the baseline, and co-stimulation of 3 μM of the reference compound, 8-(4-(bis(4-fluorophenyl)methyl) piperazin-1-yl)-5-methyl-7-nitro-6-oxo-5,6-dihydro-1,5-naphthyridine-2-carbonitrile, with anti-CD3 plus anti-CD28 as 100% activation.

8. Human CD8 T Cells pERK Assay

Frozen naïve human CD8 T cells were thawed in AIM-V media, incubated for 2 h in 37° C., and counted. The CD8 positive T cells were added to 384-well tissue culture plate at 20,000 cells per well in AIM-V media. One compound was added to each well, then bead bound anti-human CD3 and anti-CD28 mAb were added at final concentration of 0.3 μg/mL. The cells were incubated at 37° C. for 10 minutes. The reaction was stopped by adding lysis buffer from the AlphaLISA Surefire kit. (Perkin Elmer, cat #ALSU-PERK-A). Lysate (5 μL per well) was transferred into a new 384-well white assay plate for pERK activation measurement.

Compound EC₅₀ was determined as setting anti-CD3 plus anti-CD28 as baseline, and co-stimulation of 3 μM 8-(4-(bis(4-fluorophenyl)methyl)piperazin-1-yl)-5-methyl-7-nitro-6-oxo-5,6-dihydro-1,5-naphthyridine-2-carbonitrile with anti-CD3 plus anti-CD28 as 100% activation.

### 9. Human Whole Blood IFN-γ Assay

Human venous whole blood (22.5 μL per well), obtained from healthy donors, was pre-treated with compounds for one hour at 37° C. in a humidified 95% air/5% CO₂ incubator. The blood was stimulated with 2.5 μL anti-human CD3 and anti-CD28 mAb at a final concentration of 1 μg/mL each for 24 hours at 37° C. IFN-γ in the supernatants was measured using AlphLISA kit (Cat #AL217).

Compound EC₅₀ determined as setting anti-CD3 plus anti-CD28 as baseline, and co-stimulation of 3 μM of the reference compound, 8-(4-(bis(4-fluorophenyl)methyl) piperazin-1-yl)-5-methyl-7-nitro-6-oxo-5,6-dihydro-1,5-naphthyridine-2-carbonitrile, with anti-CD3 plus anti-CD28 as 100% activation.

TABLE A
In vitro DGK Inhibition IC₅₀ Activity Values

| | | DGK | | IFN-γ | pERK |
|---|---|---|---|---|---|
| | DGK | alpha | DGK | Whole | Whole |
| | alpha | ADPGLO | zeta | Blood | Blood |
| | LIPGLO | (Truncated) | LIPGLO | Normalized | Normalized |
| Ex. | IC₅₀ | IC₅₀ | IC₅₀ | EC₅₀ | IC₅₀ |
| No. | (uM) | (uM) | (uM) | (uM) | (uM) |
| 1 | 0.016 | — | 0.036 | 0.027 | 1.04 |
| 2 | 0.032 | — | 0.047 | 0.076 | 0.15 |
| 3 | 0.19 | — | 0.11 | 0.031 | — |
| 4 | 1.33 | — | 0.76 | 0.97 | — |
| 5 | 0.16 | 0.064 | 0.13 | 0.02 | 0.13 |
| 6 | 4.66 | 0.058 | 2.44 | 3.75 | 6.98 |
| 7 | 0.39 | 0.18 | 0.81 | 0.52 | — |
| 8 | 0.12 | 0.14 | 0.044 | 0.032 | 0.18 |
| 9 | 0.007 | — | 0.048 | — | — |
| 10 | 0.37 | — | 0.97 | — | — |
| 11 | 0.005 | — | 0.060 | — | — |
| 12 | 0.41 | — | 0.60 | 4.15 | 0.60 |
| 13 | 0.70 | — | 0.23 | 2.12 | — |
| 14 | 0.35 | — | 0.036 | 0.094 | 0.90 |
| 15 | 0.49 | — | 1.22 | 1.74 | — |
| 16 | 0.045 | — | 0.037 | — | — |
| 17 | 0.32 | — | 2.14 | 2.89 | — |
| 18 | 0.030 | — | 0.025 | — | — |
| 19 | 4.05 | — | 3.10 | 2.06 | — |
| 20 | 0.14 | — | 0.036 | 0.06 | 0.13 |
| 21 | 0.386 | — | 1.01 | 2.85 | — |
| 22 | 0.002 | — | 0.18 | 0.38 | — |
| 23 | 0.058 | — | 0.029 | 0.011 | 0.086 |
| 24 | 0.68 | — | 0.63 | 0.45 | — |
| 25 | 1.23 | 0.33 | 1.34 | 1.25 | — |
| 26 | 0.081 | 0.029 | 0.21 | 0.18 | 0.24 |
| 27 | 0.002 | — | 0.23 | 0.62 | — |
| 28 | 1.57 | — | 2.65 | 2.04 | — |
| 29 | 0.040 | — | 0.010 | 0.027 | 0.10 |
| 30 | 0.076 | — | 0.004 | 0.023 | 0.059 |
| 31 | 1.70 | — | 0.96 | 0.27 | — |
| 32 | 0.50 | — | 0.031 | 0.019 | — |
| 33 | 2.56 | — | 5.93 | 3.83 | — |
| 34 | 0.62 | — | 0.31 | 0.42 | — |
| 35 | 0.030 | — | 0.12 | 0.12 | 0.15 |
| 36 | 0.016 | 0.19 | 0.075 | 0.082 | 0.25 |
| 37 | 0.069 | 0.12 | 0.47 | 20 | — |
| 38 | 0.066 | — | 0.12 | 0.072 | 0.22 |
| 39 | — | — | 0.77 | 2.73 | — |
| 40 | 0.32 | — | 0.041 | 0.021 | 0.010 |
| 41 | 2.27 | — | 1.08 | 0.47 | — |
| 42 | 0.078 | 0.007 | 0.11 | 0.081 | 0.35 |
| 43 | 0.022 | 0.082 | 0.13 | 0.12 | 0.34 |
| 44 | 0.085 | — | 0.021 | 0.26 | — |
| 45 | 0.31 | — | 0.51 | 1.04 | — |
| 46 | 0.094 | — | 0.060 | 0.073 | 0.043 |
| 47 | 0.099 | — | 0.054 | 0.032 | 0.020 |
| 48 | 0.016 | — | 0.048 | 0.018 | 0.025 |
| 49 | 0.39 | — | 0.85 | 2.34 | — |
| 50 | 0.11 | — | 0.037 | — | — |
| 51 | 0.037 | — | 0.027 | 0.006 | 0.009 |
| 52 | 0.23 | — | 0.052 | 0.053 | — |
| 53 | 0.63 | — | 0.079 | 0.20 | — |
| 54 | 0.24 | — | 0.35 | 0.31 | 0.91 |
| 55 | 0.41 | — | 0.038 | 0.79 | — |
| 56 | 0.037 | — | 0.011 | 0.039 | 0.20 |
| 57 | 0.96 | — | 0.93 | 1.90 | — |
| 58 | 0.27 | — | 0.054 | 0.022 | — |
| 59 | 0.30 | — | 0.26 | 0.50 | — |
| 60 | 0.030 | — | 0.028 | 0.023 | — |
| 61 | 0.052 | — | 0.38 | 4.50 | — |
| 62 | 0.058 | — | 0.22 | 0.20 | — |
| 63 | 0.040 | — | 0.021 | 0.070 | — |
| 64 | 0.34 | — | 0.76 | 0.69 | — |
| 65 | 1.19 | — | 0.10 | 0.36 | — |
| 66 | 0.23 | 0.002 | 0.11 | 0.075 | — |
| 67 | 0.21 | — | 0.033 | — | — |
| 68 | 1.63 | — | 0.48 | 1.90 | — |
| 69 | 0.21 | — | 0.014 | 0.033 | — |
| 70 | 0.84 | — | 1.41 | 1.44 | — |
| 71 | 0.67 | — | 0.022 | 0.066 | — |
| 72 | 1.50 | — | — | 0.79 | — |
| 73 | 0.029 | — | 0.14 | 0.022 | 0.08 |
| 74 | 0.25 | — | 0.96 | 0.43 | — |
| 75 | 0.039 | — | 0.033 | 0.030 | 0.10 |
| 76 | 0.079 | — | 0.023 | 0.18 | — |
| 77 | 0.036 | — | 0.014 | — | — |
| 78 | 0.47 | — | 1.13 | 0.075 | — |
| 79 | 0.30 | — | 0.023 | 0.30 | — |
| 80 | 2.16 | — | 1.18 | — | — |
| 81 | 0.30 | — | 0.13 | 0.057 | 0.099 |
| 82 | 1.54 | — | 2.45 | 0.53 | — |
| 83 | 0.097 | 0.015 | 0.16 | 0.017 | 0.087 |
| 84 | 3.22 | 0.054 | 2.09 | 0.67 | 0.78 |
| 85 | 0.21 | 0.066 | 0.091 | 0.022 | 0.066 |
| 86 | 2.94 | 0.42 | 2.37 | 1.54 | — |
| 87 | 0.028 | — | 0.31 | 0.44 | — |
| 88 | 0.11 | — | 0.076 | 0.048 | — |
| 89 | 0.055 | 0.060 | 0.11 | 0.035 | 0.26 |
| 90 | 0.12 | 0.14 | 0.59 | — | — |
| 91 | 0.33 | — | 0.044 | 0.19 | — |
| 92 | 0.46 | — | 0.12 | 0.35 | — |
| 93 | — | 0.018 | 0.52 | 0.17 | 0.67 |
| 94 | 0.20 | 0.016 | 0.086 | 0.043 | 0.22 |
| 95 | 0.15 | — | 0.29 | 0.37 | 10.48 |
| 96 | 0.32 | 0.096 | 0.13 | 0.057 | 0.20 |
| 97 | 0.63 | 0.21 | 0.045 | 0.099 | 0.38 |
| 98 | 0.31 | 0.026 | 0.055 | 0.088 | 0.14 |
| 99 | 0.13 | 0.004 | 0.12 | 0.12 | 0.14 |
| 100 | 0.25 | 0.011 | 0.11 | 0.044 | 0.32 |
| 101 | 0.14 | — | 0.066 | 0.10 | 0.25 |
| 102 | — | — | 0.094 | — | — |
| 103 | 0.17 | — | 0.036 | 0.094 | — |
| 104 | 0.47 | — | 0.11 | 0.21 | — |
| 106 | 0.039 | — | 0.13 | 0.24 | — |
| 107 | 0.13 | — | 0.040 | 0.049 | 0.12 |
| 108 | 0.40 | — | 0.050 | 0.057 | 0.028 |
| 109 | 0.034 | — | 0.19 | 0.11 | 0.048 |
| 110 | 0.067 | — | 0.029 | 0.009 | 0.015 |
| 111 | 0.014 | — | 0.033 | 0.021 | 0.090 |
| 112 | 0.043 | 0.011 | 0.022 | 0.011 | 0.015 |
| 113 | 0.047 | 0.010 | 0.046 | 0.057 | 0.17 |
| 114 | 0.16 | — | 0.44 | 0.47 | — |
| 115 | 0.16 | — | 2.47 | 0.23 | 0.52 |
| 116 | 1.00 | — | 2.11 | 1.43 | — |
| 117 | 0.002 | — | 0.002 | 0.16 | 0.78 |
| 118 | 0.095 | — | 0.016 | — | — |
| 119 | 0.65 | — | 0.73 | — | — |
| 120 | 1.71 | — | 0.74 | — | — |
| 121 | 1.94 | — | 1.65 | — | — |
| 122 | 0.032 | — | 1.96 | — | — |
| 123 | 1.38 | — | 1.33 | 2.57 | — |
| 124 | 0.56 | — | 2.56 | 0.50 | 0.27 |
| 125 | 0.74 | — | 0.99 | 0.50 | — |
| 126 | 0.19 | — | 0.44 | 0.68 | — |
| 127 | 0.18 | — | 0.56 | 0.41 | 0.24 |
| 128 | 0.026 | — | 0.11 | — | — |
| 129 | 0.82 | — | 1.55 | 13.2 | — |
| 130 | 12.4 | — | 0.96 | 4.07 | — |
| 131 | 0.010 | — | 0.13 | 0.36 | 0.17 |
| 132 | 0.70 | — | 5.95 | 2.65 | — |
| 133 | 0.53 | — | 0.24 | 0.71 | — |
| 134 | 4.26 | — | 0.78 | 3.67 | — |
| 135 | 4.50 | — | 1.41 | 10.7 | — |
| 136 | 0.92 | — | 0.22 | 0.65 | — |
| 137 | 0.083 | — | 0.19 | 2.65 | — |
| 138 | 2.41 | — | — | — | — |
| 139 | 25.6 | — | 2.88 | 20.0 | — |
| 140 | 0.18 | — | 0.36 | 0.43 | — |
| 141 | 1.27 | — | 1.76 | 3.55 | — |
| 142 | 0.052 | — | 0.23 | 3.65 | 0.10 |
| 143 | 1.33 | — | 2.12 | — | 1.14 |
| 144 | 0.028 | — | 0.046 | — | 0.065 |
| 145 | 1.13 | — | 1.89 | 20.0 | 0.75 |
| 146 | 0.047 | — | 0.044 | 0.71 | 0.25 |
| 147 | 1.46 | — | 1.13 | — | 1.00 |
| 148 | 0.26 | — | 0.10 | 0.57 | — |
| 149 | 4.56 | — | 1.18 | 8.06 | 1.95 |
| 150 | 0.35 | — | 0.20 | 0.48 | 0.14 |
| 151 | 3.46 | — | 0.83 | — | 1.87 |
| 152 | 0.029 | — | 0.092 | 0.20 | 0.11 |
| 153 | 0.69 | — | 1.98 | 2.31 | — |
| 154 | 0.016 | — | 0.37 | 1.43 | — |
| 155 | 0.25 | — | 0.75 | — | — |
| 156 | 0.010 | — | 0.12 | — | — |
| 157 | 0.33 | — | 1.38 | 1.42 | 4.36 |
| 158 | 0.66 | — | 0.57 | 1.58 | — |
| 159 | 0.094 | — | 0.038 | 0.25 | — |
| 160 | 0.11 | — | 0.25 | 0.24 | 0.86 |
| 161 | 2.10 | — | 1.21 | 4.01 | — |
| 162 | 0.056 | — | 1.15 | 1.85 | — |
| 163 | 0.038 | — | 0.46 | 0.25 | 0.49 |
| 164 | 0.12 | — | 0.34 | 0.65 | — |
| 165 | 0.38 | — | 0.37 | 0.63 | — |
| 166 | 5.39 | — | 0.33 | 0.50 | — |
| 167 | 17.8 | — | 2.38 | 3.85 | — |
| 168 | 0.23 | — | 0.15 | 0.11 | 0.22 |
| 169 | 4.62 | — | 2.18 | 1.33 | — |
| 170 | 1.61 | — | 0.33 | 0.096 | — |
| 171 | 1.56 | — | 0.56 | 0.22 | — |
| 174 | 0.59 | — | 0.14 | 0.14 | — |
| 175 | 0.98 | — | 0.83 | 1.69 | — |
| 176 | 0.18 | — | 0.11 | 0.19 | 0.21 |
| 177 | 0.61 | — | 0.77 | 1.71 | 0.52 |
| 178 | 0.29 | — | 0.13 | 0.091 | — |
| 179 | 0.081 | — | 0.022 | 0.055 | — |
| 181 | 4.92 | — | 12.4 | 0.73 | — |
| 182 | 0.43 | — | 2.03 | 2.53 | — |
| 183 | 0.083 | — | 2.69 | 2.33 | 0.27 |
| 184 | 2.66 | — | 45.0 | 20.0 | — |
| 185 | — | 0.27 | 0.61 | — | — |
| 188 | — | 0.014 | 0.31 | 0.073 | 0.27 |
| 189 | — | 0.85 | 17.0 | 0.45 | — |
| 190 | 0.029 | — | 0.017 | 0.009 | — |
| 191 | — | 0.46 | 0.26 | 0.29 | — |
| 192 | — | 0.061 | 0.052 | 0.11 | — |
| 193 | — | 0.65 | 1.77 | 1.15 | — |
| 194 | — | — | 5.25 | — | 0.27 |
| 195 | 125 | — | 5.07 | — | — |
| 196 | 0.12 | — | 0.012 | 0.031 | 0.049 |
| 197 | 1.47 | — | 0.22 | 0.31 | — |
| 198 | — | 0.037 | 0.038 | 0.015 | — |
| 199 | — | 0.63 | 0.92 | 2.06 | 11.3 |
| 200 | — | 0.012 | 0.013 | — | — |
| 201 | — | 0.16 | 0.90 | 0.23 | — |
| 202 | — | 0.36 | 0.42 | 0.16 | — |
| 203 | — | 0.78 | 2.71 | 2.21 | — |
| 204 | — | 0.12 | 1.41 | 0.41 | — |
| 205 | — | 0.061 | 3.74 | 0.83 | — |
| 206 | — | 3.98 | 51.0 | 20.0 | 0.080 |
| 207 | — | 0.62 | 0.97 | 1.06 | — |
| 208 | — | 0.025 | 0.078 | 0.017 | — |
| 209 | — | 0.091 | 1.03 | 1.28 | — |
| 210 | — | 0.046 | 0.97 | 0.031 | — |
| 211 | — | 0.046 | 0.10 | 0.018 | — |
| 212 | — | 0.018 | 0.070 | 0.022 | — |
| 213 | — | 0.059 | 0.95 | 0.19 | — |
| 214 | — | 2.01 | 6.62 | 20.0 | 0.066 |
| 215 | — | 0.077 | 0.11 | 0.17 | 20.0 |
| 216 | — | 0.087 | 0.088 | 0.012 | — |
| 217 | — | 1.66 | 4.26 | 0.87 | — |
| 218 | — | 0.078 | 0.018 | — | — |
| 220 | — | 0.11 | 0.046 | 0.081 | — |
| 221 | — | 0.002 | 0.25 | 0.48 | — |
| 222 | — | 0.002 | 0.017 | — | — |
| 223 | — | 0.13 | 2.08 | 0.90 | — |
| 224 | — | 0.081 | 0.14 | 0.12 | — |
| 225 | — | 0.46 | 4.13 | 0.61 | — |
| 226 | — | 1.21 | 2.65 | 5.60 | — |
| 227 | — | 2.45 | 1.79 | 0.64 | — |
| 228 | — | 0.19 | 0.30 | 0.60 | — |
| 229 | — | 0.68 | 3.56 | 1.61 | — |
| 230 | — | 0.042 | 0.13 | 1.29 | — |
| 231 | — | — | — | 9.74 | — |
| 232 | — | 0.002 | 0.013 | 0.044 | 0.044 |
| 233 | — | 0.17 | 0.95 | 2.77 | — |
| 234 | — | 0.036 | 0.059 | — | 0.08 |
| 235 | — | 0.43 | 0.78 | — | — |
| 236 | — | 0.009 | 0.058 | 0.012 | 0.044 |
| 237 | — | 0.053 | 3.96 | 1.43 | — |
| 238 | — | 0.015 | 0.11 | 0.023 | — |
| 239 | — | 0.094 | 2.23 | 2.85 | — |
| 240 | — | 0.073 | 0.11 | 0.27 | 0.29 |
| 241 | — | 0.46 | 0.92 | 2.42 | 0.19 |
| 242 | — | 0.26 | 0.19 | — | — |
| 243 | — | 0.006 | 0.039 | 0.059 | — |
| 244 | — | — | — | 13.3 | — |
| 245 | — | 0.018 | 0.29 | 0.22 | 0.41 |
| 246 | — | 4.67 | 5.85 | — | 0.44 |
| 247 | — | 5.87 | 0.84 | — | — |
| 249 | — | — | 0.04 | — | 0.42 |
| 250 | — | 0.33 | 1.15 | — | 1.80 |
| 251 | — | 0.034 | 0.018 | — | 1.58 |
| 252 | — | 0.27 | 0.78 | — | 4.28 |
| 253 | — | 3.80 | 0.097 | — | — |
| 254 | — | 2.85 | 2.15 | — | — |
| 255 | — | 0.058 | 0.12 | — | 0.54 |
| 256 | — | 1.51 | 2.94 | — | 0.29 |
| 257 | — | 0.17 | 0.25 | — | — |
| 258 | — | 0.064 | 0.36 | — | — |
| 259 | — | 2.17 | 1.80 | — | — |
| 260 | — | — | 3.03 | — | — |
| 261 | — | 0.17 | 0.017 | — | 0.68 |
| 262 | — | — | — | — | 4.15 |
| 263 | — | 0.29 | 0.062 | — | — |
| 264 | — | 0.75 | 1.17 | — | — |
| 265 | — | 0.79 | 0.37 | 1.59 | — |
| 266 | — | 0.083 | 0.028 | 0.18 | — |
| 267 | — | 0.041 | 0.10 | 0.045 | 1.02 |
| 268 | — | 0.34 | 4.06 | 1.00 | — |
| 269 | — | 0.58 | 0.26 | — | — |
| 270 | — | 0.008 | 2.32 | 1.39 | — |
| 271 | — | 0.010 | 12.7 | 14.4 | — |
| 273 | — | 1.00 | 7.33 | 0.42 | — |
| 274 | — | 16.0 | 174 | 5.33 | — |
| 275 | — | 0.53 | 0.15 | 0.035 | — |
| 276 | — | 0.12 | 1.38 | 3.56 | — |
| 277 | — | 0.16 | 0.018 | — | — |
| 278 | — | 2.86 | — | 1.15 | — |
| 279 | — | 17.0 | 62.4 | — | 2.92 |
| 280 | — | 42.7 | 125 | — | 0.15 |
| 281 | — | 7.17 | 43.6 | — | — |
| 282 | — | 0.15 | 1.16 | — | — |
| 283 | — | 0.20 | 2.43 | 1.25 | 6.26 |
| 284 | — | 3.17 | 25.1 | 13.0 | — |
| 285 | — | 4.10 | 8.54 | — | — |
| 286 | — | 36.2 | 125 | — | — |
| 287 | — | 0.21 | 0.50 | — | — |
| 288 | — | 4.11 | 31.7 | — | — |
| 289 | — | 2.70 | 8.64 | 5.72 | 0.35 |
| 290 | — | 1.95 | 57.2 | — | 20.0 |
| 291 | — | 0.11 | 0.43 | — | 0.39 |
| 292 | — | 0.21 | 1.01 | — | 1.10 |
| 294 | 8.29 | — | 0.042 | 0.66 | 0.20 |
| 295 | 7.23 | 0.10 | 0.82 | 0.58 | 0.10 |
| 296 | — | 0.46 | 0.14 | 0.036 | — |
| 297 | — | 0.11 | 0.11 | 0.053 | — |
| 298 | — | 0.48 | 0.38 | 0.42 | — |
| 299 | — | 1.28 | 4.68 | 3.46 | — |
| 300 | — | 0.013 | 0.022 | — | — |
| 301 | — | 0.13 | 0.43 | — | — |
| 302 | — | 0.009 | 0.043 | — | 0.034 |
| 303 | — | 1.77 | 5.07 | — | — |
| 304 | — | 0.012 | 0.014 | 0.016 | 0.11 |
| 305 | — | 0.062 | 1.40 | 11.7 | 3.67 |
| 306 | — | 0.011 | 0.028 | — | 0.23 |
| 307 | — | 0.14 | 0.77 | — | — |
| 308 | — | 0.062 | 0.039 | 0.034 | 0.89 |
| 309 | — | 0.35 | 0.85 | 1.48 | — |
| 310 | — | 0.077 | 0.28 | — | — |
| 311 | — | 0.013 | 0.006 | — | — |
| 312 | — | 0.60 | 0.87 | — | — |
| 313 | — | 0.23 | 0.65 | — | 0.057 |
| 314 | — | 0.025 | 0.004 | — | 0.050 |
| 315 | — | 0.026 | 0.19 | — | 0.27 |
| 316 | — | 0.087 | 0.031 | — | — |
| 317 | — | 0.017 | 0.026 | — | 0.18 |
| 318 | — | 0.62 | 1.57 | — | 3.07 |
| 319 | — | 0.052 | 0.022 | — | 0.13 |
| 320 | — | 0.42 | 0.62 | — | 0.93 |
| 321 | — | 0.010 | 0.038 | — | — |
| 322 | — | 0.007 | 0.76 | — | — |
| 323 | — | 0.52 | 0.021 | — | 0.67 |
| 324 | — | 0.85 | 0.20 | — | 4.21 |
| 325 | — | 0.80 | 0.069 | — | 0.07 |
| 326 | — | — | — | — | 3.48 |
| 327 | — | 0.32 | 0.057 | — | 2.07 |
| 328 | — | 2.97 | 2.23 | — | 20.0 |
| 329 | — | 5.29 | 0.74 | — | 1.56 |
| 330 | — | 0.64 | 2.83 | — | 11.0 |
| 331 | — | 0.038 | 0.033 | — | 0.94 |
| 332 | — | 0.074 | 0.72 | — | — |
| 333 | — | 0.090 | 0.072 | 0.12 | 0.10 |
| 334 | — | 0.46 | 0.53 | 0.73 | 20.0 |
| 335 | — | 0.025 | 0.12 | — | 0.14 |
| 336 | — | 0.48 | 1.10 | — | 20.00 |
| 337 | — | 0.081 | 0.10 | — | 0.054 |
| 338 | — | 0.91 | 1.31 | — | 20.0 |
| 339 | — | 0.075 | 0.079 | — | 0.28 |
| 340 | — | 6.31 | 15.9 | — | 12.9 |
| 341 | — | 0.061 | 0.13 | — | 2.98 |
| 342 | — | 1.09 | 1.20 | — | 7.93 |
| 343 | — | 0.74 | 6.33 | — | 0.073 |
| 344 | — | 10.1 | 17.1 | — | 20.0 |
| 345 | — | 0.049 | 0.095 | — | 1.74 |
| 346 | — | 1.28 | 1.24 | — | 0.19 |
| 347 | — | 0.32 | 0.95 | — | 1.99 |
| 348 | — | 0.061 | 0.045 | — | 1.95 |
| 350 | — | 0.18 | 0.12 | — | 3.36 |
| 351 | — | 0.18 | 1.04 | — | 0.38 |
| 352 | — | 14.9 | 2.72 | — | — |
| 354 | — | — | 0.65 | — | 7.95 |
| 355 | — | — | 41.7 | — | 6.69 |
| 356 | — | 18.0 | 0.44 | — | 0.93 |
| 357 | — | 2.08 | 1.56 | — | 3.78 |
| 359 | — | 0.024 | 0.22 | — | — |
| 360 | — | 0.15 | 0.66 | — | — |
| 361 | — | 0.005 | 0.022 | 0.26 | — |
| 362 | — | 0.061 | 0.59 | 3.09 | — |
| 363 | — | 0.017 | 0.032 | 0.16 | 0.024 |
| 364 | — | 0.053 | 0.33 | — | 1.80 |
| 365 | — | 0.016 | 0.021 | — | 4.41 |
| 366 | — | 1.45 | 0.62 | — | — |
| 367 | — | 0.12 | 0.85 | — | — |
| 368 | — | 1.17 | 2.25 | — | — |
| 371 | — | 0.013 | 0.10 | 0.19 | 3.32 |
| 372 | — | 0.004 | 0.043 | 0.15 | — |
| 373 | 0.86 | — | 2.55 | 0.17 | — |
| 374 | 0.65 | — | 2.79 | 0.82 | — |
| 375 | 1.39 | — | 6.05 | 2.16 | — |
| 376 | 0.03 | — | 0.097 | 0.058 | — |
| 377 | 0.24 | — | 0.37 | 0.52 | — |
| 378 | — | 0.16 | 0.15 | — | — |
| 379 | — | 0.45 | 3.60 | — | — |
| 380 | — | 0.029 | 0.18 | — | — |
| 381 | — | 0.85 | 3.41 | — | — |
| 385 | — | 0.012 | 0.043 | 0.13 | 0.24 |
| 386 | — | 2.70 | 5.07 | 2.49 | — |
| 387 | — | 1.93 | 6.07 | 0.77 | — |
| 388 | — | 1.75 | 45.7 | 20.0 | — |
| 389 | — | 1.00 | 8.22 | — | 0.37 |
| 390 | — | 0.097 | 0.18 | 0.04 | 20.00 |
| 391 | — | 2.02 | 1.34 | 0.72 | 1.13 |
| 392 | — | 2.84 | 33.6 | — | 6.89 |
| 393 | — | 23.5 | 125 | — | 3.45 |
| 395 | — | 0.49 | 0.064 | — | 20.0 |
| 396 | — | 0.92 | 1.30 | — | — |
| 398 | — | 0.090 | 0.064 | — | — |
| 399 | — | 0.16 | 0.70 | — | 20.0 |
| 400 | — | 0.75 | 0.047 | — | 20.0 |
| 401 | — | 0.16 | 0.23 | — | 4.89 |
| 402 | 49.5 | — | 4.76 | 1.39 | 3.67 |
| 405 | 13.6 | — | 0.11 | 1.19 | 2.58 |
| 406 | — | 1.96 | 81.1 | — | 7.13 |
| 407 | — | 0.40 | 31.0 | — | 0.64 |
| 409 | — | 0.072 | 1.08 | — | 20.0 |
| 410 | — | 0.22 | 2.37 | — | — |
| 411 | — | 0.82 | 1.24 | — | 1.89 |
| 412 | — | 9.67 | 45.0 | — | 0.67 |
| 413 | — | 0.44 | 0.75 | — | — |
| 414 | — | 1.49 | 11.3 | — | 0.16 |
| 415 | — | 3.31 | 61.9 | — | 3.44 |
| 416 | — | 0.14 | 0.74 | — | 3.14 |
| 417 | — | 0.17 | 0.52 | — | 20.0 |
| 418 | — | 0.092 | 8.71 | — | 20.0 |
| 419 | — | 0.033 | 0.055 | — | 20.0 |
| 420 | — | 0.090 | 0.76 | — | — |
| 421 | — | 0.064 | 0.32 | 0.19 | — |
| 422 | — | 0.23 | 6.87 | — | — |
| 424 | — | 114.4 | 4.59 | — | — |
| 425 | — | 83.3 | 12.5 | — | — |
| 427 | — | 0.46 | 4.45 | — | — |
| 428 | — | — | 125 | 20.0 | — |
| 429 | — | — | 99.5 | 20.0 | — |
| 430 | — | — | 125 | 20.0 | 20.0 |
| 431 | — | 250 | 250 | 20.0 | 20.0 |
| 432 | — | — | 41.67 | 20.0 | |
| 433 | — | 125 | 125 | 20.0 | |
| 434 | — | — | 125 | 20.0 | 20.0 |
| 435 | — | 1.90 | 5.51 | 4.16 | 7.29 |
| 436 | — | 12.19 | 250 | 20.0 | — |
| 437 | — | 250 | 250 | — | — |
| 438 | — | 175 | 250 | — | — |
| 439 | — | 0.36 | 0.40 | — | 10.9 |
| 440 | — | 1.14 | 44.6 | — | 8.07 |
| 441 | — | 0.19 | 26.9 | — | 0.70 |
| 442 | — | 0.28 | 0.91 | — | — |
| 444 | — | 0.40 | 0.18 | — | — |
| 445 | — | 0.95 | 2.21 | — | 9.59 |
| 447 | — | 13.6 | 5.57 | — | 20.0 |
| 448 | — | 13.4 | 5.14 | — | 20.0 |
| 449 | — | 7.41 | 4.08 | — | 20.0 |
| 450 | — | 0.20 | 0.37 | — | 20.0 |
| 451 | — | — | — | — | 20.0 |
| 452 | — | — | — | — | 20.0 |
| 453 | — | 215 | 79.4 | — | — |
| 454 | — | 14.2 | 28.8 | — | — |
| 455 | — | 5.12 | 7.55 | — | — |
| 456 | — | 10.2 | 12.1 | — | — |
| 457 | — | 4.01 | 1.05 | — | — |
| 458 | — | 7.74 | 6.47 | — | — |
| 459 | — | 29.8 | 23.9 | — | — |
| 460 | — | 11.6 | 12.0 | — | — |

Table A lists in vitro DGK inhibition IC₅₀ activity values measured in the DGKα and DGKζ liposome assays.

The compounds of the present invention possess activity as an inhibitor(s) of one or both of the DGKα and DGKζ enzymes, and therefore, may be used in the treatment of diseases associated with the inhibition of DGKα and DGKζ activity.

| Nucleotide sequence encoding hDGKα-(M1-S735)-Ct-TVMV-His: | | |
|---|---|---|
| (SEQ ID NO: 1) | | |
| 1 | ATGGCCAAGG AGAGGGGCCT AATAAGCCCC AGTGATTTTG CCCAGCTGCA | |
| 51 | AAAATACATG GAATACTCCA CCAAAAAGGT CAGTGATGTC CTAAAGCTCT | |
| 101 | TCGAGGATGG CGAGATGGCT AAATATGTCC AAGGAGATGC CATTGGGTAC | |
| 151 | GAGGGATTCC AGCAATTCCT GAAAATCTAT CTCGAAGTGG ATAATGTTCC | |
| 201 | CAGACACCTA AGCCTGGCAC TGTTTCAATC CTTTGAGACT GGTCACTGCT | |
| 251 | TAAATGAGAC AAATGTGACA AAAGATGTGG TGTGTCTCAA TGATGTTTCC | |
| 301 | TGCTACTTTT CCCTTCTGGA GGGTGGTCGG CCAGAAGACA AGTTAGAATT | |
| 351 | CACCTTCAAG CTGTACGACA CGGACAGAAA TGGGATCCTG GACAGCTCAG | |
| 401 | AAGTGGACAA AATTATCCTA CAGATGATGC GAGTGGCTGA ATACCTGGAT | |
| 451 | TGGGATGTGT CTGAGCTGAG GCCGATTCTT CAGGAGATGA TGAAAGAGAT | |
| 501 | TGACTATGAT GGCAGTGGCT CTGTCTCTCA AGCTGAGTGG GTCCGGGCTG | |
| 551 | GGGCCACCAC CGTGCCACTG CTAGTGCTGC TGGGTCTGGA GATGACTCTG | |
| 601 | AAGGACGACG GACAGCACAT GTGGAGGCCC AAGAGGTTCC CCAGACCAGT | |
| 651 | CTACTGCAAT CTGTGCGAGT CAAGCATTGG TCTTGGCAAA CAGGGACTGA | |
| 701 | GCTGTAACCT CTGTAAGTAC ACTGTTCACG ACCAGTGTGC CATGAAAGCC | |
| 751 | CTGCCTTGTG AAGTCAGCAC CTATGCCAAG TCTCGGAAGG ACATTGGTGT | |
| 801 | CCAATCACAT GTGTGGGTGC GAGGAGGCTG TGAGTCCGGG CGCTGCGACC | |
| 851 | GCTGTCAGAA AAAGATCCGG ATCTACCACA GTCTGACCGG GCTGCATTGT | |
| 901 | GTATGGTGCC ACCTAGAGAT CCACGATGAC TGCCTGCAAG CGGTGGGCCA | |
| 951 | TGAGTGTGAC TGTGGGCTGC TCCGGGATCA CATCCTGCCT CCATCTTCCA | |
| 1001 | TCTATCCCAG TGTCCTGGCC TCTGGACCGG ATCGTAAAAA TAGCAAAACA | |
| 1051 | AGCCAGAAGA CCATGGATGA TTTAAATTTG AGCACCTCTG AGGCTCTGCG | |
| 1101 | GATTGACCCT GTTCCTAACA CCCACCCACT TCTCGTCTTT GTCAATCCTA | |
| 1151 | AGAGTGGCGG GAAGCAGGGG CAGAGGGTGC TCTGGAAGTT CCAGTATATA | |
| 1201 | TTAAACCCTC GACAGGTGTT CAACCTCCTA AAGGATGGTC CTGAGATAGG | |
| 1251 | GCTCCGATTA TTCAAGGATG TTCCTGATAG CCGGATTTTG GTGTGTGGTG | |
| 1301 | GAGACGGCAC AGTAGGCTGG ATTCTAGAGA CCATTGACAA AGCTAACTTG | |
| 1351 | CCAGTTTTGC CTCCTGTTGC TGTGTTGCCC CTGGGTACTG GAAATGATCT | |
| 1401 | GGCTCGATGC CTAAGATGGG GAGGAGGTTA TGAAGGACAG AATCTGGCAA | |
| 1451 | AGATCCTCAA GGATTTAGAG ATGAGTAAAG TGGTACATAT GGATCGATGG | |
| 1501 | TCTGTGGAGG TGATACCTCA ACAAACTGAA GAAAAAAGTG ACCCAGTCCC | |
| 1551 | CTTTCAAATC ATCAATAACT ACTTCTCTAT TGGCGTGGAT GCCTCTATTG | |
| 1601 | CTCATCGATT CCACATCATG CGAGAGAAAT ATCCGGAGAA GTTCAACAGC | |
| 1651 | AGAATGAAGA ACAAGCTATG GTACTTCGAA TTTGCCACAT CTGAATCCAT | |
| 1701 | CTTCTCAACA TGCAAAAAGC TGGAGGAGTC TTTGACAGTT GAGATCTGTG | |
| 1751 | GGAAACCGCT GGATCTGAGC AACCTGTCCC TAGAAGGCAT CGCAGTGCTA | |
| 1801 | AACATCCCTA GCATGCATGG TGGCTCCAAC CTCTGGGGTG ATACCAGGAG | |
| 1851 | ACCCCATGGG GATATCTATG GGATCAACCA GGCCTTAGGT GCTACAGCTA | |
| 1901 | AAGTCATCAC CGACCCTGAT ATCCTGAAAA CCTGTGTACC AGACCTAAGT | |
| 1951 | GACAAGAGAC TGGAAGTGGT TGGGCTGGAG GGTGCAATTG AGATGGGCCA | |
| 2001 | AATCTATACC AAGCTCAAGA ATGCTGGACG TCGGCTGGCC AAGTGCTCTG | |
| 2051 | AGATCACCTT CCACACCACA AAAACCCTTC CCATGCAAAT TGACGGAGAA | |
| 2101 | CCCTGGATGC AGACGCCCTG TACAATCAAG ATCACCCACA AGAACCAGAT | |
| 2151 | GCCCATGCTC ATGGGCCCAC CCCCCCGCTC CACCAATTTC TTTGGCTTCT | |
| 2201 | TGAGCGGATC CTCGGAGACA GTGCGGTTTC AGGGACACCA CCACCATCAC | |
| 2251 | CACTGA | |
| Amino acid sequence of hDGKα-(9-727)-TVMV-His: | | |
| (SEQ ID NO: 2) | | |
| 0001 | MASPSDFAQL QKYMEYSTKK VSDVLKLFED GEMAKYVQGD AIGYEGFQQF LKIYLEVDNV | 0060 |
| 0061 | PRHLSLALFQ SFETGHCLNE TNVTKDVVCL NDVSCYFSLL EGGRPEDKLE FTFKLYDTDR | 0120 |
| 0121 | NGILDSSEVD KIILQMMRVA EYLDWDVSEL RPILQEMMKE IDYDGSGSVS QAEWVRAGAT | 0180 |
| 0181 | TVPLLVLLGL EMTLKDDGQH MWRPKRFPRP VYCNLCESSI GLGKQGLSCN LCKYTVHDQC | 0240 |
| 0241 | AMKALPCEVS TYAKSRKDIG VQSHVWVRGG CESGRCDRCQ KKIRIYHSLT GLHCVWCHLE | 0300 |
| 0301 | IHDDCLQAVG HECDCGLLRD HILPPSSIYP SVLASGPDRK NSKTSQKTMD DLNLSTSEAL | 0360 |
| 0361 | RIDPVPNTHP LLVFVNPKSG GKQGQRVLWK FQYILNPRQV FNLLKDGPEI GLRLFKDVPD | 0420 |
| 0421 | SRILVCGGDG TVGWILETID KANLPVLPPV AVLPLGTGND LARCLRWGGG YEGQNLAKIL | 0480 |
| 0481 | KDLEMSKVVH MDRWSVEVIP QQTEEKSDPV PFQIINNYFS IGVDASIAHR FHIMREKYPE | 0540 |
| 0541 | KFNSRMKNKL WYFEFATSES IFSTCKKLEE SLTVEICGKP LDLSNLSLEG IAVLNIPSMH | 0600 |
| 0601 | GGSNLWGDTR RPHGDIYGIN QALGATAKVI TDPDILKTCV PDLSDKRLEV VGLEGAIEMG | 0660 |
| 0661 | QIYTKLKNAG RRLAKCSEIT FHTTKTLPMQ IDGEPWMQTP CTIKITHKNQ MPMLMGPPPR | 0720 |
| 0721 | SGSSETVRFQ GHHHHHH | 0737 |
| Nucleotide sequence encoding hDGKζ-(M1-A928)-transcript variant-2 Ct-TVMV-His: | | |
| (SEQ ID NO: 3) | | |
| 1 | ATGGAGCCGC GGGACGGTAG CCCCGAGGCC CGGAGCAGCG ACTCCGAGTC | |
| 51 | GGCTTCCGCC TCGTCCAGCG GCTCCGAGCG CGACGCCGGT CCCGAGCCGG | |
| 101 | ACAAGGCGCC GCGGCGACTC AACAAGCGGC GCTTCCCGGG GCTGCGGCTC | |
| 151 | TTCGGGCACA GGAAAGCCAT CACGAAGTCG GGCCTCCAGC ACCTGGCCCC | |
| 201 | CCCTCCGCCC ACCCCTGGGG CCCCGTGCAG CGAGTCAGAG CGGCAGATCC | |
| 251 | GGAGTACAGT GGACTGGAGC GAGTCAGCGA CATATGGGGA GCACATCTGG | |
| 301 | TTCGAGACCA ACGTGTCCGG GGACTTCTGC TACGTTGGGG AGCAGTACTG | |
| 351 | TGTAGCCAGG ATGCTGCAGA AGTCAGTGTC TCGAAGAAAG TGCGCAGCCT | |
| 401 | GCAAGATTGT GGTGCACACG CCCTGCATCG AGCAGCTGGA GAAGATAAAT | |
| 451 | TTCCGCTGTA AGCCGTCCTT CCGTGAATCA GGCTCCAGGA ATGTCCGCGA | |
| 501 | GCCAACCTTT GTACGGCACC ACTGGGTACA CAGACGACGC CAGGACGGCA | |
| 551 | AGTGTCGGCA CTGTGGGAAG GGATTCCAGC AGAAGTTCAC CTTCCACAGC | |
| 601 | AAGGAGATTG TGGCCATCAG CTGCTCGTGG TGCAAGCAGG CATACCACAG | |
| 651 | CAAGGTGTCC TGCTTCATGC TGCAGCAGAT CGAGGAGCCG TGCTCGCTGG | |
| 701 | GGGTCCACGC AGCCGTGGTC ATCCCGCCCA CCTGGATCCT CCGCGCCCGG | |
| 751 | AGGCCCCAGA ATACTCTGAA AGCAAGCAAG AAGAAGAAGA GGGCATCCTT | |
| 801 | CAAGAGGAAG TCCAGCAAGA AAGGGCCTGA GGAGGGCCGC TGGAGACCCT | |
| 851 | TCATCATCAG GCCCACCCCC TCCCCGCTCA TGAAGCCCCT GCTGGTGTTT | |
| 901 | GTGAACCCCA AGAGTGGGGG CAACCAGGGT GCAAAGATCA TCCAGTCTTT | |
| 951 | CCTCTGGTAT CTCAATCCCC GACAAGTCTT CGACCTGAGC CAGGGAGGGC | |
| 1001 | CCAAGGAGGC GCTGGAGATG TACCGCAAAG TGCACAACCT GCGGATCCTG | |
| 1051 | GCGTGCGGGG GCGACGGCAC GGTGGGCTGG ATCCTCTCCA CCCTGGACCA | |
| 1101 | GCTACGCCTG AAGCCGCCAC CCCCTGTTGC CATCCTGCCC CTGGGTACTG | |
| 1151 | GCAACGACTT GGCCCGAACC CTCAACTGGG GTGGGGGCTA CACAGATGAG | |
| 1201 | CCTGTGTCCA AGATCCTCTC CCACGTGGAG GAGGGGAACG TGGTACAGCT | |
| 1251 | GGACCGCTGG GACCTCCACG CTGAGCCCAA CCCCGAGGCA GGGCCTGAGG | |
| 1301 | ACCGAGATGA AGGCGCCACC GACCGGTTGC CCCTGGATGT CTTCAACAAC | |
| 1351 | TACTTCAGCC TGGGCTTTGA CGCCCACGTC ACCCTGGAGT TCCACGAGTC | |
| 1401 | TCGAGAGGCC AACCCAGAGA AATTCAACAG CCGCTTTCGG AATAAGATGT | |
| 1451 | TCTACGCCGG GACAGCTTTC TCTGACTTCC TGATGGGCAG CTCCAAGGAC | |
| 1501 | CTGGCCAAGC ACATCCGAGT GGTGTGTGAT GGAATGGACT TGACTCCCAA | |
| 1551 | GATCCAGGAC CTGAAACCCC AGTGTGTTGT TTTCCTGAAC ATCCCCAGGT | |
| 1601 | ACTGTGCGGG CACCATGCCC TGGGGCCACC CTGGGGAGCA CCACGACTTT | |
| 1651 | GAGCCCCAGC GGCATGACGA CGGCTACCTC GAGGTCATTG GCTTCACCAT | |
| 1701 | GACGTCGTTG GCCGCGCTGC AGGTGGGCGG ACACGGCGAG CGGCTGACGC | |
| 1751 | AGTGTCGCGA GGTGGTGCTC ACCACATCCA AGGCCATCCC GGTGCAGGTG | |
| 1801 | GATGGCGAGC CCTGCAAGCT TGCAGCCTCA CGCATCCGCA TCGCCCTGCG | |
| 1851 | CAACCAGGCC ACCATGGTGC AGAAGGCCAA GCGGCGGAGC GCCGCCCCCC | |
| 1901 | TGCACAGCGA CCAGCAGCCG GTGCCAGAGC AGTTGCGCAT CCAGGTGAGT | |
| 1951 | CGCGTCAGCA TGCACGACTA TGAGGCCCTG CACTACGACA AGGAGCAGCT | |
| 2001 | CAAGGAGGCC TCTGTGCCGC TGGGCACTGT GGTGGTCCCA GGAGACAGTG | |
| 2051 | ACCTAGAGCT CTGCCGTGCC CACATTGAGA GACTCCAGCA GGAGCCCGAT | |
| 2101 | GGTGCTGGAG CCAAGTCCCC GACATGCCAG AAACTGTCCC CCAAGTGGTG | |
| 2151 | CTTCCTGGAC GCCACCACTG CCAGCCGCTT CTACAGGATC GACCGAGCCC | |
| 2201 | AGGAGCACCT CAACTATGTG ACTGAGATCG CACAGGATGA GATTTATATC | |
| 2251 | CTGGACCCTG AGCTGCTGGG GGCATCGGCC CGGCCTGACC TCCCAACCCC | |
| 2301 | CACTTCCCCT CTCCCCACCT CACCCTGCTC ACCCACGCCC CGGTCACTGC | |
| 2351 | AAGGGGATGC TGCACCCCCT CAAGGTGAAG AGCTGATTGA GGCTGCCAAG | |
| 2401 | AGGAACGACT TCTGTAAGCT CCAGGAGCTG CACCGAGCTG GGGGCGACCT | |
| 2451 | CATGCACCGA GACGAGCAGA GTCGCACGCT CCTGCACCAC GCAGTCAGCA | |
| 2501 | CTGGCAGCAA GGATGTGGTC CGCTACCTGC TGGACCACGC CCCCCCAGAG | |
| 2551 | ATCCTTGATG CGGTGGAGGA AAACGGGGAG ACCTGTTTGC ACCAAGCAGC | |
| 2601 | GGCCCTGGGC CAGCGCACCA TCTGCCACTA CATCGTGGAG GCCGGGGCCT | |
| 2651 | CGCTCATGAA GACAGACCAG CAGGGCGACA CTCCCCGGCA GCGGGCTGAG | |
| 2701 | AAGGCTCAGG ACACCGAGCT GGCCGCCTAC CTGGAGAACC GGCAGCACTA | |
| 2751 | CCAGATGATC CAGCGGGAGG ACCAGGAGAC GGCTGTGGGA TCCTCGGAGA | |
| 2801 | CAGTGCGGTT TCAGGGACAC CACCACCATC ACCACTGA | |
| Amino acid sequence of hDGKζ-(M1-A928)-transcript variant-2 Ct-TVMV-His: | | |
| (SEQ ID NO: 4) | | |
| 0001 | MEPRDGSPEA RSSDSESASA SSSGSERDAG PEPDKAPRRL NKRRFPGIRL FGHRKAITKS | 0060 |
| 0061 | GLQHLAPPPP TPGAPCSESE RQIRSTVDWS ESATYGEHIW FETNVSGDFC YVGEQYCVAR | 0120 |
| 0121 | mLQKSVSRRK CAACKIVVHT PCIEQLEKIN FRCKPSFRES GSRNVREPTF VRHHWVHRRR | 0180 |
| 0181 | QDGKCRHCGK GFQQKFTFHS KEIVAISCSW CKQAYHSKVS CEMLQQIEEP CSLGVHAAVV | 0240 |
| 0241 | IPPTWILRAR RPQNTLKASK KKKRASFKRK SSKKGPEEGR WRPFIIRPTP SPLMKPLLVF | 0300 |
| 0301 | VNPKSGGNQG AKIIQSFLWY LNPRQVFDLS QGGPKEALEM YRKVHNLRIL ACGGDGTVGW | 0360 |
| 0361 | ILSTLDQLRL KPPPPVAILP LGTGNDLART LNWGGGYTDE PVSKILSHVE EGNVVQLDRW | 0420 |
| 0421 | DLHAEPNPEA GPEDRDEGAT DRLPLDVFNN YFSLGFDAHV TLEFHESREA NPEKFNSRFR | 0480 |
| 0481 | NKMFYAGTAF SDFLMGSSKD LAKHIRVVCD GMDLTPKIQD LKPQCVVFLN IPRYCAGTMP | 0540 |
| 0541 | WGHPGEHHDF EPQRHDDGYL EVIGFTMTSL AALQVGGHGE RLTQCREVVL TTSKAIPVQV | 0600 |
| 0601 | DGEPCKLAAS RIRIALRNQA TMVQKAKRRS AAPLHSDQQP VPEQLRIQVS RVSMHDYEAL | 0660 |
| 0661 | HYDKEQLKEA SVPLGTVVVP GDSDLELCRA HIERLQQEPD GAGAKSPTCQ KLSPKWCFLD | 0720 |
| 0721 | ATTASRFYRI DRAQEHLNYV TEIAQDEIYI LDPELLGASA RPDLPTPTSP LPTSPCSPTP | 0780 |
| 0781 | RSLQGDAAPP QGEELIEAAK RNDFCKLQEL HRAGGDLMHR DEQSRTLLHH AVSTGSKDVV | 0840 |
| 0841 | RYLLDHAPPE ILDAVEENGE TCLHQAAALG QRTICHYIVE AGASLMKTDQ QGDTPRQRAE | 0900 |
| 0901 | KAQDTELAAY LENRQHYQMI QREDQETAVG SSETVRFQGH HHHHH | 0945 |

What is claimed is:

1-50. (canceled)

51. A compound of Formula (I): 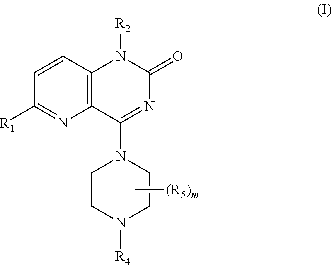
or a pharmaceutically acceptable salt thereof, wherein:
R₁ is —OCH₂CH₃;
R₂ is H, C₁₋₃ alkyl substituted with zero to 4 R₂ₐ, or C₃₋₄ cycloalkyl substituted with zero to 4 R₂ₐ;
each R₂ₐ is independently F, Cl, —CN, —OH, —O(C₁₋₂ alkyl), C₃₋₄ cycloalkyl, C₃₋₄ alkenyl, or C₃₋₄ alkynyl;
R₄ is —CHR₄ₐR_{4b};
R₄ₐ is C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₄ hydroxyalkyl, —(CH₂)₁₋₂O(C₁₋₃ alkyl), C₁₋₄ alkoxy, C₁₋₃ fluoroalkoxy, C₁₋₃ cyanoalkoxy, —O(C₁₋₄ hydroxyalkyl), —O(CRₓRₓ)₁₋₃O(C₁₋₃ alkyl), C₁₋₃ fluoroalkoxy, —O(CH₂)₁₋₃NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —CH₂NRₐRₐ, —NRₐS(O)₂(C₁₋₃ alkyl), —NRₐC(O)(C₁₋₃ alkyl), —(CRₓRₓ)₀₋₂NRₐC(O)O(C₁₋₄ alkyl), —P(O)(C₁₋₃ alkyl)₂, —S(O)₂(C₁₋₃ alkyl), —(CRₓRₓ)₁₋₂(C₃₋₄ cycloalkyl), —(CRₓRₓ)₁₋₂(morpholinyl), —(CRₓRₓ)₁₋₂(difluoromorpholinyl), —(CRₓRₓ)₁₋₂(dimethylmorpholinyl), —(CRₓRₓ)₁₋₂(oxaazabicyclo[2.2.1]heptanyl), (CRₓRₓ)₁₋₂(oxaazaspiro[3.3]heptanyl), —(CRₓRₓ)₁₋₂(methylpiperazinonyl), —(CRₓRₓ)₁₋₂(acetylpiperazinyl), —(CRₓRₓ)₁₋₂(piperidinyl), —(CRₓRₓ)₁₋₂(difluoropiperidinyl), —(CRₓRₓ)₁₋₂(methoxypiperidinyl), —(CRₓRₓ)₁₋₂(hydroxypiperidinyl), —O(CRₓRₓ)₀₋₂(C₃₋₆ cycloalkyl), —O(CRₓRₓ)₀₋₂(methylcyclopropyl), —O(CRₓRₓ)₀₋₂((ethoxycarbonyl)cyclopropyl), —O(CRₓRₓ)₀₋₂(oxetanyl), —O(CRₓRₓ)₀₋₂(methylazetidinyl), —O(CRₓRₓ)₀₋₂(tetrahydropyranyl), —O(CRₓRₓ)₁₋₂(morpholinyl), —O(CRₓRₓ)₀₋₂(thiazolyl), cyclopropyl, cyanocyclopropyl, methylazetidinyl, acetylazetidinyl, (tert-butoxycarbonyl)azetidinyl, triazolyl, tetrahydropyranyl, morpholinyl, thiophenyl, methylpiperidinyl, dioxolanyl, pyrrolidinonyl, and R_{d};
R_{4b} is —CH₃;
each R_{c} is independently H or C₁₋₂ alkyl;
R_{d} is phenyl substituted with zero to 1 substituent selected from F, Cl, —CN, —CH₃, and —OCH₃;
each R₅ is independently —CN, C₁₋₆ alkyl substituted with zero to 4 R_{g}, C₂₋₄ alkenyl substituted with zero to 4 R_{g}, C₂₋₄ alkynyl substituted with zero to 4 R_{g}, C₃₋₄ cycloalkyl substituted with zero to 4 R_{g}, phenyl substituted with zero to 4 R_{g}, oxadiazolyl substituted with zero to 3 R_{g}, pyridinyl substituted with zero to 4 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl);
each R_{g} is independently F, Cl, —CN, —OH, C₁₋₃ alkoxy, C₁₋₃ fluoroalkoxy, —O(CH₂)₁₋₂O(C₁₋₂ alkyl), or —NR_{c}R_{c};
each Rₓ is independently H or —CH₃; and
m is zero, 1, 2, or 3.

52. The compound according to claim 51 or a pharmaceutically acceptable salt thereof, wherein:
R₂ is H or C₁₋₂ alkyl substituted with zero to 2 R₂ₐ;
each R₂ₐ is independently F, Cl, —CN, —OH, —O(C₁₋₂ alkyl), cyclopropyl, C₃₋₄ alkenyl, or C₃₋₄ alkynyl;
R₄ₐ is C₃₋₆ cycloalkyl, 4- to 10-membered heterocyclyl, phenyl, or 5- to 10-membered heteroaryl, each substituted with zero to 4 substituents independently selected from F, Cl, Br, —CN, —OH, C₁₋₆ alkyl, C₁₋₃ fluoroalkyl, C₁₋₂ bromoalkyl, C₁₋₂ cyanoalkyl, C₁₋₂ hydroxyalkyl, —CH₂NRₐRₐ, —(CH₂)₁₋₂O(C₁₋₂ alkyl), —(CH₂)₁₋₂NRₓC(O)O(C₁₋₂ alkyl), C₁₋₄ alkoxy, —O(C₁₋₄ hydroxyalkyl), —O(CRₓRₓ)₁₋₂O(C₁₋₂ alkyl), C₁₋₃ fluoroalkoxy, C₁₋₃ cyanoalkoxy, —O(CH₂)₁₋₂NR_{c}R_{c}, —OCH₂CH═CH₂, —OCH₂C≡CH, —C(O)(C₁₋₄ alkyl), —C(O)OH, —C(O)O(C₁₋₄ alkyl), —NR_{c}R_{c}, —NRₐS(O)₂(C₁₋₃ alkyl), —NRₐC(O)(C₁₋₃ alkyl), —NRₐC(O)O(C₁₋₄ alkyl), —P(O)(C₁₋₂ alkyl)₂, —S(O)₂(C₁₋₃ alkyl), —(CH₂)₁₋₂(C₃₋₄ cycloalkyl), —CRₓRₓ(morpholinyl), —CRₓRₓ(difluoromorpholinyl), —CRₓRₓ(dimethylmorpholinyl), —CRₓRₓ(oxaazabicyclo[2.2.1]heptanyl), —CRₓRₓ(oxaazaspiro[3.3]heptanyl), —CRₓRₓ(methylpiperazinonyl), —CRₓRₓ(acetylpiperazinyl), —CRₓRₓ(piperidinyl), —CRₓRₓ(difluoropiperidinyl), —CRₓRₓ(methoxypiperidinyl), —CRₓRₓ(hydroxypiperidinyl), —O(CH₂)₀₋₂(C₃₋₄ cycloalkyl), —O(CH₂)₀₋₂(methylcyclopropyl), —O(CH₂)₀₋₂((ethoxycarbonyl)cyclopropyl), —O(CH₂)₀₋₂(oxetanyl), —O(CH₂)₀₋₂(methylazetidinyl), —O(CH₂)₁₋₂(morpholinyl), —O(CH₂)₀₋₂(tetrahydropyranyl), —O(CH₂)₀₋₂(thiazolyl), cyclopropyl, cyanocyclopropyl, methylazetidinyl, acetylazetidinyl, (tert-butoxycarbonyl)azetidinyl, dioxolanyl, pyrrolidinonyl, triazolyl, tetrahydropyranyl, morpholinyl, thiophenyl, methylpiperidinyl, and R_{d}; or
each R₅ is independently —CN, C₁₋₅ alkyl substituted with zero to 4 R_{g}, C₂₋₃ alkenyl substituted with zero to 4 R_{g}, C₂₋₃ alkynyl substituted with zero to 4 R_{g}, C₃₋₄ cycloalkyl substituted with zero to 4 R_{g}, phenyl substituted with zero to 3 R_{g}, oxadiazolyl substituted with zero to 3 R_{g}, pyridinyl substituted with zero to 3 R_{g}, —(CH₂)₁₋₂ (4- to 10-membered heterocyclyl substituted with zero to 4 R_{g}), —(CH₂)₁₋₂NR_{c}C(O)(C₁₋₄ alkyl), —(CH₂)₁₋₂NR_{c}C(O)O(C₁₋₄ alkyl), —(CH₂)₁₋₂NR_{c}S(O)₂(C₁₋₄ alkyl), —C(O)(C₁₋₄ alkyl), —C(O)OH, —C(O)O(C₁₋₄ alkyl), —C(O)O(C₃₋₄ cycloalkyl), —C(O)NRₐRₐ, or —C(O)NRₐ(C₃₋₄ cycloalkyl); and
m is 1, 2, or 3.

53. The compound according to claim 51 or a pharmaceutically acceptable salt thereof, wherein:
R₂ is —CH₃ or —CD₃;
R₄ₐ is cyclopropyl, cyclobutyl, cyclohexyl, phenyl, pyridinyl, pyrimidinyl, oxadiazolyl, bicyclo[1.1.1]pentanyl, benzo[d][1,3]dioxolyl, or oxodihydrobenzo[d]oxazolyl, each substituted with zero to 3 substituents independently selected from F, Cl, Br, —CN, —CH₃, —CH(CH₃)₂, —C(CH₃)₃, —CH₂OH, —CHF₂, —CF₃, —CH₂Br, —CH₂NH₂, —CH₂NHC(O)OCH₃, —C(CH₃)₂CN, —OCH₃, —OCD₃, —OCH₂CH₃, —OCH(CH₃)₂, —OCHF₂, —OCF₃, —OCH₂CH₂CF₃, —OC(CH₃)₂CN, —OC(CH₃)₂CH₂OH, —OC(CH₃)₂CH₂OCH₃, —N(CH₃)₂, —C(O)OCH₃, cyclopropyl, cyanocyclopropyl, methylcyclopropyl, —O(cyclopropyl), —O((ethoxycarbonyl)cyclopropyl), morpholinyl, pyrrolidinonyl, tetrahydropyranyl, dioxolanyl, —CH₂(morpholinyl), —CH₂(difluoromorpholinyl), —CH₂(dimethylmorpholinyl), —CH₂(oxaazabicyclo[2.2.1]heptanyl), —CH₂(oxaazaspiro[3.3]heptanyl), —CH₂(methylpiperazinonyl), —CH₂(acetylpiperazinyl), —CH₂(piperidinyl), —CH₂(difluoropiperidinyl), —CH₂(methoxypiperidinyl), —CH₂(hydroxypiperidinyl), —C(CH₃)₂(morpholinyl), —OCH₂(cyclopropyl), —OCH₂(methylcyclopropyl), —OCH₂(methylazetidinyl), —OCH₂(oxetanyl), —OCH₂(tetrahydropyranyl), —OCH₂(thiazolyl), and —OCH₂CH₂(cyclopropyl);
each R₅ is independently —CH₃, —CH₂CH₃, —CH₂CH₂CH₃, —CH₂OH, —CH₂OCH₃, —CH₂OCH₂CH₃, —CH₂NH₂, —CH₂N₃, or —CH₂NHC(O)OCH₃; and
m is 2.

54. The compound according to claim 51 or a pharmaceutically acceptable salt thereof, wherein R₄ₐ is phenyl, pyridinyl, pyrimidinyl, oxadiazolyl, benzo[d][1,3]dioxolyl, or oxodihydrobenzo[d]oxazolyl, each substituted with 1 to 3 substituents independently selected from F, Cl, Br, —CN, —CH₃, —CH₂OH, —CH₂Br, —CH₂NH₂, —CH₂NHC(O)OCH₃, —CF₃, —C(CH₃)₂CN, —OCH₃, —OCD₃, —OCH₂CH₃, —OCH(CH₃)₂, —OCHF₂, —OCF₃, —OCH₂CH₂CF₃, —OC(CH₃)₂CN, —OC(CH₃)₂CH₂OH, —OC(CH₃)₂CH₂OCH₃, —N(CH₃)₂, —C(O)OCH₃, cyclopropyl, cyanocyclopropyl, methylcyclopropyl, —O(cyclopropyl), —O((ethoxycarbonyl)cyclopropyl), —OCH₂(cyclopropyl), —CH₂(piperidinyl), morpholinyl, pyrrolidinonyl, tetrahydropyranyl, dioxolanyl, —CH₂(morpholinyl), —CH₂(difluoromorpholinyl), —CH₂(dimethylmorpholinyl), —CH₂(oxaazabicyclo[2.2.1]heptanyl), —CH₂(oxaazaspiro[3.3]heptanyl), —CH₂(methylpiperazinonyl), —CH₂(acetylpiperazinyl), —CH₂(piperidinyl), —CH₂(difluoropiperidinyl), —CH₂(methoxypiperidinyl), —CH₂(hydroxypiperidinyl), —C(CH₃)₂(morpholinyl), —OCH₂(cyclopropyl), —OCH₂(methylcyclopropyl), —OCH₂(methylazetidinyl), —OCH₂(oxetanyl), —OCH₂(tetrahydropyranyl), —OCH₂(thiazolyl), and —OCH₂CH₂(cyclopropyl).

55. The compound according to claim 51 or a pharmaceutically acceptable salt thereof, wherein R₄ₐ is phenyl, pyridinyl, or oxadiazolyl, each substituted zero to 3 substituents independently selected from with F, Cl, Br, —CN, —CH₃, —C(CH₃)₃, —CHF₂, —CF₃, —OCH₃, —OCH(CH₃)₂, —OCHF₂, —OCF₃, —OCH₂(cyclopropyl), and cyclopropyl.

56. The compound according to claim 51 or a pharmaceutically acceptable salt thereof, having the structure: 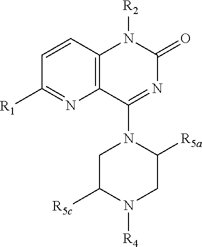
wherein:
R₅ₐ is —CH₃ or —CH₂CH₃; and
R_{5c} is —CH₃, —CH₂CH₃, or —CH₂CH₂CH₃.

57. The compound according to claim 51 or a pharmaceutically acceptable salt thereof, having the structure:

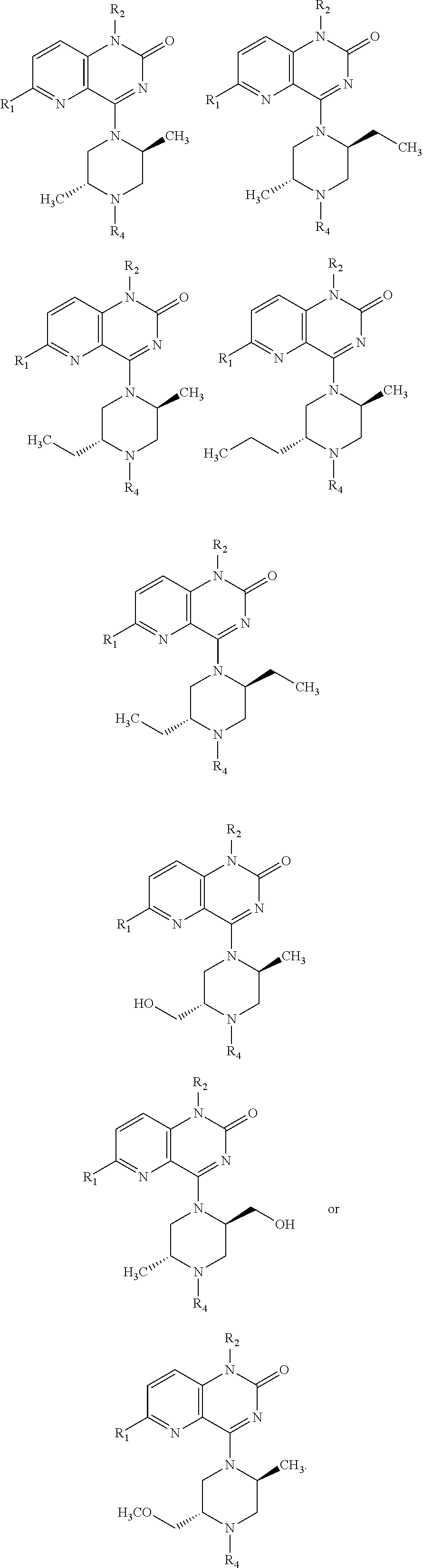

58. The compound according to claim 51 or a salt thereof, wherein said compound is:
R₂ is —CH₃;
R₄ₐ is phenyl substituted with —CF₃;
R_{4b} is —CH₃ or —CH₂CH₃;
each R₅ is —CH₂CH₃; and
m is 2.

59. The compound according to claim 56 or a salt thereof, wherein said compound is:
R₂ is —CH₃;
R₄ₐ is phenyl substituted with —CF₃;
R_{4b} is —CH₃ or —CH₂CH₃;
R₅ₐ is —CH₂CH₃; and
R_{5b} is —CH₂CH₃.

60. The compound according to claim 51 or a salt thereof, wherein said compound is:
4-((2S,5R)-2,5-diethyl-4-(1-(4-(trifluoromethyl)phenyl)ethyl)piperazin-1-yl)-6-ethoxy-1-methylpyrido[3,2-d]pyrimidin-2(1H)-one; or
4-((2S,5R)-2,5-diethyl-4-(1-(4-(trifluoromethyl)phenyl)propyl)piperazin-1-yl)-6-ethoxy-1-methylpyrido[3,2-d]pyrimidin-2(1H)-one.

61. The compound according to claim 51 or a salt thereof, wherein said compound is:

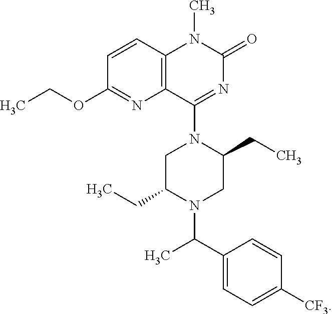 [SMILES: CCOc1ccc2c(n1)c(N1C[C@@H](CC)N(C(C)c3ccc(C(F)(F)F)cc3)C[C@@H]1CC)nc(=O)n2C]

62. The compound according to claim 51 or a salt thereof, wherein said compound is: 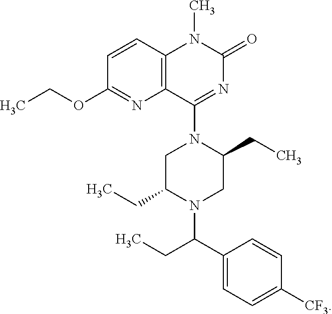 [SMILES: CCOc1ccc2c(n1)c(N1C[C@@H](CC)N(C(CC)c3ccc(C(F)(F)F)cc3)C[C@@H]1CC)nc(=O)n2C]
